(19) United States
(12) Patent Application Publication
(10) Pub. No.: US 2025/0231185 A1
(43) Pub. Date: Jul. 17, 2025

(54) Coronavirus T Cell Epitopes, Megapools and Uses Thereof

(71) Applicant: La Jolla Institute for Immunology, La Jolla, CA (US)

(72) Inventors: Alessandro Sette, La Jolla, CA (US); Shane Crotty, La Jolla, CA (US); Bjoern Peters, La Jolla, CA (US); John Sidney, La Jolla, CA (US); Alba Grifoni, La Jolla, CA (US); Daniela Weiskopf, La Jolla, CA (US); Ricardo Da Silva Antunes, La Jolla, CA (US)

(21) Appl. No.: 18/555,376

(22) PCT Filed: May 12, 2022
(86) PCT No.: PCT/US2022/028982

§ 371 Date: Oct. 23, 2023

Related U.S. Application Data
(60) Provisional application No. 63/188,220, filed on May 13, 2021.
(60) Provisional application No. 63/286,537, filed on Dec. 6, 2021.
(60) Provisional application No. 63/293,229, filed on Dec. 23, 2021.

(51) Int. Cl. G01N 33/569; C07K 14/005; C07K 14/74; G01N 33/50; G01N 33/68

(52) U.S. Cl. CPC G01N 33/56972; C07K 14/005; C07K 14/70539; G01N 33/505; G01N 33/56983; G01N 33/6863; G01N 33/6878; C07K 2319/00; C12N 2770/20022; G01N 2333/165; G01N 2333/70539; G01N 2333/715

(57) ABSTRACT

The present disclosure includes compositions and methods for detecting the presence of: a coronavirus or an immune response relevant to a coronavirus infection including T cells responsive to one or more coronavirus peptides or proteins comprising, consisting of, or consisting essentially of: one or more amino acid sequences selected from those sequences set forth in Tables 1 to 10 (SEQ ID NOS: 1 to 3522), or a subsequence, portion, homologue, variant or derivative thereof; a fusion protein; a pool of 2 or more peptides; or a polynucleotide that encodes one or more peptides or proteins, comprising, consisting of, or consisting essentially of one or more amino acid sequences set forth in Tables 1 to 10 (SEQ ID NOS: 1 to 3522), or a subsequence, portion, homologue, variant or derivative thereof. The disclosure further provides vaccines, diagnostics, therapies, and kits, comprising such proteins or peptides.

### CROSS-REFERENCE TO RELATED APPLICATIONS

This application is a National Stage of International Application No. PCT/US2022/028982, filed May 12, 2022, which claims the benefit of U.S. Provisional Application Ser. No. 63/188,220, filed May 13, 2021, 63/286,537, filed Dec. 7, 2021, and 63/293,229, filed Dec. 23, 2021. The contents of each of which are incorporated by reference in their entirety.

### STATEMENT OF FEDERALLY FUNDED RESEARCH

This invention was made with government support under grant and contract numbers U19 AI142742, 75N93019C00065, and 75N93019C00001 awarded by the National Institutes of Health/NIAID. The government has certain rights in the invention.

### TECHNICAL FIELD OF THE INVENTION

The present invention relates in general to the field of peptides that are T cell epitopes for coronavirus, and more particularly, to compositions and methods for the prevention, treatment, diagnosis, kits, and uses of such T cell epitopes.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jun. 23, 2022, is named LJII2014WO_ST25.txt and is 717,449 bytes in size.

### BACKGROUND OF THE INVENTION

Without limiting the scope of the invention, its background is described in connection with coronaviruses.

As of April 2022, SARS-CoV-2 infections are associated with more than 6.15 million deaths and over 491 million cases worldwide, and over 80 million cases in the United States alone (coronavirus.jhu.edu/map.html). The severity of the associated Coronavirus Disease 2019 (COVID-19) ranges from asymptomatic or mild self-limiting disease, to severe pneumonia and acute respiratory distress syndrome (WHO; www.who.int/publications/i/item/clinical-management-of-covid-19). The present inventors and others have started to delineate the role of SARS-CoV-2-specific T cell immunity in COVID-19 clinical outcomes (Altmann and Boyton, 2020; Braun et al., 2020; Grifoni et al., 2020; Le Bert et al., 2020; Meckiff et al., 2020; Rydyznski Moderbacher et al., 2020; Sekine et al., 2020; Weiskopf et al., 2020). A growing body of evidence points to a key role for SARS-CoV-2-specific T cell responses in COVID-19 disease resolution and modulation of disease severity (Rydyznski Moderbacher et al., 2020; Schub et al., 2020; Weiskopf et al., 2020). Milder cases of acute COVID-19 were associated with coordinated antibody, CD4+ and CD8+ T cell responses, whereas severe cases correlated with a lack of coordination of cellular and antibody responses, and delayed kinetics of adaptive responses (Rydyznski Moderbacher et al., 2020; Weiskopf et al., 2020). Now, the emergence of SARS-CoV-2 variants highlights the better need to better understand adaptive immune responses to this virus.

Despite these advances, and in light of variants of SARS-CoV-2 being identified globally, a need remains for identifying T cell epitopes for use in diagnostics, treatments, vaccines, kits, etc.

### SUMMARY OF THE INVENTION

As embodied and broadly described herein, an aspect of the present disclosure relates to a composition comprising: one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof, a fusion protein comprising one or more amino acid sequences selected from those sequences set forth in Tables 1 to 10 (SEQ ID NOS: 1 to 3522); or a pool of 2 or more peptides comprising, consisting of, or consisting essentially of amino acid sequences selected from those sequences set forth in Tables 1 to 10 (SEQ ID NOS: 1 to 3522); or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof. In one aspect, the one or more peptides or proteins comprises, or wherein the fusion protein comprises 2 or more or more amino acid sequences selected from those sequences set forth in Tables 1 to 10 (SEQ ID NOS: 1 to 3522), or a subsequence, portion, homologue, variant or derivative thereof. In another aspect, the amino acid sequence is selected from a coronavirus T cell epitope selected from those sequences set forth in Tables 1, 2, 3, 4, 5, 6, 7, 8, 9, or 10 (SEQ ID NOS: 1 to 3522). In another aspect, the composition comprises one or more SARS-CoV-2 peptides amino acid sequences selected from those sequences set forth in Tables 1 to 10 (SEQ ID NOS: 1 to 3522), or a subsequence, portion, homologue, variant or derivative thereof, a fusion protein comprising one or more amino acid sequences selected from those sequences set forth in Tables 1 to 10 (SEQ ID NOS: 1 to 3522); or a pool of 2 or more peptides selected from those sequences set forth in Tables 1 to 10 (SEQ ID NOS: 1 to 3522); or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof. In another aspect, the peptide or protein comprises a coronavirus T cell epitope. In another aspect, the wherein the one or more peptides or proteins comprises a coronavirus CD8+ or CD4+ T cell epitope. In another aspect, the coronavirus is SARS-CoV-2 and the SARS-CoV-2 T cell epitope is not conserved in another coronavirus. In another aspect, the coronavirus is SARS-CoV-2 and the SARS-CoV-2 T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to a coronavirus. In another aspect, the one or more peptides or proteins that elicits, stimulates, induces, promotes, increases or enhances the T cell response to the coronavirus is a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

As embodied and broadly described herein, an aspect of the present disclosure relates to a composition comprising monomers or multimers of: peptides or proteins comprising, consisting of, or consisting essentially of: one or more amino acid sequences selected from those sequences set forth in Tables 1 to 10 (SEQ ID NOS: 1 to 3522), concatemers, subsequences, portions, homologues, variants or derivatives thereof; a fusion protein comprising one or more amino acid sequences selected from those sequences set forth in Tables 1 to 10 (SEQ ID NOS: 1 to 3522); or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof.

As embodied and broadly described herein, an aspect of the present disclosure relates to a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 to 10 (SEQ ID NOS: 1 to 3522), in a groove of the MHC monomer or multimer.

As embodied and broadly described herein, an aspect of the present disclosure relates to a composition comprising: one or more peptides or proteins comprising, consisting of, or consisting essentially of an amino acid sequence selected from those sequences set forth in Tables 1 to 10 (SEQ ID NOS: 1 to 3522), or a subsequence, portion, homologue, variant or derivative thereof, a fusion protein comprising one or more amino acid sequences selected from those sequences set forth in Tables 1 to 10 (SEQ ID NOS: 1 to 3522); a pool of 2 or more peptides selected from those sequences set forth in Tables 1 to 10 (SEQ ID NOS: 1 to 3522); or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof. In one aspect, the one or more peptides or proteins comprises, or wherein the fusion protein comprises, 2 or more amino acid sequences selected from those sequences set forth in Tables 1 to 10 (SEQ ID NOS: 1 to 3522), or a subsequence, portion, homologue, variant or derivative thereof. In another aspect, the protein or peptide comprises a SARS-CoV-2 T cell epitope. In another aspect, the one or more peptides or proteins comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to SARS-CoV-2. In another aspect, the one or more peptides or proteins that elicits, stimulates, induces, promotes, increases or enhances the T cell response to SARS-CoV-2 is a SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, AS04, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-g), Transforming growth factor beta (TGF-B), or Interleukin-10 (IL-10), or an agonist or antagonist thereof. In another aspect, the one or more peptides or proteins include the amino acid sequences selected from Tables 1 to 10 (SEQ ID NOS: 1 to 3522).

As embodied and broadly described herein, an aspect of the present disclosure relates to a composition comprising monomers or multimers of: one or more peptides or proteins comprising, consisting of, or consisting essentially of: one or more SARS-CoV-2 amino acid sequences selected from those sequences set forth in Tables 1 to 10 (SEQ ID NOS: 1 to 3522), concatemers, subsequences, portions, homologues, variants or derivatives thereof, a fusion protein comprising one or more amino acid sequences selected from those sequences set forth in Tables 1 to 10 (SEQ ID NOS: 1 to 3522); or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof.

As embodied and broadly described herein, an aspect of the present disclosure relates to a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 to 10 (SEQ ID NOS: 1 to 3522), in a groove of the (MHC) monomer or multimer. In one aspect, the compositions include those amino acid sequences selected from Tables 1, 2, 3, 4, 5, 6, 7, 8, 9, or 10 (SEQ ID NOS: 1 to 3522).

As embodied and broadly described herein, an aspect of the present disclosure relates to a method for detecting the presence of: (i) a coronavirus or (ii) an immune response relevant to coronavirus infections, vaccines or therapies, including T cells responsive to one or more coronavirus peptides, comprising: providing one or more proteins or peptides for detection of an amount or a relative amount of, and/or the activity of, and/or the state of antigen-specific T-cells; contacting a biological sample suspected of having coronavirus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in Tables 1 to 10 (SEQ ID NOS: 1 to 3522), or comprise a pool of 2 or more amino acid sequences set forth in Tables 1 to 10 (SEQ ID NOS: 1 to 3522). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from Tables 1 to 10 (SEQ ID NOS: 1 to 3522). In another aspect, detecting the amount or a relative amount of, and/or activity of antigen-specific T-cells comprises indirect detection and/or direct detection. In another aspect, the method of detecting an immune response relevant to the coronavirus comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, MHC monomer or MHC multimer comprises a protein or peptide of the coronavirus. In another aspect, protein or peptide comprises a CD8+ or CD4+ T cell epitope. In another aspect, T cell epitope is not conserved in another coronavirus. In another aspect, T cell epitope is conserved in another coronavirus. In another aspect, protein or peptide has a length from about 9-15, 15-20, 20-25, 25-30, 30-40, 40-50, 50-75 or 75-100 amino acids. In another aspect, proteins or peptides comprise 2 or more amino acid sequences selected from those sequences set forth in Tables 1, 2, 3, 4, 5, 6, 7, 8, 9, or 10 (SEQ ID NOS: 1 to 3522), or a subsequence, portion, homologue, variant or derivative thereof. In another aspect, the method further comprises detecting the presence or amount of the one or more peptides in a biological sample, or a response thereto, which is diagnostic of a coronavirus infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising any of the compositions described hereinabove to the subject from which the biological sample was drawn that increases the amount or relative amount of, and/or activity of the antigen-specific T-cells.

As embodied and broadly described herein, an aspect of the present disclosure relates to a method for detecting the presence of: (i) SARS-CoV-2 or (ii) an immune response relevant to SARS-CoV-2 infections, vaccines or therapies, including T cells responsive to one or more SARS-CoV-2 peptides, comprising: providing one or more proteins or peptides for detection of an amount or a relative amount of, and/or the activity of, and/or the state of antigen-specific T-cells; contacting a biological sample suspected of having SARS-CoV-2-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in Tables 1 to 10 (SEQ ID NOS: 1 to 3522), or comprise a pool of 2 or more amino acid sequences set forth in those sequences set forth in Tables 1 to 10 (SEQ ID NOS: 1 to 3522). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from those sequences set forth in Tables 1, 2, 3, 4, 5, 6, 7, 8, 9 or 10 (SEQ ID NOS: 1 to 3522). In another aspect, the detecting the amount or a relative amount of, and/or activity of antigen-specific T-cells comprises indirect detection and/or direct detection. In another aspect, the method of detecting an immune response relevant to SARS-CoV-2 comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of SARS-CoV-2. In another aspect, the protein or peptide comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Tables 1, 2, 3, 4, 5, 6, 7, 8, 9, or 10 (SEQ ID NOS: 1 to 3522), or a subsequence, portion, homologue, variant or derivative thereof. In another aspect, the method further comprises detecting the presence or amount of the one or more peptides in a biological sample, or a response thereto, which is diagnostic of a SARS-CoV-2 infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition describe hereinabove to the subject from which the biological sample was drawn that increases the amount or relative amount of, and/or activity of the antigen-specific T-cells.

As embodied and broadly described herein, an aspect of the present disclosure relates to a method detecting a coronavirus infection or exposure in a subject, the method comprising, consisting of, or consisting essentially of: contacting a biological sample from a subject with a composition described hereinabove; and determining if the composition elicits an immune response from the contacted cells, wherein the presence of an immune response indicates that the subject has been exposed to or infected with coronavirus. In one aspect, the sample comprises T cells. In another aspect, the response comprises inducing, increasing, promoting or stimulating anti-coronavirus activity of T cells. In another aspect, the T cells are CD8+ or CD4+ T cells. In another aspect, the method comprises determining whether the subject has been infected by or exposed to the coronavirus more than once by determining if the subject elicits a secondary T cell immune response profile that is different from a primary T cell immune response profile. In another aspect, the method further comprises diagnosing a coronavirus infection or exposure in a subject, the method comprising contacting a biological sample from a subject with a composition described hereinabove, and determining if the composition elicits a T cell immune response, wherein the T cell immune response identifies that the subject has been infected with or exposed to a coronavirus. In another aspect, the method is conducted three or more days following the date of suspected infection by or exposure to a coronavirus.

As embodied and broadly described herein, an aspect of the present disclosure relates to a method detecting SARS-CoV-2 infection or exposure in a subject, the method comprising, consisting of, or consisting essentially of: contacting a biological sample from a subject with a composition describe hereinabove; and determining if the composition elicits an immune response from the contacted cells, wherein the presence of an immune response indicates that the subject has been exposed to or infected with SARS-CoV-2. In one aspect, the sample comprises T cells. In another aspect, the response comprises inducing, increasing, promoting or stimulating anti-SARS-CoV-2 activity of T cells. In another aspect, the T cells are CD8+ or CD4+ T cells. In another aspect, the method comprises determining whether the subject has been infected by or exposed to SARS-CoV-2 more than once by determining if the subject elicits a secondary T cell immune response profile that is different from a primary T cell immune response profile. In another aspect, the method further comprises diagnosing a SARS-CoV-2 infection or exposure in a subject, the method comprising contacting a biological sample from a subject with a composition described hereinabove; and determining if the composition elicits a T cell immune response, wherein the T cell immune response identifies that the subject has been infected with or exposed to SARS-CoV-2. In another aspect, the method is conducted three or more days following the date of suspected infection by or exposure to a coronavirus.

29. A kit for the detection of coronavirus or an immune response to coronavirus in a subject comprising, consisting of or consisting essentially of: one or more T cells that specifically detect the presence of: one or more amino acid sequences selected from those sequences set forth in Tables 1 to 10 (SEQ ID NOS: 1 to 3522), or a subsequence, portion, homologue, variant or derivative thereof, or a fusion protein comprising one or more amino acid sequences selected from those sequences set forth in Tables 1 to 10 (SEQ ID NOS: 1 to 3522); or a pool of 2 or more peptides selected from the amino acid sequences set forth in Tables 1 to 10 (SEQ ID NOS: 1 to 3522). In one aspect, the one or more amino acid sequences are selected from a coronavirus T cell epitope set forth in Tables 1 to 10 (SEQ ID NOS: 1 to 3522). In another aspect, the composition comprises: one or more amino acid sequences selected from those sequences set forth in Tables 1 to 10 (SEQ ID NOS: 1 to 3522), or a subsequence, portion, homologue, variant or derivative thereof, fusion protein comprising one or more amino acid sequences selected from those sequences set forth in Tables 1 to 10 (SEQ ID NOS: 1 to 3522); or a pool of 2 or more peptides selected from the amino acid sequences set forth in those sequences set forth in Tables 1 to 10 (SEQ ID NOS: 1 to 3522). In another aspect, the amino acid sequence comprises a coronavirus CD8+ or CD4+ T cell epitope. In another aspect, the T cell epitope is not conserved in another coronavirus. In another aspect, the T cell epitope is conserved in another coronavirus. In another aspect, the fusion protein has a length from about 9-15, 15-20, 20-25, 25-30, 30-40, 40-50, 50-75 or 75-100 amino acids. In another aspect, the part thereof for the detection of: (i) coronavirus or (ii) an immune response relevant to coronavirus infections, vaccines or therapies, including T cells responsive to coronavirus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to coronavirus.

As embodied and broadly described herein, an aspect of the present disclosure relates to a kit for the detection of SARS-CoV-2 or an immune response to SARS-CoV-2 in a subject comprising, consisting of or consisting essentially of: one or more T cells that specifically detect the presence of: one or more amino acid sequences selected from those sequences set forth in Tables 1 to 10 (SEQ ID NOS: 1 to 3522), or a subsequence, portion, homologue, variant or derivative thereof, a fusion protein comprising one or more amino acid sequences selected from those sequences set forth in Tables 1 to 10 (SEQ ID NOS: 1 to 3522); or a pool of 2 or more peptides selected from the amino acid sequences set forth in those sequences set forth in Tables 1 to 10 (SEQ ID NOS: 1 to 3522). In one aspect, the one or more amino acid sequences is selected from a SARS-CoV-2 CD4 T cell epitope selected from Tables 1 to 10 (SEQ ID NOS: 1 to 3522). In another aspect, the one or more amino acid sequences include amino acid sequences selected from Tables 1 to 10 (SEQ ID NOS: 1 to 3522). In another aspect, the amino acid sequence comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) SARS-CoV-2 or (ii) an immune response relevant to SARS-CoV-2 infections, vaccines or therapies, including T cells responsive to SARS-CoV-2. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to SARS-CoV-2.

As embodied and broadly described herein, an aspect of the present disclosure relates to a method of stimulating, inducing, promoting, increasing, or enhancing an immune response against a coronavirus in a subject, comprising: administering a composition describe hereinabove, in an amount sufficient to stimulate, induce, promote, increase, or enhance an immune response against the coronavirus in the subject. In one aspect, the immune response provides the subject with protection against a coronavirus infection or pathology, or one or more physiological conditions, disorders, illnesses, diseases or symptoms caused by or associated with coronavirus infection or pathology. In another aspect, the acid sequences set forth in Tables 1 to 10 (SEQ ID NOS: 1 to 3522), or a subsequence, portion, homologue, variant or derivative thereof.

As embodied and broadly described herein, an aspect of the present disclosure relates to a method of stimulating, inducing, promoting, increasing, or enhancing an immune response against SARS-CoV-2 in a subject, comprising: administering a composition described hereinabove, in an amount sufficient to stimulate, induce, promote, increase, or enhance an immune response against SARS-CoV-2 in the subject. In one aspect, the immune response provides the subject with protection against a SARS-CoV-2 infection or pathology, or one or more physiological conditions, disorders, illnesses, diseases or symptoms caused by or associated with SARS-CoV-2 infection or pathology. In another aspect, the immune response is specific to: one or more SARS-CoV-2 peptides selected from the amino acid sequences set forth in those sequences set forth in Tables 1 to 10 (SEQ ID NOS: 1 to 3522), or a subsequence, portion, homologue, variant or derivative thereof. In another aspect, the one or more SARS-CoV-2 peptides selected from the amino acid sequences set forth in those sequences set forth in Tables 1, 2, 3, 4, 5, 6, 7, 8, 9, or 10 (SEQ ID NOS: 1 to 3522), or a subsequence, portion, homologue, variant or derivative thereof, include the amino acid sequences selected from Tables 1, 2, 3, 4, 5, 6, 7, 8, 9, or 10 (SEQ ID NOS: 1 to 3522).

As embodied and broadly described herein, an aspect of the present disclosure relates to a method of stimulating, inducing, promoting, increasing, or enhancing an immune response against SARS-CoV-2 in a subject, comprising: administering to a subject an amount of a protein or peptide or a polynucleotide that expresses the protein or peptide comprising, consisting of or consisting essentially of an amino acid sequence of the SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in Tables 1 to 10 (SEQ ID NOS: 1 to 3522), or both or a subsequence, portion, homologue, variant or derivative thereof, in an amount sufficient to prevent, stimulate, induce, promote, increase, immunize against, or enhance an immune response against SARS-CoV-2 in the subject. In one aspect, the immune response provides the subject with protection against SARS-CoV-2 infection or pathology, or one or more physiological conditions, disorders, illnesses, diseases or symptoms caused by or associated with SARS-CoV-2 infection or pathology.

As embodied and broadly described herein, an aspect of the present disclosure relates to a method of treating, preventing, or immunizing a subject against SARS-CoV-2 infection, comprising administering to a subject an amount of a protein, peptide or a polynucleotide that expresses the protein or peptide comprising, consisting of, or consisting essentially of an amino acid sequence of a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two amino acid sequences selected from Tables 1 to 10 (SEQ ID NOS: 1 to 3522), or both, or a subsequence, portion, homologue, variant or derivative thereof, in an amount sufficient to treat, prevent, or immunize the subject for SARS-CoV-2 infection, wherein the protein or peptide comprises or consists of a coronavirus T cell epitope that elicits, stimulates, induces, promotes, increases, or enhances an anti-SARS-CoV-2 T cell immune response. In one aspect, the one or more amino acid sequences are selected from those sequences set forth in Tables 1 to 10 (SEQ ID NOS: 1 to 3522), or a subsequence, portion, homologue, variant or derivative thereof; a fusion protein comprising one or more amino acid sequences selected from those sequences set forth in Tables 1 to 10 (SEQ ID NOS: 1 to 3522); or a pool of 2 or more peptides selected from the amino acid sequences set forth in those sequences set forth in Tables 1 to 10 (SEQ ID NOS: 1 to 3522). In one aspect, the anti-SARS-CoV-2 T cell response is a CD8+, a CD4+ T cell response, or both. In another aspect, the T cell epitope is conserved across two or more clinical isolates of SARS-CoV-2, two or more circulating forms of SARS-CoV-2, or two or more coronaviruses. In another aspect, the SARS-CoV-2 infection is an acute infection. In another aspect, the subject is a mammal or a human. In another aspect, the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid. In another aspect, the method reduces one or more adverse physiological conditions, disorders, illness, diseases, symptoms or complications caused by or associated with SARS-CoV-2 infection or pathology. In another aspect, the method improves one or more adverse physiological conditions, disorders, illness, diseases, symptoms or complications caused by or associated with SARS-CoV-2 infection or pathology. In another aspect, the symptom is fever or chills, cough, shortness of breath or difficulty breathing, fatigue, muscle or body aches, headache, new loss of taste or smell, sore throat, congestion or runny nose, nausea or vomiting, or diarrhea. In another aspect, the method reduces or inhibits susceptibility to SARS-CoV-2 infection or pathology. In another aspect, the protein or peptide, or a subsequence, portion, homologue, variant or derivative thereof, is administered prior to, substantially contemporaneously with or following exposure to or infection of the subject with SARS-CoV-2. In another aspect, a plurality of SARS-CoV-2 T cell epitopes are administered prior to, substantially contemporaneously with or following exposure to or infection of the subject with SARS-CoV-2. In another aspect, the protein or peptide, or a subsequence, portion, homologue, variant or derivative thereof is administered within 2-72 hours, 2-48 hours, 4-24 hours, 4-18 hours, or 6-12 hours after a symptom of SARS-CoV-2 infection or exposure develops. In another aspect, the protein or peptide, or a subsequence, portion, homologue, variant or derivative thereof is administered prior to exposure to or infection of the subject with SARS-CoV-2. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-β, or IL-10, or an agonist or antagonist thereof. In another aspect, the one or amino acid sequences include amino acid sequences selected from Tables 1 to 10 (SEQ ID NOS: 1 to 3522).

As embodied and broadly described herein, an aspect of the present disclosure relates to a method of treating, preventing, or immunizing a subject against SARS-CoV-2 infection, comprising administering to a subject the composition described hereinabove in an amount sufficient to treat, prevent, or immunize the subject for SARS-CoV-2 infection. In one aspect, the SARS-CoV-2 infection is an acute infection. In another aspect, the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid. In another aspect, the method reduces one or more adverse physiological conditions, disorders, illness, diseases, symptoms or complications caused by or associated with SARS-CoV-2 infection or pathology. In another aspect, the method improves one or more adverse physiological conditions, disorders, illness, diseases, symptoms or complications caused by or associated with SARS-CoV-2 infection or pathology. In another aspect, the symptom is fever or chills, cough, shortness of breath or difficulty breathing, fatigue, muscle or body aches, headache, new loss of taste or smell, sore throat, congestion or runny nose, nausea, vomiting, or diarrhea. In another aspect, the method reduces or inhibits susceptibility to SARS-CoV-2 infection or pathology. In another aspect, the composition is administered prior to, substantially contemporaneously with or following exposure to or infection of the subject with SARS-CoV-2. In another aspect, the composition is administered prior to, substantially contemporaneously with or following exposure to or infection of the subject with SARS-CoV-2. In another aspect, the composition is administered within 2-72 hours, 2-48 hours, 4-24 hours, 4-18 hours, or 6-12 hours after a symptom of SARS-CoV-2 infection or exposure develops. In another aspect, the composition is administered prior to exposure to or infection of the subject with SARS-CoV-2.

As embodied and broadly described herein, an aspect of the present disclosure relates to a peptide or peptides that are immunoprevalent or immunodominant in a virus obtained by a method consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-CoV-2. In another aspect, the immunodominant peptides are selected from 1, 2 or more peptides selected from the amino acid sequences set forth in Tables 1 to 10 (SEQ ID NOS: 1 to 3522). In another aspect, the immunodominant peptides are selected from 1, 2 or more peptides selected from the amino acid sequences set forth in those sequences set forth in Tables 1 to 10 (SEQ ID NOS: 1 to 3522). In another aspect, the peptide or peptides include amino acid sequences set forth in Tables 1 to 10 (SEQ ID NOS: 1 to 3522).

As embodied and broadly described herein, an aspect of the present disclosure relates to a method of selecting an immunoprevalent or immunodominant peptide or protein of a virus comprising, consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-CoV-2. In another aspect, the immunodominant peptides are selected from 1, 2 or more peptides selected from the amino acid sequences set forth in Tables 1 to 10 (SEQ ID NOS: 1 to 3522). In another aspect, the immunodominant peptides are selected from 1, 2 or more peptides selected from the amino acid sequences set forth in those sequences set forth in Tables 1 to 10 (SEQ ID NOS: 1 to 3522). In another aspect, the peptide or peptides include amino acid sequences set forth in Tables 1 to 10 (SEQ ID NOS: 1 to 3522).

As embodied and broadly described herein, an aspect of the present disclosure relates to a polynucleotide that expresses one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof, a fusion protein comprising one or more amino acid sequences selected from those sequences set forth in Tables 1 to 10 (SEQ ID NOS: 1 to 3522); or a pool of 2 or more peptides comprising, consisting of, or consisting essentially of amino acid sequences selected from those sequences set forth in Tables 1 to 10 (SEQ ID NOS: 1 to 3522).

As embodied and broadly described herein, an aspect of the present disclosure relates to a vector that comprises the polynucleotide hereinabove. In one aspect, the vector is a viral vector.

As embodied and broadly described herein, an aspect of the present disclosure relates to a host cell that comprises the vector hereinabove.

As embodied and broadly described herein, an aspect of the present disclosure relates to a polynucleotide that expresses: one or more peptides or proteins comprising, consisting of, or consisting essentially of an amino acid sequence selected from those sequences set forth in Tables 1 to 10 (SEQ ID NOS: 1 to 3522), or a subsequence, portion, homologue, variant or derivative thereof, a fusion protein comprising one or more amino acid sequences selected from those sequences set forth in Tables 1 to 10 (SEQ ID NOS: 1 to 3522); or a pool of 2 or more peptides selected from those sequences set forth in Tables 1 to 10 (SEQ ID NOS: 1 to 3522).

As embodied and broadly described herein, an aspect of the present disclosure relates to a vector that comprises the polynucleotide hereinabove. In one aspect, the vector is a viral vector.

As embodied and broadly described herein, an aspect of the present disclosure relates to a host cell that comprises the vector hereinabove.

As embodied and broadly described herein, an aspect of the present disclosure relates to a peptide-major histocompatibility complex (MHC)/peptide multimer comprising at least two MHC/peptide monomers, wherein at least one MHC/peptide monomer comprises a peptide derived from SARS-CoV-2.

As embodied and broadly described herein, an aspect of the present disclosure relates to a peptide-major histocompatibility complex (MHC)/peptide multimer comprising at least two MHC/peptide monomers, wherein at least one MHC/peptide monomer comprises a peptide that comprises, consists of, or consists essentially of an amino acid sequence selected from the sequences set forth in Tables 1 to 10 (SEQ ID NOS: 1 to 3522). In one aspect, at least one MHC/peptide monomer comprises a peptide derived from SARS-CoV-2 Spike (S) protein such as a SARS-CoV-2 Spike (S) protein derived peptide set forth in Tables 1 to 10 (SEQ ID NOS: 1 to 3522). In one aspect, at least one MHC/peptide monomer comprises a peptide derived from SARS-CoV-2 Membrane (M) protein such as a SARS-CoV-2 Membrane (M) protein derived peptide set forth in Tables 1 to 10 (SEQ ID NOS: 1 to 3522). In one aspect, at least one MHC/peptide monomer comprises a peptide derived from SARS-CoV-2 Nucleocapsid (N) protein such as a SARS-CoV-2 Nucleocapsid (N) protein derived peptide set forth in Tables 1 to 10 (SEQ ID NOS: 1 to 3522). In one aspect, at least one MHC/peptide monomer comprises a peptide derived from SARS-CoV-2 Envelope (E) protein such as a SARS-CoV-2 Envelope (E) protein derived peptide set forth in Tables 1 to 10 (SEQ ID NOS: 1 to 3522). In one aspect, at least one MHC/peptide monomer comprises a peptide derived from SARS-CoV-2 ORF3a protein such as a SARS-CoV-2 ORF3a protein derived peptide set forth in Tables 1 to 10 (SEQ ID NOS: 1 to 3522). In one aspect, at least one MHC/peptide monomer comprises a peptide derived from SARS-CoV-2 ORF6 protein such as a SARS-CoV-2 ORF6 protein derived peptide set forth in Tables 1 to 10 (SEQ ID NOS: 1 to 3522). In one aspect, at least one MHC/peptide monomer comprises a peptide derived from SARS-CoV-2 ORF7a protein such as a SARS-CoV-2 ORF7a protein derived peptide set forth in Tables 1 to 10 (SEQ ID NOS: 1 to 3522). In one aspect, at least one MHC/peptide monomer comprises a peptide derived from SARS-CoV-2 ORF7b protein such as a SARS-CoV-2 ORF7b protein derived peptide set forth in Tables 1 to 10 (SEQ ID NOS: 1 to 3522). In one aspect, at least one MHC/peptide monomer comprises a peptide derived from SARS-CoV-2 ORF8 protein such as a SARS-CoV-2 ORF8 protein derived peptide set forth in Tables 1 to 10 (SEQ ID NOS: 1 to 3522). In one aspect, at least one MHC/peptide monomer comprises a peptide derived from SARS-CoV-2 ORF10 protein such as a SARS-CoV-2 ORF10 protein derived peptide set forth in Tables 1 to 10 (SEQ ID NOS: 1 to 3522). In one aspect, at least one MHC/peptide monomer comprises a peptide derived from SARS-CoV-2 nsp1 protein such as a SARS-CoV-2 nsp1 protein derived peptide set forth in Tables 1 to 10 (SEQ ID NOS: 1 to 3522). In one aspect, at least one MHC/peptide monomer comprises a peptide derived from SARS-CoV-2 nsp2 protein such as a SARS-CoV-2 nsp2 protein derived peptide set forth in Tables 1 to 10 (SEQ ID NOS: 1 to 3522). In one aspect, at least one MHC/peptide monomer comprises a peptide derived from SARS-CoV-2 nsp3 protein such as a SARS-CoV-2 nsp3 protein derived peptide set forth in Tables 1 to 10 (SEQ ID NOS: 1 to 3522). In one aspect, at least one MHC/peptide monomer comprises a peptide derived from SARS-CoV-2 nsp4 protein such as a SARS-CoV-2 nsp4 protein derived peptide set forth in Tables 1 to 10 (SEQ ID NOS: 1 to 3522). In one aspect, at least one MHC/peptide monomer comprises a peptide derived from SARS-CoV-2 nsp5 protein such as a SARS-CoV-2 nsp5 protein derived peptide set forth in Tables 1 to 10 (SEQ ID NOS: 1 to 3522). In one aspect, at least one MHC/peptide monomer comprises a peptide derived from SARS-CoV-2 nsp6 protein such as a SARS-CoV-2 nsp6 protein derived peptide set forth in Tables 1 to 10 (SEQ ID NOS: 1 to 3522). In one aspect, at least one MHC/peptide monomer comprises a peptide derived from SARS-CoV-2 nsp7 protein such as a SARS-CoV-2 nsp7 protein derived peptide set forth in Tables 1 to 10 (SEQ ID NOS: 1 to 3522). In one aspect, at least one MHC/peptide monomer comprises a peptide derived from SARS-CoV-2 nsp8 protein such as a SARS-CoV-2 nsp8 protein derived peptide set forth in Tables 1 to 10 (SEQ ID NOS: 1 to 3522). In one aspect, at least one MHC/peptide monomer comprises a peptide derived from SARS-CoV-2 nsp9 protein such as a SARS-CoV-2 nsp9 protein derived peptide set forth in Tables 1 to 10 (SEQ ID NOS: 1 to 3522). In one aspect, at least one MHC/peptide monomer comprises a peptide derived from SARS-CoV-2 nsp10 protein such as a SARS-CoV-2 nsp10 protein derived peptide set forth in Tables 1 to 10 (SEQ ID NOS: 1 to 3522). In one aspect, at least one MHC/peptide monomer comprises a peptide derived from SARS-CoV-2 nsp12 protein such as a SARS-CoV-2 nsp12 protein derived peptide set forth in Tables 1 to 10 (SEQ ID NOS: 1 to 3522). In one aspect, at least one MHC/peptide monomer comprises a peptide derived from SARS-CoV-2 nsp13 protein such as a SARS-CoV-2 nsp13 protein derived peptide set forth in Tables 1 to 10 (SEQ ID NOS: 1 to 3522). In one aspect, at least one MHC/peptide monomer comprises a peptide derived from SARS-CoV-2 nsp14 protein such as a SARS-CoV-2 nsp14 protein derived peptide set forth in Tables 1 to 10 (SEQ ID NOS: 1 to 3522). In one aspect, at least one MHC/peptide monomer comprises a peptide derived from SARS-CoV-2 nsp15 protein such as a SARS-CoV-2 nsp15 protein derived peptide set forth in Tables 1 to 10 (SEQ ID NOS: 1 to 3522). In one aspect, at least one MHC/peptide monomer comprises a peptide derived from SARS-CoV-2 nsp16 protein such as a SARS-CoV-2 nsp16 protein derived peptide set forth in Tables 1 to 10 (SEQ ID NOS: 1 to 3522). In one aspect, at least one MHC/peptide monomer comprises a peptide comprising, consisting of, or consisting essentially of an amino acid sequence selected from the SARS-CoV-2 B.1.1.529 derived sequences set forth in Table 8 (SEQ ID NOS: 2571 to 2615). In one aspect, at least one MHC/peptide monomer comprises a peptide comprising, consisting of, or consisting essentially of an amino acid sequence selected from the SARS-CoV-2 derived sequences set forth in Table 1 (SEQ ID NOS: 1 to 1468). In one aspect, at least one MHC/peptide monomer comprises a peptide comprising, consisting of, or consisting essentially of an amino acid sequence selected from the SARS-CoV-2 derived sequences set forth in Table 2 (SEQ ID NOS: 1469 to 1521) (CD8S(D) megapool). In one aspect, at least one MHC/peptide monomer comprises a peptide comprising, consisting of, or consisting essentially of an amino acid sequence selected from the SARS-CoV-2 derived sequences set forth in Table 3 (SEQ ID NOS: 1522 to 1665) (CD8S (ND) megapool). In one aspect, at least one MHC/peptide monomer comprises a peptide comprising, consisting of, or consisting essentially of an amino acid sequence selected from the SARS-CoV-2 derived sequences set forth in Table 4 (SEQ ID NOS: 1666 to 1818) (CD8R(D) megapool). In one aspect, at least one MHC/peptide monomer comprises a peptide comprising, consisting of, or consisting essentially of an amino acid sequence selected from the SARS-CoV-2 derived sequences set forth in Table 5 (SEQ ID NOS: 1819 to 2286) (CD8R(ND) megapool). In one aspect, at least one MHC/peptide monomer comprises a peptide comprising, consisting of, or consisting essentially of an amino acid sequence selected from the SARS-CoV-2 derived sequences set forth in Table 6 (SEQ ID NOS: 2287 to 2355) (CD4R(D) megapool). In one aspect, at least one MHC/peptide monomer comprises a peptide comprising, consisting of, or consisting essentially of an amino acid sequence selected from the SARS-CoV-2 derived sequences set forth in Table 7 (SEQ ID NOS: 2356 to 2570) (CD4R(ND) megapool). In one aspect, at least one MHC/peptide monomer comprises a peptide comprising, consisting of, or consisting essentially of an amino acid sequence selected from the SARS-CoV-2 derived sequences set forth in Table 9 (SEQ ID NOS: 2616 to 2900) (CD4RE megapool). In one aspect, at least one MHC/peptide monomer comprises a peptide comprising, consisting of, or consisting essentially of an amino acid sequence selected from the SARS-CoV-2 derived sequences set forth in Table 10 (SEQ ID NOS: 2901 to 3522) (CD8RE megapool). In one aspect, the at least two MHC/peptide monomers are identical. In one aspect, the MHC/peptide multimer comprise at least 3, such as at least 4, 5, 6, 7, 8, 9, 10, 11, 12, 13, 14, 15, 16, 17, 18, 19 or 20 MHC/peptide monomers. In one aspect, the MHC/peptide multimer comprise at least 3, such as at least 4, 5, 6, 7, 8, 9, 10, 11, 12, 13, 14, 15, 16, 17, 18, 19 or 20 identical MHC/peptide monomers. In one aspect, the MHC/peptide multimer comprise at least 3, such as at least 4, 5, 6, 7, 8, 9, 10, 11, 12, 13, 14, 15, 16, 17, 18, 19 or 20 different MHC/peptide monomers. In one aspect, at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the sequences set forth in Tables 1 to 10 (SEQ ID NOS: 1 to 3522). In one aspect, at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 B.1.1.529 derived sequences set forth in Table 8 (SEQ ID NOS: 2571 to 2615). In one aspect, at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 Spike (S) protein, Membrane (M) protein, Nucleocapsid (N) protein, Envelope (E) protein, ORF3a, ORF7a, ORF8, nsp1, nsp2, nsp3, nsp6, nsp9, nsp10, nsp12, nsp13, nsp14 and/or nsp15 derived sequences set forth in Tables 1 to 10 (SEQ ID NOS: 1 to 3522). In one aspect, each MHC/peptide monomer of the MHC/peptide multimer is associated with one or more multimerization domains such as a multimerization domain selected from the group consisting of proteins, peptides, albumins, immunoglobulins, coiled-coil helixes, polynucleotides, IgG, streptavidin, avidin, streptactin, micelles, cells, polymers, dextran, polysaccharides, beads and other types of solid support, and small organic molecules carrying reactive groups or carrying chemical motifs that can bind MHC/peptide monomers. In one aspect, the multimer comprises no more than 30 MHC/peptide monomers in total, such as no more than 25 MHC/peptide monomers, such as no more than 20 MHC/peptide monomers, such as no more than 15 MHC/peptide monomers, or no more than 10 MHC/peptide monomers in total. In one aspect, the MHC/peptide multimer comprises from 2 to 50 MHC/peptide monomers, such as from 2 to 4 MHC/peptide monomers, such as from 4 to 6 MHC/peptide monomers, such as from 6 to 8 MHC/peptide monomers, such as from 8 to 10 MHC/peptide monomers, such as from 10 to 12 MHC/peptide monomers, such as from 12 to 14 MHC/peptide monomers, such as from 14 to 16 MHC/peptide monomers, such as from 16 to 18 MHC/peptide monomers, such as from 18 to 20 MHC/peptide monomers, such as from 20 to 25 MHC/peptide monomers, such as from 25 to 30 MHC/peptide monomers, such as from 30 to 40 MHC/peptide monomers, such as from 40 to 50 MHC/peptide monomers, such as from 10 to 20 MHC/peptide monomers or any combination of these intervals. In one aspect, MHC/peptide multimer comprises at least 2, 3, 4, 5, 6, 7, 8, 9, 10, 11, 12, 13, 14, 15, 16, 17, 18, 19, 20, 21, 22, 23, 24 or 25 MHC/peptide monomers or has 2, 3, 4, 5, 6, 7, 8, 9, 10, 11, 12, 13, 14, 15, 16, 17, 18, 19, 20, 21, 22, 23, 24 or 25 MHC/peptide monomers in total. In one aspect, MHC/peptide multimer comprises MHC Class I/peptide monomers or wherein all MHC monomers of the MHC/peptide multimer are MHC Class I/peptide monomers. In one aspect, MHC/peptide multimer comprises MHC Class II/peptide monomers or wherein all MHC/peptide monomers of the MHC/peptide multimer are MHC Class II/peptide monomers. In one aspect, MHC/peptide multimer comprises MHC Class I/peptide and MHC Class II/peptide monomers or wherein all MHC/peptide monomers of the MHC/peptide multimer are either MHC Class I/peptide monomers or MHC Class II/peptide monomers. In one aspect, some of the MHC/peptide monomers or all of the MHC/peptide monomers have identical peptides. In one aspect, some of the MHC/peptide monomers or all of the MHC/peptide monomers have different peptides. In one aspect, at least 2, such as at least 3, 4, 5, 6, 7, 8, 9, 10, 11, 12, 13, 1, 15, 16, 17, 18, 19 or 20 of the MHC/peptide monomers comprise different peptides. In one aspect, the MHC/peptide multimer further comprise one or more labels such as at least two labels. In one aspect, the labels are different or at least some of the labels are different. In one aspect, the labels comprise at least one fluorescent label. In one aspect, the labels comprise at least one oligonucleotide label such as a nucleic acid molecule comprises or consists of DNA, RNA, and/or artificial nucleotides such as PLA or LNA. In one aspect, labels comprise at least one fluorescent label and at least one oligonucleotide label. In one aspect, the label is a oligonucleotide comprising one or more of: barcode region, 5′ first primer region (forward), 3′ second primer region (reverse), random nucleotide region, connector molecule, stability-increasing components, short nucleotide linkers in between any of the above-mentioned components, adaptors for sequencing, and annealing region. In one aspect, the labels comprise at least one such as one or more labels selected from the group consisting of APC, APC-Cy7, ABC-H7, APC-R700, Alexa Flours™ 488, Alexa Flours™555, Alexa Flours™647, Alexa Flours™700, AmCyan, BB151, BB700, BUV395, BUV496, BUV563, BUV615, BUV661, BUV737, BUV805, BV421, BV480, BV510, BV605, BV711, BV750, BV786, FITC, PE, PE-CF594, PE-Cy5, PE-CY5.5, PE-cy7, Pasific Blue, PERCP, pPerCp-Cy5.5, PE, R718, RY586, V450, V500, cFluor®B515, cFluor®B532, cFluor®B548, cFluor®B675, cFluor®B690, cFluor®BY575, cFluor®BY610, cFluor®BY667, cFluor®BY710, cFluor®BY750, cFluor®BY781, cFluor®B250, cFluor®R659, cFluor®R668, cFluor®R685, cFluor®R720, cFluor®R780, cFluor®R840, cFluor®v420, cFluor®v547, cFluor®v450, cFluor®v610 and cFluor®YG610. In one aspect, the one or more labels is a chemiluminescent label such as a label selected from the group consisting of luminol, isoluminol, theromatic acridinium ester, imidazole, acridinium salt and oxalate ester. In one aspect, one or more labels is a bioluminescent label such as a label selected from the group consisting of luciferin, luciferase and aequorin. In one aspect, the one or more labels is an enzyme label, such as an enzyme label selected from the group peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. In one aspect, the one or more labels is a chromophore label. In one aspect, the one or more labels is a metal label. In one aspect, the one or more labels is a radioactive label such as a label selected from the group consisting of a radionuclide, an isotope, a label comprising α rays, a label comprising β rays or a label comprising γ rays.

As embodied and broadly described herein, an aspect of the present disclosure relates to a composition comprising at least two MHC/peptide multimers describe hereinabove, such as at least 2, 3, 4, 5, 6, 7, 8, 9, 10, 20, 30, 40, 50, 100 or 1000 MHC/peptide multimers. In one aspect, composition comprises different MHC/peptide multimers, such as at least 2, 3, 4, 5, 6, 7, 8, 9, 10, 15, 20, 25, 50, 100, 500 or 1000 different MHC/peptide multimers. In another aspect, at least 2, 3, 4, 5, 6, 7, 8, 9, 10, 20, 30, 40, 50, 100 or 1000 MHC/peptide multimers of the composition are different each comprising one or more peptides selected from one or more of the following groups: i) one or more peptides derived from SARS-CoV-2 B.1.1.7, such as one or more SARS-CoV-2 B.1.1.7 derived peptides set forth in Tables 1 to 10 (SEQ ID NOS: 1 to 3522), such as one or more peptides set forth in Table 1, such as one or more peptides set forth in Table 2, such as one or more peptides set forth in Table 3, such as one or more peptides set forth in Table 4, such as one or more peptides set forth in Table 5, such as one or more peptides set forth in Table 6, such as one or more peptides set forth in Table 7, such as one or more peptides set forth in Table 8, such as one or more peptides set forth in Table 9 and/or such as one or more peptides set forth in Table 10, or any combination thereof, ii) one or more peptides derived from SARS-CoV-2 B1.351. such as one or more SARS-CoV-2 B1.351 derived peptides set forth in Tables 1 to 10 (SEQ ID NOS: 1 to 3522), iii) one or more peptides derived from SARS-CoV-2 P.1, such as one or more SARS-CoV-2 P.1 derived peptides set forth in Tables 1 to 10 (SEQ ID NOS: 1 to 3522), iv) one or more peptides derived from SARS-CoV-2 CAL.20C, such as one or more SARS-CoV-2 CAL.20C derived peptides set forth in Tables 1 to 10 (SEQ ID NOS: 1 to 3522), v) one or more peptides set forth in Tables 1 to 10 (SEQ ID NOS: 1 to 3522), vi) one or more peptides derived from the SARS-CoV-2 Spike (S) protein such as one or more SARS-CoV-2 Spike (S) protein derived peptides set forth in Tables 1 to 10 (SEQ ID NOS: 1 to 3522), vii) one or more peptides derived from the SARS-CoV-2 Membrane (M) protein such as one or more SARS-CoV-2 Membrane (M) protein derived peptides set forth in Tables 1 to 10 (SEQ ID NOS: 1 to 3522), viii) one or more peptides derived from the SARS-CoV-2 Nucleocapsid (N) protein such as one or more SARS-CoV-2 Nucleocapsid (N) protein derived peptides set forth in Tables 1 to 10 (SEQ ID NOS: 1 to 3522), ix) one or more peptides derived from the SARS-CoV-2 Envelope (E) protein such as one or more SARS-CoV-2 Envelope (E) protein derived peptides set forth in Tables 1 to 10 (SEQ ID NOS: 1 to 3522), x) one or more peptides derived from the SARS-CoV-2 ORF3a protein such as one or more SARS-CoV-2 ORF3a protein derived peptides set forth in Tables 1 to 10 (SEQ ID NOS: 1 to 3522), xi) one or more peptides derived from the SARS-CoV-2 ORF6 protein such as one or more SARS-CoV-2 ORF6 protein derived peptides set forth in Tables 1 to 10 (SEQ ID NOS: 1 to 3522), xii) one or more peptides derived from the SARS-CoV-2 ORF7a protein such as one or more SARS-CoV-2 ORF7a protein derived peptides set forth in Tables 1 to 10 (SEQ ID NOS: 1 to 3522), xiii) one or more peptides derived from the SARS-CoV-2 ORF7b protein such as one or more SARS-CoV-2 ORF7b protein derived peptides set forth in Tables 1 to 10 (SEQ ID NOS: 1 to 3522), xiv) one or more peptides derived from the SARS-CoV-2 ORF8 protein such as one or more SARS-CoV-2 ORF8 protein derived peptides set forth in Tables 1 to 10 (SEQ ID NOS: 1 to 3522), xv) one or more peptides derived from the SARS-CoV-2 ORF10 protein such as one or more SARS-CoV-2 ORF10 protein derived peptides set forth in Tables 1 to 10 (SEQ ID NOS: 1 to 3522), xvi) one or more peptides derived from the SARS-CoV-2 nsp1 protein such as one or more SARS-CoV-2 nsp1 protein derived peptides Tables 1 to 10 (SEQ ID NOS: 1 to 3522), xvii) one or more peptides derived from the SARS-CoV-2 nsp2 protein such as one or more SARS-CoV-2 nsp2 protein derived peptides set forth in Tables 1 to 10 (SEQ ID NOS: 1 to 3522), xviii) one or more peptides derived from the SARS-CoV-2 nsp3 protein such as one or more SARS-CoV-2 nsp3 protein derived peptides set forth in Tables 1 to 10 (SEQ ID NOS: 1 to 3522), xix) one or more peptides derived from the SARS-CoV-2 nsp4 protein such as one or more SARS-CoV-2 nsp4 protein derived peptides set forth in Tables 1 to 10 (SEQ ID NOS: 1 to 3522), xx) one or more peptides derived from the SARS-CoV-2 nsp5 protein such as one or more SARS-CoV-2 nsp5 protein derived peptides set forth in Tables 1 to 10 (SEQ ID NOS: 1 to 3522), xxi) one or more peptides derived from the SARS-CoV-2 nsp6 protein such as one or more SARS-CoV-2 nsp6 protein derived peptides set forth in Tables 1 to 10 (SEQ ID NOS: 1 to 3522), xxii) one or more peptides derived from the SARS-CoV-2 nsp7 protein such as one or more SARS-CoV-2 nsp7 protein derived peptides set forth in Tables 1 to 10 (SEQ ID NOS: 1 to 3522), xxiii) one or more peptides derived from the SARS-CoV-2 nsp8 protein such as one or more SARS-CoV-2 nsp8 protein derived peptides set forth in Tables 1 to 10 (SEQ ID NOS: 1 to 3522), xxiv) one or more peptides derived from the SARS-CoV-2 nsp9 protein such as one or more SARS-CoV-2 nsp9 protein derived peptides set forth in Tables 1 to 10 (SEQ ID NOS: 1 to 3522), xxv) one or more peptides derived from the SARS-CoV-2 nsp10 protein such as one or more SARS-CoV-2 nsp10 protein derived peptide set forth in Tables 1 to 10 (SEQ ID NOS: 1 to 3522), xxvi) one or more peptides derived from the SARS-CoV-2 nsp12 protein such as one or more SARS-CoV-2 nsp12 protein derived peptides set forth in Tables 1 to 10 (SEQ ID NOS: 1 to 3522), xxvii) one or more peptides derived from the SARS-CoV-2 nsp13 protein such as one or more SARS-CoV-2 nsp13 protein derived peptides set forth in Tables 1 to 10 (SEQ ID NOS: 1 to 3522), xxviii) one or more peptides derived from the SARS-CoV-2 nsp14 protein such as one or more SARS-CoV-2 nsp14 protein derived peptides set forth in Tables 1 to 10 (SEQ ID NOS: 1 to 3522), xxix) one or more peptides derived from the SARS-CoV-2 nsp15 protein such as one or more SARS-CoV-2 nsp15 protein derived peptides set forth in Tables 1 to 10 (SEQ ID NOS: 1 to 3522), and xxx) one or more peptides derived from the SARS-CoV-2 nsp16 protein such as one or more SARS-CoV-2 nsp16 protein derived peptides set forth in Tables 1 to 10 (SEQ ID NOS: 1 to 3522).

As embodied and broadly described herein, an aspect of the present disclosure relates to a method for monitoring an immune response relevant to a coronavirus infection comprising one or more steps of: i) providing one or more MHC/peptide multimers describe hereinabove, ii) providing a sample comprising a population of T cells, and iii) measuring the presence, frequency, number, activity and/or state of T cells specific for said one or more MHC/peptide multimers, thereby monitoring said immune response relevant to a coronavirus infection.

As embodied and broadly described herein, an aspect of the present disclosure relates to a method for diagnosing a coronavirus infection comprising one or more steps of: i) providing one or more MHC/peptide multimers of any of the compositions described hereinabove, ii) providing a sample comprising a population of T cells, and iii) measuring the presence, frequency, number, activity and/or state of T cells specific for said one or more MHC/peptide multimers, thereby diagnosing said coronavirus infection.

As embodied and broadly described herein, an aspect of the present disclosure relates to a method for isolation of one or more antigen-specific T cells, said method comprising one or more steps of:
i) providing a sample comprising a population of T cells, ii) providing one or more MHC/peptide multimers described hereinabove, iii) contacting said MHC/peptide multimers or composition with said sample comprising a population of T cells, and iv) isolating T cells specific for said MHC/peptide multimers or composition.

As embodied and broadly described herein, an aspect of the present disclosure relates to a method for detecting an antigen-specific T cell response comprising one or more steps of: i) providing a sample comprising a population of T cells, ii) providing one or more MHC/peptide multimers of any composition described hereinabove, iii) contacting said MHC/peptide multimers or composition with said sample, and iv) measuring the presence, frequency, number, activity and/or state of T cells specific for said MHC/peptide multimers or composition, thereby detecting said antigen-specific T cell response.

As embodied and broadly described herein, an aspect of the present disclosure relates to a method of distinguishing an immune response from a subject that has been: a) vaccinated against but not exposed to SARS-COV-2, b) exposed to SARS-COV-2 but not vaccinated against SARS-COV-2, c) vaccinated against and exposed to SARS-COV-2, or d) neither vaccinated against nor exposed to SARS-COV-2, the method comprising, consisting of, or consisting essentially of: contacting a biological sample from a subject with a composition described hereinabove; and determining if the composition elicits an immune response from the contacted cells, wherein the level of elicited immune response indicates whether the subject falls into category a), b), c), or d). In one aspect, determining whether the subject falls into category a), b), c), or d) further comprises determining whether the immune response is predominantly to a SARS-CoV-2 Spike protein, or is to one or more SARS-CoV-2antigens other than the Spike protein, wherein: i) a predominant response to SARS-CoV-2 Spike protein and minimal response to one or more SARS-CoV-2 antigens other than Spike is indicative that a subject falls into category a), ii) a response to coronavirus Spike protein and one or more SARS-CoV-2 antigens other than Spike is indicative that the subject falls into category b), iii) a strong response to SARS-CoV-2 Spike protein and one or more SARS-CoV-2 antigens other than Spike is indicative that the subject falls into category c), and iv) a weak or no response to SARS-CoV-2 Spike or one or more SARS-CoV-2 antigens other than Spike is indicative that the subject falls into category d). In one aspect, the SARS-CoV-2 Spike protein or SARS-CoV-2 antigen is a protein or peptide comprising an amino acid sequence set forth in Tables 1 to 10 (SEQ ID NOS: 1 to 3522). In another aspect, the sample comprises T cells. In another aspect, the response comprises inducing, increasing, promoting or stimulating anti-SARS-CoV-2 activity of T cells. In another aspect, the T cells are CD8+ or CD4+ T cells. In another aspect, the method comprises determining whether the subject has been infected by or exposed to SARS-CoV-2 more than once by determining if the subject elicits a secondary T cell immune response profile that is different from a primary T cell immune response profile. In another aspect, the method further comprises diagnosing a SARS-CoV-2 infection or exposure in a subject, the method comprising contacting a biological sample from a subject with a composition described hereinabove; and determining if the composition elicits a T cell immune response, wherein the T cell immune response identifies that the subject has been infected with or exposed to SARS-CoV-2. In another aspect, the method is conducted three or more days following the date of suspected infection by or exposure to a coronavirus.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show a non-limiting example of the Distribution of CD4 and CD8 epitopes by SARS-CoV-2 antigen. The fraction of known CD4 and CD8 epitopes derived from recognized SARS-CoV-2 antigens is shown in (FIG. 1A) and (FIG. 1), respectively. The number of epitopes derived from each antigen as a function of antigen size is plotted in panels (FIG. 1C) and (FIG. 1D) for CD4 and CD8, respectively; p values were calculated using a simple linear regression. Panels (FIG. 1E) and (FIG. 1F) indicated the number of studies that probed responses to each antigen.

FIGS. 2A to 2F show a non-limiting example of the identification of immunodominant antigenic regions. The IEDB's Immunome Browser tool was utilized to identify potential antigenic regions across the entire SARS-CoV-2 proteome. After searching for SARS-CoV-2-derived CD4 or CD8 epitopes, individual antigens were selected for further evaluation. From the antigen-specific Immunome Browser link, data was downloaded as an Excel file to obtain position-specific lower bound response frequency scores (RF), defined as the number of individuals and assays reporting positive responses to a peptide including that particular residue. For visualization, RF scores for each residue were recalculated to represent a sliding 10 residue window. Position specific RF values for CD4 (top) and CD8 (bottom) epitopes are shown for the most dominant antigens respectively, to include spike (FIG. 2A and FIG. 2B), M and N (FIG. 2C and FIG. 2D), nsp3 and nsp12 (FIG. 2E and FIG. 2F).

FIGS. 3A to 3D show a non-limiting example of the Defined HLA class I and class II restrictions. HLA restricted epitopes have been identified for 30 class I (FIG. 3A) and 45 class II (FIG. 3B) molecules. The number of epitopes associated with each allele is plotted. FIG. 3C shows CD8 responses and FIG. 3D shows CD4 responses induced by Spike CD8 and CD4 megapools, respectively.

FIGS. 4A to 4D. SARS-CoV-2-specific CD4+ and CD8+ T cell responses in the study groups SARS-CoV-2-specific T cell responses were measured as percentage of AIM+ (OX40+CD137+) CD4+ T cells or AIM+(CD69+CD137+) CD8+ T cells after stimulation of PBMCs with peptides pools encompassing spike only (Spike) MP or the experimentally defined CD4RE and CD8RE MPs representing all the proteome without spike. EVB MP was used as a control. Graphs show individual response of spike, CD4RE or CD8RE and the combination of both (Total CD4+ or Total CD8+) plotted as background subtracted (FIG. 4A, 4C) or as SI (FIG. 4B, 4D) against DMSO negative control. Geometric mean for the 4 different groups is shown. Kruskal-Wallis test adjusted with Dunn's test for multiple comparisons was performed and p values <0.05 considered statistically significant. I−V−, unexposed and unvaccinated (n=30); I+V−, infected and non-vaccinated (n=30); I+V+, infected and then vaccinated (n=30); I−V+, non-infected and vaccinated (n=30). Threshold of positivity (TP) is indicated. Median response, and the number or percentage of positive responding donors for each group is shown.

FIGS. 5A and 5B. COVID-19 clinical classification scheme using SARS-CoV-2-specific CD4+ T cell responses. CD4+ T cell responses to spike and CD4RE MPs were measured as percentage of AIM+(OX40+CD137+) CD4+ T cells and plotted in two dimensions as absolute magnitude in order to discriminate the 4 study groups with known COVID-19 status of infection, and/or vaccination in 2 independent cohorts: (FIG. 5A) Exploratory cohort (n=120) and (FIG. 5B) Validation cohort (n=96). I−V−, unexposed and unvaccinated (n=30 and n=20); I+V−, infected and non-vaccinated (n=30 and n=20); I+V+, infected and then vaccinated (n=30 and n=20); I−V+, non-infected and vaccinated (n=30 and n=36). Dotted lines indicate specific cutoffs. Table inserts depict the diagnostic exam results in 4×4 matrix. Sensitivity, specificity, PPV, NPV and overall percentage of subjects classified correctly is shown.

FIGS. 6A to 6C COVID-19 clinical classification scheme is applicable to different mRNA vaccines and different lengths of time post-infection/post-vaccination. CD4+ T cell responses to spike and CD4RE MPs were measured as percentage of AIM+(OX40+CD137+) CD4+ T cells and plotted in two dimensions as absolute magnitude in order to discriminated between: (FIG. 6A) different types of mRNA vaccines (Moderna vs Pfzier) among vaccinated groups (I-V+ and I+V+); (FIG. 6B) different lengths of time post-infection among infected groups (I+V− and I+V+); (FIG. 6C) different lengths of time post-vaccination among vaccinated groups (I−V+ and I+V+). Early infection: PSO≤180; Late infection: PSO≥180; Early post-vaccination: PVD≤30; Late post-vaccination: PVD>30. I−V+, non-infected and vaccinated (n=66); I+V−, infected and non-vaccinated (n=50); I+V+, infected and then vaccinated (n=50). Dotted lines indicate specific cutoffs. Table inserts depict the overall percentage of subjects classified correctly.

FIGS. 7A to 7D. SARS-CoV-2 T cell and antibody response in breakthrough infection cases. Comparison to other study groups SARS-CoV-2-specific T cell responses were measured as percentage of (FIG. 7A) AIM+(OX40+CD137+) CD4+ T cells or (FIG. 7B) AIM+(CD69+CD137+) CD8+ T cells after stimulation of PBMCs with Spike and CD4RE or CD8RE peptide pools. (FIG. 7C) Comparison of anti-spike RBD IgG titers in the plasma of the different study groups. For both T cell and antibody determinations only donors matching the V+I+ intervals of vaccination and infection (55-271 and 18-93 days, respectively) were plotted. Graph bars show geometric mean. Threshold of positivity (TP), median response, and the number or percentage of positive responding donors for each group is indicated. Kruskal-Wallis test adjusted with Dunn's test for multiple comparisons was performed and p values <0.05 considered statistically significant. (FIG. 7D) V+I+CD4+ T cell responses plotted using the two-dimensional classification scheme with the specific cutoffs attributed to the different study groups (dotted lines). Unexposed and unvaccinated (n=50); I+V−, infected and non-vaccinated (n=50); I+V+, infected and then vaccinated (n=50); I-V+, non-infected and vaccinated (n=66); V+I+, vaccinated and then infected (n=23).

FIGS. 8A and 8B. Overall COVID-19 clinical classification scheme. CD4+ T cell responses to spike and CD4RE MPs were measured as percentage of AIM+(OX40+CD137+) CD4+ T cells and plotted in two dimensions as (FIG. 8A) SFCs per million PBMCs or (FIG. 8B) stimulation index (SI), in order to discriminate the 5 study groups with known COVID-19 status of infection, and/or vaccination. I−V−, unexposed and unvaccinated (n=50); I+V−, infected and non-vaccinated (n=50); I−V+, non-infected and vaccinated (n=66); I+V+/V+I+, infected and then vaccinated (I+V+, n=50) merged with vaccinated and then infected (V+I+, n=23). Dotted lines indicate specific cutoffs. Table inserts depict the diagnostic exam results in 4×4 matrix. Sensitivity, specificity, PPV, NPV of all the subjects that participated in this study (n=239) and overall percentage classified correctly is shown.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention.

It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

As used herein, the term “gene” refers to a segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

As used herein, the terms “expression” or “expressed” reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “polypeptide,” “peptide,” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or virus.

As used herein, the term “peptide mimetic” or “peptidomimetic” refer to protein-like chain designed to mimic a peptide or protein. Peptide mimetics may be generated by modifying an existing peptide or by designing a compound that mimic peptides, including peptoids and β-peptides.

As used herein, the phrase “conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

As used herein, a “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the terms “identical” or percent “identity” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

As used herein, the terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, the term “multimer” refers to a complex comprising multiple monomers (e.g., a protein complex) associated by covalent and/or noncovalent bonds. The monomers can be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a multimeric complex such as a stable multimeric complex composed of or comprising MHC protein(s) subunits loaded with a peptide (MHC/peptide monomers) of the present disclosure. For example, an MHC/peptide multimer (also called herein MHC/peptide complex) include, but are not limited to, an MHC/peptide dimer, trimer, tetramer, pentamer or higher valency multimer, e.g., comprising 2, 3, 4, 5, 6, 7, 8, 9, 10, 11, 12, 13, 14, 15, 16, 17, 18, 19, 20, 21, 22, 23, 24 or more than 24 MHC/peptide monomers. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present disclosure. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In one embodiment the term “MHC/peptide multimer” refers to a complex comprising multiple MHC/peptide monomers (i.e., at least two MHC/peptide monomers) associated by covalent and/or noncovalent bonds. The MHC/peptide monomers can be substantially identical MHC/peptide monomers, or the MHC/peptide monomers may be different. In one embodiment the MHC/peptide multimer comprises a peptide comprising, consisting of, or consisting essentially of an amino acid sequence selected from those sequences set forth in Tables 1 to 10 (SEQ ID NOS: 1 to 3522), preferably in a groove of the MHC monomer. Each MHC/peptide monomer of the MHC/peptide multimer can be associated with one or more multimerization domains such as a multimerization domain selected from the group consisting of IgG, streptavidin, avidin, streptactin, micelles, cells, polymers, dextran, polysaccharides, beads and other types of solid support, and small organic molecules carrying reactive groups or carrying chemical motifs that can bind MHC complexes.

In one embodiment the MHC/peptide multimer comprises at least 2 MHC/peptide monomers, such as at least 3 MHC/peptide monomers such as at least 4 MHC/peptide monomers, such as at least 5 MHC/peptide monomers, such as at least 6 MHC/peptide monomers, such as at least 8 MHC/peptide monomers, such as at least 10 MHC/peptide monomers, such as at least 12 MHC/peptide monomers, such as at least 14 MHC/peptide monomers, such as at least 16 MHC/peptide monomers, such as at least 18 MHC/peptide monomers or such as at least 20 MHC/peptide monomers. In another embodiment the MHC/peptide multimer comprises from 2 to 50 MHC/peptide monomers, such as from 2 to 4 MHC/peptide monomers, such as from 4 to 6 MHC/peptide monomers, such as from 6 to 8 MHC/peptide monomers, such as from 8 to 10 MHC/peptide monomers, such as from 10 to 12 MHC/peptide monomers, such as from 12 to 14 MHC/peptide monomers, such as from 14 to 16 MHC/peptide monomers, such as from 16 to 18 MHC/peptide monomers, such as from 18 to 20 MHC/peptide monomers, such as from 20 to 25 MHC/peptide monomers, such as from 25 to 30 MHC/peptide monomers, such as from 30 to 40 MHC/peptide monomers, such as from 40 to 50 MHC/peptide monomers, or any combination of these intervals. In one aspect the MHC/peptide multimer comprises no more than 30 MHC/peptide monomers in total, such as no more than 25 MHC/peptide monomers, such as no more than 20 MHC/peptide monomers, such as no more than 15 MHC/peptide monomers, or no more than 10 MHC/peptide monomers in total.

In a specific embodiment, the MHC/peptide multimer comprises at least 2, 3, 4, 5, 6, 7, 8, 9, 10, 11, 12, 13, 14, 15, 16, 17, 18, 19, 20, 21, 22, 23, 24 or 25 MHC/peptide monomers or has 2, 3, 4, 5, 6, 7, 8, 9, 10, 11, 12, 13, 14, 15, 16, 17, 18, 19, 20, 21, 22, 23, 24 or 25 MHC/peptide monomers in total. The MHC/peptide multimer can comprise identical MHC/peptide monomers or all MHC/peptide monomers of the MHC/peptide multimer can be identical. In another embodiment the MHC/peptide multimer comprises different MHC/peptide monomers or all MHC/peptide monomers of the MHC/peptide multimer are different. The MHC/peptide multimer can comprise MHC Class I monomers or all MHC/peptide monomers of the MHC/peptide multimer can be MHC Class I monomers. Alternatively, the MHC/peptide multimer can comprise MHC Class II monomers or all MHC/peptide monomers of the MHC/peptide multimer can be MHC Class II monomers. In another embodiment the MHC/peptide multimer comprises MHC Class I and MHC Class II monomers or all MHC/peptide monomers of the MHC/peptide multimer are either MHC Class I monomers or MHC Class II monomers. In one embodiment some of the MHC/peptide monomers or all of the MHC/peptide monomers on a MHC/peptide multimer have identical peptides. In another embodiment some of the MHC/peptide monomers or all of the MHC/peptide monomers on a MHC/peptide multimer have different peptides. The MHC/peptide multimer can comprise at least 2, such as at least 3, 4, 5, 6, 7, 8, 9, 10, 11, 12, 13, 14, 15, 16, 17, 18, 19 or 20 MHC/peptide monomers which comprise different peptides.

The MHC/peptide multimer may comprise one or more labels such as at least two labels. These labels can all be different or identical or some the labels can be identical and some different. In one embodiment the labels comprise at least one fluorescent label and/or at least one oligonucleotide label. In a specific embodiment the at least one oligonucleotide on a MHC/peptide multimer comprises one or more of: barcode region, 5′ first primer region (forward), 3′ second primer region (reverse), random nucleotide region, connector molecule, stability-increasing components, short nucleotide linkers in between any of the above-mentioned components, adaptors for sequencing and annealing region. MHC/peptide multimers are described in detail in WO02072631, WO2008116468, WO2009003492 and WO2020127222, which hereby are incorporated by reference.

The present disclosure relates to peptide-major histocompatibility complex (MHC)/peptide multimers comprising at least two MHC/peptide monomers, wherein at least one MHC/peptide monomer comprises a peptide derived from SARS-CoV-2. In a preferred embodiment the MHC/peptide multimer comprises at least two MHC/peptide monomers, wherein at least one MHC/peptide monomer comprises a peptide that comprises, consists of, or consists essentially of an amino acid sequence selected from the sequences set forth in Tables 1 to 10 (SEQ ID NOS: 1 to 3522). The MHC/peptide multimer can comprise at least one MHC/peptide monomer comprising a peptide derived from SARS-CoV-2 Spike (S) protein such as a SARS-CoV-2 Spike (S) protein-derived peptide set forth in Tables 1 to 10 (SEQ ID NOS: 1 to 3522). The MHC/peptide multimer can comprise at least one MHC/peptide monomer comprising a peptide derived from SARS-CoV-2 Membrane (M) protein such as a SARS-CoV-2 Membrane (M) protein-derived peptide set forth in Tables 1 to 10 (SEQ ID NOS: 1 to 3522). The MHC/peptide multimer can comprise at least one MHC/peptide monomer comprising a peptide derived from SARS-CoV-2 Nucleocapsid (N) protein such as a SARS-CoV-2 Nucleocapsid (N) protein-derived peptide set forth in Tables 1 to 10 (SEQ ID NOS: 1 to 3522). The MHC/peptide multimer can comprise at least one MHC/peptide monomer comprising a peptide derived from SARS-CoV-2 Envelope (E) protein such as a SARS-CoV-2 Envelope (E) protein-derived peptide set forth in Tables 1 to 10 (SEQ ID NOS: 1 to 3522). The MHC/peptide multimer can comprise at least one MHC/peptide monomer comprising a peptide derived from SARS-CoV-2 ORF3a protein such as a SARS-CoV-2 ORF3a protein-derived peptide set forth in Tables 1 to 10 (SEQ ID NOS: 1 to 3522). The MHC/peptide multimer can comprise at least one MHC/peptide monomer comprising a peptide derived from SARS-CoV-2 ORF6 protein such as a SARS-CoV-2 ORF6 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 ORF7a protein such as a SARS-CoV-2 ORF7a protein-derived peptide set forth in Tables 1 to 10 (SEQ ID NOS: 1 to 3522). The MHC/peptide multimer can comprise at least one MHC/peptide monomer comprising a peptide derived from SARS-CoV-2 ORF7b protein such as a SARS-CoV-2 ORF7b protein-derived peptide set forth in Tables 1 to 10 (SEQ ID NOS: 1 to 3522). The MHC/peptide multimer can comprise at least one MHC/peptide monomer comprising a peptide derived from SARS-CoV-2 ORF8 protein such as a SARS-CoV-2 ORF8 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 ORF10 protein such as a SARS-CoV-2 ORF10 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp1 protein such as a SARS-CoV-2 nsp1 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp2 protein such as a SARS-CoV-2 nsp2 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp3 protein such as a SARS-CoV-2 nsp3 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp6 protein such as a SARS-CoV-2 nsp6 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp9 protein such as a SARS-CoV-2 nsp9 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp10 protein such as a SARS-CoV-2 nsp10 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp12 protein such as a SARS-CoV-2 nsp12 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp13 protein such as a SARS-CoV-2 nsp13 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp4 protein such as a SARS-CoV-2 nsp4 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp5 protein such as a SARS-CoV-2 nsp5 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp14 protein such as a SARS-CoV-2 nsp14 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp7 protein such as a SARS-CoV-2 nsp7 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp8 protein such as a SARS-CoV-2 nsp8 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp15 protein such as a SARS-CoV-2 nsp15 protein-derived peptide set forth in Tables 1 to 10 (SEQ ID NOS: 1 to 3522). The MHC/peptide multimer can comprise at least one MHC/peptide monomer comprising a peptide derived from SARS-CoV-2 nsp16 protein such as a SARS-CoV-2 nsp16 protein-derived peptide set forth in Tables 1 to 10 (SEQ ID NOS: 1 to 3522). The MHC/peptide multimer can comprise more than one of the different MHC/peptide monomers listed above, e.g., comprise at least 2, 3, 4, 5, 6, 7, 8, 9, 10 or more than 10 different MHC/peptide monomers by combining any of the above embodiments.

In certain embodiments, the at least two MHC/peptide monomers can be identical and/or different. In one embodiment the MHC/peptide multimer comprises some identical and some different MHC/peptide monomers or alternatively all the MHC/peptide monomers can be different. In a specific embodiment the MHC/peptide multimer comprises at least one MHC/peptide monomer which comprises a peptide comprising, consisting of, or consisting essentially of an amino acid sequence selected from the SARS-CoV-2 B.1.1.529 derived sequences set forth in Table 8 (SEQ ID NOS: 2571 to 2615). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 1 (SEQ ID NOS: 1 to 1468). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 2 (SEQ ID NOS: 1469 to 1521) (CD8S(D) megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 3 (SEQ ID NOS: 1522 to 1665) (CD8S(ND) megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 4 (SEQ ID NOS: 1666 to 1818) (CD8R (D) megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 5 (SEQ ID NOS: 1819 to 2286) (CD8R (ND) megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 6 (SEQ ID NOS: 2287 to 2355) (CD4R(D) megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 7 (SEQ ID NOS: 2356 to 2570) (CD4R (ND) megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 9 (SEQ ID NOS: 2616 to 2900) (CD4RE megapool). In a specific embodiment the MHC/peptide multimer comprises at least one MHC/peptide monomer which comprises a peptide comprising, consisting of, or consisting essentially of an amino acid sequence selected from the SARS-CoV-2 derived sequences set forth in Table 10 (SEQ ID NOS: 2901 to 3522) (CD8RE megapool).

In a further embodiment, the MHC/peptide multimer comprises at least one MHC/peptide monomer which comprises a peptide comprising, consisting of, or consisting essentially of an amino acid sequence selected from the SARS-CoV-2 derived sequences set forth in Tables 1 to 10 (SEQ ID NOS: 1 to 3522). The MHC/peptide multimer can comprise more than one of the different MHC/peptide monomers listed above, e.g., comprise at least 2, 3, 4, 5, 6, 7, 8, 9, 10 or more than 10 different MHC/peptide monomers by combining any of the above embodiments.

The MHC/peptide multimer can comprise at least 3, such as at least 4, 5, 6, 7, 8, 9, 10, 11, 12, 13, 14, 15, 16, 17, 18, 19 or 20 MHC/peptide monomers. The MHC/peptide multimer can in one embodiment comprise at least 3, such as at least 4, 5, 6, 7, 8, 9, 10, 11, 12, 13, 14, 15, 16, 17, 18, 19 or 20 identical MHC/peptide monomers. The MHC/peptide multimer can in another embodiment comprise at least 3, such as at least 4, 5, 6, 7, 8, 9, 10, 11, 12, 13, 14, 15, 16, 17, 18, 19 or 20 different MHC/peptide monomers. In a particular embodiment the MHC/peptide multimer comprises at least 3, such as at least 4, 5, 6, 7, 8, 9, 10, 11, 12, 13, 14, 15, 16, 17, 18, 19 or 20 of the MHC/peptide monomers which comprises a peptide which comprises, consists of, or consists essentially of an amino acid sequence selected from the sequences set forth in Tables 1 to 10 (SEQ ID NOS: 1 to 3522).

In a specific embodiment, the MHC/peptide multimer comprises at least 3, such as at least 4, 5, 6, 7, 8, 9, 10, 11, 12, 13, 14, 15, 16, 17, 18, 19 or 20 MHC/peptide monomers that comprises a peptide which comprises, consists of, or consists essentially of an amino acid sequence selected from the SARS-CoV-2 derived sequences set forth in Tables 1 to 10 (SEQ ID NOS: 1 to 3522). In another embodiment the MHC/peptide multimer comprises at least 3, such as at least 4, 5, 6, 7, 8, 9, 10, 11, 12, 13, 14, 15, 16, 17, 18, 19 or 20 MHC/peptide monomers that comprises a peptide which comprises, consists of, or consists essentially of an amino acid sequence selected from the SARS-CoV-2 B.1.1.529 derived sequences set forth in Table 8 (SEQ ID NOS: 2571 to 2615). In another embodiment the MHC/peptide multimer comprises at least 3, such as at least 4, 5, 6, 7, 8, 9, 10, 11, 12, 13, 14, 15, 16, 17, 18, 19 or 20 MHC/peptide monomers that comprises a peptide which comprises, consists of, or consists essentially of an amino acid sequence selected from the SARS-CoV-2 Spike (S) protein, Membrane (M) protein, Nucleocapsid (N) protein, Envelope (E) protein, ORF3a, ORF7a, ORF8, nsp1, nsp2, nsp3, nsp6, nsp9, nsp10, nsp12, nsp13, nsp14 and/or nsp15 derived sequences set forth in Tables 1 to 10 (SEQ ID NOS: 1 to 3522). The MHC/peptide multimer can comprise more than one of the different MHC/peptide monomers listed above, e.g., comprise at least 2, 3, 4, 5, 6, 7, 8, 9, 10 or more than 10 different MHC/peptide monomers by combining any of the above embodiments.

This disclosure further relates to a composition comprising at least two MHC/peptide multimers as described above, such as at least 3, 4, 5, 6, 7, 8, 9, 10, 20, 30, 40, 50, 100 or 1000 MHC/peptide multimers. The MHC/peptide multimers in the composition can all be identical or different. Alternatively, some MHC/peptide multimers in the composition are identical and some are different. The composition can comprise different MHC/peptide multimers, such as at least 2, 3, 4, 5, 6, 7, 8, 9, 10, 15, 20, 25, 50, 100, 500 or 1000 different MHC/peptide multimers. The composition can in one embodiment comprise at least 2, 3, 4, 5, 6, 7, 8, 9, 10, 20, 30, 40, 50, 100 or 1000 different MHC/peptide multimers each comprising one or more peptides selected from one or more such as 2, 3, 4, 5, 6, 7, 8, 9 or 10 of the following groups:
one or more peptides derived from SARS-CoV-2, such as one or more SARS-CoV-2 derived peptides set forth Tables 1 to 10 (SEQ ID NOS: 1 to 3522), such as one or more peptides set forth in Table 1, such as one or more peptides set forth in Table 2, such as one or more peptides set forth in Table 3, such as one or more peptides set forth in Table 4, such as one or more peptides set forth in Table 5, such as one or more peptides set forth in Table 6, such as one or more peptides set forth in Table 7, such as one or more peptides set forth in Table 8, such as one or more peptides set forth in Table 9 and/or such as one or more peptides set forth in Table 10, or any combination thereof,
one or more peptides derived from SARS-CoV-2, such as one or more SARS-CoV-2 derived peptides set forth Tables 1 to 10 (SEQ ID NOS: 1 to 3522),
one or more peptides set forth in Tables 1 to 10 (SEQ ID NOS: 1 to 3522),
one or more peptides derived from the SARS-CoV-2 Spike (S) protein such as one or more SARS-CoV-2 Spike (S) protein derived peptides set forth in Tables 1 to 10 (SEQ ID NOS: 1 to 3522),
one or more peptides derived from the SARS-CoV-2 Membrane (M) protein such as one or more SARS-CoV-2 Membrane (M) protein derived peptides set forth in Tables 1 to 10 (SEQ ID NOS: 1 to 3522),
one or more peptides derived from the SARS-CoV-2 Nucleocapsid (N) protein such as one or more SARS-CoV-2 Nucleocapsid (N) protein derived peptides set forth in Tables 1 to 10 (SEQ ID NOS: 1 to 3522),
one or more peptides derived from the SARS-CoV-2 Envelope (E) protein such as one or more SARS-CoV-2 Envelope (E) protein derived peptides set forth in Tables 1 to 10 (SEQ ID NOS: 1 to 3522),
one or more peptides derived from the SARS-CoV-2 ORF3a protein such as one or more SARS-CoV-2 ORF3a protein derived peptides set forth in Tables 1 to 10 (SEQ ID NOS: 1 to 3522),
one or more peptides derived from the SARS-CoV-2 ORF6 protein such as one or more SARS-CoV-2 ORF6 protein derived peptides set forth in Tables 1 to 10 (SEQ ID NOS: 1 to 3522),
one or more peptides derived from the SARS-CoV-2 ORF7a protein such as one or more SARS-CoV-2 ORF7a protein derived peptides set forth in Tables 1 to 10 (SEQ ID NOS: 1 to 3522),
one or more peptides derived from the SARS-CoV-2 ORF7b protein such as one or more SARS-CoV-2 ORF7b protein derived peptides set forth in Tables 1 to 10 (SEQ ID NOS: 1 to 3522),
one or more peptides derived from the SARS-CoV-2 ORF8 protein such as one or more SARS-CoV-2 ORF8 protein derived peptides set forth in Tables 1 to 10 (SEQ ID NOS: 1 to 3522),
one or more peptides derived from the SARS-CoV-2 ORF10 protein such as one or more SARS-CoV-2 ORF10 protein derived peptides set forth in Tables 1 to 10 (SEQ ID NOS: 1 to 3522),
one or more peptides derived from the SARS-CoV-2 nsp1 protein such as one or more SARS-CoV-2 nsp1 protein derived peptides set forth in Tables 1 to 10 (SEQ ID NOS: 1 to 3522),
one or more peptides derived from the SARS-CoV-2 nsp2 protein such as one or more SARS-CoV-2 nsp2 protein derived peptides set forth in Tables 1 to 10 (SEQ ID NOS: 1 to 3522),
one or more peptides derived from the SARS-CoV-2 nsp3 protein such as one or more SARS-CoV-2 nsp3 protein derived peptides set forth in Tables 1 to 10 (SEQ ID NOS: 1 to 3522),
one or more peptides derived from the SARS-CoV-2 nsp4 protein such as one or more SARS-CoV-2 nsp4 protein derived peptides set forth in Tables 1 to 10 (SEQ ID NOS: 1 to 3522),
one or more peptides derived from the SARS-CoV-2 nsp5 protein such as one or more SARS-CoV-2 nsp5 protein derived peptides set forth in Tables 1 to 10 (SEQ ID NOS: 1 to 3522),
one or more peptides derived from the SARS-CoV-2 nsp6 protein such as one or more SARS-CoV-2 nsp6 protein derived peptides set forth in Tables 1 to 10 (SEQ ID NOS: 1 to 3522),
one or more peptides derived from the SARS-CoV-2 nsp7 protein such as one or more SARS-CoV-2 nsp7 protein derived peptides set forth in Tables 1 to 10 (SEQ ID NOS: 1 to 3522),
one or more peptides derived from the SARS-CoV-2 nsp8 protein such as one or more SARS-CoV-2 nsp8 protein derived peptides set forth in Tables 1 to 10 (SEQ ID NOS: 1 to 3522),
one or more peptides derived from the SARS-CoV-2 nsp9 protein such as one or more SARS-CoV-2 nsp9 protein derived peptides set forth in Tables 1 to 10 (SEQ ID NOS: 1 to 3522),
one or more peptides derived from the SARS-CoV-2 nsp10 protein such as one or more SARS-CoV-2 nsp10 protein derived peptide set forth in Tables 1 to 10 (SEQ ID NOS: 1 to 3522),
one or more peptides derived from the SARS-CoV-2 nsp12 protein such as one or more SARS-CoV-2 nsp12 protein derived peptides set forth in Tables 1 to 10 (SEQ ID NOS: 1 to 3522),
one or more peptides derived from the SARS-CoV-2 nsp13 protein such as one or more SARS-CoV-2 nsp13 protein derived peptides set forth in Tables 1 to 10 (SEQ ID NOS: 1 to 3522),
one or more peptides derived from the SARS-CoV-2 nsp14 protein such as one or more SARS-CoV-2 nsp14 protein derived peptides set forth in Tables 1 to 10 (SEQ ID NOS: 1 to 3522),
one or more peptides derived from the SARS-CoV-2 nsp15 protein such as one or more SARS-CoV-2 nsp15 protein derived peptides set forth in Tables 1 to 10 (SEQ ID NOS: 1 to 3522) and
one or more peptides derived from the SARS-CoV-2 nsp16 protein such as one or more SARS-CoV-2 nsp16 protein derived peptides set forth in Tables 1 to 10 (SEQ ID NOS: 1 to 3522).

In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from SARS-CoV-2, such as one or more SARS-CoV-2 derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Spike (S) protein such as one or more SARS-CoV-2 Spike (S) protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Membrane (M) protein such as one or more SARS-CoV-2 Membrane (M) protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Nucleocapsid (N) protein such as one or more SARS-CoV-2 Nucleocapsid (N) protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Envelope (E) protein such as one or more SARS-CoV-2 Envelope (E) protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 ORF3a protein such as one or more SARS-CoV-2 ORF3a protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 ORF7a protein such as one or more SARS-CoV-2 ORF7a protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 ORF8 protein such as one or more SARS-CoV-2 ORF8 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp1 protein such as one or more SARS-CoV-2 nsp1 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp2 protein such as one or more SARS-CoV-2 nsp2 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp3 protein such as one or more SARS-CoV-2 nsp3 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp6 protein such as one or more SARS-CoV-2 nsp6 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp9 protein such as one or more SARS-CoV-2 nsp9 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp10 protein such as one or more SARS-CoV-2 nsp10 protein derived peptide set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp12 protein such as one or more SARS-CoV-2 nsp12 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp13 protein such as one or more SARS-CoV-2 nsp13 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp14 protein such as one or more SARS-CoV-2 nsp14 protein derived peptides set forth in Tables 1 to 10 (SEQ ID NOS: 1 to 3522). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp15 protein such as one or more SARS-CoV-2 nsp15 protein derived peptides set forth in Tables 1 to 10 (SEQ ID NOS: 1 to 3522). Any of the above composition embodiments can be combined in any order.

In humans, there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-0) are also encompassed by the present disclosure. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present disclosure is particularly suitable for isolating and/or identifying a population of CD8+ T cells having specificity for the peptide of the present disclosure (in a flow cytometry assay).

The peptides or MHC class I or class II multimer as described herein is particularly suitable for detecting T cells specific for one or more peptides of the present disclosure. The peptide(s) and/or the MHC/multimer complex of the present disclosure is particularly suitable for diagnosing coronavirus infection in a subject. For example, the method comprises obtaining a blood or PBMC sample obtained from the subject with an amount of a least peptide of the present disclosure and detecting at least one T cell displaying a specificity for the peptide. Another diagnostic method of the present disclosure involves the use of a peptide of the present disclosure that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting.

The MHC class I or class II peptide multimers as described herein can also be used as therapeutic agents. The peptide and/or the MHC class I or class II peptide multimers of the present disclosure are suitable for treating or preventing a coronavirus infection in a subject. The MHC Class I or Class II multimers can be administered in soluble form or loaded on nanoparticles.

As used herein, the term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

As used herein, the phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜-150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

As used herein, the term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present disclosure includes, in one embodiment, pre-processed peptides that are attached to the anti-CD40 antibody (or fragment thereof) that directs the peptides against which an enhanced immune response is sought directly to antigen presenting cells.

The present disclosure includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or virus).

The present disclosure provides nucleic acid molecules, specifically polynucleotides, primary constructs and/or mRNA that encode one or more polynucleotides that express one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present disclosure includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof. A variety of well-known vectors can be used to deliver to cells the one or more polynucleotides or the peptides or proteins expressed by the polynucleotides, including but not limited to adenoviral vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalovirus, adenovirus, or SV40), inducible promoters (e.g., a cytokine promoter such as the interleukin-1, tumor necrosis factor-alpha, or interleukin-6 promoter), and tissue specific promoters to express the immunogenic peptides or proteins of the present disclosure.

The immunization may include adenovirus, adeno-associated virus, herpes virus, vaccinia virus, retroviruses, or other viral vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from those sequences set forth in Tables 1 to 10 (SEQ ID NOS: 1 to 3522), or a subsequence, portion, homologue, variant or derivative thereof, gene expression construct. Viral vectors which do not require that the target cell be actively dividing, such as adenoviral and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof into the viral vector, along with another gene which encodes the ligand for a receptor on a specific target cell, for example, such that the vector is now target specific. Viral vectors can be made target specific by attaching, for example, a sugar, a glycolipid, or a protein. Targeting can also be accomplished by using an antibody to target the viral vector. Those of skill in the art will know of, or can readily ascertain without undue experimentation, specific polynucleotide sequences which can be inserted into the viral genome or attached to a viral envelope to allow target specific delivery of the viral vector containing the gene.

Since recombinant viruses are defective, they require assistance in order to produce infectious vector particles. This assistance can be provided, for example, by using helper cell lines that contain plasmids encoding all of the structural genes of the virus under the control of regulatory sequences within the viral genome. These plasmids are missing a nucleotide sequence which enables the packaging mechanism to recognize a polynucleotide transcript for encapsidation. These cell lines produce empty virions, since no genome is packaged. If a viral vector is introduced into such cells in which the packaging signal is intact, but the structural genes are replaced by other genes of interest, the vector can be packaged and vector virion produced.

Viral or non-viral approaches may also be employed for the introduction of one or more therapeutic polynucleotides that express the one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present disclosure, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof. One non-limiting example of a colloidal system for use with the present disclosure is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a coronavirus or interact with the proteins, peptides, and/or gene products of a coronavirus, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant disclosure, polynucleotide expression is directed from any suitable promoter (e.g., the human cytomegalovirus, simian virus 40, actin or adenovirus constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from those sequences set Tables 1 to 10, or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present disclosure contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present disclosure as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 700 to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present disclosure are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif, and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the disclosure sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the disclosure alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a coronavirus infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes of the disclosure, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the disclosure can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavivirus infection or reduces at least one symptom thereof. Peptide and protein T cell epitopes of the disclosure can stimulate the T cell responses that, for example, neutralize infectious agents, kill virus infected cells, blocks infectious agents from entering cells, blocks replication of said infectious agents, and/or protect host cells from infection and destruction.

As used herein, the terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

As used herein, the terms “virus” or “virus particle” are used according to their plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins. In embodiments, the virus is a coronavirus. Non-limiting examples of coronaviruses (CoV) from which T cell epitopes can be identified include, e.g., SARS-CoV (SARS-CoV-1), MERS-CoV, and SARS-CoV-2, but also betacoronaviruses, e.g., HCoV-OC43, HCoVHKU1, HCoV-229E and alphacoronaviuses such as HCoV-NL63, and/or other coronaviruses endemic in humans. The viral genome of coronaviruses encodes at least the following structure proteins, the spike (S), envelope (E), membrane (M), and nucleocapsid (N) proteins. The S glycoprotein is responsible for binding the host receptor via the receptor-binding domain (RBD) in its S1 subunit, as well as the subsequent membrane fusion and viral entry driven by its S2 subunit. Gene sequencing of SARS-CoV-2 showed that this novel coronavirus, a betacoronavirus, is related to the MERS-CoV and the SARS-CoV. SARS-CoV, MERS-CoV, and SARS-CoV-2 belong to the betacoronavirus genus and are highly pathogenic zoonotic viruses. Thus, the present disclosure can be used not only to determine antigenic peptides from the three highly pathogenic betacoronaviruses, but also low-pathogenicity betacoronaviruses, such as, HCoV-OC43, HCoVHKU1, HCoV-NL63 and HCoV-229E, are also endemic in humans. In certain specific embodiments, the coronavirus is SARS-CoV-2, including novel mutants of SARS-CoV-2 that include mutants from five clades (19A, 19B, 20A, 20B, and 20C) according to Nextstrain, in GISAID nomenclature which divides them into seven clades (L, O, V, S, G, GH, and GR), and/or PANGOLIN nomenclature which divides them into six major lineages (A, B, B.1, B.1.1, B.1.177, B.1.1.7). Notable mutations of SARS-CoV-2 include, e.g., D614G, P681H, N501Y, 69-70del, P681H, Y453F, 69-70deltaHV, N501Y, K417N, E484K, N501Y, and E484K.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated; however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the term “modulator” refers to a composition that increases or decreases the level of a target molecule or the function of a target molecule or the physical state of the target of the molecule relative to the absence of the modulator.

As used herein, the term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

As used herein, the terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

As used herein, the term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

As used herein, the terms “subject” or “subject in need thereof” refer to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the terms “disease” or “condition” refer to a state of being or health status of a patient or subject capable of being treated with a compound, pharmaceutical composition, or method provided herein. In embodiments, a patient or subject is human. In embodiments, the disease is coronavirus infection. In certain alternative embodiments, the disease is SARS-CoV-2 infection. In still other embodiments, the disease is COVID-19.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from viral infection. Also, a therapeutic benefit is achieved with the eradication or amelioration of one or more of the physiological symptoms associated with viral infection or the underlying disorder such that an improvement is observed in the patient, notwithstanding that the patient may still be afflicted with the underlying disorder or may still be infected. For prophylactic benefit, the compositions may be administered to a patient at risk of viral infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a coronavirus. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to the recognition of an infection, disease or condition by signs and symptoms. Diagnosing can refer to the determination of whether a subject has an infection or disease. Diagnosis may refer to the determination of the type of disease or condition a subject has or the type of virus the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The peptide(s) or protein(s) of the present disclosure can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

As used herein, the terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the disclosure can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

As used herein, the term “adjuvant” refers to a compound that when administered in conjunction with the compositions provided herein including embodiments thereof, augments the composition's immune response. Generally, adjuvants are non-toxic, have high-purity, are degradable, and are stable. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit andAdjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent. Other adjuvants contemplated for the disclosure are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this disclosure, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the terms “pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the disclosure. One of skill in the art will recognize that other pharmaceutical excipients are useful in the present disclosure.

As used herein, the term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

As used herein, the term “multimerization domain” refers to any type of molecule that is directly or indirectly associated with one or more MHC/peptide monomers. A multimerization domain is a molecule, a complex of molecules, or solid support, to which one or more MHC and/or MHC/peptide monomers can be attached. A multimerization domain can consist of one or more carriers and/or one or more scaffolds and may also contain one or more linkers connecting carrier to scaffold, carrier to carrier, and/or scaffold to scaffold. The multimerization domain may also contain one or more linkers that can be used for attachment of MHC/peptide monomers and/or other molecules to the multimerization domain. In this disclosure, a multimerization domain will in one embodiment refer to a functionalized polymer (e.g., dextran) that is capable of reacting with MHC/peptide monomers, thus covalently attaching the MHC/peptide monomer to the multimerization domain, or that is capable of reacting with scaffold molecules (e.g., streptavidin), thus covalently attaching streptavidin to the multimerization domain; the streptavidin then may bind MHC/peptide monomers. Multimerization domains include IgG, streptavidin, avidin, streptactin, micelles, cells, polymers, dextran, polysaccharides, beads and other types of solid support, and small organic molecules carrying reactive groups or carrying chemical motifs that can bind MHC/peptide monomers and other molecules, such as identified in detail herein elsewhere.

Non-limiting examples of suitable multimerization domain(s) are polysaccharides including dextran molecules, carboxy methyl dextran, dextran polyaldehyde, carboxymethyl dextran lactone, and cyclodextrins, pullulans, schizophyllan, scleroglucan, xanthan, gellan, O-ethylamino guaran, chitins and chitosans including 6-O-carboxymethyl chitin and N-carboxymethyl chitosan, derivatized cellolosics including carboxymethyl cellulose, carboxymethyl hydroxyethyl cellulose, hydroxy-ethyl cellulose, 6-amino-6-deoxy cellulose and O-ethyl-amine cellulose, hydroxylated starch, hydroxypropyl starch, hydroxyethyl starch, carrageenans, alginates, and agarose, synthetic polysaccharides including ficoll and carboxy-methylated ficoll, vinyl polymers including poly (acrylic acid), poly (acryl amides), poly (acrylic esters), poly (2-hydroxy ethyl methacrylate), poly (methyl methacrylate), poly (maleic acid), poly (maleic anhydride), poly (acrylamide), poly (ethyl-co-vinyl acetate), poly (methacrylic acid), poly (vinyl-alcohol), poly (vinyl alcohol-co-vinyl chloroacetate), aminated poly (vinyl alcohol), and co block polymers thereof, poly ethylene glycol (PEG) or polypropylene glycol or poly (ethylene oxide-co-propylene oxides) comprising polymer backbones including linear, comb-shaped or starburst dendrimers, poly amino acids including polylysines, polyglutamic acid, polyurethanes, poly (ethylene imines), pluriol, proteins including peptides, polypeptides, antigen binding peptides, albumins, immunoglobulins, coiled-coil helixes e.g. Fos-Jun or Fos-Jun like or coiled-coiled dimers/trimers/tetramers/pentamers, Streptavidin, Avidin, STREP-TACTIN®, T-cell receptors other protein receptors and virus-like proteins (VLP), and polynucleotides, DNA, RNA, PNA, LNA, oligonucleotides and oligonucleotide dendrimer constructs and small organic molecules including but not limited to steroids, peptides, linear or cyclic structures, aromatic structures, aliphatic structures.

As used herein, the term “dextran” refers to a complex, branched polysaccharide made of many glucose molecules joined into chains of varying lengths. The straight chain consists of α1->6 glycosidic linkages between glucose molecules, while branches begin from α1->3 linkages (and in some cases, α1->2 and α1->4 linkages as well).

The term “label” is used interchangeable with labeling molecule. Label as described herein is an identifiable substance that is detectable in an assay and that can be attached to a molecule creating a labeled molecule. The behavior of the labeled molecule can then be studied. Labels may be organic or inorganic molecules or particles. Labels may be organic or inorganic molecules or particles. Examples of labels include, but are not limited to, polymers, nucleic acids, DNA, RNA, oligonucleotides, peptides, fluorescent labels, phosphorescent labels, enzyme labels, chemiluminescent labels, bioluminescent labels, haptens, antibodies, dyes, nanoparticle labels, elements, metal particles, heavy metal labels, isotope labels, radioisotopes, stable isotopes, chains of isotopes and single atoms, or combination thereof. The labelling compound may suitably be selected from fluorescent labels such as 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, Green fluorescent protein (GFP) and analogues thereof, and conjugates of R-phycoerythrin or allophycoerythrin and e.g. Cy5 or Texas Red, and inorganic fluorescent labels based on semiconductor nanocrystals (like quantum dot and Qdot™ nanocrystals), and time-resolved fluorescent labels based on lanthanides like Eu3+ and Sm3+. In one embodiment a MHC monomer or MHC multimer as defined herein comprises at least one nucleic acid label, such as a nucleotide label, for example an oligonucleotide label. Such nucleic acids labels are disclosed in WO 2015/188839 and WO 2015/185067 (which are hereby incorporated by reference).

The MHC/peptide multimer can comprise one or more labels such as only a singly label. The one or more labels can be directly attached to the MHC/peptide multimer or indirectly to the MHC/peptide multimer such as via one or more marker molecules carrying one or more labels. The one or more labels can be used for combinatorial use of labelling. The one or more labels can result in positive selection of said MHC/peptide multimer or alternatively in negative selection of said MHC/peptide multimer. The one or more labels can comprise one or more covalently attached labels and/or one or more non-covalently attached labels. The one or more labels can be covalently attached to polypeptide a of the MHC monomer, covalently attached to polypeptide b of the MHC monomer, covalently attached to the peptide and/or covalently attached to the one or more multimerization domains. Alternatively, the one or more labels can be non-covalently attached to polypeptide a of the MHC monomer, non-covalently attached to polypeptide b of the MHC monomer, non-covalently attached to the peptide and/or non-covalently attached to the one or more multimerization domains. In another embodiment the one or more labels can be covalently and/or non-covalently attached to the multimerization domain via a molecule, wherein the molecule e.g., can be selected from the group consisting of an antibody, an aptamer, a protein, a sugar residue and a nucleotide such as DNA. In a specific embodiment the one or more labels are attached to the MHC/peptide multimer via a streptavidin-biotin linkage.

In a particular embodiment the label is an oligonucleotide, such as a nucleic acid molecule comprises or consists of DNA, RNA, and/or artificial nucleotides such as PLA or LNA. In one embodiment the nucleic acid label comprises one or more of the following components: a barcode region, 5′ first primer region (forward), 3′ second primer region (reverse), random nucleotide region, connector molecule, stability-increasing components, short nucleotide linkers in between any of the above-mentioned components, adaptors for sequencing and annealing region. Preferably the nucleic acid label comprises at least a barcode region; where the barcode region comprises a sequence of consecutive nucleic acids. In one embodiment the nucleic acid label comprises or consists of DNA, RNA, artificial nucleic acids and/or Xeno nucleic acid (XNA). In one embodiment at least two different labels are attached to a MHC monomer or a MHC multimer, such as at least two different labels such as one fluorescent label and one nucleic acid label. The MHC/peptide multimer can comprise one or more fluorescent labels selected from the group of fluorescein isothiocyanate, rhodamine, phycoerythrin, phycocyanin, allophycocyanin, o-phthaldehyde, fluorescamine, 2-4′-maleimidylanilino)naphthalene-6-sulfonic acid sodium salt, 5-((((2-iodoacetyl)amino)ethyl)amino), naphthalene-1-sulfonic acid, Pyrene-1-butanoic acid, AlexaFluor 350 (7-amino-6-sulfonic acid-4-methyl coumarin-3-acetic acid, AMCA (7-amino-4-methyl coumarin-3-acetic acid), 7-hydroxy-4-methyl coumarin-3-acetic acid, Marina Blue (6,8-difluoro-7-hydroxy-4-methyl coumarin-3-acetic acid), 7-dimethylamino-coumarin-4-acetic acid, Fluorescamin-N-butyl amine adduct, 7-hydroxy-coumarine-3-carboxylic acid, CascadeBlue (pyrene-trisulphonic acid acetyl azide), Cascade Yellow, Pacific Blue (6,8 difluoro-7-hydroxy coumarin-3-carboxylic acid), 7-diethylamino-coumarin-3-carboxylic acid, N-(((4-azidobenzoyl)amino)ethyl)-4-amino-3,6-disulfo-1,8-naphthalimide, dipotassium salt), Alexa Fluor 430, 3-perylenedodecanoic acid, 8-hydroxypyrene-1,3,6-trisulfonic acid trisodium salt, 12-(N-(7-nitrobenz-2-oxa-1,3-diazol-4-yl)amino)dodecanoic acid, N,N′-dimethyl-N-(iodoacetyl)-N′-(7-nitrobenz-2-oxa-1,3-diazol-4-yl)ethylenediamine, Oregon Green 488 (difluoro carboxy fluorescein), 5-iodoacetamidofluorescein, propidium iodide-DNA adduct, Carboxy fluorescein, fluor dyes, Pacific Blue™, Pacific Orange™, Cascade Yellow™ AlexaFluor®(AF), AF®350, AF405, AF430, AF488, AF500, AF514, AF532, AF546, AF555, AF568, AF594, AF610, AF633, AF635, AF647, AF680, AF700, AF710, AF750, AF800, Quantum Dotbased dyes, QDot® Nanocrystals (Invitrogen, MolecularProbs), Qdot®525, Qdot®565, Qdot®585, Qdot®605, Qdot®655, Qdot®705, Qdot®800, DyLight™ Dyes (Pierce) (DL); DL549, DL649, DL680, DL800, Fluorescein (Flu) or any derivate of that, such as FITC, Cy-Dyes, Cy2, Cy3, Cy3.5, Cy5, Cy5.5, Cy7, 7-AAD, TO-Pro-3, fluorescent Proteins, R-Phycoerythrin (RPE), Phycobili Proteins, Allophycocyani (APC), PerCp, B-Phycoerythrin, C-Phycocyanin, APC, fluorescent proteins, Green fluorescent proteins; GFP and GFP derivated mutant proteins; BFP, CFP, YFP, DsRed, DSred-2, T1, Dimer2, mRFP1, MBanana, mOrange, dTomato, tdTomato, mTangerine, mStrawberry, mCherry, Tandem dyes, RPE-Cy5, RPE-Cy5.5, RPE-Cy7, RPE-AlexaFluor® tandem conjugates; RPE-Alexa610, RPE-TxRed, Tandem dyes with APC, APC-Aleca600, APC-Alexa610, APC-Alexa750, APC-Cy5, APC-Cy5.5, multi fluorochrome assemblies, FRET-based dyes (Fluorescence resonance energy transfer), ionophors; ion chelating fluorescent props, props that change wavelength when binding a specific ion, such as Calcium, props that change intensity when binding to a specific ion, such as Calcium, Calcium dyes, Indo-1-Ca2+, Indo-2-Ca2+.

The one or more labels can in a specific embodiment be selected from the group consisting of APC, APC-Cy7, ABC-H7, APC-R700, Alexa Flours™ 488, Alexa Flours™555, Alexa Flours™647, Alexa Flours™700, AmCyan, BB151, BB700, BUV395, BUV496, BUV563, BUV615, BUV661, BUV737, BUV805, BV421, BV480, BV510, BV605, BV711, BV750, BV786, FITC, PE, PE-CF594, PE-Cy5, PE-CY5.5, PE-cy7, Pasific Blue, PERCP, pPerCp-Cy5.5, PE, R718, RY586, V450 and V500 (wherein in BV means Brilliant violet, wherein BUV means Brilliant ultra violet and PE means R-Phycoerythrin). In another embodiment the one or more labels can be selected from the group consisting of cFluor®B515, cFluor®B532, cFluor®B548, cFluor®B675, cFluor®B690, cFluor®BY575, cFluor®BY610, cFluor®BY667, cFluor®BY710, cFluor®BY750, cFluor®BY781, cFluor®B250, cFluor®R659, cFluor®R668, cFluor®R685, cFluor®R720, cFluor®R780, cFluor®R840, cFluor®v420, cFluor®v547, cFluor®v450, cFluor®v610 and cFluor®YG610. The MHC/peptide multimer can comprise one or more labels which are one or more chemiluminescent labels such as one or more labels selected from the group consisting of luminol, isoluminol, theromatic acridinium ester, imidazole, acridinium salt, and oxalate ester. The MHC/peptide multimer can comprise one or more labels which are one or more bioluminescent labels such as one or more labels selected from the group consisting of luciferin, luciferase, and aequorin. The MHC/peptide multimer can comprise one or more labels which are one or more enzyme labels, such as one or more enzyme labels selected from the group peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholinesterase. The MHC/peptide multimer can comprise one or more labels which are one or more chromophore labels. In another embodiment, the MHC/peptide multimer comprises one or more labels which are one or more metal labels. In yet another embodiment the MHC/peptide multimer comprises one or more labels which are one or more radioactive labels such as one or more labels selected from the group consisting of a radionuclide, an isotope, a label comprising α rays, a label comprising β rays or a label comprising γ rays. Any of the above embodiments regarding labels can be combined in any order.

### Example 1: SARS-CoV-2 Human CD4 and CD8 T-Cell Epitope Data

The present invention provides SARS-CoV-2 human CD4 and CD8 T-cell epitope data from 870 SARS-CoV-2 infected donors and 327 unexposed subjects using a variety of screening designs and assay methodologies. Epitopes have been identified from throughout the SARS-CoV-2 proteome, with a significant correlation between the number of epitopes defined and size of the antigen of provenance. Further analysis revealed discrete immunodominant regions and certain epitopes that are more prevalently recognized. 75 different HLA alleles have been identified as MHC restriction elements, and several studies addressed pre-existing reactivity and sequence conservation with endemic coronaviruses and other viruses. This remarkable breadth of epitope repertoire has implications for immune escape by SARS-CoV-2 mutants and variants.

TABLE 1
Listing of unique SARS-COV-2-derived CD4 and CD8 epitopes.

| SEQ | | | | | |
|---|---|---|---|---|---|
| ID | | | | | |
| NO: | Description | Ag | Dominant | Restriction | Allele(s) |
| 1 | SEETGTLIVNSVLLF | E | | CD4 | DQB1*03:01 |
| 2 | FLLVTLAILTALRLC | E | | CD4 | |
| 3 | LAILTALRLCAYCCN | E | | CD4 | DRB1*01:01, DRB1*11:01 |
| 4 | FYVYSRVKNLNSSRV | E | Yes | CD4 | |
| 5 | EELKKLLEQWNLVIG | M | | CD4 | DQB1*05:01, DQB1*05:02 |
| 6 | LLEQWNLVIGFLFLT | M | | CD4 | DQB1*02:01, DQB1*05:03 |
| 7 | NLVIGFLFLTWICLL | M | | CD4 | DQB1*05:01, DRB1*01:02 |
| 8 | FLFLTWICLLQFAYA | M | | CD4 | DQB1*05:01, DQB1*05:03, |
| | | | | | DRB1*01:02 |
| 9 | WICLLQFAYANRNRF | M | | CD4 | DQB1*05:03, DRB1*03:01, |
| | | | | | DRB1*14:01 |
| 10 | QFAYANRNRFLYIIK | M | Yes | CD4 | DRB1*14:01 |
| 11 | NRNRFLYIIKLIFLW | M | | CD4 | DQB1*02:01, DQB1*05:03, |
| | | | | | DRB1*03:01, DRB1*12:01 |
| 12 | LYIIKLIFLWLLWPV | M | | CD4 | DQB1*02:01, DRB1*12:01 |
| 13 | LIFLWLLWPVTLACF | M | | CD4 | DQB1*05:01, DQB1*05:03, |
| | | | | | DRB1*01:02 |
| 14 | LLWPVTLACFVLAAV | M | | CD4 | DQB1*05:01, DQB1*05:03, |
| | | | | | DQB1*06:03 |
| 15 | VLAAVYRINWITGGI | M | Yes | CD4 | DQB1*05:01, DQB1*05:03, |
| | | | | | DRB1*12:02, DRB1*14:01 |
| 16 | YRINWITGGIAIAMA | M | Yes | CD4 | DQB1*02:02, DQB1*03:01, |
| | | | | | DQB1*05:01, DQB1*05:02, |
| | | | | | DQB1*05:03, DQB1*06:02, |
| | | | | | DQB1*06:03, DRB1*07:01, |
| | | | | | DRB1*10:01, DRB1*12:01, |
| | | | | | DRB1*13:01, DRB1*14:01, |
| | | | | | DRB1*15:01, DRB1*16:01 |
| 17 | ITGGIAIAMACLVGL | M | | CD4 | DQB1*03:01 |
| 18 | AIAMACLVGLMWLSY | M | | CD4 | |
| 19 | CLVGLMWLSYFIASF | M | Yes | CD4 | DQB1*05:01, DQB1*05:03, |
| | | | | | DRB1*12:01, DRB1*12:02 |
| 20 | MWLSYFIASFRLFAR | M | Yes | CD4 | DQB1*02:01, DQB1*05:01, |
| | | | | | DQB1*05:02, DQB1*05:03, |
| | | | | | DRB1*03:01, DRB1*14:01, |
| | | | | | DRB1*14:06, DRB1*16:01, |
| | | | | | DRB1*16:02 |
| 21 | FIASFRLFARTRSMW | M | | CD4 | DQB1*05:03, DRB1*03:01, |
| | | | | | DRB1*14:01 |
| 22 | RLFARTRSMWSFNPE | M | | CD4 | DQB1*05:03, DRB1*14:01 |
| 23 | TRSMWSFNPETNILL | M | | CD4 | DQB1*05:01, DQB1*05:02, |
| | | | | | DQB1*05:03, DRB1*01:02 |
| 24 | SFNPETNILLNVPLH | M | | CD4 | DRB1*01:02 |
| 25 | TNILLNVPLHGTILT | M | Yes | CD4 | DRB1*10:01, DRB1*12:02, |
| | | | | | DRB1*14:01, DRB1*14:06, |
| | | | | | DRB1*15:01, DRB1*16:02 |
| 26 | NVPLHGTILTRPLLE | M | | CD4 | |
| 27 | GTILTRPLLESELVI | M | | CD4 | DRB1*13:01 |
| 28 | RPLLESELVIGAVIL | M | | CD4 | DQB1*02:02 |
| 29 | SELVIGAVILRGHLR | M | Yes | CD4 | DQB1*06:02, DQB1*06:03, |
| | | | | | DRB1*01:02, DRB1*13:01, |
| | | | | | DRB1*14:01, DRB1*15:01, |
| | | | | | DRB1*16:01 |
| 30 | GAVILRGHLRIAGHH | M | Yes | CD4 | |
| | LGR | | | | |
| 31 | GAVILRGHLRIAGHH | M | | CD4 | DRB1*13:01, DRB1*15:01 |
| 32 | LRGHLRIAGHHLGRC | M | Yes | CD4 | DRB1*11:01, DRB1*11:04 |
| 33 | RGHLRIAGHHLGRCD | M | Yes | CD4 | DRB1*07:01, DRB1*13:01, |
| | | | | | DRB1*14:01, DRB1*15:01 |
| 34 | LRIAGHHLGRCDIKD | M | | CD4 | |
| 35 | IAGHHLGRCDIKDLP | M | Yes | CD4 | |
| 36 | LGRCDIKDLPKEITV | M | Yes | CD4 | |
| 37 | IKDLPKEITVATSRT | M | Yes | CD4 | |
| 38 | KEITVATSRTLSYYK | M | Yes | CD4 | DQB1*06:03, DRB1*03:01, |
| | | | | | DRB1*07:01, DRB1*14:01, |
| | | | | | DRB1*14:06, DRB1*15:01, |
| | | | | | DRB1*16:02 |
| 39 | ATSRTLSYYKLGASQ | M | | CD4 | DQB1*05:01, DQB1*05:03, |
| | | | | | DRB1*16:01 |
| 40 | TSRTLSYYKLGASQR | M | Yes | CD4 | |
| | VA | | | | |
| 41 | SRTLSYYKLGASQRV | M | Yes | CD4 | DRB5*01:01, DRB5*01:02 |
| 42 | LSYYKLGASQRVAGD | M | Yes | CD4 | DQB1*03:01, DQB1*06:02, |
| | | | | | DQB1*06:03, DRB1*01:01, |
| | | | | | DRB1*07:01, DRB1*14:06, |
| | | | | | DRB1*15:01, DRB1*16:01, |
| | | | | | DRB1*16:02 |
| 43 | SYYKLGASQRVAGDS | M | Yes | CD4 | DQB1*03:01, DRB1*01:01, |
| | | | | | DRB1*07:01 |
| 44 | LGASQRVAGDSGFAA | M | Yes | CD4 | |
| 45 | RVAGDSGFAAYSRYR | M | | CD4 | |
| 46 | SGFAAYSRYRIGNYK | M | Yes | CD4 | DRB1*15:01 |
| 47 | YSRYRIGNYKLNTDH | M | | CD4 | |
| 48 | IGNYKLNTDHSSSSD | M | | CD4 | DRB1*03:01 |
| 49 | LNTDHSSSSDNIALL | M | | CD4 | |
| 50 | PQNQRNAPRITFGGP | N | | CD4 | |
| 51 | NAPRITFGGPSDSTG | N | | CD4 | DQB1*03:01 |
| 52 | TFGGPSDSTGSNQNG | N | | CD4 | DQB1*03:01 |
| 53 | ASWFTALTQHGKEDL | N | Yes | CD4 | |
| 54 | SWFTALTQHGKEDLK | N | Yes | CD4 | |
| 55 | LTQHGKEDLKFPRGQ | N | | CD4 | |
| 56 | FPRGQGVPINTNSSP | N | | CD4 | |
| 57 | GVPINTNSSPDDQIG | N | | CD4 | |
| 58 | DDQIGYYRRATRRIR | N | Yes | CD4 | DRB1*01:03, DRB1*07:01, |
| | | | | | DRB1*13:01, DRB1*14:01, |
| | | | | | DRB1*14:06, DRB1*15:01, |
| | | | | | DRB1*16:02 |
| 59 | IGYYRRATRRIRGGD | N | Yes | CD4 | |
| 60 | YYRRATRRIRGGDGK | N | Yes | CD4 | DRB1*13:01, DRB1*14:06 |
| 61 | TRRIRGGDGKMKDLS | N | | CD4 | |
| 62 | GGDGKMKDLSPRWYF | N | | CD4 | |
| 63 | MKDLSPRWYFYYLGT | N | | CD4 | |
| | GPEAG | | | | |
| 64 | MKDLSPRWYFYYLGT | N | Yes | CD4 | |
| 65 | PRWYFYYLGTGPEAG | N | | CD4 | DQB1*02:01, DQB1*05:03 |
| 66 | RWYFYYLGTGPEAGL | N | Yes | CD4 | |
| 67 | LPYGANKDGIIWVAT | N | | CD4 | |
| 68 | NKDGIIWVATEGALN | N | Yes | CD4 | DQB1*02:01, DQB1*02:02, |
| | | | | | DQB1*03:02, DQB1*05:03, |
| | | | | | DRB1*04:04, DRB1*07:01 |
| 69 | KDGIIWVATEGALNT | N | Yes | CD4 | |
| 70 | IWVATEGALNTPKDH | N | | CD4 | |
| 71 | EGALNTPKDHIGTRN | N | | CD4 | |
| 72 | AIVLQLPQGTTLPKG | N | Yes | CD4 | |
| 73 | AGNGGDAALALLLLD | N | Yes | CD4 | DQB1*03:01, DQB1*06:02 |
| 74 | DAALALLLLDRLNQL | N | Yes | CD4 | DQB1*05:01, DQB1*05:03, |
| | | | | | DQB1*06:02, DRB1*03:01, |
| | | | | | DRB1*11:01, DRB1*12:01, |
| | | | | | DRB1*14:01, DRB1*15:01 |
| 75 | LLLLDRLNQLESKMS | N | Yes | CD4 | DQB1*05:01, DRB1*03:01, |
| | | | | | DRB1*11:01, DRB1*12:01, |
| | | | | | DRB1*14:01, DRB1*15:01 |
| 76 | GKGQQQQGQTVTKKS | N | | CD4 | |
| 77 | AAEASKKPRQKRTAT | N | Yes | CD4 | |
| 78 | KKPRQKRTATKAYNV | N | Yes | CD4 | |
| 79 | KPRQKRTATKAYNVT | N | | CD4 | DPB1*14:01 |
| 80 | KRTATKAYNVTQAFG | N | Yes | CD4 | DQB1*06:02, DQB1*06:03 |
| 81 | KAYNVTQAFGRRGPE | N | Yes | CD4 | |
| 82 | TQAFGRRGPEQTQGN | N | | CD4 | |
| 83 | RRGPEQTQGNFGDQE | N | | CD4 | |
| 84 | FGDQELIRQGTDYKH | N | | CD4 | |
| 85 | LIRQGTDYKHWPQIA | N | Yes | CD4 | |
| 86 | TDYKHWPQIAQFAPS | N | | CD4 | |
| 87 | YKHWPQIAQFAPSAS | N | Yes | CD4 | |
| 88 | WPQIAQFAPSASAFF | N | Yes | CD4 | DQB1*02:02, DQB1*05:03, |
| | | | | | DRB1*04:04, DRB1*07:01, |
| | | | | | DRB1*15:01 |
| 89 | QFAPSASAFFGMSRI | N | | CD4 | DRB1*07:01 |
| 90 | ASAFFGMSRIGMEVT | N | Yes | CD4 | DQB1*05:03, DRB1*01:02, |
| | | | | | DRB1*14:01 |
| 91 | AFFGMSRIGMEVTPS | N | | CD4 | |
| 92 | GMEVTPSGTWLTYTG | N | Yes | CD4 | |
| | AIKLD | | | | |
| 93 | GMEVTPSGTWLTYTG | N | | CD4 | |
| 94 | PSGTWLTYTGAIKLD | N | Yes | CD4 | DQB1*06:03, DRB1*01:02, |
| | | | | | DRB1*01:03, DRB1*07:01, |
| | | | | | DRB1*15:01 |
| 95 | GTWLTYTGAIKLDDK | N | Yes | CD4 | |
| 96 | TWLTYTGAIKLDDKD | N | | CD4 | |
| | PNF | | | | |
| 97 | LTYTGAIKLDDKDPN | N | | CD4 | DRB1*07:01 |
| 98 | AIKLDDKDPNFKDQV | N | | CD4 | DRB1*03:01 |
| 99 | PNFKDQVILLNKHID | N | | CD4 | |
| | AYK | | | | |
| 100 | FKDQVILLNKHIDAY | N | Yes | CD4 | DRB1*03:01, DRB1*14:01, |
| | | | | | DRB1*14:06, DRB1*15:01, |
| | | | | | DRB1*16:02 |
| 101 | ILLNKHIDAYKTFPP | N | Yes | CD4 | DRB1*14:06, DRB1*15:01 |
| 102 | HIDAYKTFPPTEPKK | N | | CD4 | |
| 103 | QKKQQTVTLLPAADL | N | | CD4 | DRB1*01:02 |
| 104 | KQQTVILLPAADLDD | N | | CD4 | |
| | F | | | | |
| 105 | TVTLLPAADLDDFSK | N | | CD4 | DQB1*05:03, DRB1*01:02 |
| 106 | AADLDDFSKQLQQSM | N | | CD4 | |
| 107 | PLNSIIKTIQPRVEK | NSP2 | | CD4 | DRB1*01:01, DRB1*07:01 |
| 108 | EEIAIILASFSASTS | NSP2 | | CD4 | DQB1*02:02, DQB1*03:02, |
| | | | | | DRB1*04:10, DRB1*07:01 |
| 109 | SPLYAFASEAARVVR | NSP2 | | CD4 | DQB1*02:02, DRB1*07:01 |
| 110 | AITILDGISQYSLRL | NSP2 | | CD4 | |
| 111 | QTFFKLVNKFLALCA | NSP2 | | CD4 | DQB1*05:01, DRB1*01:03, |
| | | | | | DRB1*07:01 |
| 112 | GETFVTHSKGLYRKC | NSP2 | | CD4 | DRB1*07:01 |
| 113 | ADAVIKTLQPVSELL | NSP3 | | CD4 | DRB1*14:01 |
| 114 | ESDDYIATNGPLKVG | NSP3 | | CD4 | |
| 115 | IATNGPLKVGGSCVL | NSP3 | | CD4 | DRB1*13:02 |
| 116 | SGHNLAKHCLHVVGP | NSP3 | | CD4 | |
| 117 | NLYDKLVSSFLEMKS | NSP3 | | CD4 | DQB1*05:02, DRB1*14:01 |
| 118 | ENLLLYIDINGNLHP | NSP3 | | CD4 | |
| 119 | KSAFYILPSIISNEK | NSP3 | | CD4 | |
| 120 | RFYFYTSKTTVASLI | NSP3 | | CD4 | DQB1*06:01, DRB1*14:01, |
| | | | | | DRB1*15:02 |
| 121 | EAARYMRSLKVPATV | NSP3 | | CD4 | DRB1*07:01 |
| 122 | LPNDDTLRVEAFEYY | NSP3 | | CD4 | DQB1*03:02 |
| 123 | TLRVEAFEYYHTTDP | NSP3 | | CD4 | DQB1*05:03 |
| 124 | HTTDPSFLGRYMSAL | NSP3 | | CD4 | |
| 125 | SFLGRYMSALNHTKK | NSP3 | | CD4 | DRB1*04:04 |
| 126 | YMSALNHTKKWKYPQ | NSP3 | | CD4 | |
| 127 | NHTKKWKYPQVNGLT | NSP3 | | CD4 | |
| 128 | ESPFVMMSAPPAQYE | NSP3 | | CD4 | |
| 129 | YCIDGALLTKSSEYK | NSP3 | | CD4 | DRB1*15:02 |
| 130 | DNFKFVCDNIKFADD | NSP3 | | CD4 | DQB1*05:01 |
| 131 | LNQLTGYKKPASREL | NSP3 | | CD4 | |
| 132 | GYKKPASRELKVTFF | NSP3 | | CD4 | |
| 133 | ASRELKVTFFPDLNG | NSP3 | | CD4 | DQB1*05:03 |
| 134 | KVTFFPDLNGDVVAI | NSP3 | Yes | CD4 | DQB1*02:02, DQB1*05:01, |
| | | | | | DQB1*05:03 |
| 135 | PDLNGDVVAIDYKHY | NSP3 | | CD4 | |
| 136 | TPSFKKGAKLLHKPI | NSP3 | | CD4 | |
| 137 | VWHVNNATNKATYKP | NSP3 | | CD4 | |
| 138 | MAAYVDNSSLTIKKP | NSP3 | | CD4 | DQB1*05:03 |
| 139 | NELSRVLGLKTLATH | NSP3 | | CD4 | DRB1*07:01 |
| 140 | TFTRSTNSRIKASMP | NSP3 | | CD4 | DRB1*14:01 |
| 141 | TNSRIKASMPTTIAK | NSP3 | | CD4 | DRB1*04:04, DRB1*07:01 |
| 142 | NTVKSVGKFCLEASF | NSP3 | | CD4 | |
| 143 | LEASFNYLKSPNFSK | NSP3 | | CD4 | DRB1*04:10, DRB1*07:01 |
| 144 | PNFSKLINIIIWFLL | NSP3 | | CD4 | DRB1*07:01, DRB1*15:01 |
| 145 | GSLIYSTAALGVLMS | NSP3 | | CD4 | DQB1*03:01, DRB1*01:01, |
| | | | | | DRB1*11:01 |
| 146 | ISSFKWDLTAFGLVA | NSP3 | | CD4 | DQB1*02:02, DRB1*07:01 |
| 147 | WDLTAFGLVAEWFLA | NSP3 | | CD4 | DQB1*05:03 |
| 148 | FGLVAEWFLAYILFT | NSP3 | | CD4 | DQB1*05:03, DRB1*12:01 |
| 149 | FDAYVNTFSSTFNVP | NSP3 | | CD4 | DQB1*02:02, DRB1*07:01 |
| 150 | SHNIALIWNVKDFMS | NSP3 | | CD4 | DQB1*02:02, DQB1*03:02, |
| | | | | | DQB1*04:02, DRB1*04:10 |
| 151 | KGGKIVNNWLKQLIK | NSP4 | | CD4 | DRB1*13:01 |
| 152 | LFVAAIFYLITPVHV | NSP4 | | CD4 | DQB1*02:02, DQB1*05:01, |
| 153 | AVITREVGFVVPGLP | NSP4 | | CD4 | DRB1*01:03, DRB1*07:01 |
| 154 | VPGLPGTILRTINGD | NSP4 | | CD4 | |
| 155 | FLHFLPRVFSAVGNI | NSP4 | | CD4 | DRB1*15:01 |
| 156 | DTRYVLMDGSIIQFP | NSP4 | | CD4 | DQB1*03:02, DQB1*05:01, |
| | | | | | DRB1*04:04, DRB1*10:01 |
| 157 | SIVAGGIVAIVVTCL | NSP4 | | CD4 | DQB1*03:02, DQB1*04:02 |
| 158 | FGEYSHVVAFNTLLF | NSP4 | | CD4 | DQB1*02:02, DQB1*03:02, |
| | | | | | DRB1*04:10, DRB1*07:01 |
| 159 | NTLLFLMSFTVLCLT | NSP4 | | CD4 | |
| 160 | PVYSFLPGVYSVIYL | NSP4 | | CD4 | DRB1*15:01 |
| 161 | YLTFYLTNDVSFLAH | NSP4 | | CD4 | DQB1*05:02, DRB1*14:01, |
| | | | | | DRB1*15:01, DRB1*16:01 |
| 162 | SFLAHIQWMVMFTPL | NSP4 | | CD4 | DQB1*06:02, DRB1*15:01 |
| 163 | IQWMVMFTPLVPFWI | NSP4 | | CD4 | DQB1*02:02, DRB1*04:04, |
| | | | | | DRB1*07:01 |
| 164 | MFTPLVPFWITIAYI | NSP4 | | CD4 | DRB1*15:01 |
| 165 | TIAYIICISTKHFYW | NSP4 | | CD4 | DRB1*01:03, DRB1*07:01 |
| 166 | KHFYWFFSNYLKRRV | NSP4 | Yes | CD4 | DQB1*02:02, DQB1*04:02, |
| | | | | | DRB1*04:10, DRB1*07:01, |
| | | | | | DRB1*09:01, DRB1*15:01 |
| 167 | CTFLLNKEMYLKLRS | NSP4 | | CD4 | DQB1*05:01, DRB1*01:01, |
| | | | | | DRB1*11:01 |
| 168 | LTQYNRYLALYNKYK | NSP4 | | CD4 | DRB1*15:01 |
| 169 | RYLALYNKYKYFSGA | NSP4 | | CD4 | DRB1*15:01 |
| 170 | YREAACCHLAKALND | NSP4 | | CD4 | |
| 171 | CCHLAKALNDFSNSG | NSP4 | | CD4 | |
| 172 | FSNSGSDVLYQPPQT | NSP4 | | CD4 | DQB1*06:02 |
| 173 | SDVLYQPPQTSITSA | NSP4 | | CD4 | |
| 174 | NHNFLVQAGNVQLRV | NSP5 | | CD4 | DQB1*03:01, DQB1*06:02, |
| | | | | | DRB1*01:01, DRB1*07:01, |
| | | | | | DRB1*08:03, DRB1*15:01 |
| 175 | QNCVLKLKVDTANPK | NSP5 | | CD4 | DRB1*04:10 |
| 176 | LLVLVQSTQWSLFFF | NSP6 | | CD4 | DQB1*02:02, DRB1*07:01 |
| 177 | SLFFFLYENAFLPFA | NSP6 | | CD4 | DQB1*05:01, DRB1*01:01, |
| | | | | | DRB1*15:01 |
| 178 | LCLFLLPSLATVAYF | NSP6 | | CD4 | DQB1*05:01, DQB1*06:02, |
| | | | | | DRB1*01:01, DRB1*15:01 |
| 179 | TLVYKVYYGNALDQA | NSP6 | | CD4 | DQB1*05:01, DRB1*01:01, |
| | | | | | DRB1*15:01 |
| 180 | NRYFRLTLGVYDYLV | NSP6 | Yes | CD4 | DQB1*02:02, DQB1*05:01, |
| | | | | | DQB1*06:02, DRB1*01:01, |
| | | | | | DRB1*01:03, DRB1*07:01, |
| | | | | | DRB1*08:03, DRB1*15:01 |
| 181 | DAFKLNIKLLGVGGK | NSP6 | | CD4 | DRB1*01:01, DRB1*11:01 |
| 182 | RVESSSKLWAQCVQL | NSP7 | | CD4 | |
| 183 | SKLWAQCVQLHNDIL | NSP7 | | CD4 | |
| 184 | VLKKLKKSLNVAKSE | NSP8 | | CD4 | DRB1*08:03, DRB1*12:02 |
| 185 | LIVTALRANSAVKLQ | NSP8 | | CD4 | DQB1*06:03, DRB1*07:01, |
| | | | | | DRB1*13:01 |
| 186 | VLSFCAFAVDAAKAY | NSP10 | | CD4 | DQB1*02:02, DQB1*05:01, |
| | | | | | DRB1*01:03, DRB1*07:01 |
| 187 | PDILRVYANLGERVR | NSP12 | | CD4 | DQB1*06:01, DRB1*08:03, |
| | | | | | DRB1*12:02 |
| 188 | SLLMPILTLTRALTA | NSP12 | | CD4 | DQB1*06:02, DRB1*08:03, |
| | | | | | DRB1*15:01 |
| 189 | HCANFNVLFSTVFPP | NSP12 | | CD4 | DQB1*02:02, DQB1*06:02, |
| | | | | | DRB1*07:01 |
| 190 | NVLFSTVFPPTSFGP | NSP12 | | CD4 | DQB1*02:02 |
| 191 | QDALFAYTKRNVIPT | NSP12 | | CD4 | |
| 192 | KLLKSIAATRGATVV | NSP12 | | CD4 | DQB1*03:03, DRB1*04:04, |
| | | | | | DRB1*07:01, DRB1*10:01 |
| 193 | IAATRGATVVIGTSK | NSP12 | | CD4 | DQB1*03:03, DRB1*07:01 |
| 194 | YPKCDRAMPNMLRIM | NSP12 | | CD4 | |
| 195 | RAMPNMLRIMASLVL | NSP12 | | CD4 | DQB1*06:02, DRB1*15:01 |
| 196 | SHRFYRLANECAQVL | NSP12 | | CD4 | DRB1*01:01, DRB1*08:01 |
| 197 | SEMVMCGGSLYVKPG | NSP12 | | CD4 | |
| 198 | FNICQAVTANVNALL | NSP12 | | CD4 | DQB1*03:01, DRB1*14:01 |
| 199 | AVTANVNALLSTDGN | NSP12 | | CD4 | |
| 200 | EFYAYLRKHFSMMIL | NSP12 | | CD4 | DQB1*04:02, DRB1*07:01, |
| | | | | | DRB1*09:01, DRB1*13:01 |
| 201 | LRKHFSMMILSDDAV | NSP12 | | CD4 | DRB1*01:03, DRB1*11:02, |
| | | | | | DRB1*13:03 |
| 202 | GLVASIKNFKSVLYY | NSP12 | | CD4 | DQB1*06:01, DRB1*08:03, |
| | | | | | DRB1*12:02 |
| 203 | KTDGTLMIERFVSLA | NSP12 | | CD4 | |
| 204 | LMIERFVSLAIDAYP | NSP12 | Yes | CD4 | DQB1*02:02, DQB1*03:01, |
| | | | | | DQB1*03:02, DQB1*04:02, |
| | | | | | DQB1*05:01, DQB1*06:01, |
| | | | | | DQB1*06:04, DRB1*01:01, |
| | | | | | DRB1*01:03, DRB1*04:04, |
| | | | | | DRB1*07:01, DRB1*08:03, |
| | | | | | DRB1*10:01, DRB1*12:02, |
| | | | | | DRB1*13:02, DRB1*15:01 |
| 205 | FVSLAIDAYPLTKHP | NSP12 | | CD4 | DQB1*02:02, DQB1*03:02, |
| | | | | | DQB1*06:04, DRB1*15:01 |
| 206 | IDAYPLTKHPNQEYA | NSP12 | | CD4 | |
| 207 | DVFHLYLQYIRKLHD | NSP12 | | CD4 | DRB1*04:04, DRB1*13:02 |
| 208 | TSHKLVLSVNPYVCN | NSP13 | | CD4 | DQB1*06:03, DRB1*07:01, |
| | | | | | DRB1*13:01 |
| 209 | ELHLSWEVGKPRPPL | NSP13 | | CD4 | |
| 210 | PRPPLNRNYVFTGYR | NSP13 | | CD4 | |
| 211 | FTGYRVTKNSKVQIG | NSP13 | | CD4 | DRB1*07:01 |
| 212 | VTKNSKVQIGEYTFE | NSP13 | | CD4 | |
| 213 | VNARLRAKHYVYIGD | NSP13 | | CD4 | |
| 214 | ISPYNSQNAVASKIL | NSP13 | | CD4 | DQB1*06:02, DRB1*01:01 |
| 215 | NVNRFNVAITRAKVG | NSP13 | | CD4 | DQB1*03:01, DQB1*06:02, |
| | | | | | DRB1*08:03, DRB1*15:01 |
| 216 | NMFITREEAIRHVRA | NSP14 | | CD4 | DRB1*01:01, DRB1*11:01 |
| 217 | REEAIRHVRAWIGFD | NSP14 | | CD4 | DRB1*01:03, DRB1*07:01 |
| 218 | PLMYKGLPWNVVRIK | NSP14 | | CD4 | DQB1*03:01, DQB1*06:01, |
| | | | | | DRB1*08:03, DRB1*12:02 |
| 219 | LDDFVEIIKSQDLSV | NSP15 | Yes | CD4 | |
| 220 | EIIKSQDLSVVSKVV | NSP15 | | CD4 | DRB1*04:04 |
| 221 | TQLCQYLNTLTLAVP | NSP16 | | CD4 | DQB1*05:01, DQB1*06:02, |
| | | | | | DRB1*01:01, DRB1*15:01 |
| 222 | AVMSLKEGQINDMIL | NSP16 | | CD4 | |
| 223 | KEGQINDMILSLLSK | NSP16 | | CD4 | |
| 224 | RENNRVVISSDVLVN | NSP16 | | CD4 | DQB1*02:02, DRB1*07:01 |
| 225 | FMRIFTIGTVTLKQG | ORF3a | Yes | CD4 | |
| 226 | SDFVRATATIPIQAS | ORF3a | | CD4 | DQB1*02:02, DRB1*07:01 |
| 227 | ALLAVFQSASKIITL | ORF3a | | CD4 | DQB1*03:01, DQB1*06:03, |
| | | | | | DRB1*07:01, DRB1*11:01, |
| | | | | | DRB1*13:01 |
| 228 | KIITLKKRWQLALSK | ORF3a | | CD4 | DRB1*11:01 |
| 229 | KKRWQLALSKGVHFV | ORF3a | Yes | CD4 | DQB1*04:02, DRB1*07:01, |
| | | | | | DRB1*09:01 |
| 230 | CNLLLLFVTVYSHLL | ORF3a | | CD4 | DQB1*02:01, DRB1*07:01, |
| | | | | | DRB1*11:01 |
| 231 | LVAAGLEAPFLYLYA | ORF3a | | CD4 | DQB1*02:01, DQB1*02:02 |
| 232 | LEAPFLYLYALVYFL | ORF3a | | CD4 | DQB1*02:01, DRB1*11:01 |
| 233 | LYLYALVYFLQSINF | ORF3a | Yes | CD4 | DQB1*02:01, DQB1*03:02, |
| | | | | | DQB1*05:03, DRB1*11:01, |
| | | | | | DRB1*12:01 |
| 234 | LVYFLQSINFVRIIM | ORF3a | | CD4 | DQB1*05:03, DQB1*06:03, |
| | | | | | DRB1*07:01, DRB1*12:01, |
| | | | | | DRB1*13:01, DRB1*14:01 |
| 235 | QSINFVRIIMRLWLC | ORF3a | Yes | CD4 | DQB1*06:02, DRB1*03:01, |
| | | | | | DRB1*07:01, DRB1*11:01, |
| | | | | | DRB1*12:01, DRB1*13:01, |
| | | | | | DRB1*14:01, DRB1*15:01 |
| 236 | VRIIMRLWLCWKCRS | ORF3a | | CD4 | DRB1*11:01 |
| 237 | RLWLCWKCRSKNPLL | ORF3a | | CD4 | |
| 238 | KNPLLYDANYFLCWH | ORF3a | | CD4 | DQB1*02:02, DQB1*05:03, |
| | | | | | DRB1*15:01 |
| 239 | YDANYFLCWHTNCYD | ORF3a | | CD4 | |
| 240 | FLCWHTNCYDYCIPY | ORF3a | | CD4 | DQB1*05:03 |
| 241 | TNCYDYCIPYNSVTS | ORF3a | | CD4 | DQB1*05:01 |
| 242 | YFTSDYYQLYSTQLS | ORF3a | | CD4 | DRB1*16:02 |
| 243 | TDTGVEHVTFFIYNK | ORF3a | | CD4 | |
| 244 | EHVTFFIYNKIVDEP | ORF3a | | CD4 | DQB1*02:01, DQB1*05:01, |
| | | | | | DRB1*08:01, DRB1*11:01 |
| 245 | FIYNKIVDEPEEHVQ | ORF3a | | CD4 | DQB1*05:01, DRB1*08:01 |
| 246 | GSSGVVNPVMEPIYD | ORF3a | | CD4 | DQB1*04:02 |
| 247 | MFHLVDFQVTIAEIL | ORF6 | | CD4 | DRB1*13:03 |
| 248 | IAEILLIIMRTFKVS | ORF6 | | CD4 | DRB1*01:03, DRB1*07:01 |
| 249 | AEILLIIMRTFKVSI | ORF6 | | CD4 | |
| 250 | LIIMRTFKVSIWNLD | ORF6 | | CD4 | DQB1*05:01, DRB1*01:01, |
| | | | | | DRB1*11:01 |
| 251 | TFKVSIWNLDYIINL | ORF6 | | CD4 | DQB1*02:02, DQB1*05:01, |
| | | | | | DRB1*01:03, DRB1*07:01, |
| | | | | | DRB1*15:01 |
| 252 | IWNLDYIINLIIKNL | ORF6 | Yes | CD4 | DQB1*05:01, DRB1*01:01, |
| | | | | | DRB1*15:01 |
| 253 | YIINLIIKNLSKSLT | ORF6 | | CD4 | DRB1*11:02, DRB1*13:03 |
| 254 | MKIILFLALITLATC | ORF7a | | CD4 | |
| 255 | IILFLALITLATCEL | ORF7a | | CD4 | |
| 256 | DGVKHVYQLRARSVS | ORF7a | | CD4 | |
| | PKL | | | | |
| 257 | VKHVYQLRARSVSPK | ORF7a | | CD4 | DRB1*01:03, DRB1*07:01 |
| 258 | QEEVQELYSPIFLIV | ORF7a | Yes | CD4 | |
| 259 | LYSPIFLIVAAIVFI | ORF7a | | CD4 | |
| 260 | SPIFLIVAAIVFITL | ORF7a | | CD4 | |
| 261 | DFYLCFLAFLLFLVL | ORF7b | | CD4 | |
| 262 | MKFLVFLGIITTVAA | ORF8 | | CD4 | |
| 263 | FLGIITTVAAFHQEC | ORF8 | | CD4 | DQB1*02:02, DQB1*03:02, |
| | | | | | DQB1*06:03, DRB1*01:03, |
| | | | | | DRB1*04:02, DRB1*07:01 |
| 264 | TTVAAFHQECSLQSC | ORF8 | | CD4 | DQB1*05:02 |
| 265 | FHQECSLQSCTQHQP | ORF8 | | CD4 | |
| 266 | SLQSCTQHQPYVVDD | ORF8 | | CD4 | |
| 267 | TQHQPYVVDDPCPIH | ORF8 | Yes | CD4 | DRB1*13:02 |
| 268 | YVVDDPCPIHFYSKW | ORF8 | Yes | CD4 | DRB1*13:02, DRB1*15:02 |
| 269 | PCPIHFYSKWYIRVG | ORF8 | Yes | CD4 | DRB1*08:02, DRB1*13:01, |
| | | | | | DRB1*15:01, DRB1*15:02, |
| | | | | | DRB1*16:01 |
| 270 | FYSKWYIRVGARKSA | ORF8 | Yes | CD4 | DQB1*06:01, DRB1*01:01, |
| | | | | | DRB1*04:02, DRB1*07:01, |
| | | | | | DRB1*11:01, DRB1*11:04, |
| | | | | | DRB1*13:03, DRB1*14:01 |
| 271 | SKWYIRVGARKSAPL | ORF8 | Yes | CD4 | |
| 272 | YIRVGARKSAPLIEL | ORF8 | Yes | CD4 | DQB1*03:01, DRB1*01:01, |
| | | | | | DRB1*01:03, DRB1*07:01, |
| | | | | | DRB1*11:04, DRB1*13:02, |
| | | | | | DRB1*15:01, DRB1*16:01 |
| 273 | ARKSAPLIELCVDEA | ORF8 | | CD4 | |
| 274 | PLIELCVDEAGSKSP | ORF8 | | CD4 | |
| 275 | CVDEAGSKSPIQYID | ORF8 | | CD4 | |
| 276 | IQYIDIGNYTVSCLP | ORF8 | | CD4 | DRB1*11:04, DRB1*13:02 |
| 277 | IGNYTVSCLPFTINC | ORF8 | Yes | CD4 | DQB1*03:03, DRB1*15:02 |
| 278 | FTINCQEPKLGSLVV | ORF8 | Yes | CD4 | |
| 279 | QEPKLGSLVVRCSFY | ORF8 | | CD4 | DRB1*11:04 |
| 280 | GSLVVRCSFYEDFLE | ORF8 | Yes | CD4 | DQB1*05:02, DQB1*05:03 |
| 281 | RCSFYEDFLEYHDVR | ORF8 | Yes | CD4 | DQB1*05:02 |
| 282 | EDFLEYHDVRVVLDF | ORF8 | | CD4 | DQB1*05:02, DQB1*06:04, |
| | | | | | DRB1*13:02, DRB1*15:02 |
| 283 | DFLEYHDVRVVLDFI | ORF8 | | CD4 | DQB1*05:02, DQB1*06:04, |
| | | | | | DRB1*15:02 |
| 284 | INVFAFPFTIYSLLL | ORF10 | Yes | CD4 | |
| 285 | MFVFLVLLPLVSS | S | | CD4 | |
| 286 | VLLPLVSSQCVNLTT | S | | CD4 | DRB1*07:01 |
| 287 | VSSQCVNLTTRTQLP | S | | CD4 | |
| 288 | VNLTTRTQLPPAYTN | S | | CD4 | |
| 289 | SFTRGVYYPDKVFRS | S | | CD4 | |
| 290 | VYYPDKVFRSSVLHS | S | | CD4 | DRB1*03:01, DRB1*13:01, |
| | | | | | DRB1*14:01 |
| 291 | SVLHSTQDLFLPFFS | S | Yes | CD4 | DQB1*02:02, DQB1*05:01, |
| | | | | | DRB1*07:01 |
| 292 | LPFFSNVTWFHAIHV | S | | CD4 | DQB1*05:02, DRB1*15:01, |
| | | | | | DRB1*16:01 |
| 293 | PFFSNVTWFHAIHVS | S | | CD4 | |
| 294 | NVTWFHAIHVSGTNG | S | | CD4 | |
| 295 | NPVLPFNDGVYFAST | S | | CD4 | |
| 296 | EKSNIIRGWIFGTTL | S | | CD4 | DRB1*15:01 |
| 297 | IRGWIFGTTLDSKTQ | S | | CD4 | |
| 298 | FGTTLDSKTQSLLIV | S | | CD4 | |
| 299 | DSKTQSLLIVNNATN | S | | CD4 | |
| 300 | SLLIVNNATNVVIKV | S | | CD4 | DQB1*06:03, DRB1*07:01, |
| | | | | | DRB1*12:01, DRB1*13:01, |
| | | | | | DRB1*14:01 |
| 301 | NNATNVVIKVCEFQF | S | Yes | CD4 | |
| 302 | VVIKVCEFQFCNDPF | S | | CD4 | DQB1*05:01 |
| 303 | CEFQFCNDPFLGVYY | S | Yes | CD4 | DQB1*02:02, DQB1*05:01, |
| | | | | | DQB1*05:02, DQB1*05:03 |
| 304 | LGVYYHKNNKSWMES | S | | CD4 | DRB1*13:03 |
| 305 | HKNNKSWMESEFRVY | S | | CD4 | DQB1*05:03 |
| 306 | SSANNCTFEYVSQPF | S | Yes | CD4 | |
| 307 | CTFEYVSQPFLMDLE | S | Yes | CD4 | DQB1*02:01, DQB1*02:02, |
| | | | | | DQB1*05:02, DQB1*05:03, |
| | | | | | DQB1*06:04, DRB1*07:01, |
| | | | | | DRB1*16:01 |
| 308 | VSQPFLMDLEGKQGN | S | | CD4 | DRB1*03:01 |
| 309 | LMDLEGKQGNFKNLR | S | Yes | CD4 | |
| 310 | GKQGNFKNLREFVFK | S | | CD4 | |
| 311 | EFVFKNIDGYFKIYS | S | Yes | CD4 | DQB1*05:03, DRB1*14:01, |
| | | | | | DRB1*15:01 |
| 312 | NIDGYFKIYSKHTPI | S | Yes | CD4 | DRB1*07:01, DRB1*15:01, |
| | | | | | DRB1*16:01 |
| 313 | FKIYSKHTPINLVRD | S | | CD4 | DRB1*07:01, DRB1*13:01 |
| 314 | SKHTPINLVRDLPQG | S | | CD4 | |
| 315 | KHTPINLVRDLPQGF | S | Yes | CD4 | DRB1*03:01 |
| 316 | PINLVRDLPQGFSAL | S | | CD4 | DRB1*03:01, DRB3*01:01 |
| 317 | NLVRDLPQGFSALEP | S | Yes | CD4 | DRB1*03:01 |
| 318 | LPQGFSALEPLVDLP | S | Yes | CD4 | DQB1*02:02, DQB1*03:03, |
| | | | | | DQB1*05:03 |
| 319 | SALEPLVDLPIGINI | S | | CD4 | |
| 320 | IGINITRFQTLLALH | S | Yes | CD4 | DQB1*06:03, DRB1*07:01, |
| | | | | | DRB1*11:02, DRB1*13:01, |
| | | | | | DRB1*13:03 |
| 321 | ITRFQTLLALHRSYL | S | Yes | CD4 | |
| 322 | TRFQTLLALHRSYLT | S | Yes | CD4 | DQB1*05:01, DQB1*05:03, |
| | | | | | DQB1*06:02, DQB1*06:03, |
| | | | | | DRB1*01:01, DRB1*07:01, |
| | | | | | DRB1*08:03, DRB1*11:02, |
| | | | | | DRB1*12:01, DRB1*13:01, |
| | | | | | DRB1*13:02, DRB1*13:03, |
| | | | | | DRB1*14:01, DRB1*15:01 |
| 323 | LLALHRSYLTPGDSS | S | Yes | CD4 | DRB1*01:01, DRB1*07:01, |
| | | | | | DRB1*11:02, DRB1*15:01 |
| 324 | YVGYLQPRTFLLKYN | S | | CD4 | |
| 325 | CTLKSFTVEKGIYQT | S | | CD4 | DRB1*15:01 |
| 326 | FTVEKGIYQTSNFRV | S | | CD4 | DRB1*07:01 |
| 327 | GIYQTSNFRVQPTES | S | | CD4 | DRB1*01:01, DRB1*13:03 |
| 328 | SNFRVQPTESIVRFP | S | | CD4 | DRB1*03:01, DRB1*07:01 |
| 329 | QPTESIVRFPNITNL | S | Yes | CD4 | DRB1*08:03, DRB1*15:01 |
| 330 | IVRFPNITNLCPFGE | S | Yes | CD4 | DRB1*15:01 |
| 331 | CPFGEVENATRFASV | S | Yes | CD4 | DQB1*03:01, DQB1*06:02, |
| | | | | | DQB1*06:03, DRB1*01:01, |
| | | | | | DRB1*07:01, DRB1*08:03, |
| | | | | | DRB1*11:01, DRB1*11:02, |
| | | | | | DRB1*13:01, DRB1*13:03 |
| 332 | VFNATRFASVYAWNR | S | Yes | CD4 | DQB1*03:01, DQB1*06:02, |
| | | | | | DRB1*01:01, DRB1*08:03, |
| | | | | | DRB1*11:01, DRB1*13:03, |
| | | | | | DRB1*15:01 |
| 333 | RFASVYAWNRKRISN | S | Yes | CD4 | DRB1*07:01, DRB1*11:02, |
| | | | | | DRB1*13:01, DRB1*13:03, |
| | | | | | DRB1*14:01 |
| 334 | YAWNRKRISNCVADY | S | | CD4 | DRB1*13:01 |
| 335 | KRISNCVADYSVLYN | S | | CD4 | DQB1*02:01, DQB1*02:02, |
| | | | | | DRB1*03:01 |
| 336 | CVADYSVLYNSASFS | S | | CD4 | DQB1*05:03, DQB1*06:02 |
| 337 | SVLYNSASFSTFKCY | S | | CD4 | DRB1*15:01 |
| 338 | SASFSTFKCYGVSPT | S | | CD4 | DRB1*15:01 |
| 339 | TFKCYGVSPTKLNDL | S | | CD4 | |
| 340 | GVSPTKLNDLCFTNV | S | | CD4 | |
| 341 | KLNDLCFTNVYADSF | S | | CD4 | DQB1*02:01, DQB1*02:02, |
| | | | | | DRB1*07:01 |
| 342 | YADSFVIRGDEVRQI | S | | CD4 | DQB1*02:02, DRB1*13:01 |
| 343 | VIRGDEVRQIAPGQT | S | | CD4 | DRB1*13:01 |
| 344 | GKIADYNYKLPDDFT | S | | CD4 | |
| 345 | GCVIAWNSNNLDSKV | S | Yes | CD4 | DQB1*02:02, DQB1*06:03, |
| | | | | | DRB1*07:01, DRB1*13:01 |
| 346 | WNSNNLDSKVGGNYN | S | | CD4 | |
| 347 | GGNYNYLYRLFRKSN | S | Yes | CD4 | DRB1*11:02, DRB1*13:03 |
| 348 | YNYLYRLFRKSNLKP | S | | CD4 | DPB1*02:01 |
| 349 | YLYRLFRKSNLKPFE | S | | CD4 | |
| 350 | LKPFERDISTEIYQA | S | | CD4 | |
| 351 | EIYQAGSTPCNGVEG | S | | CD4 | |
| 352 | NGVEGENCYFPLQSY | S | | CD4 | |
| 353 | FNCYFPLQSYGFQPT | S | | CD4 | DQB1*05:01, DQB1*05:03, |
| | | | | | DRB1*01:01, DRB1*12:01 |
| 354 | QPYRVVVLSFELLHA | S | Yes | CD4 | DQB1*05:03, DRB1*14:01 |
| 355 | VVLSFELLHAPATVC | S | | CD4 | DQB1*05:03, DRB1*12:01, |
| | | | | | DRB1*14:01 |
| 356 | FNFNGLTGTGVLTES | S | | CD4 | DQB1*06:02, DRB1*01:01 |
| 357 | LTGTGVLTESNKKFL | S | | CD4 | |
| 358 | TLEILDITPCSFGGV | S | | CD4 | |
| 359 | VNNSYECDIPIGAGI | S | | CD4 | |
| 360 | ECDIPIGAGICASYQ | S | | CD4 | |
| 361 | SIIAYTMSLGAENSV | S | Yes | CD4 | DQB1*02:02, DQB1*05:01, |
| | | | | | DRB1*01:03, DRB1*07:01 |
| 362 | AENSVAYSNNSIAIP | S | Yes | CD4 | DQB1*03:01, DQB1*06:02, |
| | | | | | DRB1*08:03, DRB1*15:01 |
| 363 | AYSNNSIAIPTNFTI | S | | CD4 | DQB1*03:01, DQB1*06:03 |
| 364 | SIAIPTNFTISVTTE | S | Yes | CD4 | DQB1*02:02, DQB1*06:02 |
| 365 | TNFTISVTTEILPVS | S | Yes | CD4 | DQB1*02:02, DQB1*03:01, |
| | | | | | DQB1*06:03, DRB1*01:01, |
| | | | | | DRB1*01:03, DRB1*07:01, |
| | | | | | DRB1*14:01 |
| 366 | SVTTEILPVSMTKTS | S | | CD4 | |
| 367 | STECSNLLLQYGSFC | S | Yes | CD4 | DQB1*06:02 |
| 368 | NLLLQYGSFCTQLNR | S | Yes | CD4 | DQB1*05:01, DQB1*05:03, |
| | | | | | DRB1*01:01, DRB1*04:04, |
| | | | | | DRB1*15:01 |
| 369 | YGSFCTQLNRALTGI | S | | CD4 | DQB1*03:03, DQB1*04:02, |
| | | | | | DRB1*09:01 |
| 370 | TQLNRALTGIAVEQD | S | Yes | CD4 | DQB1*03:03, DQB1*04:02, |
| | | | | | DQB1*06:02, DQB1*06:04, |
| | | | | | DRB1*01:01, DRB1*09:01 |
| 371 | KNTQEVFAQVKQIYK | S | | CD4 | |
| 372 | VFAQVKQIYKTPPIK | S | | CD4 | DRB1*15:01 |
| 373 | TPPIKDFGGFNFSQI | S | | CD4 | DRB1*15:01 |
| 374 | NFSQILPDPSKPSKR | S | Yes | CD4 | DRB1*03:01 |
| 375 | LPDPSKPSKRSFIED | S | | CD4 | |
| 376 | KPSKRSFIEDLLFNK | S | Yes | CD4 | DQB1*02:01, DQB1*02:02, |
| | | | | | DQB1*05:01, DQB1*05:03, |
| | | | | | DRB1*03:01 |
| 377 | SFIEDLLFNKVTLAD | S | Yes | CD4 | DQB1*05:01, DQB1*05:02, |
| | | | | | DQB1*05:03, DRB1*01:01, |
| | | | | | DRB1*01:03, DRB1*03:01, |
| | | | | | DRB1*04:04, DRB1*11:02, |
| | | | | | DRB1*12:01, DRB1*13:03, |
| | | | | | DRB1*14:01, DRB1*15:01, |
| | | | | | DRB1*16:02 |
| 378 | LLFNKVTLADAGFIK | S | | CD4 | DQB1*03:01, DQB1*06:02, |
| | | | | | DRB1*01:01 |
| 379 | VTLADAGFIKQYGDC | S | | CD4 | |
| 380 | AGFIKQYGDCLGDIA | S | Yes | CD4 | DQB1*05:03 |
| 381 | QYGDCLGDIAARDLI | S | | CD4 | |
| 382 | LGDIAARDLICAQKF | S | | CD4 | |
| 383 | ARDLICAQKENGLTV | S | | CD4 | |
| 384 | CAQKFNGLTVLPPLL | S | | CD4 | DQB1*05:01, DQB1*05:02, |
| | | | | | DQB1*06:02, DRB1*01:01, |
| | | | | | DRB1*15:01, DRB1*16:01 |
| 385 | FNGLTVLPPLLTDEM | S | Yes | CD4 | |
| 386 | LPPLLTDEMIAQYTS | S | | CD4 | DQB1*04:02 |
| 387 | TDEMIAQYTSALLAG | S | Yes | CD4 | DQB1*02:02, DQB1*03:02, |
| | | | | | DQB1*06:02, DRB1*04:04, |
| | | | | | DRB1*04:10, DRB1*07:01, |
| | | | | | DRB1*15:01 |
| 388 | AQYTSALLAGTITSG | S | | CD4 | DQB1*06:02 |
| 389 | ALLAGTITSGWTFGA | S | | CD4 | DQB1*06:02 |
| 390 | TITSGWTFGAGAALQ | S | | CD4 | DQB1*04:02, DQB1*06:02 |
| 391 | WTFGAGAALQIPFAM | S | | CD4 | DQB1*03:01, DRB1*01:01 |
| 392 | IPFAMQMAYRENGIG | S | Yes | CD4 | DQB1*04:02, DQB1*05:01, |
| | | | | | DQB1*05:03, DRB1*01:01, |
| | | | | | DRB1*11:01, DRB1*12:01, |
| | | | | | DRB1*14:01, DRB1*15:01 |
| 393 | QMAYRFNGIGVTQNV | S | Yes | CD4 | DQB1*03:01, DQB1*06:03, |
| | | | | | DRB1*01:01, DRB1*07:01 |
| 394 | VTQNVLYENQKLIAN | S | | CD4 | DRB1*03:01, DRB1*12:01 |
| 395 | LYENQKLIANQENSA | S | | CD4 | DRB1*12:01 |
| 396 | GKLQDVVNQNAQALN | S | | CD4 | |
| 397 | AQALNTLVKQLSSNF | S | | CD4 | DQB1*06:02, DRB1*11:01, |
| | | | | | DRB1*13:03 |
| 398 | TLVKQLSSNFGAISS | S | | CD4 | DQB1*03:01, DRB1*01:01, |
| | | | | | DRB1*11:01 |
| 399 | GAISSVLNDILSRLD | S | | CD4 | DQB1*05:01 |
| 400 | VQIDRLITGRLQSLQ | S | Yes | CD4 | DRB1*01:01, DRB1*03:01, |
| | | | | | DRB1*11:01, DRB1*13:03 |
| 401 | LITGRLQSLQTYVTQ | S | | CD4 | DQB1*06:02, DRB1*01:01, |
| | | | | | DRB1*15:01 |
| 402 | APHGVVFLHVTYVPA | S | | CD4 | DQB1*05:01, DRB1*04:04, |
| | | | | | DRB1*10:01, DRB1*12:01 |
| 403 | TTAPAICHDGKAHFP | S | | CD4 | |
| 404 | KAHFPREGVFVSNGT | S | | CD4 | |
| 405 | REGVFVSNGTHWFVT | S | | CD4 | DRB1*01:03, DRB1*07:01 |
| 406 | HWFVTQRNFYEPQII | S | | CD4 | DQB1*05:01 |
| 407 | QRNFYEPQIITTDNT | S | | CD4 | |
| 408 | FVSGNCDVVIGIVNN | S | | CD4 | |
| 409 | GIVNNTVYDPLQPEL | S | | CD4 | DQB1*02:02, DQB1*03:02 |
| 410 | LQPELDSFKEELDKY | S | | CD4 | |
| 411 | ELDKYFKNHTSPDVD | S | Yes | CD4 | |
| 412 | LGDISGINASVVNIQ | S | | CD4 | DQB1*03:01 |
| 413 | GINASVVNIQKEIDR | S | Yes | CD4 | DQB1*03:01 |
| 414 | LNEVAKNLNESLIDL | S | | CD4 | DRB1*13:02 |
| 415 | QELGKYEQYIKWPWY | S | | CD4 | |
| 416 | YEQYIKWPWYIWLGF | S | | CD4 | |
| 417 | SEETGTLIV | E | | CD8 | |
| 418 | FLAFVVFL | E | | CD8 | A*02:01 |
| 419 | FLLVTLAIL | E | | CD8 | A*02:01 |
| 420 | SLVKPSFYV | E | Yes | CD8 | |
| 421 | LVKPSFYVY | E | | CD8 | C*07:02 |
| 422 | FYVYSRVKNL | E | | CD8 | A*24:02 |
| 423 | YVYSRVKNL | E | Yes | CD8 | C*06:02 |
| 424 | NGTITVEELK | M | | CD8 | A*68:01 |
| 425 | GTITVEELK | M | Yes | CD8 | A*68:01 |
| 426 | KLLEQWNLV | M | Yes | CD8 | A*02:01 |
| 427 | FLFLTWICL | M | Yes | CD8 | A*02:01 |
| 428 | WICLLQFAY | M | | CD8 | |
| 429 | LQFAYANRNRFLY | M | Yes | CD8 | |
| 430 | FAYANRNRF | M | | CD8 | B*15:01, B*35:01 |
| 431 | AYANRNRF | M | | CD8 | A*24:02 |
| 432 | AYANRNRFL | M | | CD8 | A*24:02 |
| 433 | YANRNRFLY | M | | CD8 | A*01:01, B*35:01 |
| 434 | ANRNRFLYI | M | | CD8 | B*08:01 |
| 435 | RNRFLYIIK | M | | CD8 | A*30:01 |
| 436 | RNRFLYIIKL | M | | CD8 | C*07:01 |
| 437 | NRFLYIIKL | M | Yes | CD8 | B*08:01, C*07:02 |
| 438 | RFLYIIKLIF | M | | CD8 | A*24:02 |
| 439 | FLWLLWPVTL | M | | CD8 | A*02:01 |
| 440 | WLLWPVTLA | M | | CD8 | A*02:01 |
| 441 | LLWPVTLAC | M | | CD8 | A*02:01 |
| 442 | TLACFVLAAV | M | Yes | CD8 | A*02:01 |
| 443 | FVLAAVYRI | M | Yes | CD8 | A*02:01, A*68:02 |
| 444 | LAAVYRINW | M | | CD8 | B*57:01 |
| 445 | LAAVYRINWI | M | | CD8 | |
| 446 | AAVYRINW | M | | CD8 | B*57:01 |
| 447 | AIAMACLVGLM | M | | CD8 | |
| 448 | MACLVGLMW | M | | CD8 | B*57:01 |
| 449 | GLMWLSYFI | M | | CD8 | A*02:01 |
| 450 | LSYFIASFR | M | | CD8 | A*31:01, A*68:01 |
| 451 | SYFIASFRLFA | M | Yes | CD8 | |
| 452 | IASFRLFAR | M | | CD8 | A*33:01 |
| 453 | FRLFARTRSM | M | | CD8 | B*08:01 |
| 454 | RLFARTRSM | M | | CD8 | A*30:01 |
| 455 | RLFARTRSMW | M | Yes | CD8 | |
| 456 | RTRSMWSF | M | | CD8 | B*57:01 |
| 457 | SMWSFNPET | M | Yes | CD8 | A*02:01 |
| 458 | SMWSFNPETNIL | M | | CD8 | |
| 459 | SFNPETNIL | M | | CD8 | B*08:01 |
| 460 | VPLHGTIL | M | | CD8 | B*07:02 |
| 461 | RPLLESEL | M | | CD8 | B*07:02 |
| 462 | SELVIGAVI | M | | CD8 | B*44:02 |
| 463 | SELVIGAVIL | M | Yes | CD8 | B*40:01 |
| 464 | ELVIGAVILR | M | | CD8 | A*68:01 |
| 465 | LVIGAVILR | M | | CD8 | A*68:01 |
| 466 | AVILRGHLR | M | Yes | CD8 | A*68:01 |
| 467 | HLRIAGHHLGR | M | Yes | CD8 | |
| 468 | HLRIAGHHL | M | | CD8 | B*08:01 |
| 469 | RIAGHHLGR | M | | CD8 | A*03:01 |
| 470 | LPKEITVAT | M | | CD8 | B*07:02 |
| 471 | TVATSRTLSYYK | M | Yes | CD8 | |
| 472 | TVATSRTLSY | M | | CD8 | A*01:01 |
| 473 | VATSRTLSYY | M | | CD8 | A*01:01 |
| 474 | VATSRTLSY | M | | CD8 | A*01:01, B*35:01 |
| 475 | ATSRTLSYYK | M | Yes | CD8 | A*11:01, A*30:01 |
| 476 | ATSRTLSYY | M | Yes | CD8 | A*01:01, A*11:01, B*57:01 |
| 477 | ASQRVAGDSGFAAY | M | Yes | CD8 | |
| 478 | SQRVAGDSGF | M | | CD8 | B*15:01 |
| 479 | RVAGDSGFAAYSRY | M | | CD8 | A*30:02 |
| 480 | RVAGDSGFAAY | M | | CD8 | A*01:01, B*15:01 |
| 481 | AGDSGFAAY | M | | CD8 | A*01:01 |
| 482 | DSGFAAYSR | M | | CD8 | A*68:01 |
| 483 | YSRYRIGNYK | M | | CD8 | A*30:01 |
| 484 | RYRIGNYKL | M | Yes | CD8 | A*24:02, A*30:01 |
| 485 | SSSDNIALL | M | | CD8 | A*68:02 |
| 486 | GPQNQRNAPRITF | N | | CD8 | B*07:02 |
| 487 | QRNAPRITF | N | Yes | CD8 | B*27:05, C*07:01, C*07:02 |
| 488 | RPQGLPNNTA | N | | CD8 | B*07:02 |
| 489 | QGLPNNTASW | N | | CD8 | B*57:01 |
| 490 | LPNNTASWF | N | | CD8 | B*07:02 |
| 491 | NTASWFTAL | N | Yes | CD8 | A*02:01 |
| 492 | KFPRGQGVPI | N | Yes | CD8 | |
| 493 | FPRGQGVPI | N | Yes | CD8 | B*07:02, B*08:01 |
| 494 | NTNSSPDDQIGYY | N | Yes | CD8 | A*01:01 |
| 495 | SSPDDQIGYYR | N | | CD8 | A*01:01 |
| 496 | SPDDQIGYY | N | | CD8 | B*35:01 |
| 497 | YYRRATRRIR | N | Yes | CD8 | |
| 498 | RIRGGDGKMK | N | Yes | CD8 | |
| 499 | KMKDLSPRW | N | Yes | CD8 | B*57:01 |
| 500 | MKDLSPRWY | N | | CD8 | C*07:01 |
| 501 | LSPRWYFYYL | N | Yes | CD8 | |
| 502 | SPRWYFYYL | N | Yes | CD8 | B*07:02, B*08:01 |
| 503 | YLGTGPEAGL | N | | CD8 | A*02:01 |
| 504 | GTGPEAGLPY | N | Yes | CD8 | |
| 505 | AGLPYGANK | N | | CD8 | A*30:01 |
| 506 | LPYGANKDGI | N | | CD8 | B*51:01 |
| 507 | YGANKDGIIW | N | | CD8 | B*57:01 |
| 508 | IIWVATEGA | N | | CD8 | A*02:01 |
| 509 | ATEGALNTPK | N | Yes | CD8 | A*11:01 |
| 510 | ATEGALNTPKDHI | N | Yes | CD8 | |
| 511 | GTRNPANNA | N | | CD8 | A*30:01 |
| 512 | RNPANNAAIVL | N | Yes | CD8 | |
| 513 | NPANNAAIVL | N | Yes | CD8 | B*07:02 |
| 514 | VLQLPQGTTL | N | Yes | CD8 | A*02:01 |
| 515 | AEGSRGGSQA | N | | CD8 | |
| 516 | SQASSRSSSR | N | | CD8 | |
| 517 | SSRGTSPAR | N | | CD8 | |
| 518 | LALLLLDRL | N | Yes | CD8 | A*02:01 |
| 519 | LLLLDRLNQL | N | Yes | CD8 | A*02:01 |
| 520 | LLLDRLNQL | N | Yes | CD8 | A*02:01 |
| 521 | QQQQGQTVTK | N | Yes | CD8 | |
| 522 | KSAAEASKK | N | | CD8 | A*11:01 |
| 523 | KPRQKRTAT | N | Yes | CD8 | B*07:02, B*08:01 |
| 524 | RTATKAYNV | N | | CD8 | A*02:01 |
| 525 | KAYNVTQAF | N | Yes | CD8 | B*35:01, B*57:01 |
| 526 | RRGPEQTQGNF | N | | CD8 | C*07:01 |
| 527 | QELIRQGTDY | N | | CD8 | B*44:02 |
| 528 | QELIRQGTDYKHW | N | Yes | CD8 | |
| 529 | ELIRQGTDY | N | | CD8 | A*26:01 |
| 530 | DYKHWPQIAQF | N | | CD8 | A*24:02 |
| 531 | HWPQIAQF | N | | CD8 | A*24:02 |
| 532 | AQFAPSASA | N | Yes | CD8 | A*02:01 |
| 533 | AQFAPSASAF | N | Yes | CD8 | A*24:02, B*15:01 |
| 534 | QFAPSASAFF | N | | CD8 | A*24:02 |
| 535 | FAPSASAFF | N | | CD8 | B*35:01 |
| 536 | APSASAFFGM | N | | CD8 | B*07:02 |
| 537 | SASAFFGMSR | N | | CD8 | A*68:01 |
| 538 | ASAFFGMSR | N | Yes | CD8 | A*11:01, A*68:01 |
| 539 | GMSRIGMEV | N | Yes | CD8 | A*02:01 |
| 540 | GMEVTPSGTWLTY | N | Yes | CD8 | |
| 541 | MEVTPSGTW | N | Yes | CD8 | B*44:03 |
| 542 | MEVTPSGTWL | N | Yes | CD8 | B*40:01 |
| 543 | EVTPSGTWLTY | N | | CD8 | A*26:01 |
| 544 | VTPSGTWLTY | N | | CD8 | A*30:02 |
| 545 | TPSGTWLTY | N | | CD8 | B*35:01 |
| 546 | KLDDKDPNF | N | | CD8 | A*02:01 |
| 547 | ILLNKHIDA | N | Yes | CD8 | A*02:01 |
| 548 | LLNKHIDAY | N | | CD8 | B*15:01 |
| 549 | AYKTFPPTEPK | N | Yes | CD8 | |
| 550 | YKTFPPTEPK | N | | CD8 | A*68:01 |
| 551 | KTFPPTEPKK | N | Yes | CD8 | A*03:01, A*11:01 |
| 552 | KTFPPTEPK | N | Yes | CD8 | A*03:01, A*11:01, A*68:01 |
| 553 | KTFPPTEPKKDKKK | N | | CD8 | A*03:01 |
| 554 | KKQQTVTLL | N | | CD8 | C*07:01 |
| 555 | LPAADLDDF | N | Yes | CD8 | B*35:01 |
| 556 | AADLDDFSKQL | N | | CD8 | A*02:01 |
| 557 | FSKQLQQSM | N | Yes | CD8 | |
| 558 | VLSEARQHL | NSP1 | Yes | CD8 | A*02:01 |
| 559 | HLKDGTCGL | NSP1 | | CD8 | B*08:01 |
| 560 | APHGHVMVEL | NSP1 | | CD8 | B*07:02 |
| 561 | VMVELVAEL | NSP1 | | CD8 | A*02:01 |
| 562 | VPHVGEIPV | NSP1 | | CD8 | B*07:02 |
| 563 | HVGEIPVAY | NSP1 | | CD8 | B*15:01 |
| 564 | IPVAYRKVLL | NSP1 | | CD8 | B*07:02 |
| 565 | TFNGECPNF | NSP2 | | CD8 | A*24:02 |
| 566 | KTIQPRVEK | NSP2 | Yes | CD8 | A*03:01, A*11:01 |
| 567 | GFMGRIRSV | NSP2 | | CD8 | C*06:02 |
| 568 | SEVGPEHSLAEY | NSP2 | Yes | CD8 | |
| 569 | EEIAIILASF | NSP2 | | CD8 | B*44:03 |
| 570 | AIILASFSAST | NSP2 | | CD8 | |
| 571 | ILSPLYAFA | NSP2 | | CD8 | A*02:01 |
| 572 | VRSIFSRTL | NSP2 | | CD8 | C*06:02 |
| 573 | ITILDGISQY | NSP2 | | CD8 | B*15:01 |
| 574 | RLIDAMMFT | NSP2 | | CD8 | A*02:01 |
| 575 | TSDLATNNLVVMAY | NSP2 | Yes | CD8 | |
| 576 | TVYEKLKPV | NSP2 | | CD8 | A*02:01 |
| 577 | YEKLKPVL | NSP2 | | CD8 | B*08:01 |
| 578 | EIKESVQTF | NSP2 | | CD8 | B*15:01 |
| 579 | KLVNKFLAL | NSP2 | Yes | CD8 | A*02:01 |
| 580 | APKEIIFLEGETL | NSP2 | Yes | CD8 | |
| 581 | APKEIIFL | NSP2 | | CD8 | B*07:02 |
| 582 | GETLPTEVL | NSP2 | | CD8 | B*40:01 |
| 583 | VTNNTFTLK | NSP2 | Yes | CD8 | A*03:01, A*11:01 |
| 584 | FGDDTVIEV | NSP3 | Yes | CD8 | A*02:01 |
| 585 | DTVIEVQGYK | NSP3 | | CD8 | A*68:01 |
| 586 | QGYKSVNITF | NSP3 | | CD8 | A*24:02 |
| 587 | FELDERIDKV | NSP3 | | CD8 | A*02:01 |
| 588 | FELDERIDKVL | NSP3 | | CD8 | B*40:01 |
| 589 | ELDERIDKV | NSP3 | | CD8 | A*02:01 |
| 590 | VLNEKCSAY | NSP3 | | CD8 | B*15:01 |
| 591 | VELGTEVNEF | NSP3 | | CD8 | B*44:02 |
| 592 | SELLTPLGI | NSP3 | | CD8 | B*40:01 |
| 593 | YLFDESGEF | NSP3 | | CD8 | B*15:01 |
| 594 | YLFDESGEFKL | NSP3 | Yes | CD8 | A*02:01 |
| 595 | FEPSTQYEY | NSP3 | | CD8 | B*44:02 |
| 596 | DDYQGKPLEF | NSP3 | | CD8 | A*24:02 |
| 597 | KPLEFGATSAAL | NSP3 | | CD8 | |
| 598 | LEFGATSAAL | NSP3 | | CD8 | B*40:01 |
| 599 | VEVQPQLEM | NSP3 | | CD8 | B*40:01 |
| 600 | LEMELTPVV | NSP3 | | CD8 | B*40:01 |
| 601 | MELTPVVQTI | NSP3 | | CD8 | B*40:01 |
| 602 | TPVVQTIEV | NSP3 | | CD8 | B*07:02 |
| 603 | TIEVNSFSGY | NSP3 | | CD8 | A*01:01 |
| 604 | IEVNSFSGY | NSP3 | | CD8 | B*44:02 |
| 605 | NSFSGYLKL | NSP3 | | CD8 | C*06:02 |
| 606 | YLKLTDNVY | NSP3 | | CD8 | B*15:01 |
| 607 | YLKLTDNVYIK | NSP3 | | CD8 | |
| 608 | EAKKVKPTV | NSP3 | | CD8 | B*51:01 |
| 609 | VVVNAANVY | NSP3 | | CD8 | B*35:01 |
| 610 | YIATNGPLK | NSP3 | | CD8 | A*11:01 |
| 611 | YENFNQHEV | NSP3 | | CD8 | B*40:01 |
| 612 | LLSAGIFGA | NSP3 | | CD8 | |
| 613 | GADPIHSLR | NSP3 | | CD8 | A*68:01 |
| 614 | RTNVYLAVF | NSP3 | | CD8 | B*57:01 |
| 615 | AVFDKNLYDK | NSP3 | | CD8 | A*03:01, A*11:01 |
| 616 | NLYDKLVSSFL | NSP3 | | CD8 | A*02:01 |
| 617 | LYDKLVSSF | NSP3 | | CD8 | A*24:02 |
| 618 | KLVSSFLEM | NSP3 | | CD8 | B*15:01 |
| 619 | KIAEIPKEEV | NSP3 | | CD8 | A*02:01 |
| 620 | EVKPFITESK | NSP3 | | CD8 | A*68:01 |
| 621 | ESKPSVEQR | NSP3 | | CD8 | A*68:01 |
| 622 | FLTENLLLYI | NSP3 | | CD8 | A*02:01 |
| 623 | LVSDIDITF | NSP3 | | CD8 | B*35:01 |
| 624 | APYIVGDVV | NSP3 | | CD8 | B*51:01 |
| 625 | LTAVVIPTK | NSP3 | | CD8 | A*68:01 |
| 626 | ALRKVPTDNY | NSP3 | | CD8 | B*15:01 |
| 627 | ALRKVPTDNYITTY | NSP3 | Yes | CD8 | |
| 628 | PTDNYITTY | NSP3 | Yes | CD8 | A*01:01 |
| 629 | TDNYITTY | NSP3 | Yes | CD8 | A*01:01 |
| 630 | TTYPGQGLNGY | NSP3 | | CD8 | A*01:01 |
| 631 | KQEILGTVSW | NSP3 | | CD8 | B*44:02, B*44:03 |
| 632 | QEILGTVSW | NSP3 | Yes | CD8 | B*44:03 |
| 633 | MLAHAEETR | NSP3 | | CD8 | A*68:01 |
| 634 | AHAEETRKL | NSP3 | | CD8 | C*06:02 |
| 635 | KLMPVCVET | NSP3 | | CD8 | A*02:01 |
| 636 | AIVSTIQRKYK | NSP3 | | CD8 | A*03:01 |
| 637 | AIVSTIQRK | NSP3 | | CD8 | A*03:01 |
| 638 | VVDYGARFY | NSP3 | | CD8 | A*01:01 |
| 639 | SLINTLNDL | NSP3 | | CD8 | A*02:01 |
| 640 | VSSPDAVTAY | NSP3 | | CD8 | A*01:01, B*57:01 |
| 641 | TISLAGSYK | NSP3 | | CD8 | A*03:01, A*11:01, A*68:01 |
| 642 | ISLAGSYKDW | NSP3 | | CD8 | B*57:01 |
| 643 | SVYYTSNPTTF | NSP3 | | CD8 | A*24:02 |
| 644 | YYTSNPTTF | NSP3 | | CD8 | A*24:02 |
| 645 | YYTSNPTTFHL | NSP3 | | CD8 | A*24:02 |
| 646 | TSNPTTFHL | NSP3 | | CD8 | B*57:01 |
| 647 | HLDGEVITF | NSP3 | | CD8 | C*07:02 |
| 648 | GEVITFDNL | NSP3 | | CD8 | B*40:01 |
| 649 | ITFDNLKTL | NSP3 | | CD8 | B*57:01 |
| 650 | NLKTLLSL | NSP3 | | CD8 | B*08:01 |
| 651 | RTIKVFTTV | NSP3 | | CD8 | A*02:01 |
| 652 | NINLHTQVV | NSP3 | | CD8 | B*08:01 |
| 653 | HTQVVDMSMTY | NSP3 | | CD8 | A*01:01 |
| 654 | QVVDMSMTY | NSP3 | | CD8 | A*01:01, A*11:01 |
| 655 | MSMTYGQQF | NSP3 | | CD8 | B*57:01 |
| 656 | KPHNSHEGKTF | NSP3 | | CD8 | B*07:02 |
| 657 | LRVEAFEYY | NSP3 | | CD8 | C*07:01 |
| 658 | FEYYHTTDPSF | NSP3 | | CD8 | A*24:02 |
| 659 | HTTDPSFLGR | NSP3 | | CD8 | A*68:01 |
| 660 | HTTDPSFLGRY | NSP3 | Yes | CD8 | A*01:01 |
| 661 | TTDPSFLGRY | NSP3 | Yes | CD8 | A*01:01 |
| 662 | TTDPSFLGRYM | NSP3 | Yes | CD8 | A*01:01 |
| 663 | FLGRYMSAL | NSP3 | | CD8 | |
| 664 | YMSALNHTK | NSP3 | | CD8 | A*03:01 |
| 665 | MSALNHTKK | NSP3 | | CD8 | A*30:01 |
| 666 | MSALNHTKKW | NSP3 | | CD8 | B*57:01 |
| 667 | SALNHTKKW | NSP3 | | CD8 | B*57:01 |
| 668 | WKYPQVNGL | NSP3 | | CD8 | C*07:01 |
| 669 | YPQVNGLTSI | NSP3 | | CD8 | B*51:01 |
| 670 | YLATALLTL | NSP3 | Yes | CD8 | A*02:01 |
| 671 | YYRARAGEAANF | NSP3 | Yes | CD8 | |
| 672 | ARAGEAANF | NSP3 | | CD8 | C*07:01 |
| 673 | GEAANFCAL | NSP3 | | CD8 | B*40:01 |
| 674 | GELGDVRETMSYLF | NSP3 | | CD8 | B*44:02 |
| 675 | LGDVRETMSY | NSP3 | | CD8 | A*01:01 |
| 676 | VRETMSYLF | NSP3 | | CD8 | C*07:01 |
| 677 | VMYMGTLSY | NSP3 | | CD8 | A*03:01 |
| 678 | SAPPAQYEL | NSP3 | Yes | CD8 | C*07:01 |
| 679 | FTCASEYTGNY | NSP3 | | CD8 | A*01:01 |
| 680 | SEYTGNYQCGHY | NSP3 | | CD8 | B*44:02 |
| 681 | YTGNYQCGHY | NSP3 | | CD8 | A*01:01 |
| 682 | SEYKGPITDVFY | NSP3 | | CD8 | B*44:02 |
| 683 | ITDVFYKENSY | NSP3 | Yes | CD8 | A*01:01 |
| 684 | DVFYKENSY | NSP3 | | CD8 | A*26:01 |
| 685 | KENSYTTTIKPVTY | NSP3 | | CD8 | B*44:02 |
| 686 | YTTTIKPVTY | NSP3 | | CD8 | A*01:01, A*26:01 |
| 687 | CTEIDPKLDNY | NSP3 | Yes | CD8 | A*01:01 |
| 688 | TEIDPKLDNYY | NSP3 | | CD8 | A*01:01, B*44:02 |
| 689 | EIDPKLDNY | NSP3 | | CD8 | A*01:01, A*26:01 |
| 690 | NYYKKDNSY | NSP3 | | CD8 | C*07:02 |
| 691 | YYKKDNSY | NSP3 | | CD8 | C*07:02 |
| 692 | YYKKDNSYF | NSP3 | | CD8 | A*24:02 |
| 693 | FTEQPIDLVPNQPY | NSP3 | Yes | CD8 | |
| 694 | ASFDNFKFV | NSP3 | | CD8 | A*02:06, C*06:02 |
| 695 | KFADDLNQL | NSP3 | | CD8 | C*07:02 |
| 696 | FADDLNQLTGY | NSP3 | Yes | CD8 | A*01:01 |
| 697 | ASRELKVTF | NSP3 | | CD8 | A*30:01, B*57:01 |
| 698 | DVVAIDYKHY | NSP3 | | CD8 | A*26:01 |
| 699 | VVAIDYKHY | NSP3 | | CD8 | B*15:01 |
| 700 | DYKHYTPSF | NSP3 | | CD8 | A*24:02 |
| 701 | KLLHKPIVWHV | NSP3 | | CD8 | A*02:01 |
| 702 | LHKPIVWHV | NSP3 | | CD8 | C*06:02 |
| 703 | NKATYKPNTW | NSP3 | | CD8 | B*57:01 |
| 704 | KPVETSNSFDVL | NSP3 | | CD8 | B*07:02 |
| 705 | SEDAQGMDNL | NSP3 | | CD8 | B*40:01 |
| 706 | EEVVENPTI | NSP3 | | CD8 | B*44:03 |
| 707 | TEVVGDIIL | NSP3 | | CD8 | B*40:01 |
| 708 | ITEEVGHTDLMAAY | NSP3 | | CD8 | A*01:01 |
| 709 | EEVGHTDLMAAY | NSP3 | | CD8 | B*44:03 |
| 710 | EVGHTDLMAAY | NSP3 | | CD8 | A*26:01 |
| 711 | AYVDNSSLTI | NSP3 | | CD8 | A*24:02 |
| 712 | LTIKKPNEL | NSP3 | | CD8 | B*08:01 |
| 713 | KPNELSRVL | NSP3 | | CD8 | B*07:02, B*08:01 |
| 714 | NELSRVLGL | NSP3 | | CD8 | B*40:01, B*44:02 |
| 715 | SRVLGLKTL | NSP3 | | CD8 | C*07:01 |
| 716 | SVPWDTIANY | NSP3 | | CD8 | A*26:01 |
| 717 | DTIANYAKPF | NSP3 | | CD8 | A*26:01 |
| 718 | YAKPFLNKV | NSP3 | | CD8 | C*06:02 |
| 719 | NYMPYFFTL | NSP3 | Yes | CD8 | A*24:02 |
| 720 | MPYFFTLL | NSP3 | | CD8 | B*51:01 |
| 721 | RIKASMPTT | NSP3 | | CD8 | A*30:01 |
| 722 | KASMPTTIA | NSP3 | | CD8 | A*30:01 |
| 723 | ASMPTTIAK | NSP3 | Yes | CD8 | A*11:01, A*30:01 |
| 724 | MPTTIAKNTV | NSP3 | | CD8 | B*51:01 |
| 725 | TTIAKNTVK | NSP3 | | CD8 | A*30:01 |
| 726 | NTVKSVGKF | NSP3 | | CD8 | A*26:01 |
| 727 | KFCLEASFNY | NSP3 | | CD8 | A*29:02 |
| 728 | CLEASFNYL | NSP3 | | CD8 | A*02:01 |
| 729 | ASFNYLKSPNFSK | NSP3 | | CD8 | |
| 730 | KLINIIIWF | NSP3 | | CD8 | A*32:01 |
| 731 | SLIYSTAAL | NSP3 | | CD8 | A*02:01 |
| 732 | STAALGVLM | NSP3 | | CD8 | A*26:01 |
| 733 | MSNLGMPSY | NSP3 | | CD8 | B*15:01, B*57:01 |
| 734 | EGYLNSTNV | NSP3 | | CD8 | B*51:01 |
| 735 | NSTNVTIATY | NSP3 | | CD8 | A*26:01 |
| 736 | STNVTIATY | NSP3 | | CD8 | A*01:01, A*32:01 |
| 737 | SLDTYPSLETI | NSP3 | Yes | CD8 | A*02:01 |
| 738 | ETIQITISSF | NSP3 | | CD8 | A*26:01 |
| 739 | IQITISSFK | NSP3 | | CD8 | A*03:01 |
| 740 | LTAFGLVAEW | NSP3 | | CD8 | B*57:01 |
| 741 | LVAEWFLAY | NSP3 | | CD8 | A*26:01, A*29:02 |
| 742 | AEWFLAYIL | NSP3 | | CD8 | B*40:01, B*44:02 |
| 743 | AEWFLAYILF | NSP3 | | CD8 | B*44:02 |
| 744 | AYILFTRF | NSP3 | | CD8 | A*24:02 |
| 745 | AYILFTRFFYV | NSP3 | Yes | CD8 | |
| 746 | AYILFTRFF | NSP3 | | CD8 | A*24:02 |
| 747 | ILFTRFFYV | NSP3 | Yes | CD8 | A*02:01 |
| 748 | AVHFISNSW | NSP3 | | CD8 | B*57:01 |
| 749 | HFISNSWLMW | NSP3 | | CD8 | A*24:02 |
| 750 | WLMWLIINL | NSP3 | | CD8 | A*02:01 |
| 751 | LVQMAPISAM | NSP3 | | CD8 | B*15:01 |
| 752 | SAMVRMYIF | NSP3 | | CD8 | B*08:01 |
| 753 | RMYIFFASFY | NSP3 | | CD8 | A*03:01 |
| 754 | YIFFASFYY | NSP3 | Yes | CD8 | A*29:02 |
| 755 | SFYYVWKSY | NSP3 | | CD8 | A*29:02 |
| 756 | FYYVWKSYV | NSP3 | | CD8 | C*06:02, C*07:02 |
| 757 | FYYVWKSY | NSP3 | | CD8 | C*07:02 |
| 758 | YVYANGGKGF | NSP3 | | CD8 | A*26:01, B*15:01 |
| 759 | DTFCAGSTF | NSP3 | | CD8 | A*26:01 |
| 760 | EVARDLSLQF | NSP3 | | CD8 | A*26:01 |
| 761 | VARDLSLQF | NSP3 | | CD8 | B*57:01 |
| 762 | VTVKNGSIHLY | NSP3 | | CD8 | A*01:01 |
| 763 | TVKNGSIHLY | NSP3 | | CD8 | A*26:01 |
| 764 | VKNGSIHLY | NSP3 | | CD8 | C*06:02 |
| 765 | YFDKAGQKTY | NSP3 | | CD8 | C*07:02 |
| 766 | TYERHSLSHF | NSP3 | | CD8 | A*24:02 |
| 767 | YERHSLSHF | NSP3 | | CD8 | B*44:02 |
| 768 | ERHSLSHFV | NSP3 | | CD8 | C*06:02 |
| 769 | EESSAKSASVY | NSP3 | | CD8 | B*44:02 |
| 770 | SSAKSASVY | NSP3 | | CD8 | B*15:01 |
| 771 | SAKSASVYY | NSP3 | | CD8 | B*57:01 |
| 772 | QLMCQPILLL | NSP3 | Yes | CD8 | |
| 773 | DSAEVAVKM | NSP3 | | CD8 | A*26:01 |
| 774 | AEVAVKMF | NSP3 | | CD8 | B*44:02 |
| 775 | EVAVKMFDAY | NSP3 | | CD8 | A*26:01 |
| 776 | KMFDAYVNTF | NSP3 | | CD8 | A*24:02 |
| 777 | MFDAYVNTF | NSP3 | | CD8 | B*08:01 |
| 778 | AYVNTFSSTF | NSP3 | | CD8 | A*24:02 |
| 779 | YVNTFSSTF | NSP3 | | CD8 | A*26:01 |
| 780 | SSTFNVPMEKLK | NSP3 | Yes | CD8 | |
| 781 | STFNVPMEK | NSP3 | Yes | CD8 | A*11:01 |
| 782 | VPMEKLKTL | NSP3 | | CD8 | B*51:01 |
| 783 | AEAELAKNV | NSP3 | | CD8 | B*44:02, B*44:03 |
| 784 | AELAKNVSL | NSP3 | | CD8 | B*44:02 |
| 785 | AELAKNVSLDNVL | NSP3 | | CD8 | |
| 786 | SLDNVLSTF | NSP3 | | CD8 | A*32:01 |
| 787 | TFISAARQGF | NSP3 | | CD8 | A*24:02 |
| 788 | QSDIEVTGDSCNNY | NSP3 | | CD8 | A*01:01 |
| 789 | EVTGDSCNNYMLTY | NSP3 | | CD8 | A*26:01 |
| 790 | DSCNNYMLTY | NSP3 | | CD8 | A*01:01 |
| 791 | VENMTPRDL | NSP3 | | CD8 | B*44:03 |
| 792 | TPRDLGACI | NSP3 | | CD8 | B*07:02 |
| 793 | VAKSHNIAL | NSP3 | | CD8 | B*07:02 |
| 794 | AKSHNIALIW | NSP3 | | CD8 | B*57:01 |
| 795 | NVKDFMSL | NSP3 | | CD8 | B*08:01 |
| 796 | QVVNVVTTK | NSP3 | | CD8 | A*03:01 |
| 797 | VVTTKIALK | NSP3 | | CD8 | A*03:01 |
| 798 | KQLIKVTLVF | NSP4 | | CD8 | B*15:01 |
| 799 | FYLITPVHV | NSP4 | | CD8 | C*07:02 |
| 800 | YLITPVHV | NSP4 | | CD8 | A*02:01 |
| 801 | YLITPVHVM | NSP4 | | CD8 | A*02:01, B*15:01, C*07:01 |
| 802 | HTDFSSEIIGY | NSP4 | | CD8 | |
| 803 | SEIIGYKAI | NSP4 | | CD8 | B*40:01 |
| 804 | IAAVITREV | NSP4 | | CD8 | B*51:01 |
| 805 | FVVPGLPGT | NSP4 | | CD8 | A*02:06 |
| 806 | VPGLPGTIL | NSP4 | | CD8 | B*07:02 |
| 807 | RTTNGDFLHF | NSP4 | | CD8 | B*57:01 |
| 808 | FLPRVFSAV | NSP4 | Yes | CD8 | A*02:01 |
| 809 | LPRVFSAV | NSP4 | | CD8 | B*51:01 |
| 810 | FSAVGNICY | NSP4 | Yes | CD8 | A*01:01 |
| 811 | TPSKLIEY | NSP4 | | CD8 | B*35:01 |
| 812 | VLAAECTIF | NSP4 | | CD8 | B*15:01 |
| 813 | DASGKPVPY | NSP4 | | CD8 | B*35:01 |
| 814 | TNVLEGSVAY | NSP4 | | CD8 | B*35:01 |
| 815 | GSVAYESLR | NSP4 | | CD8 | A*31:01 |
| 816 | SLRPDTRYVL | NSP4 | Yes | CD8 | C*07:02 |
| 817 | LRPDTRYV | NSP4 | | CD8 | C*06:02 |
| 818 | RPDTRYVL | NSP4 | Yes | CD8 | B*07:02 |
| 819 | RPDTRYVLM | NSP4 | | CD8 | B*07:02, B*35:01 |
| 820 | SIIQFPNTY | NSP4 | | CD8 | B*35:01 |
| 821 | WVLNNDYYR | NSP4 | | CD8 | A*31:01 |
| 822 | YRSLPGVF | NSP4 | | CD8 | C*07:01 |
| 823 | SLPGVFCGV | NSP4 | | CD8 | A*02:01 |
| 824 | DAVNLLTNM | NSP4 | | CD8 | B*51:01 |
| 825 | IVAGGIVAI | NSP4 | | CD8 | A*02:01 |
| 826 | LAYYFMRFR | NSP4 | | CD8 | A*31:01 |
| 827 | YFMRFRRAF | NSP4 | | CD8 | A*24:02 |
| 828 | FGEYSHVVAF | NSP4 | | CD8 | B*40:01 |
| 829 | SFLPGVYSV | NSP4 | | CD8 | A*24:02 |
| 830 | FLPGVYSV | NSP4 | | CD8 | A*02:01 |
| 831 | LPGVYSVI | NSP4 | | CD8 | B*51:01 |
| 832 | IYLYLTFYL | NSP4 | | CD8 | A*24:02 |
| 833 | TFYLTNDVSFL | NSP4 | Yes | CD8 | |
| 834 | YLTNDVSFLA | NSP4 | | CD8 | A*02:01 |
| 835 | FLAHIQWMV | NSP4 | | CD8 | A*02:01, A*02:06 |
| 836 | MFTPLVPFW | NSP4 | | CD8 | A*24:02 |
| 837 | VPFWITIAY | NSP4 | | CD8 | B*35:01 |
| 838 | ISTKHFYW | NSP4 | | CD8 | B*57:01 |
| 839 | WFFSNYLKR | NSP4 | | CD8 | A*31:01 |
| 840 | YLKRRVVF | NSP4 | | CD8 | B*08:01 |
| 841 | FLLNKEMYL | NSP4 | Yes | CD8 | A*02:01 |
| 842 | DVLLPLTQY | NSP4 | | CD8 | B*35:01 |
| 843 | LPLTQYNRY | NSP4 | | CD8 | B*35:01 |
| 844 | GAMDTTSYR | NSP4 | | CD8 | A*31:01 |
| 845 | SNSGSDVLY | NSP4 | | CD8 | A*01:01 |
| 846 | LYQPPQTSI | NSP4 | | CD8 | A*24:02, C*07:01 |
| 847 | AVLQSGFRK | NSP5 | | CD8 | |
| 848 | TSEDMLNPNY | NSP5 | Yes | CD8 | A*01:01 |
| 849 | TANPKTPKY | NSP5 | | CD8 | C*07:01 |
| 850 | NPKTPKYKF | NSP5 | | CD8 | B*07:02 |
| 851 | TPKYKFVRI | NSP5 | Yes | CD8 | B*08:01 |
| 852 | VRIQPGQTF | NSP5 | Yes | CD8 | C*07:01 |
| 853 | IQPGQTFSV | NSP5 | | CD8 | |
| 854 | QPGQTFSVL | NSP5 | | CD8 | B*07:02 |
| 855 | SPSGVYQCAM | NSP5 | | CD8 | B*07:02 |
| 856 | FLNGSCGSV | NSP5 | Yes | CD8 | |
| 857 | YMHHMELPTGV | NSP5 | | CD8 | A*02:01 |
| 858 | GTDLEGNFY | NSP5 | Yes | CD8 | A*01:01 |
| 859 | TDLEGNFY | NSP5 | | CD8 | A*01:01 |
| 860 | VLAWLYAAV | NSP5 | Yes | CD8 | |
| 861 | FLNRFTTTL | NSP5 | Yes | CD8 | |
| 862 | ILTSLLVLV | NSP6 | | CD8 | A*02:01 |
| 863 | FLYENAFLP | NSP6 | | CD8 | A*02:01 |
| 864 | FLYENAFLPFA | NSP6 | | CD8 | A*02:01 |
| 865 | LPFAMGIIAM | NSP6 | | CD8 | B*07:02 |
| 866 | SAFAMMFVK | NSP6 | Yes | CD8 | A*11:01 |
| 867 | MFVKHKHAF | NSP6 | | CD8 | C*07:02 |
| 868 | FVKHKHAFL | NSP6 | | CD8 | B*08:01 |
| 869 | FLCLFLLPSLATV | NSP6 | Yes | CD8 | |
| 870 | LFLLPSLATV | NSP6 | | CD8 | A*02:01 |
| 871 | FLLPSLATVA | NSP6 | | CD8 | A*02:01 |
| 872 | FLLPSLATV | NSP6 | Yes | CD8 | A*02:01 |
| 873 | VYMPASWVM | NSP6 | | CD8 | A*24:02 |
| 874 | MPASWVMRI | NSP6 | Yes | CD8 | B*07:02 |
| 875 | RIMTWLDMV | NSP6 | | CD8 | A*02:01 |
| 876 | WLDMVDTSL | NSP6 | | CD8 | A*02:01 |
| 877 | KLKDCVMYA | NSP6 | Yes | CD8 | A*02:01 |
| 878 | VMYASAVVLL | NSP6 | | CD8 | A*24:02 |
| 879 | MYASAVVLL | NSP6 | | CD8 | C*07:02 |
| 880 | YASAVVLLI | NSP6 | | CD8 | C*06:02 |
| 881 | TLMNVLTLV | NSP6 | | CD8 | A*02:01 |
| 882 | SMWALIISV | NSP6 | Yes | CD8 | A*02:01 |
| 883 | NYSGVVTTVMF | NSP6 | Yes | CD8 | |
| 884 | FLARGIVFM | NSP6 | | CD8 | A*02:01 |
| 885 | IFFITGNTL | NSP6 | | CD8 | A*24:02 |
| 886 | NRYFRLTL | NSP6 | | CD8 | C*06:02, C*07:02 |
| 887 | TLGVYDYLVST | NSP6 | Yes | CD8 | |
| 888 | GVYDYLVST | NSP6 | | CD8 | A*02:01 |
| 889 | YDYLVSTQEF | NSP6 | | CD8 | C*07:02 |
| 890 | FRYMNSQGL | NSP6 | | CD8 | |
| 891 | GLLPPKNSI | NSP6 | | CD8 | A*02:01 |
| 892 | KLNIKLLGV | NSP6 | | CD8 | A*02:01 |
| 893 | KLWAQCVQL | NSP7 | Yes | CD8 | A*02:01 |
| 894 | EAFEKMVSL | NSP7 | | CD8 | B*08:01 |
| 895 | SLLSVLLSM | NSP7 | | CD8 | A*02:01 |
| 896 | SEFSSLPSY | NSP8 | Yes | CD8 | B*44:03 |
| 897 | LKKLKKSL | NSP8 | | CD8 | B*08:01 |
| 898 | KMADQAMTQMY | NSP8 | | CD8 | B*15:01 |
| 899 | LMVVIPDYNTY | NSP8 | | CD8 | B*15:01 |
| 900 | TYKNTCDGTTF | NSP8 | | CD8 | A*24:02 |
| 901 | NTCDGTTFTY | NSP8 | Yes | CD8 | A*01:01 |
| 902 | TFTYASALW | NSP8 | | CD8 | A*24:02 |
| 903 | FTYASALWEI | NSP8 | Yes | CD8 | |
| 904 | SALWEIQQVV | NSP8 | Yes | CD8 | A*02:01 |
| 905 | ALWEIQQV | NSP8 | Yes | CD8 | A*02:01 |
| 906 | ALWEIQQVV | NSP8 | Yes | CD8 | A*02:01 |
| 907 | SEISMDNSPNL | NSP8 | | CD8 | |
| 908 | AWPLIVTAL | NSP8 | | CD8 | A*24:02 |
| 909 | ALRANSAVK | NSP8 | Yes | CD8 | |
| 910 | CTDDNALAYY | NSP9 | Yes | CD8 | A*01:01 |
| 911 | CTDDNALAY | NSP9 | Yes | CD8 | A*01:01 |
| 912 | SDGTGTIY | NSP9 | | CD8 | A*01:01 |
| 913 | YTELEPPCRF | NSP9 | Yes | CD8 | A*01:01 |
| 914 | VTDTPKGPK | NSP9 | Yes | CD8 | A*11:01 |
| 915 | GPKVKYLYF | NSP9 | | CD8 | B*08:01 |
| 916 | YLYFIKGLNNL | NSP9 | | CD8 | A*02:01 |
| 917 | YFIKGLNNL | NSP9 | | CD8 | C*07:02 |
| 918 | VPANSTVL | NSP10 | | CD8 | B*07:02 |
| 919 | VPANSTVLSF | NSP10 | | CD8 | B*07:02 |
| 920 | TVLSFCAFAV | NSP10 | Yes | CD8 | |
| 921 | YLASGGQPI | NSP10 | | CD8 | A*02:01 |
| 922 | DLKGKYVQI | NSP10 | Yes | CD8 | B*08:01 |
| 923 | VVYRAFDIY | NSP12 | | CD8 | B*15:01 |
| 924 | FDIYNDKVAGF | NSP12 | | CD8 | A*24:02 |
| 925 | KVAGFAKFL | NSP12 | | CD8 | A*32:01 |
| 926 | KVAGFAKFLK | NSP12 | | CD8 | A*11:01 |
| 927 | NLIDSYFVV | NSP12 | | CD8 | A*02:01 |
| 928 | YFVVKRHTF | NSP12 | | CD8 | A*24:02, B*08:01 |
| 929 | VPHISRQRL | NSP12 | | CD8 | B*07:02 |
| 930 | YTMADLVYAL | NSP12 | Yes | CD8 | |
| 931 | TMADLVYAL | NSP12 | Yes | CD8 | A*02:01 |
| 932 | TLKEILVTY | NSP12 | | CD8 | A*29:02 |
| 933 | FVENPDILRV | NSP12 | | CD8 | A*02:06 |
| 934 | VENPDILRV | NSP12 | | CD8 | B*44:03 |
| 935 | VENPDILRVY | NSP12 | | CD8 | B*44:02 |
| 936 | VRQALLKTV | NSP12 | | CD8 | C*06:02 |
| 937 | DAMRNAGIV | NSP12 | | CD8 | B*51:01 |
| 938 | SLLMPILTL | NSP12 | | CD8 | A*02:01 |
| 939 | LLMPILTLT | NSP12 | Yes | CD8 | |
| 940 | AESHVDTDLTKPY | NSP12 | | CD8 | B*44:02 |
| 941 | HVDTDLTKPY | NSP12 | | CD8 | A*01:01 |
| 942 | KPYIKWDLL | NSP12 | | CD8 | B*07:02 |
| 943 | KLFDRYFKY | NSP12 | Yes | CD8 | A*03:01 |
| 944 | RYFKYWDQTY | NSP12 | | CD8 | A*24:02 |
| 945 | ILHCANFNV | NSP12 | | CD8 | |
| 946 | STVFPPTSF | NSP12 | | CD8 | B*57:01 |
| 947 | FPPTSFGPL | NSP12 | | CD8 | |
| 948 | TSFGPLVRK | NSP12 | | CD8 | A*03:01 |
| 949 | SFGPLVRKI | NSP12 | | CD8 | A*24:02 |
| 950 | KIFVDGVPFV | NSP12 | | CD8 | A*02:01 |
| 951 | FVDGVPFVV | NSP12 | Yes | CD8 | A*02:01 |
| 952 | FVVSTGYHFR | NSP12 | | CD8 | A*68:01 |
| 953 | VVSTGYHFR | NSP12 | | CD8 | A*11:01 |
| 954 | NLHSSRLSF | NSP12 | | CD8 | B*08:01 |
| 955 | QTVKPGNFNK | NSP12 | | CD8 | A*11:01 |
| 956 | DFYDFAVSKGFFK | NSP12 | | CD8 | A*33:01 |
| 957 | FAVSKGFFK | NSP12 | | CD8 | A*11:01 |
| 958 | AAISDYDYYR | NSP12 | | CD8 | A*68:01 |
| 959 | AAISDYDYY | NSP12 | | CD8 | A*01:01 |
| 960 | AISDYDYYR | NSP12 | | CD8 | A*11:01 |
| 961 | ISDYDYYRY | NSP12 | Yes | CD8 | |
| 962 | YRYNLPTMC | NSP12 | | CD8 | C*06:02 |
| 963 | YRYNLPTM | NSP12 | | CD8 | C*07:02 |
| 964 | RQLLFVVEV | NSP12 | Yes | CD8 | A*02:01 |
| 965 | FVVEVVDKY | NSP12 | | CD8 | B*15:01 |
| 966 | VVDKYFDCY | NSP12 | Yes | CD8 | |
| 967 | KSAGFPFNKW | NSP12 | | CD8 | B*57:01 |
| 968 | KSAGFPFNK | NSP12 | | CD8 | A*03:01 |
| 969 | ARLYYDSMSY | NSP12 | | CD8 | C*07:02 |
| 970 | RLYYDSMSY | NSP12 | Yes | CD8 | B*15:01 |
| 971 | FAYTKRNVI | NSP12 | | CD8 | B*51:01 |
| 972 | NVIPTITQM | NSP12 | | CD8 | C*07:01 |
| 973 | IPTITQMNL | NSP12 | | CD8 | B*07:02 |
| 974 | TITQMNLKY | NSP12 | | CD8 | A*01:01 |
| 975 | YAISAKNRAR | NSP12 | | CD8 | A*68:01 |
| 976 | SAKNRARTV | NSP12 | | CD8 | C*06:02 |
| 977 | SICSTMTNR | NSP12 | | CD8 | A*33:01 |
| 978 | IAATRGATV | NSP12 | | CD8 | B*51:01 |
| 979 | ATVVIGTSK | NSP12 | | CD8 | A*11:01 |
| 980 | YSDVENPHLMGW | NSP12 | | CD8 | B*44:02 |
| 981 | VENPHLMGWD | NSP12 | | CD8 | B*44:02 |
| 982 | MPNMLRIMASL | NSP12 | | CD8 | B*07:02 |
| 983 | NMLRIMASL | NSP12 | | CD8 | |
| 984 | MLRIMASL | NSP12 | | CD8 | B*08:01 |
| 985 | LRIMASLVL | NSP12 | | CD8 | C*07:02 |
| 986 | IMASLVLAR | NSP12 | | CD8 | A*33:01 |
| 987 | MASLVLARK | NSP12 | | CD8 | A*68:01 |
| 988 | RLANECAQV | NSP12 | | CD8 | A*02:01 |
| 989 | MVMCGGSLYV | NSP12 | Yes | CD8 | |
| 990 | TSSGDATTAY | NSP12 | | CD8 | A*01:01 |
| 991 | VRNLQHRLY | NSP12 | | CD8 | C*07:01 |
| 992 | DTDFVNEFY | NSP12 | Yes | CD8 | A*01:01 |
| 993 | FVNEFYAYLR | NSP12 | | CD8 | A*33:01 |
| 994 | FVNEFYAYL | NSP12 | | CD8 | A*02:01 |
| 995 | FYAYLRKHF | NSP12 | Yes | CD8 | A*24:02 |
| 996 | YAYLRKHF | NSP12 | | CD8 | B*08:01 |
| 997 | LRKHFSMMI | NSP12 | | CD8 | C*06:02 |
| 998 | LSDDAVVCFNSTY | NSP12 | Yes | CD8 | |
| 999 | LYYQNNVFM | NSP12 | | CD8 | A*24:02 |
| 1000 | TETDLTKGPHEF | NSP12 | | CD8 | B*44:02 |
| 1001 | LVKQGDDYVY | NSP12 | | CD8 | B*15:01 |
| 1002 | KQGDDYVYL | NSP12 | | CD8 | A*02:01 |
| 1003 | DYVYLPYPDPSRI | NSP12 | | CD8 | B*51:01 |
| 1004 | VYLPYPDPSRI | NSP12 | | CD8 | B*51:01 |
| 1005 | YLPYPDPSRI | NSP12 | | CD8 | B*51:01 |
| 1006 | YLPYPDPSRIL | NSP12 | | CD8 | A*24:02 |
| 1007 | LPYPDPSRIL | NSP12 | | CD8 | B*07:02, B*51:01 |
| 1008 | RILGAGCFV | NSP12 | Yes | CD8 | |
| 1009 | LMIERFVSL | NSP12 | Yes | CD8 | A*02:01 |
| 1010 | QEYADVFHLY | NSP12 | | CD8 | A*29:02, B*44:03 |
| 1011 | YADVFHLYL | NSP12 | | CD8 | C*07:02 |
| 1012 | LYLQYIRKL | NSP12 | Yes | CD8 | C*07:02 |
| 1013 | LTNDNTSRYW | NSP12 | | CD8 | B*57:01 |
| 1014 | NTSRYWEPEFY | NSP12 | | CD8 | A*01:01 |
| 1015 | SRYWEPEF | NSP12 | | CD8 | C*07:01 |
| 1016 | WEPEFYEAM | NSP12 | | CD8 | B*40:01 |
| 1017 | AMYTPHTVL | NSP12 | | CD8 | A*32:01 |
| 1018 | TPHTVLQAV | NSP12 | | CD8 | B*51:01 |
| 1019 | VLQAVGACV | NSP13 | Yes | CD8 | |
| 1020 | LVLSVNPYV | NSP13 | Yes | CD8 | |
| 1021 | QLYLGGMSYY | NSP13 | | CD8 | B*15:01 |
| 1022 | KPPISFPL | NSP13 | | CD8 | B*07:02 |
| 1023 | KLFAAETLK | NSP13 | Yes | CD8 | A*03:01 |
| 1024 | KLSYGIATV | NSP13 | Yes | CD8 | A*02:01 |
| 1025 | VVYRGTTTYK | NSP13 | Yes | CD8 | A*03:01, A*11:01 |
| 1026 | VVYRGTTTY | NSP13 | Yes | CD8 | |
| 1027 | VYRGTTTYKL | NSP13 | Yes | CD8 | A*24:02 |
| 1028 | YRGTTTYKL | NSP13 | | CD8 | C*06:02 |
| 1029 | KLNVGDYFV | NSP13 | Yes | CD8 | A*02:01 |
| 1030 | APTLVPQEHYV | NSP13 | | CD8 | A*02:01 |
| 1031 | TLVPQEHYV | NSP13 | | CD8 | |
| 1032 | VPQEHYVRI | NSP13 | | CD8 | B*08:01 |
| 1033 | SSNVANYQK | NSP13 | Yes | CD8 | |
| 1034 | YQKVGMQKY | NSP13 | | CD8 | |
| 1035 | FAIGLALYY | NSP13 | | CD8 | C*07:02 |
| 1036 | YYPSARIVY | NSP13 | | CD8 | A*24:02 |
| 1037 | IPARARVEC | NSP13 | | CD8 | B*07:02 |
| 1038 | IPARARVECF | NSP13 | | CD8 | B*07:02 |
| 1039 | YVFCTVNAL | NSP13 | | CD8 | |
| 1040 | VVNARLRAK | NSP13 | | CD8 | A*11:01 |
| 1041 | VYIGDPAQL | NSP13 | Yes | CD8 | A*24:02, C*07:01 |
| 1042 | IVDTVSALV | NSP13 | Yes | CD8 | |
| 1043 | RPQIGVVREF | NSP13 | | CD8 | B*15:01 |
| 1044 | AVASKILGL | NSP13 | | CD8 | A*02:01 |
| 1045 | ILGLPTQTV | NSP13 | | CD8 | A*02:01 |
| 1046 | LEIPRRNVATL | NSP13 | | CD8 | B*07:02 |
| 1047 | IPRRNVATL | NSP13 | Yes | CD8 | B*07:02, B*08:01 |
| 1048 | EEAIRHVRAW | NSP14 | | CD8 | B*44:03 |
| 1049 | LQLGFSTGV | NSP14 | | CD8 | |
| 1050 | MYKGLPWNV | NSP14 | | CD8 | C*06:02 |
| 1051 | KNLSDRVVFV | NSP14 | | CD8 | A*02:01 |
| 1052 | VLWAHGFEL | NSP14 | Yes | CD8 | |
| 1053 | TYACWHHSI | NSP14 | Yes | CD8 | A*24:02 |
| 1054 | PFMIDVQQW | NSP14 | | CD8 | A*24:02 |
| 1055 | AIMTRCLAV | NSP14 | Yes | CD8 | |
| 1056 | IEYPIIGDEL | NSP14 | Yes | CD8 | B*40:01 |
| 1057 | ALLADKFPV | NSP14 | Yes | CD8 | A*02:01 |
| 1058 | LLADKFPVL | NSP14 | | CD8 | B*08:01 |
| 1059 | YKIEELFYSY | NSP14 | | CD8 | A*01:01 |
| 1060 | KIEELFYSY | NSP14 | | CD8 | B*15:01 |
| 1061 | SYATHSDKF | NSP14 | Yes | CD8 | A*24:02 |
| 1062 | FTDGVCLFW | NSP14 | | CD8 | A*01:01 |
| 1063 | SLYVNKHAF | NSP14 | | CD8 | B*08:01 |
| 1064 | YLDAYNMMI | NSP14 | Yes | CD8 | A*02:01 |
| 1065 | MMISAGFSL | NSP14 | Yes | CD8 | A*02:01 |
| 1066 | AGFSLWVYK | NSP14 | | CD8 | A*11:01 |
| 1067 | KQFDTYNLW | NSP14 | Yes | CD8 | B*15:01 |
| 1068 | YNLWNTFTRL | NSP14 | | CD8 | A*02:01 |
| 1069 | NLWNTFTRL | NSP14 | Yes | CD8 | A*02:01 |
| 1070 | NTFTRLQSL | NSP14 | | CD8 | C*07:01 |
| 1071 | SLENVAFNV | NSP15 | | CD8 | A*02:01 |
| 1072 | KVDGVDVEL | NSP15 | Yes | CD8 | A*02:01 |
| 1073 | TTLPVNVAF | NSP15 | | CD8 | |
| 1074 | TICAPLTVF | NSP15 | | CD8 | B*15:01 |
| 1075 | GRVDGQVDL | NSP15 | | CD8 | C*07:01 |
| 1076 | KVDGVVQQL | NSP15 | | CD8 | A*02:01 |
| 1077 | VVQQLPETY | NSP15 | | CD8 | B*15:01 |
| 1078 | KPRSQMEIDF | NSP15 | | CD8 | B*07:02 |
| 1079 | FIERYKLEGY | NSP15 | | CD8 | A*01:01 |
| 1080 | FIPMDSTVKNY | NSP15 | | CD8 | A*01:01 |
| 1081 | LLLDDFVEI | NSP15 | | CD8 | A*02:01 |
| 1082 | LLLDDFVEII | NSP15 | Yes | CD8 | |
| 1083 | SVVSKVVKV | NSP15 | | CD8 | A*02:01 |
| 1084 | MLWCKDGHV | NSP15 | Yes | CD8 | |
| 1085 | GVAMPNLYK | NSP16 | Yes | CD8 | |
| 1086 | VAMPNLYKM | NSP16 | | CD8 | B*57:01 |
| 1087 | KMQRMLLEK | NSP16 | Yes | CD8 | |
| 1088 | LPKGIMMNV | NSP16 | | CD8 | B*07:02 |
| 1089 | YLNTLTLAV | NSP16 | Yes | CD8 | |
| 1090 | GVAPGTAVLRQW | NSP16 | | CD8 | B*57:01 |
| 1091 | TLIGDCATV | NSP16 | Yes | CD8 | |
| 1092 | CATVHTANKW | NSP16 | | CD8 | B*57:01 |
| 1093 | KLMGHFAWW | NSP16 | | CD8 | A*32:01 |
| 1094 | SSEAFLIGCNY | NSP16 | | CD8 | A*01:01 |
| 1095 | REQIDGYVMHANY | NSP16 | | CD8 | B*44:02 |
| 1096 | YVMHANYIF | NSP16 | | CD8 | A*32:01 |
| 1097 | FWRNTNPIQL | NSP16 | | CD8 | C*07:01 |
| 1098 | WRNTNPIQL | NSP16 | | CD8 | C*07:01 |
| 1099 | NPIQLSSYSL | NSP16 | | CD8 | B*07:02 |
| 1100 | SYSLFDMSKF | NSP16 | | CD8 | A*24:02 |
| 1101 | FPLKLRGTA | NSP16 | | CD8 | B*07:02 |
| 1102 | FPLKLRGTAV | NSP16 | | CD8 | B*08:01 |
| 1103 | LRGTAVMSL | NSP16 | | CD8 | C*07:01 |
| 1104 | MDLFMRIFTI | ORF3a | Yes | CD8 | |
| 1105 | RIFTIGTVTLK | ORF3a | Yes | CD8 | |
| 1106 | FTIGTVTLK | ORF3a | | CD8 | A*68:01 |
| 1107 | FVRATATIPI | ORF3a | Yes | CD8 | |
| 1108 | ATIPIQASL | ORF3a | | CD8 | B*57:01 |
| 1109 | IPIQASLPF | ORF3a | Yes | CD8 | B*35:01, B*51:01 |
| 1110 | LPFGWLIV | ORF3a | | CD8 | B*51:01 |
| 1111 | IVGVALLAVF | ORF3a | Yes | CD8 | |
| 1112 | ALLAVFQSA | ORF3a | | CD8 | A*02:01 |
| 1113 | QSASKIITL | ORF3a | | CD8 | B*08:01 |
| 1114 | SASKIITLKKRW | ORF3a | | CD8 | B*57:01 |
| 1115 | SASKIITLK | ORF3a | | CD8 | A*03:01, A*11:01 |
| 1116 | ITLKKRWQL | ORF3a | | CD8 | B*08:01, B*57:01 |
| 1117 | ITLKKRWQLAL | ORF3a | Yes | CD8 | |
| 1118 | TLKKRWQLA | ORF3a | | CD8 | B*08:01 |
| 1119 | ALSKGVHFV | ORF3a | Yes | CD8 | A*02:01 |
| 1120 | NLLLLFVTV | ORF3a | | CD8 | A*02:01 |
| 1121 | FVTVYSHLL | ORF3a | | CD8 | A*02:01 |
| 1122 | TVYSHLLLV | ORF3a | | CD8 | A*02:01 |
| 1123 | VAAGLEAPF | ORF3a | | CD8 | B*35:01 |
| 1124 | AAGLEAPFLYLY | ORF3a | | CD8 | B*44:02 |
| 1125 | AAGLEAPFLY | ORF3a | | CD8 | A*01:01 |
| 1126 | AGLEAPFLY | ORF3a | | CD8 | A*29:02 |
| 1127 | GLEAPFLYL | ORF3a | | CD8 | A*02:01 |
| 1128 | LEAPFLYLY | ORF3a | | CD8 | A*29:02 |
| 1129 | APFLYLYAL | ORF3a | | CD8 | B*07:02, B*08:01 |
| 1130 | FLYLYALVY | ORF3a | | CD8 | C*07:02 |
| 1131 | LYLYALVYF | ORF3a | | CD8 | A*24:02 |
| 1132 | YLYALVYFL | ORF3a | Yes | CD8 | A*02:01 |
| 1133 | VYFLQSINF | ORF3a | Yes | CD8 | A*24:02 |
| 1134 | FVRIIMRLW | ORF3a | | CD8 | B*57:01 |
| 1135 | VRIIMRLWL | ORF3a | | CD8 | C*07:02 |
| 1136 | CRSKNPLLY | ORF3a | | CD8 | C*06:02 |
| 1137 | NPLLYDANY | ORF3a | | CD8 | B*53:01 |
| 1138 | NPLLYDANYFLCW | ORF3a | Yes | CD8 | |
| 1139 | NPLLYDANYFL | ORF3a | | CD8 | A*02:01 |
| 1140 | LLYDANYFL | ORF3a | Yes | CD8 | A*02:01 |
| 1141 | IPYNSVTSSIVI | ORF3a | | CD8 | B*51:01 |
| 1142 | IPYNSVTSSI | ORF3a | | CD8 | |
| 1143 | TTSPISEHDY | ORF3a | | CD8 | |
| 1144 | SEHDYQIGGYTEKW | ORF3a | Yes | CD8 | |
| 1145 | VLHSYFTSDYYQLY | ORF3a | Yes | CD8 | |
| 1146 | HSYFTSDYY | ORF3a | | CD8 | A*29:02 |
| 1147 | SYFTSDYYQLY | ORF3a | | CD8 | A*24:02 |
| 1148 | YFTSDYYQLY | ORF3a | | CD8 | A*01:01, A*29:02 |
| 1149 | YFTSDYYQL | ORF3a | | CD8 | C*07:02 |
| 1150 | FTSDYYQLY | ORF3a | Yes | CD8 | A*01:01, A*24:02 |
| 1151 | DYYQLYSTQL | ORF3a | | CD8 | A*24:02 |
| 1152 | YYQLYSTQL | ORF3a | Yes | CD8 | A*24:02, C*07:02 |
| 1153 | HVTFFIYNK | ORF3a | | CD8 | A*68:01 |
| 1154 | EEHVQIHTI | ORF3a | | CD8 | |
| 1155 | IYDEPTTTT | ORF3a | | CD8 | C*07:02 |
| 1156 | HLVDFQVTIA | ORF6 | | CD8 | A*02:01 |
| 1157 | HLVDFQVTI | ORF6 | Yes | CD8 | A*02:01 |
| 1158 | VTIAEILLI | ORF6 | | CD8 | |
| 1159 | LIIMRTFKV | ORF6 | | CD8 | B*08:01 |
| 1160 | RTFKVSIWNLDY | ORF6 | | CD8 | A*01:01 |
| 1161 | TFKVSIWNL | ORF6 | | CD8 | B*08:01 |
| 1162 | SIWNLDYIINL | ORF6 | | CD8 | A*02:01 |
| 1163 | LDYIINLII | ORF6 | | CD8 | |
| 1164 | IIKNLSKSLTENKY | ORF6 | | CD8 | |
| 1165 | QECVRGTTVL | ORF7a | | CD8 | |
| 1166 | LLKEPCSSGTY | ORF7a | | CD8 | B*15:01 |
| 1167 | YEGNSPFHPL | ORF7a | | CD8 | B*40:01 |
| 1168 | EGNSPFHPL | ORF7a | | CD8 | B*08:01 |
| 1169 | SPFHPLADNKFAL | ORF7a | | CD8 | |
| 1170 | HPLADNKFAL | ORF7a | | CD8 | B*08:01 |
| 1171 | KFALTCFSTQF | ORF7a | | CD8 | A*24:02 |
| 1172 | FACPDGVKHVY | ORF7a | | CD8 | A*01:01 |
| 1173 | QLRARSVSPK | ORF7a | Yes | CD8 | A*03:01 |
| 1174 | RARSVSPKL | ORF7a | Yes | CD8 | B*07:02 |
| 1175 | RARSVSPKLFIR | ORF7a | Yes | CD8 | |
| 1176 | KLFIRQEEV | ORF7a | Yes | CD8 | |
| 1177 | FIRQEEVQELY | ORF7a | | CD8 | A*01:01, B*15:01 |
| 1178 | VQELYSPIFLIV | ORF7a | Yes | CD8 | |
| 1179 | SPIFLIVAA | ORF7a | | CD8 | B*07:02 |
| 1180 | VFITLCFTLK | ORF7a | Yes | CD8 | |
| 1181 | IMLIIFWFSL | ORF7b | Yes | CD8 | |
| 1182 | FLGIITTV | ORF8 | | CD8 | A*02:01 |
| 1183 | QSCTQHQPY | ORF8 | | CD8 | A*01:01 |
| 1184 | YVVDDPCPIHFY | ORF8 | Yes | CD8 | |
| 1185 | VDDPCPIHFY | ORF8 | | CD8 | A*01:01 |
| 1186 | RVGARKSAPL | ORF8 | | CD8 | |
| 1187 | IQYIDIGNY | ORF8 | Yes | CD8 | |
| 1188 | GNYTVSCLPFTI | ORF8 | Yes | CD8 | |
| 1189 | EPKLGSLVV | ORF8 | | CD8 | B*07:02 |
| 1190 | KLGSLVVRC | ORF8 | Yes | CD8 | A*02:01 |
| 1191 | YEDFLEYHDVRVVL | ORF8 | Yes | CD8 | |
| 1192 | LEYHDVRVVL | ORF8 | Yes | CD8 | B*40:01 |
| 1193 | EYHDVRVVL | ORF8 | | CD8 | A*24:02 |
| 1194 | EYHDVRVVLDF | ORF8 | | CD8 | A*24:02 |
| 1195 | YINVFAFPF | ORF10 | | CD8 | A*02:01 |
| 1196 | NVFAFPFTI | ORF10 | | CD8 | A*02:01 |
| 1197 | SLLLCRMNSRNYIA | ORF10 | Yes | CD8 | |
| 1198 | NYIAQVDVVNFNL | ORF10 | Yes | CD8 | |
| 1199 | FVFLVLLPL | S | | CD8 | A*02:01 |
| 1200 | LPLVSSQCV | S | | CD8 | B*51:01 |
| 1201 | NLTTRTQL | S | | CD8 | |
| 1202 | TRTQLPPAY | S | | CD8 | C*07:02 |
| 1203 | LPPAYTNSF | S | | CD8 | B*07:02, B*35:01, B*53:01 |
| 1204 | YTNSFTRGVY | S | Yes | CD8 | A*01:01, A*26:01 |
| 1205 | YTNSFTRGVYY | S | | CD8 | A*01:01 |
| 1206 | NSFTRGVYY | S | | CD8 | A*26:01, A*29:02, A*68:01 |
| 1207 | GVYYPDKVFR | S | | CD8 | A*03:01, A*68:01 |
| 1208 | VYYPDKVFRSSVLH | S | Yes | CD8 | |
| 1209 | VYYPDKVF | S | | CD8 | A*24:02 |
| 1210 | KVFRSSVLH | S | | CD8 | A*03:01, A*30:01 |
| 1211 | FRSSVLHST | S | | CD8 | C*07:01 |
| 1212 | STQDLFLPFF | S | Yes | CD8 | A*01:01 |
| 1213 | STQDLFLPF | S | | CD8 | A*01:01 |
| 1214 | TQDLFLPFF | S | | CD8 | A*24:02 |
| 1215 | FLPFFSNVTW | S | | CD8 | B*53:01 |
| 1216 | FLPFFSNVTWFHAI | S | Yes | CD8 | |
| 1217 | FLPFFSNV | S | | CD8 | A*02:01 |
| 1218 | PFFSNVTWF | S | | CD8 | A*24:02 |
| 1219 | HVSGTNGTK | S | | CD8 | A*68:01 |
| 1220 | RFDNPVLPF | S | Yes | CD8 | A*24:02, C*07:02 |
| 1221 | FDNPVLPFNDGVYF | S | | CD8 | B*35:01 |
| 1222 | VLPFNDGVYFA | S | | CD8 | A*02:01 |
| 1223 | LPFNDGVYF | S | | CD8 | B*07:02, B*35:01, B*51:01 |
| 1224 | DGVYFASTEK | S | | CD8 | A*68:01 |
| 1225 | GVYFASTEK | S | Yes | CD8 | A*03:01, A*11:01, A*68:01 |
| 1226 | TEKSNIIRGW | S | Yes | CD8 | B*44:02, B*44:03 |
| 1227 | WIFGTTLDSK | S | | CD8 | |
| 1228 | TLDSKTQSL | S | Yes | CD8 | A*02:01, B*08:01, C*07:02 |
| 1229 | FCNDPFLGVYY | S | Yes | CD8 | A*01:01 |
| 1230 | CNDPFLGVY | S | | CD8 | A*01:01 |
| 1231 | GVYYHKNNK | S | | CD8 | A*03:01 |
| 1232 | YYHKNNKSW | S | | CD8 | A*24:02 |
| 1233 | KSWMESEFRVY | S | Yes | CD8 | |
| 1234 | SWMESEFRV | S | | CD8 | A*24:02 |
| 1235 | SWMESEFRVY | S | | CD8 | A*29:02 |
| 1236 | VYSSANNCTFEY | S | Yes | CD8 | |
| 1237 | VYSSANNCTF | S | | CD8 | A*24:02 |
| 1238 | SSANNCTFEY | S | Yes | CD8 | A*01:01 |
| 1239 | SANNCTFEY | S | | CD8 | A*29:02, B*35:01 |
| 1240 | CTFEYVSQPFLM | S | Yes | CD8 | |
| 1241 | TFEYVSQPFLM | S | | CD8 | A*24:02 |
| 1242 | FEYVSQPFLM | S | | CD8 | B*40:01 |
| 1243 | EYVSQPFLM | S | | CD8 | A*24:02 |
| 1244 | FKNLREFVF | S | | CD8 | C*07:01 |
| 1245 | FVFKNIDGY | S | Yes | CD8 | A*26:01, A*29:02 |
| 1246 | VFKNIDGYF | S | | CD8 | A*24:02 |
| 1247 | KIYSKHTPI | S | | CD8 | B*08:01 |
| 1248 | IYSKHTPINL | S | Yes | CD8 | |
| 1249 | TPINLVRDL | S | Yes | CD8 | B*07:02 |
| 1250 | LPQGFSALEPL | S | | CD8 | B*07:02 |
| 1251 | LPQGFSAL | S | Yes | CD8 | B*07:02, B*08:01 |
| 1252 | LEPLVDLPI | S | | CD8 | |
| 1253 | EPLVDLPI | S | | CD8 | B*51:01 |
| 1254 | DLPIGINITRFQTL | S | Yes | CD8 | |
| 1255 | LPIGINITRF | S | | CD8 | B*07:02, B*35:01 |
| 1256 | INITRFQTL | S | | CD8 | B*08:01 |
| 1257 | NITRFQTL | S | | CD8 | B*08:01 |
| 1258 | LLALHRSYL | S | Yes | CD8 | |
| 1259 | WTAGAAAYY | S | Yes | CD8 | A*26:01, A*29:02, B*35:01 |
| 1260 | WTAGAAAYYVGY | S | Yes | CD8 | |
| 1261 | GAAAYYVGY | S | | CD8 | A*29:02 |
| 1262 | YYVGYLQPRTFLL | S | Yes | CD8 | |
| 1263 | YYVGYLQPRTF | S | Yes | CD8 | A*24:02 |
| 1264 | VGYLQPRTFL | S | | CD8 | A*24:02 |
| 1265 | GYLQPRTFLL | S | | CD8 | A*24:02 |
| 1266 | YLQPRTFLL | S | Yes | CD8 | A*02:01, A*24:02, B*08:01 |
| 1267 | CALDPLSETK | S | | CD8 | |
| 1268 | ALDPLSETK | S | | CD8 | A*03:01 |
| 1269 | SETKCTLKSF | S | | CD8 | B*44:02, B*44:03 |
| 1270 | SETKCTLKSFTVEK | S | Yes | CD8 | |
| 1271 | ETKCTLKSF | S | | CD8 | A*26:01 |
| 1272 | IYQTSNFRV | S | | CD8 | A*24:02 |
| 1273 | RVQPTESIVRF | S | | CD8 | B*07:02 |
| 1274 | QPTESIVRF | S | | CD8 | B*07:02, B*35:01 |
| 1275 | TESIVRFPNITNL | S | | CD8 | B*40:01 |
| 1276 | RFPNITNLCPF | S | | CD8 | A*24:02 |
| 1277 | FGEVFNATRFASVY | S | Yes | CD8 | |
| 1278 | GEVFNATRF | S | | CD8 | B*40:01, B*44:02, B*44:03 |
| 1279 | EVFNATRFASVY | S | | CD8 | A*26:01 |
| 1280 | NATRFASVY | S | | CD8 | B*35:01 |
| 1281 | TRFASVYAW | S | | CD8 | C*07:02 |
| 1282 | RFASVYAWNR | S | | CD8 | A*31:01 |
| 1283 | FASVYAWNR | S | | CD8 | A*68:01 |
| 1284 | SVYAWNRKR | S | | CD8 | A*03:01 |
| 1285 | YAWNRKRI | S | | CD8 | B*51:01 |
| 1286 | RISNCVADY | S | Yes | CD8 | |
| 1287 | CVADYSVLY | S | Yes | CD8 | A*01:01, A*26:01, A*29:02, |
| | | | | | B*15:01 |
| 1288 | YSVLYNSASFSTFK | S | Yes | CD8 | |
| 1289 | NSASFSTFK | S | | CD8 | A*68:01 |
| 1290 | SASFSTFKCY | S | | CD8 | A*01:01 |
| 1291 | KCYGVSPTK | S | Yes | CD8 | A*03:01 |
| 1292 | KLNDLCFTNV | S | | CD8 | A*02:01 |
| 1293 | NVYADSFVIR | S | | CD8 | A*68:01 |
| 1294 | FVIRGDEVR | S | | CD8 | A*68:01 |
| 1295 | QIAPGQTGK | S | | CD8 | A*68:01 |
| 1296 | KIADYNYKL | S | Yes | CD8 | A*02:01 |
| 1297 | KLPDDFTGCV | S | Yes | CD8 | A*02:01 |
| 1298 | NLDSKVGGNY | S | Yes | CD8 | |
| 1299 | DSKVGGNYNY | S | | CD8 | A*26:01 |
| 1300 | KVGGNYNYLY | S | | CD8 | A*29:02 |
| 1301 | VGGNYNYLY | S | | CD8 | A*29:02 |
| 1302 | GNYNYLYRLF | S | | CD8 | A*24:02 |
| 1303 | NYNYLYRLF | S | Yes | CD8 | A*24:02 |
| 1304 | NYNYLYRLFRK | S | Yes | CD8 | |
| 1305 | YNYLYRLFR | S | | CD8 | A*31:01 |
| 1306 | RLFRKSNLK | S | | CD8 | A*03:01, A*31:01 |
| 1307 | KSNLKPFER | S | | CD8 | A*31:01 |
| 1308 | KPFERDISTEI | S | | CD8 | B*07:02 |
| 1309 | KPFERDISTEIY | S | Yes | CD8 | |
| 1310 | FERDISTEIY | S | | CD8 | B*15:01, B*44:03 |
| 1311 | FERDISTEI | S | | CD8 | B*40:01 |
| 1312 | NGVEGENCY | S | | CD8 | |
| 1313 | YFPLQSYGF | S | Yes | CD8 | A*24:02 |
| 1314 | YGFQPTNGV | S | | CD8 | B*51:01 |
| 1315 | FQPTNGVGY | S | Yes | CD8 | |
| 1316 | VGYQPYRVV | S | | CD8 | C*06:02 |
| 1317 | GYQPYRVVVLSF | S | Yes | CD8 | |
| 1318 | YQPYRVVVL | S | | CD8 | C*06:02 |
| 1319 | QPYRVVVL | S | Yes | CD8 | B*07:02, B*08:01 |
| 1320 | QPYRVVVLSF | S | | CD8 | B*07:02 |
| 1321 | SFELLHAPATV | S | Yes | CD8 | A*02:01 |
| 1322 | FELLHAPATV | S | | CD8 | A*02:01 |
| 1323 | ELLHAPATV | S | | CD8 | A*02:01 |
| 1324 | GPKKSTNLV | S | Yes | CD8 | |
| 1325 | LVKNKCVNF | S | | CD8 | B*15:01 |
| 1326 | VLTESNKKF | S | | CD8 | B*15:01 |
| 1327 | TESNKKFLPFQQF | S | | CD8 | B*44:02 |
| 1328 | KFLPFQQFGR | S | | CD8 | A*31:01 |
| 1329 | DAVRDPQTL | S | | CD8 | B*51:01 |
| 1330 | EILDITPCSF | S | Yes | CD8 | A*26:01 |
| 1331 | TPCSFGGVSV | S | | CD8 | B*07:02 |
| 1332 | NTSNQVAVLY | S | | CD8 | A*26:01 |
| 1333 | TSNQVAVLY | S | | CD8 | A*26:01, B*35:01, B*57:01 |
| 1334 | YQDVNCTEV | S | | CD8 | A*02:06 |
| 1335 | VAIHADQLTPTW | S | | CD8 | B*57:01 |
| 1336 | IHADQLTPTW | S | | CD8 | B*53:01 |
| 1337 | QLTPTWRVY | S | | CD8 | A*30:02 |
| 1338 | RVYSTGSNV | S | | CD8 | A*30:01 |
| 1339 | RVYSTGSNVF | S | | CD8 | A*24:02, B*15:01 |
| 1340 | VYSTGSNVFQTR | S | Yes | CD8 | |
| 1341 | STGSNVFQTR | S | | CD8 | A*31:01, A*68:01 |
| 1342 | IGAEHVNNSY | S | Yes | CD8 | |
| 1343 | AEHVNNSY | S | | CD8 | B*44:02, B*44:03 |
| 1344 | YECDIPIGAGI | S | | CD8 | B*40:01 |
| 1345 | IPIGAGICASY | S | Yes | CD8 | |
| 1346 | QTNSPRRAR | S | | CD8 | A*31:01 |
| 1347 | SPRRARSVA | S | Yes | CD8 | B*07:02 |
| 1348 | SPRRARSV | S | | CD8 | B*07:02, B*08:01 |
| 1349 | RSVASQSII | S | | CD8 | B*57:01 |
| 1350 | SVASQSIIAY | S | Yes | CD8 | A*26:01, A*29:02 |
| 1351 | VASQSIIAY | S | Yes | CD8 | A*29:02, B*15:01, B*35:01 |
| 1352 | SIIAYTMSL | S | Yes | CD8 | A*02:01, B*08:01 |
| 1353 | YTMSLGAENSVAY | S | | CD8 | A*26:01 |
| 1354 | LGAENSVAY | S | | CD8 | B*35:01 |
| 1355 | LGAENSVAYSNN | S | Yes | CD8 | |
| 1356 | AYSNNSIAI | S | | CD8 | A*24:02 |
| 1357 | NSIAIPTNF | S | | CD8 | B*57:01 |
| 1358 | IAIPTNFTI | S | | CD8 | B*51:01, B*53:01 |
| 1359 | IPTNFTISV | S | | CD8 | B*07:02, B*51:01 |
| 1360 | FTISVTTEI | S | | CD8 | A*26:01 |
| 1361 | FTISVTTEIL | S | Yes | CD8 | |
| 1362 | TEILPVSMTK | S | Yes | CD8 | |
| 1363 | EILPVSMTK | S | | CD8 | A*03:01, A*68:01 |
| 1364 | KTSVDCTMYI | S | | CD8 | |
| 1365 | STECSNLLLQY | S | Yes | CD8 | A*01:01 |
| 1366 | TECSNLLLQY | S | Yes | CD8 | B*44:02, B*44:03 |
| 1367 | LLQYGSFCT | S | Yes | CD8 | |
| 1368 | GSFCTQLNR | S | | CD8 | |
| 1369 | TQLNRALTGI | S | | CD8 | |
| 1370 | QEVFAQVKQIY | S | | CD8 | B*44:02, B*44:03 |
| 1371 | QEVFAQVKQIYK | S | Yes | CD8 | |
| 1372 | EVFAQVKQI | S | | CD8 | B*51:01 |
| 1373 | EVFAQVKQIY | S | | CD8 | A*26:01 |
| 1374 | VFAQVKQIY | S | | CD8 | A*29:02, C*07:01 |
| 1375 | KQIYKTPPIKDF | S | Yes | CD8 | |
| 1376 | IYKTPPIKDF | S | | CD8 | C*07:02 |
| 1377 | ILPDPSKPSK | S | | CD8 | |
| 1378 | SFIEDLLF | S | | CD8 | A*24:02 |
| 1379 | LLFNKVTLA | S | Yes | CD8 | A*02:01 |
| 1380 | TLADAGFIK | S | | CD8 | A*03:01 |
| 1381 | LADAGFIKQY | S | | CD8 | A*01:01 |
| 1382 | ADAGFIKQY | S | | CD8 | B*44:02 |
| 1383 | GLTVLPPLL | S | | CD8 | A*02:01 |
| 1384 | LPPLLTDEM | S | | CD8 | B*35:01 |
| 1385 | LLTDEMIAQY | S | | CD8 | A*01:01 |
| 1386 | LTDEMIAQY | S | Yes | CD8 | A*01:01, A*29:02, B*35:01, |
| | | | | | C*07:02 |
| 1387 | LTDEMIAQYT | S | | CD8 | A*01:01 |
| 1388 | TDEMIAQY | S | | CD8 | A*01:01 |
| 1389 | MIAQYTSAL | S | Yes | CD8 | B*07:02, B*08:01 |
| 1390 | GTITSGWTF | S | Yes | CD8 | A*24:02, B*57:01 |
| 1391 | WTFGAGAAL | S | | CD8 | A*26:01 |
| 1392 | LQIPFAMQMAY | S | Yes | CD8 | |
| 1393 | QIPFAMQMAY | S | | CD8 | B*35:01 |
| 1394 | IPFAMQMAY | S | | CD8 | B*35:01, B*51:01 |
| 1395 | FAMQMAYRF | S | | CD8 | B*35:01 |
| 1396 | TQNVLYENQK | S | Yes | CD8 | |
| 1397 | YENQKLIANQF | S | | CD8 | B*44:02 |
| 1398 | NQKLIANQF | S | Yes | CD8 | |
| 1399 | IANQFNSAI | S | | CD8 | B*51:01 |
| 1400 | LSSTASALGK | S | | CD8 | |
| 1401 | AQALNTLVKQL | S | Yes | CD8 | |
| 1402 | ALNTLVKQL | S | | CD8 | A*02:01 |
| 1403 | SVLNDILSR | S | | CD8 | A*68:01 |
| 1404 | SVLNDILSRL | S | | CD8 | |
| 1405 | VLNDILSRL | S | Yes | CD8 | A*02:01 |
| 1406 | RLDKVEAEVQI | S | | CD8 | A*02:01 |
| 1407 | RLDKVEAEV | S | Yes | CD8 | A*02:01 |
| 1408 | VEAEVQIDRLITGR | S | Yes | CD8 | |
| 1409 | AEVQIDRL | S | | CD8 | B*40:01 |
| 1410 | AEVQIDRLI | S | | CD8 | B*44:02, B*44:03 |
| 1411 | LITGRLQSLQTYV | S | Yes | CD8 | |
| 1412 | ITGRLQSLQTY | S | | CD8 | A*01:01 |
| 1413 | LITGRLQSL | S | | CD8 | A*02:01 |
| 1414 | RLQSLQTY | S | | CD8 | B*15:01 |
| 1415 | RLQSLQTYV | S | Yes | CD8 | A*02:01 |
| 1416 | AEIRASANLAATK | S | Yes | CD8 | |
| 1417 | AEIRASANL | S | | CD8 | B*40:01, B*44:02, B*44:03 |
| 1418 | RASANLAATK | S | | CD8 | A*03:01 |
| 1419 | SKRVDFCGKGY | S | | CD8 | A*01:01 |
| 1420 | RVDFCGKGY | S | | CD8 | |
| 1421 | YHLMSFPQSA | S | Yes | CD8 | |
| 1422 | HLMSFPQSA | S | Yes | CD8 | A*02:01 |
| 1423 | SFPQSAPHGVVF | S | | CD8 | A*24:02 |
| 1424 | FPQSAPHGV | S | | CD8 | B*07:02, B*53:01 |
| 1425 | FPQSAPHGVVF | S | Yes | CD8 | B*08:01, B*35:01 |
| 1426 | QSAPHGVVF | S | | CD8 | C*07:02 |
| 1427 | APHGVVFLHVTYV | S | Yes | CD8 | |
| 1428 | APHGVVFLHV | S | Yes | CD8 | B*07:02 |
| 1429 | APHGVVFL | S | | CD8 | B*07:02 |
| 1430 | GVVFLHVTY | S | | CD8 | A*32:01 |
| 1431 | VVFLHVTYV | S | Yes | CD8 | A*02:01, A*02:06 |
| 1432 | HVTYVPAQEK | S | | CD8 | A*68:01 |
| 1433 | HVTYVPAQEKNF | S | Yes | CD8 | |
| 1434 | VTYVPAQEK | S | | CD8 | A*03:01, A*30:01 |
| 1435 | TYVPAQEKNFT | S | | CD8 | A*24:02 |
| 1436 | FPREGVFV | S | | CD8 | B*07:02, B*51:01 |
| 1437 | REGVFVSNGTHW | S | Yes | CD8 | B*44:03 |
| 1438 | GVFVSNGTHW | S | | CD8 | B*57:01 |
| 1439 | VFVSNGTHW | S | | CD8 | A*24:02 |
| 1440 | VFVSNGTHWF | S | | CD8 | A*24:02 |
| 1441 | FVSNGTHWF | S | | CD8 | A*26:01, B*35:01 |
| 1442 | GTHWFVTQR | S | Yes | CD8 | A*11:01, A*31:01 |
| 1443 | HWFVTQRNF | S | | CD8 | A*24:02 |
| 1444 | WFVTQRNFY | S | | CD8 | A*29:02 |
| 1445 | IITTDNTFV | S | | CD8 | |
| 1446 | TVYDPLQPELDSFK | S | Yes | CD8 | A*03:01 |
| 1447 | VYDPLQPELDSF | S | Yes | CD8 | A*24:02 |
| 1448 | DSFKEELDKY | S | | CD8 | |
| 1449 | SFKEELDKY | S | | CD8 | A*29:02 |
| 1450 | NASVVNIQK | S | | CD8 | A*68:01 |
| 1451 | KEIDRLNEV | S | | CD8 | B*40:01, B*44:02, B*44:03 |
| 1452 | RLNEVAKNL | S | Yes | CD8 | A*02:01 |
| 1453 | NLNESLIDL | S | Yes | CD8 | A*02:01 |
| 1454 | NESLIDLQEL | S | | CD8 | B*40:01 |
| 1455 | SLIDLQEL | S | | CD8 | A*02:01 |
| 1456 | LQELGKYEQY | S | | CD8 | A*01:01 |
| 1457 | QELGKYEQY | S | | CD8 | B*44:02, B*44:03 |
| 1458 | YEQYIKWPWYI | S | Yes | CD8 | A*24:02 |
| 1459 | YEQYIKWPW | S | | CD8 | B*44:02, B*44:03 |
| 1460 | QYIKWPWYIW | S | | CD8 | A*23:01 |
| 1461 | QYIKWPWYI | S | Yes | CD8 | A*24:02, C*06:02 |
| 1462 | KWPWYIWLGF | S | Yes | CD8 | A*24:02 |
| 1463 | FIAGLIAIV | S | Yes | CD8 | A*02:01 |
| 1464 | CMTSCCSCLK | S | | CD8 | |
| 1465 | FDEDDSEPVL | S | | CD8 | B*40:01 |
| 1466 | SEPVLKGVKL | S | Yes | CD8 | B*40:01 |
| 1467 | EPVLKGVKL | S | | CD8 | B*07:02 |
| 1468 | VLKGVKLHY | S | | CD8 | A*29:02 |

A set of 399 more dominant epitopes are defined by being recognized by 3 or more donors/different studies (110 CD4, 289 CD8). Accordingly new pools have been designed (the number of epitopes in each pool is indicated after the =sign)

CD8S (D) = 53 (Table 2). CD8S (D) Megapool

| SEQ | | | | | |
|---|---|---|---|---|---|
| ID | | Anti- | | | |
| NO: | Description | gen | Start | End | Dominant |
| 1469 | YTNSFTRGVY | S | 28 | 37 | Yes |
| 1470 | STQDLFLPFF | S | 50 | 59 | Yes |
| 1471 | RFDNPVLPF | S | 78 | 86 | Yes |
| 1472 | GVYFASTEK | S | 89 | 97 | Yes |
| 1473 | TEKSNIIRGW | S | 95 | 104 | Yes |
| 1474 | TLDSKTQSL | S | 109 | 117 | Yes |
| 1475 | SSANNCTFEY | S | 161 | 170 | Yes |
| 1476 | FVFKNIDGY | S | 192 | 200 | Yes |
| 1477 | IYSKHTPINL | S | 203 | 212 | Yes |
| 1478 | TPINL VRDL | S | 208 | 216 | Yes |
| 1479 | LLALHRSYL | S | 241 | 249 | Yes |
| 1480 | WTAGAAAYY | S | 258 | 266 | Yes |
| 1481 | YLQPRTFLL | S | 269 | 277 | Yes |
| 1482 | RISNCVADY | S | 357 | 365 | Yes |
| 1483 | CVADYSVLY | S | 361 | 369 | Yes |
| 1484 | KCYGVSPTK | S | 378 | 386 | Yes |
| 1485 | KIADYNYKL | S | 417 | 425 | Yes |
| 1486 | KLPDDFTGCV | S | 424 | 433 | Yes |
| 1487 | NLDSKVGGNY | S | 440 | 449 | Yes |
| 1488 | NYNYLYRLF | S | 448 | 456 | Yes |
| 1489 | YFPLQSYGF | S | 489 | 497 | Yes |
| 1490 | FQPTNGVGY | S | 497 | 505 | Yes |
| 1491 | GPKKSTNLV | S | 526 | 534 | Yes |
| 1492 | EILDITPCSF | S | 583 | 592 | Yes |
| 1493 | IGAEHVNNSY | S | 651 | 660 | Yes |
| 1494 | SPRRARSVA | S | 680 | 688 | Yes |
| 1495 | SVASQSIIAY | S | 686 | 695 | Yes |
| 1496 | VASQSIIAY | S | 687 | 695 | Yes |
| 1497 | SIIAYTMSL | S | 691 | 699 | Yes |
| 1498 | FTISVTTEIL | S | 718 | 727 | Yes |
| 1499 | TEILPVSMTK | S | 724 | 733 | Yes |
| 1500 | TECSNLLLQY | S | 747 | 756 | Yes |
| 1501 | LLQYGSFCT | S | 753 | 761 | Yes |
| 1502 | LLFNKVTLA | S | 821 | 829 | Yes |
| 1503 | LTDEMIAQY | S | 865 | 873 | Yes |
| 1504 | MIAQYTSAL | S | 869 | 877 | Yes |
| 1505 | GTITSGWTF | S | 880 | 888 | Yes |
| 1506 | TQNVLYENQK | S | 912 | 921 | Yes |
| 1507 | NQKLIANQF | S | 919 | 927 | Yes |
| 1508 | VLNDILSRL | S | 976 | 984 | Yes |
| 1509 | RLDKVEAEV | S | 983 | 991 | Yes |
| 1510 | RLQSLQTYV | S | 1000 | 1008 | Yes |
| 1511 | YHLMSFPQSA | S | 1047 | 1056 | Yes |
| 1512 | HLMSFPQSA | S | 1048 | 1056 | Yes |
| 1513 | APHGVVFLHV | S | 1056 | 1065 | Yes |
| 1514 | VVFLHVTYV | S | 1060 | 1068 | Yes |
| 1515 | GTHWFVTQR | S | 1099 | 1107 | Yes |
| 1516 | RLNEVAKNL | S | 1185 | 1193 | Yes |
| 1517 | NLNESLIDL | S | 1192 | 1200 | Yes |
| 1518 | QYIKWPWYI | S | 1208 | 1216 | Yes |
| 1519 | KWPWYIWLGF | S | 1211 | 1220 | Yes |
| 1520 | FIAGLIAIV | S | 1220 | 1228 | Yes |
| 1521 | SEPVLKGVKL | S | 1261 | 1270 | Yes |

•CD8S (ND) = 144 (Table 3). CD8S (ND) Megapool

| SEQ ID NO: | Description | Antigen | Start | End | Dominant |
|---|---|---|---|---|---|
| 1522 | FVFLVLLPL | S | 2 | 10 | No |
| 1523 | LPLVSSQCV | S | 8 | 16 | No |
| 1524 | TRTQLPPAY | S | 20 | 28 | No |
| 1525 | LPPAYTNSF | S | 24 | 32 | No |
| 1526 | NSFTRGVYY | S | 30 | 38 | No |
| 1527 | GVYYPDKVFR | S | 35 | 44 | No |
| 1528 | KVFRSSVLH | S | 41 | 49 | No |
| 1529 | FRSSVLHST | S | 43 | 51 | No |
| 1530 | STQDLFLPF | S | 50 | 58 | No |
| 1531 | TQDLFLPFF | S | 51 | 59 | No |
| 1532 | FLPFFSNVTW | S | 55 | 64 | No |
| 1533 | PFFSNVTWF | S | 57 | 65 | No |
| 1534 | HVSGTNGTK | S | 69 | 77 | No |
| 1535 | LPFNDGVYF | S | 84 | 92 | No |
| 1536 | DGVYFASTEK | S | 88 | 97 | No |
| 1537 | WIFGTTLDSK | S | 104 | 113 | No |
| 1538 | CNDPFLGVY | S | 136 | 144 | No |
| 1539 | GVYYHKNNK | S | 142 | 150 | No |
| 1540 | YYHKNNKSW | S | 144 | 152 | No |
| 1541 | SWMESEFRV | S | 151 | 159 | No |
| 1542 | SWMESEFRVY | S | 151 | 160 | No |
| 1543 | VYSSANNCTF | S | 159 | 168 | No |
| 1544 | SANNCTFEY | S | 162 | 170 | No |
| 1545 | FEYVSQPFLM | S | 168 | 177 | No |
| 1546 | EYVSQPFLM | S | 169 | 177 | No |
| 1547 | FKNLREFVF | S | 186 | 194 | No |
| 1548 | VFKNIDGYF | S | 193 | 201 | No |
| 1549 | KIYSKHTPI | S | 202 | 210 | No |
| 1550 | LEPLVDLPI | S | 223 | 231 | No |
| 1551 | LPIGINITRF | S | 229 | 238 | No |
| 1552 | INITRFQTL | S | 233 | 241 | No |
| 1553 | GAAAYYVGY | S | 261 | 269 | No |
| 1554 | VGYLQPRTFL | S | 267 | 276 | No |
| 1555 | GYLQPRTFLL | S | 268 | 277 | No |
| 1556 | CALDPLSETK | S | 291 | 300 | No |
| 1557 | ALDPLSETK | S | 292 | 300 | No |
| 1558 | SETKCTLKSF | S | 297 | 306 | No |
| 1559 | ETKCTLKSF | S | 298 | 306 | No |
| 1560 | IYQTSNFRV | S | 312 | 320 | No |
| 1561 | QPTESIVRF | S | 321 | 329 | No |
| 1562 | GEVFNATRF | S | 339 | 347 | No |
| 1563 | NATRFASVY | S | 343 | 351 | No |
| 1564 | TRFASVYAW | S | 345 | 353 | No |
| 1565 | RFASVYAWNR | S | 346 | 355 | No |
| 1566 | FASVYAWNR | S | 347 | 355 | No |
| 1567 | SVYAWNRKR | S | 349 | 357 | No |
| 1568 | NSASFSTFK | S | 370 | 378 | No |
| 1569 | SASFSTFKCY | S | 371 | 380 | No |
| 1570 | KLNDLCFTNV | S | 386 | 395 | No |
| 1571 | NVYADSFVIR | S | 394 | 403 | No |
| 1572 | FVIRGDEVR | S | 400 | 408 | No |
| 1573 | QIAPGQTGK | S | 409 | 417 | No |
| 1574 | DSKVGGNYNY | S | 442 | 451 | No |
| 1575 | KVGGNYNYLY | S | 444 | 453 | No |
| 1576 | VGGNYNYLY | S | 445 | 453 | No |
| 1577 | GNYNYLYRLF | S | 447 | 456 | No |
| 1578 | YNYL YRLFR | S | 449 | 457 | No |
| 1579 | RLFRKSNLK | S | 454 | 462 | No |
| 1580 | KSNLKPFER | S | 458 | 466 | No |
| 1581 | FERDISTEI | S | 464 | 472 | No |
| 1582 | FERDISTEIY | S | 464 | 473 | No |
| 1583 | NGVEGENCY | S | 481 | 489 | No |
| 1584 | YGFQPTNGV | S | 495 | 503 | No |
| 1585 | VGYQPYRVV | S | 503 | 511 | No |
| 1586 | YQPYRVVVL | S | 505 | 513 | No |
| 1587 | QPYRVVVLSF | S | 506 | 515 | No |
| 1588 | FELLHAPATV | S | 515 | 524 | No |
| 1589 | ELLHAPATV | S | 516 | 524 | No |
| 1590 | LVKNKCVNF | S | 533 | 541 | No |
| 1591 | VLTESNKKF | S | 551 | 559 | No |
| 1592 | KFLPFQQFGR | S | 558 | 567 | No |
| 1593 | DAVRDPQTL | S | 574 | 582 | No |
| 1594 | TPCSFGGVSV | S | 588 | 597 | No |
| 1595 | NTSNQVAVLY | S | 603 | 612 | No |
| 1596 | TSNQVAVLY | S | 604 | 612 | No |
| 1597 | YQDVNCTEV | S | 612 | 620 | No |
| 1598 | IHADQLTPTW | S | 624 | 633 | No |
| 1599 | QLTPTWRVY | S | 628 | 636 | No |
| 1600 | RVYSTGSNV | S | 634 | 642 | No |
| 1601 | RVYSTGSNVF | S | 634 | 643 | No |
| 1602 | STGSNVFQTR | S | 637 | 646 | No |
| 1603 | QTNSPRRAR | S | 677 | 685 | No |
| 1604 | RSVASQSII | S | 685 | 693 | No |
| 1605 | LGAENSVAY | S | 699 | 707 | No |
| 1606 | AYSNNSIAI | S | 706 | 714 | No |
| 1607 | NSIAIPTNF | S | 710 | 718 | No |
| 1608 | IAIPTNFTI | S | 712 | 720 | No |
| 1609 | IPTNFTISV | S | 714 | 722 | No |
| 1610 | FTISVTTEI | S | 718 | 726 | No |
| 1611 | EILPVSMTK | S | 725 | 733 | No |
| 1612 | KTSVDCTMYI | S | 733 | 742 | No |
| 1613 | GSFCTQLNR | S | 757 | 765 | No |
| 1614 | TQLNRALTGI | S | 761 | 770 | No |
| 1615 | EVFAQVKQI | S | 780 | 788 | No |
| 1616 | EVFAQVKQIY | S | 780 | 789 | No |
| 1617 | VFAQVKQIY | S | 781 | 789 | No |
| 1618 | IYKTPPIKDF | S | 788 | 797 | No |
| 1619 | ILPDPSKPSK | S | 805 | 814 | No |
| 1620 | TLADAGFIK | S | 827 | 835 | No |
| 1621 | LADAGFIKQY | S | 828 | 837 | No |
| 1622 | ADAGFIKQY | S | 829 | 837 | No |
| 1623 | GLTVLPPLL | S | 857 | 865 | No |
| 1624 | LPPLLTDEM | S | 861 | 869 | No |
| 1625 | LLTDEMIAQY | S | 864 | 873 | No |
| 1626 | LTDEMIAQYT | S | 865 | 874 | No |
| 1627 | WTFGAGAAL | S | 886 | 894 | No |
| 1628 | QIPFAMQMAY | S | 895 | 904 | No |
| 1629 | IPFAMQMAY | S | 896 | 904 | No |
| 1630 | FAMQMAYRF | S | 898 | 906 | No |
| 1631 | IANQFNSAI | S | 923 | 931 | No |
| 1632 | LSSTASALGK | S | 938 | 947 | No |
| 1633 | ALNTL VKQL | S | 958 | 966 | No |
| 1634 | SVLNDILSR | S | 975 | 983 | No |
| 1635 | SVLNDILSRL | S | 975 | 984 | No |
| 1636 | AEVQIDRLI | S | 989 | 997 | No |
| 1637 | LITGRLQSL | S | 997 | 1005 | No |
| 1638 | AEIRASANL | S | 1016 | 1024 | No |
| 1639 | RASANLAATK | S | 1019 | 1028 | No |
| 1640 | RVDFCGKGY | S | 1039 | 1047 | No |
| 1641 | FPQSAPHGV | S | 1052 | 1060 | No |
| 1642 | QSAPHGVVF | S | 1054 | 1062 | No |
| 1643 | GVVFLHVTY | S | 1059 | 1067 | No |
| 1644 | HVTYVPAQEK | S | 1064 | 1073 | No |
| 1645 | VTYVPAQEK | S | 1065 | 1073 | No |
| 1646 | GVFVSNGTHW | S | 1093 | 1102 | No |
| 1647 | VFVSNGTHW | S | 1094 | 1102 | No |
| 1648 | VFVSNGTHWF | S | 1094 | 1103 | No |
| 1649 | FVSNGTHWF | S | 1095 | 1103 | No |
| 1650 | HWFVTQRNF | S | 1101 | 1109 | No |
| 1651 | WFVTQRNFY | S | 1102 | 1110 | No |
| 1652 | IITTDNTFV | S | 1114 | 1122 | No |
| 1653 | DSFKEELDKY | S | 1146 | 1155 | No |
| 1654 | SFKEELDKY | S | 1147 | 1155 | No |
| 1655 | NASVVNIQK | S | 1173 | 1181 | No |
| 1656 | KEIDRLNEV | S | 1181 | 1189 | No |
| 1657 | NESLIDLQEL | S | 1194 | 1203 | No |
| 1658 | LQELGKYEQY | S | 1200 | 1209 | No |
| 1659 | QELGKYEQY | S | 1201 | 1209 | No |
| 1660 | YEQYIKWPW | S | 1206 | 1214 | No |
| 1661 | QYIKWPWYIW | S | 1208 | 1217 | No |
| 1662 | CMTSCCSCLK | S | 1236 | 1245 | No |
| 1663 | FDEDDSEPVL | S | 1256 | 1265 | No |
| 1664 | EPVLKGVKL | S | 1262 | 1270 | No |
| 1665 | VLKGVKLHY | S | 1264 | 1272 | No |

•CD8R (D) = 153 (Table 4). CD8R (D) Megapool

| SEQ ID NO: | Description | Antigen | Start | End | Dominant |
|---|---|---|---|---|---|
| 1666 | SLVKPSFYV | E | 50 | 58 | Yes |
| 1667 | YVYSRVKNL | E | 57 | 65 | Yes |
| 1668 | GTITVEELK | M | 6 | 14 | Yes |
| 1669 | KLLEQWNLV | M | 15 | 23 | Yes |
| 1670 | FLFLTWICL | M | 26 | 34 | Yes |
| 1671 | NRFLYIIKL | M | 43 | 51 | Yes |
| 1672 | TLACFVLAAV | M | 61 | 70 | Yes |
| 1673 | FVLAAVYRI | M | 65 | 73 | Yes |
| 1674 | RLFARTRSMW | M | 101 | 110 | Yes |
| 1675 | SMWSFNPET | M | 108 | 116 | Yes |
| 1676 | SELVIGAVIL | M | 136 | 145 | Yes |
| 1677 | AVILRGHLR | M | 142 | 150 | Yes |
| 1678 | ATSRTLSYY | M | 171 | 179 | Yes |
| 1679 | ATSRTLSYYK | M | 171 | 180 | Yes |
| 1680 | RYRIGNYKL | M | 198 | 206 | Yes |
| 1681 | QRNAPRITF | N | 9 | 17 | Yes |
| 1682 | NTASWFTAL | N | 48 | 56 | Yes |
| 1683 | KFPRGQGVPI | N | 65 | 74 | Yes |
| 1684 | FPRGQGVPI | N | 66 | 74 | Yes |
| 1685 | YYRRATRRIR | N | 86 | 95 | Yes |
| 1686 | RIRGGDGKMK | N | 93 | 102 | Yes |
| 1687 | KMKDLSPRW | N | 100 | 108 | Yes |
| 1688 | LSPRWYFYYL | N | 104 | 113 | Yes |
| 1689 | SPRWYFYYL | N | 105 | 113 | Yes |
| 1690 | GTGPEAGLPY | N | 114 | 123 | Yes |
| 1691 | ATEGALNTPK | N | 134 | 143 | Yes |
| 1692 | NPANNAAIVL | N | 150 | 159 | Yes |
| 1693 | VLQLPQGTTL | N | 158 | 167 | Yes |
| 1694 | LALLLLDRL | N | 219 | 227 | Yes |
| 1695 | LLLLDRLNQL | N | 221 | 230 | Yes |
| 1696 | LLLDRLNQL | N | 222 | 230 | Yes |
| 1697 | QQQQGQTVTK | N | 239 | 248 | Yes |
| 1698 | KPRQKRTAT | N | 257 | 265 | Yes |
| 1699 | KAYNVTQAF | N | 266 | 274 | Yes |
| 1700 | AQFAPSASA | N | 305 | 313 | Yes |
| 1701 | AQFAPSASAF | N | 305 | 314 | Yes |
| 1702 | ASAFFGMSR | N | 311 | 319 | Yes |
| 1703 | GMSRIGMEV | N | 316 | 324 | Yes |
| 1704 | MEVTPSGTW | N | 322 | 330 | Yes |
| 1705 | MEVTPSGTWL | N | 322 | 331 | Yes |
| 1706 | ILLNKHIDA | N | 351 | 359 | Yes |
| 1707 | KTFPPTEPK | N | 361 | 369 | Yes |
| 1708 | KTFPPTEPKK | N | 361 | 370 | Yes |
| 1709 | LPAADLDDF | N | 395 | 403 | Yes |
| 1710 | FSKQLQQSM | N | 403 | 411 | Yes |
| 1711 | VLSEARQHL | NSP1 | 38 | 46 | Yes |
| 1712 | TVLSFCAFAV | NSP10 | 12 | 21 | Yes |
| 1713 | DLKGKYVQI | NSP10 | 91 | 99 | Yes |
| 1714 | YTMADLVYAL | NSP12 | 122 | 131 | Yes |
| 1715 | TMADLVYAL | NSP12 | 123 | 131 | Yes |
| 1716 | LLMPILTLT | NSP12 | 240 | 248 | Yes |
| 1717 | KLFDRYFKY | NSP12 | 281 | 289 | Yes |
| 1718 | FVDGVPFVV | NSP12 | 334 | 342 | Yes |
| 1719 | ISDYDYYRY | NSP12 | 450 | 458 | Yes |
| 1720 | RQLLFVVEV | NSP12 | 467 | 475 | Yes |
| 1721 | VVDKYFDCY | NSP12 | 475 | 483 | Yes |
| 1722 | RLYYDSMSY | NSP12 | 513 | 521 | Yes |
| 1723 | MVMCGGSLYV | NSP12 | 666 | 675 | Yes |
| 1724 | DTDFVNEFY | NSP12 | 738 | 746 | Yes |
| 1725 | FYAYLRKHF | NSP12 | 745 | 753 | Yes |
| 1726 | RILGAGCFV | NSP12 | 836 | 844 | Yes |
| 1727 | LMIERFVSL | NSP12 | 854 | 862 | Yes |
| 1728 | LYLQYIRKL | NSP12 | 883 | 891 | Yes |
| 1729 | VLQAVGACV | NSP13 | −2 | 6 | Yes |
| 1730 | LVLSVNPYV | NSP13 | 41 | 49 | Yes |
| 1731 | KLFAAETLK | NSP13 | 131 | 139 | Yes |
| 1732 | KLSYGIATV | NSP13 | 146 | 154 | Yes |
| 1733 | VVYRGTTTY | NSP13 | 209 | 217 | Yes |
| 1734 | VVYRGTTTYK | NSP13 | 209 | 218 | Yes |
| 1735 | VYRGTTTYKL | NSP13 | 210 | 219 | Yes |
| 1736 | KLNVGDYFV | NSP13 | 218 | 226 | Yes |
| 1737 | SSNVANYQK | NSP13 | 263 | 271 | Yes |
| 1738 | VYIGDPAQL | NSP13 | 397 | 405 | Yes |
| 1739 | IVDTVSALV | NSP13 | 448 | 456 | Yes |
| 1740 | IPRRNVATL | NSP13 | 592 | 600 | Yes |
| 1741 | VLWAHGFEL | NSP14 | 184 | 192 | Yes |
| 1742 | TYACWHHSI | NSP14 | 223 | 231 | Yes |
| 1743 | AIMTRCLAV | NSP14 | 274 | 282 | Yes |
| 1744 | IEYPIIGDEL | NSP14 | 294 | 303 | Yes |
| 1745 | ALLADKFPV | NSP14 | 320 | 328 | Yes |
| 1746 | SYATHSDKF | NSP14 | 369 | 377 | Yes |
| 1747 | YLDAYNMMI | NSP14 | 494 | 502 | Yes |
| 1748 | MMISAGFSL | NSP14 | 500 | 508 | Yes |
| 1749 | KQFDTYNLW | NSP14 | 512 | 520 | Yes |
| 1750 | NLWNTFTRL | NSP14 | 518 | 526 | Yes |
| 1751 | KVDGVDVEL | NSP15 | 34 | 42 | Yes |
| 1752 | LLLDDFVEII | NSP15 | 297 | 306 | Yes |
| 1753 | MLWCKDGHV | NSP15 | 330 | 338 | Yes |
| 1754 | GVAMPNLYK | NSP16 | 8 | 16 | Yes |
| 1755 | KMQRMLLEK | NSP16 | 16 | 24 | Yes |
| 1756 | YLNTLTLAV | NSP16 | 53 | 61 | Yes |
| 1757 | TLIGDCATV | NSP16 | 110 | 118 | Yes |
| 1758 | KTIQPRVEK | NSP2 | 102 | 110 | Yes |
| 1759 | KLVNKFLAL | NSP2 | 500 | 508 | Yes |
| 1760 | VTNNTFTLK | NSP2 | 628 | 636 | Yes |
| 1761 | FGDDTVIEV | NSP3 | 7 | 15 | Yes |
| 1762 | PTDNYITTY | NSP3 | 503 | 511 | Yes |
| 1763 | QEILGTVSW | NSP3 | 547 | 555 | Yes |
| 1764 | TTDPSFLGRY | NSP3 | 819 | 828 | Yes |
| 1765 | YLATALLTL | NSP3 | 857 | 865 | Yes |
| 1766 | SAPPAQYEL | NSP3 | 990 | 998 | Yes |
| 1767 | NYMPYFFTL | NSP3 | 1349 | 1357 | Yes |
| 1768 | ASMPTTIAK | NSP3 | 1374 | 1382 | Yes |
| 1769 | ILFTRFFYV | NSP3 | 1514 | 1522 | Yes |
| 1770 | YIFFASFYY | NSP3 | 1566 | 1574 | Yes |
| 1771 | QLMCQPILLL | NSP3 | 1745 | 1754 | Yes |
| 1772 | STFNVPMEK | NSP3 | 1782 | 1790 | Yes |
| 1773 | FLPRVFSAV | NSP4 | 121 | 129 | Yes |
| 1774 | FSAVGNICY | NSP4 | 126 | 134 | Yes |
| 1775 | SLRPDTRYVL | NSP4 | 184 | 193 | Yes |
| 1776 | FLLNKEMYL | NSP4 | 420 | 428 | Yes |
| 1777 | TSEDMLNPNY | NSP5 | 45 | 54 | Yes |
| 1778 | TPKYKFVRI | NSP5 | 98 | 106 | Yes |
| 1779 | VRIQPGQTF | NSP5 | 104 | 112 | Yes |
| 1780 | FLNGSCGSV | NSP5 | 140 | 148 | Yes |
| 1781 | GTDLEGNFY | NSP5 | 174 | 182 | Yes |
| 1782 | VLAWLYAAV | NSP5 | 204 | 212 | Yes |
| 1783 | FLNRFTTTL | NSP5 | 219 | 227 | Yes |
| 1784 | SAFAMMFVK | NSP6 | 53 | 61 | Yes |
| 1785 | FLLPSLATV | NSP6 | 70 | 78 | Yes |
| 1786 | MPASWVMRI | NSP6 | 86 | 94 | Yes |
| 1787 | KLKDCVMYA | NSP6 | 109 | 117 | Yes |
| 1788 | SMWALIISV | NSP6 | 163 | 171 | Yes |
| 1789 | KLWAQCVQL | NSP7 | 27 | 35 | Yes |
| 1790 | SEFSSLPSY | NSP8 | 4 | 12 | Yes |
| 1791 | NTCDGTTFTY | NSP8 | 140 | 149 | Yes |
| 1792 | FTYASALWEI | NSP8 | 147 | 156 | Yes |
| 1793 | SALWEIQQVV | NSP8 | 151 | 160 | Yes |
| 1794 | ALWEIQQVV | NSP8 | 152 | 160 | Yes |
| 1795 | ALRANSAVK | NSP8 | 188 | 196 | Yes |
| 1796 | CTDDNALAY | NSP9 | 23 | 31 | Yes |
| 1797 | CTDDNALAYY | NSP9 | 23 | 32 | Yes |
| 1798 | YTELEPPCRF | NSP9 | 66 | 75 | Yes |
| 1799 | VTDTPKGPK | NSP9 | 76 | 84 | Yes |
| 1800 | MDLFMRIFTI | ORF3a | 1 | 10 | Yes |
| 1801 | FVRATATIPI | ORF3a | 28 | 37 | Yes |
| 1802 | IPIQASLPF | ORF3a | 35 | 43 | Yes |
| 1803 | IVGVALLAVF | ORF3a | 47 | 56 | Yes |
| 1804 | ALSKGVHFV | ORF3a | 72 | 80 | Yes |
| 1805 | YLYALVYFL | ORF3a | 107 | 115 | Yes |
| 1806 | VYFLQSINF | ORF3a | 112 | 120 | Yes |
| 1807 | LLYDANYFL | ORF3a | 139 | 147 | Yes |
| 1808 | FTSDYYQLY | ORF3a | 207 | 215 | Yes |
| 1809 | YYQLYSTQL | ORF3a | 211 | 219 | Yes |
| 1810 | HLVDFQVTI | ORF6 | 3 | 11 | Yes |
| 1811 | QLRARSVSPK | ORF7a | 76 | 85 | Yes |
| 1812 | RARSVSPKL | ORF7a | 78 | 86 | Yes |
| 1813 | KLFIRQEEV | ORF7a | 85 | 93 | Yes |
| 1814 | VFITLCFTLK | ORF7a | 108 | 117 | Yes |
| 1815 | IMLIIFWFSL | ORF7b | 23 | 32 | Yes |
| 1816 | IQYIDIGNY | ORF8 | 71 | 79 | Yes |
| 1817 | KLGSLVVRC | ORF8 | 94 | 102 | Yes |
| 1818 | LEYHDVRVVL | ORF8 | 109 | 118 | Yes |

•CD8 (ND) = 468 (Table 5). CD8R (ND) Megapool

| SEQ ID NO: | Description | Antigen | Start | End | Dominant |
|---|---|---|---|---|---|
| 1819 | SEETGTLIV | E | 6 | 14 | No |
| 1820 | FLLVTLAIL | E | 26 | 34 | No |
| 1821 | LVKPSFYVY | E | 51 | 59 | No |
| 1822 | FYVYSRVKNL | E | 56 | 65 | No |
| 1823 | NGTITVEELK | M | 5 | 14 | No |
| 1824 | WICLLQFAY | M | 31 | 39 | No |
| 1825 | FAYANRNRF | M | 37 | 45 | No |
| 1826 | AYANRNRFL | M | 38 | 46 | No |
| 1827 | YANRNRFLY | M | 39 | 47 | No |
| 1828 | ANRNRFLYI | M | 40 | 48 | No |
| 1829 | RNRFLYIIK | M | 42 | 50 | No |
| 1830 | RNRFLYIIKL | M | 42 | 51 | No |
| 1831 | RFLYIIKLIF | M | 44 | 53 | No |
| 1832 | FLWLLWPVTL | M | 53 | 62 | No |
| 1833 | WLLWPVTLA | M | 55 | 63 | No |
| 1834 | LLWPVTLAC | M | 56 | 64 | No |
| 1835 | LAAVYRINW | M | 67 | 75 | No |
| 1836 | LAAVYRINWI | M | 67 | 76 | No |
| 1837 | MACLVGLMW | M | 84 | 92 | No |
| 1838 | GLMWLSYFI | M | 89 | 97 | No |
| 1839 | LSYFIASFR | M | 93 | 101 | No |
| 1840 | IASFRLFAR | M | 97 | 105 | No |
| 1841 | FRLFARTRSM | M | 100 | 109 | No |
| 1842 | RLFARTRSM | M | 101 | 109 | No |
| 1843 | SFNPETNIL | M | 111 | 119 | No |
| 1844 | SELVIGAVI | M | 136 | 144 | No |
| 1845 | ELVIGAVILR | M | 137 | 146 | No |
| 1846 | LVIGAVILR | M | 138 | 146 | No |
| 1847 | HLRIAGHHL | M | 148 | 156 | No |
| 1848 | RIAGHHLGR | M | 150 | 158 | No |
| 1849 | LPKEITVAT | M | 164 | 172 | No |
| 1850 | TVATSRTLSY | M | 169 | 178 | No |
| 1851 | VATSRTLSY | M | 170 | 178 | No |
| 1852 | VATSRTLSYY | M | 170 | 179 | No |
| 1853 | SQRVAGDSGF | M | 184 | 193 | No |
| 1854 | AGDSGFAAY | M | 188 | 196 | No |
| 1855 | DSGFAAYSR | M | 190 | 198 | No |
| 1856 | YSRYRIGNYK | M | 196 | 205 | No |
| 1857 | SSSDNIALL | M | 212 | 220 | No |
| 1858 | RPQGLPNNTA | N | 41 | 50 | No |
| 1859 | QGLPNNTASW | N | 43 | 52 | No |
| 1860 | LPNNTASWF | N | 45 | 53 | No |
| 1861 | SPDDQIGYY | N | 79 | 87 | No |
| 1862 | MKDLSPRWY | N | 101 | 109 | No |
| 1863 | YLGTGPEAGL | N | 112 | 121 | No |
| 1864 | AGLPYGANK | N | 119 | 127 | No |
| 1865 | LPYGANKDGI | N | 121 | 130 | No |
| 1866 | YGANKDGIIW | N | 123 | 132 | No |
| 1867 | IIWVATEGA | N | 130 | 138 | No |
| 1868 | GTRNPANNA | N | 147 | 155 | No |
| 1869 | AEGSRGGSQA | N | 173 | 182 | No |
| 1870 | SQASSRSSSR | N | 180 | 189 | No |
| 1871 | SSRGTSPAR | N | 201 | 209 | No |
| 1872 | KSAAEASKK | N | 249 | 257 | No |
| 1873 | RTATKAYNV | N | 262 | 270 | No |
| 1874 | QELIRQGTDY | N | 289 | 298 | No |
| 1875 | ELIRQGTDY | N | 290 | 298 | No |
| 1876 | QFAPSASAFF | N | 306 | 315 | No |
| 1877 | FAPSASAFF | N | 307 | 315 | No |
| 1878 | APSASAFFGM | N | 308 | 317 | No |
| 1879 | SASAFFGMSR | N | 310 | 319 | No |
| 1880 | VTPSGTWLTY | N | 324 | 333 | No |
| 1881 | TPSGTWLTY | N | 325 | 333 | No |
| 1882 | KLDDKDPNF | N | 338 | 346 | No |
| 1883 | LLNKHIDAY | N | 352 | 360 | No |
| 1884 | YKTFPPTEPK | N | 360 | 369 | No |
| 1885 | KKQQTVTLL | N | 387 | 395 | No |
| 1886 | HLKDGTCGL | NSP1 | 45 | 53 | No |
| 1887 | APHGHVMVEL | NSP1 | 79 | 88 | No |
| 1888 | VMVELVAEL | NSP1 | 84 | 92 | No |
| 1889 | VPHVGEIPV | NSP1 | 108 | 116 | No |
| 1890 | HVGEIPVAY | NSP1 | 110 | 118 | No |
| 1891 | IPVAYRKVLL | NSP1 | 114 | 123 | No |
| 1892 | VPANSTVLSF | NSP10 | 7 | 16 | No |
| 1893 | YLASGGQPI | NSP10 | 30 | 38 | No |
| 1894 | VVYRAFDIY | NSP12 | 30 | 38 | No |
| 1895 | KVAGFAKFL | NSP12 | 41 | 49 | No |
| 1896 | KVAGFAKFLK | NSP12 | 41 | 50 | No |
| 1897 | NLIDSYFVV | NSP12 | 64 | 72 | No |
| 1898 | YFVVKRHTF | NSP12 | 69 | 77 | No |
| 1899 | VPHISRQRL | NSP12 | 111 | 119 | No |
| 1900 | TLKEILVTY | NSP12 | 141 | 149 | No |
| 1901 | FVENPDILRV | NSP12 | 165 | 174 | No |
| 1902 | VENPDILRV | NSP12 | 166 | 174 | No |
| 1903 | VENPDILRVY | NSP12 | 166 | 175 | No |
| 1904 | VRQALLKTV | NSP12 | 182 | 190 | No |
| 1905 | DAMRNAGIV | NSP12 | 194 | 202 | No |
| 1906 | SLLMPILTL | NSP12 | 239 | 247 | No |
| 1907 | HVDTDLTKPY | NSP12 | 256 | 265 | No |
| 1908 | KPYIKWDLL | NSP12 | 263 | 271 | No |
| 1909 | RYFKYWDQTY | NSP12 | 285 | 294 | No |
| 1910 | ILHCANFNV | NSP12 | 307 | 315 | No |
| 1911 | STVFPPTSF | NSP12 | 318 | 326 | No |
| 1912 | FPPTSFGPL | NSP12 | 321 | 329 | No |
| 1913 | TSFGPLVRK | NSP12 | 324 | 332 | No |
| 1914 | SFGPLVRKI | NSP12 | 325 | 333 | No |
| 1915 | KIFVDGVPFV | NSP12 | 332 | 341 | No |
| 1916 | FVVSTGYHFR | NSP12 | 340 | 349 | No |
| 1917 | VVSTGYHFR | NSP12 | 341 | 349 | No |
| 1918 | NLHSSRLSF | NSP12 | 360 | 368 | No |
| 1919 | QTVKPGNFNK | NSP12 | 408 | 417 | No |
| 1920 | FAVSKGFFK | NSP12 | 422 | 430 | No |
| 1921 | AAISDYDYY | NSP12 | 448 | 456 | No |
| 1922 | AAISDYDYYR | NSP12 | 448 | 457 | No |
| 1923 | AISDYDYYR | NSP12 | 449 | 457 | No |
| 1924 | YRYNLPTMC | NSP12 | 456 | 464 | No |
| 1925 | FVVEVVDKY | NSP12 | 47 | 479 | No |
| 1926 | KSAGFPFNK | NSP12 | 500 | 508 | No |
| 1927 | KSAGFPFNKW | NSP12 | 500 | 509 | No |
| 1928 | ARLYYDSMSY | NSP12 | 512 | 521 | No |
| 1929 | FAYTKRNVI | NSP12 | 528 | 536 | No |
| 1930 | NVIPTITQM | NSP12 | 534 | 542 | No |
| 1931 | IPTITQMNL | NSP12 | 536 | 544 | No |
| 1932 | TITQMNLKY | NSP12 | 538 | 546 | No |
| 1933 | YAISAKNRAR | NSP12 | 546 | 555 | No |
| 1934 | SAKNRARTV | NSP12 | 549 | 557 | No |
| 1935 | SICSTMTNR | NSP12 | 561 | 569 | No |
| 1936 | IAATRGATV | NSP12 | 579 | 587 | No |
| 1937 | ATVVIGTSK | NSP12 | 585 | 593 | No |
| 1938 | VENPHLMGWD | NSP12 | 609 | 618 | No |
| 1939 | NMLRIMASL | NSP12 | 628 | 636 | No |
| 1940 | LRIMASLVL | NSP12 | 630 | 638 | No |
| 1941 | IMASLVLAR | NSP12 | 632 | 640 | No |
| 1942 | MASLVLARK | NSP12 | 633 | 641 | No |
| 1943 | RLANECAQV | NSP12 | 654 | 662 | No |
| 1944 | TSSGDATTAY | NSP12 | 680 | 689 | No |
| 1945 | VRNLQHRLY | NSP12 | 720 | 728 | No |
| 1946 | FVNEFYAYL | NSP12 | 741 | 749 | No |
| 1947 | FVNEFYAYLR | NSP12 | 741 | 750 | No |
| 1948 | LRKHFSMMI | NSP12 | 749 | 757 | No |
| 1949 | LYYQNNVFM | NSP12 | 786 | 794 | No |
| 1950 | LVKQGDDYVY | NSP12 | 819 | 828 | No |
| 1951 | KQGDDYVYL | NSP12 | 821 | 829 | No |
| 1952 | YLPYPDPSRI | NSP12 | 828 | 837 | No |
| 1953 | LPYPDPSRIL | NSP12 | 829 | 838 | No |
| 1954 | QEYADVFHLY | NSP12 | 875 | 884 | No |
| 1955 | YADVFHLYL | NSP12 | 877 | 885 | No |
| 1956 | LTNDNTSRYW | NSP12 | 907 | 916 | No |
| 1957 | WEPEFYEAM | NSP12 | 916 | 924 | No |
| 1958 | AMYTPHTVL | NSP12 | 923 | 931 | No |
| 1959 | TPHTVLQAV | NSP12 | 926 | 934 | No |
| 1960 | QLYLGGMSYY | NSP13 | 62 | 71 | No |
| 1961 | YRGTTTYKL | NSP13 | 21. | 219 | No |
| 1962 | TLVPQEHYV | NSP13 | 239 | 247 | No |
| 1963 | VPQEHYVRI | NSP13 | 241 | 249 | No |
| 1964 | YQKVGMQKY | NSP13 | 269 | 277 | No |
| 1965 | FAIGLALYY | NSP13 | 291 | 299 | No |
| 1966 | YYPSARIVY | NSP13 | 298 | 306 | No |
| 1967 | IPARARVEC | NSP13 | 334 | 342 | No |
| 1968 | IPARARVECF | NSP13 | 334 | 343 | No |
| 1969 | YVFCTVNAL | NSP13 | 355 | 363 | No |
| 1970 | VVNARLRAK | NSP13 | 386 | 394 | No |
| 1971 | RPQIGVVREF | NSP13 | 490 | 499 | No |
| 1972 | AVASKILGL | NSP13 | 520 | 528 | No |
| 1973 | ILGLPTQTV | NSP13 | 525 | 533 | No |
| 1974 | EEAIRHVRAW | NSP14 | 77 | 86 | No |
| 1975 | LQLGFSTGV | NSP14 | 107 | 115 | No |
| 1976 | MYKGLPWNV | NSP14 | 153 | 161 | No |
| 1977 | KNLSDRVVFV | NSP14 | 175 | 184 | No |
| 1978 | PFMIDVQQW | NSP14 | 239 | 247 | No |
| 1979 | LLADKFPVL | NSP14 | 321 | 329 | No |
| 1980 | YKIEELFYSY | NSP14 | 361 | 370 | No |
| 1981 | KIEELFYSY | NSP14 | 362 | 370 | No |
| 1982 | FTDGVCLFW | NSP14 | 377 | 385 | No |
| 1983 | SLYVNKHAF | NSP14 | 418 | 426 | No |
| 1984 | AGFSLWVYK | NSP14 | 504 | 512 | No |
| 1985 | YNLWNTFTRL | NSP14 | 517 | 526 | No |
| 1986 | NTFTRLQSL | NSP14 | 521 | 529 | No |
| 1987 | SLENVAFNV | NSP15 | 1 | 9 | No |
| 1988 | TTLPVNVAF | NSP15 | 47 | 55 | No |
| 1989 | TICAPLTVF | NSP15 | 114 | 122 | No |
| 1990 | GRVDGQVDL | NSP15 | 125 | 133 | No |
| 1991 | KVDGVVQQL | NSP15 | 181 | 189 | No |
| 1992 | VVQQLPETY | NSP15 | 185 | 193 | No |
| 1993 | KPRSQMEIDF | NSP15 | 204 | 213 | No |
| 1994 | FIERYKLEGY | NSP15 | 221 | 230 | No |
| 1995 | LLLDDFVEI | NSP15 | 297 | 305 | No |
| 1996 | SVVSKVVKV | NSP15 | 312 | 320 | No |
| 1997 | VAMPNLYKM | NSP16 | 9 | 17 | No |
| 1998 | LPKGIMMNV | NSP16 | 36 | 44 | No |
| 1999 | CATVHTANKW | NSP16 | 115 | 124 | No |
| 2000 | KLMGHFAWW | NSP16 | 182 | 190 | No |
| 2001 | YVMHANYIF | NSP16 | 222 | 230 | No |
| 2002 | FWRNTNPIQL | NSP16 | 230 | 239 | No |
| 2003 | WRNTNPIQL | NSP16 | 231 | 239 | No |
| 2004 | NPIQLSSYSL | NSP16 | 235 | 244 | No |
| 2005 | SYSLFDMSKF | NSP16 | 241 | 250 | No |
| 2006 | FPLKLRGTA | NSP16 | 250 | 258 | No |
| 2007 | FPLKLRGTAV | NSP16 | 250 | 259 | No |
| 2008 | LRGTAVMSL | NSP16 | 254 | 262 | No |
| 2009 | TFNGECPNF | NSP2 | 85 | 93 | No |
| 2010 | GFMGRIRSV | NSP2 | 115 | 123 | No |
| 2011 | EEIAIILASF | NSP2 | 291 | 300 | No |
| 2012 | ILSPLYAFA | NSP2 | 349 | 357 | No |
| 2013 | VRSIFSRTL | NSP2 | 364 | 372 | No |
| 2014 | ITILDGISQY | NSP2 | 387 | 396 | No |
| 2015 | RLIDAMMFT | NSP2 | 399 | 407 | No |
| 2016 | TVYEKLKPV | NSP2 | 439 | 447 | No |
| 2017 | EIKESVQTF | NSP2 | 490 | 498 | No |
| 2018 | GETLPTEVL | NSP2 | 564 | 572 | No |
| 2019 | DTVIEVQGYK | NSP3 | 10 | 19 | No |
| 2020 | QGYKSVNITF | NSP3 | 16 | 25 | No |
| 2021 | FELDERIDKV | NSP3 | 25 | 34 | No |
| 2022 | ELDERIDKV | NSP3 | 26 | 34 | No |
| 2023 | VLNEKCSAY | NSP3 | 34 | 42 | No |
| 2024 | VELGTEVNEF | NSP3 | 44 | 53 | No |
| 2025 | SELLTPLGI | NSP3 | 69 | 77 | No |
| 2026 | YLFDESGEF | NSP3 | 88 | 96 | No |
| 2027 | FEPSTQYEY | NSP3 | 123 | 131 | No |
| 2028 | DDYQGKPLEF | NSP3 | 135 | 144 | No |
| 2029 | LEFGATSAAL | NSP3 | 142 | 151 | No |
| 2030 | VEVQPQLEM | NSP3 | 188 | 196 | No |
| 2031 | LEMELTPVV | NSP3 | 194 | 202 | No |
| 2032 | MELTPVVQTI | NSP3 | 196 | 205 | No |
| 2033 | TPVVQTIEV | NSP3 | 199 | 207 | No |
| 2034 | TIEVNSFSGY | NSP3 | 204 | 213 | No |
| 2035 | IEVNSFSGY | NSP3 | 205 | 213 | No |
| 2036 | NSFSGYLKL | NSP3 | 208 | 216 | No |
| 2037 | YLKLTDNVY | NSP3 | 213 | 221 | No |
| 2038 | EAKKVKPTV | NSP3 | 230 | 238 | No |
| 2039 | VVVNAANVY | NSP3 | 238 | 246 | No |
| 2040 | YIATNGPLK | NSP3 | 272 | 280 | No |
| 2041 | YENFNQHEV | NSP3 | 317 | 325 | No |
| 2042 | LLSAGIFGA | NSP3 | 330 | 338 | No |
| 2043 | GADPIHSLR | NSP3 | 337 | 345 | No |
| 2044 | RTNVYLAVF | NSP3 | 352 | 360 | No |
| 2045 | AVFDKNLYDK | NSP3 | 358 | 367 | No |
| 2046 | LYDKLVSSF | NSP3 | 364 | 372 | No |
| 2047 | KLVSSFLEM | NSP3 | 367 | 375 | No |
| 2048 | KIAEIPKEEV | NSP3 | 384 | 393 | No |
| 2049 | EVKPFITESK | NSP3 | 392 | 401 | No |
| 2050 | ESKPSVEQR | NSP3 | 399 | 407 | No |
| 2051 | FLTENLLLYI | NSP3 | 430 | 439 | No |
| 2052 | LVSDIDITF | NSP3 | 452 | 460 | No |
| 2053 | APYIVGDVV | NSP3 | 465 | 473 | No |
| 2054 | LTAVVIPTK | NSP3 | 478 | 486 | No |
| 2055 | ALRKVPTDNY | NSP3 | 498 | 507 | No |
| 2056 | KQEILGTVSW | NSP3 | 546 | 555 | No |
| 2057 | MLAHAEETR | NSP3 | 560 | 568 | No |
| 2058 | AHAEETRKL | NSP3 | 562 | 570 | No |
| 2059 | KLMPVCVET | NSP3 | 569 | 577 | No |
| 2060 | AIVSTIQRK | NSP3 | 579 | 587 | No |
| 2061 | VVDYGARFY | NSP3 | 597 | 605 | No |
| 2062 | SLINTLNDL | NSP3 | 615 | 623 | No |
| 2063 | VSSPDAVTAY | NSP3 | 659 | 668 | No |
| 2064 | TISLAGSYK | NSP3 | 686 | 694 | No |
| 2065 | ISLAGSYKDW | NSP3 | 687 | 696 | No |
| 2066 | YYTSNPTTF | NSP3 | 718 | 726 | No |
| 2067 | TSNPTTFHL | NSP3 | 720 | 728 | No |
| 2068 | HLDGEVITF | NSP3 | 727 | 735 | No |
| 2069 | GEVITFDNL | NSP3 | 730 | 738 | No |
| 2070 | ITFDNLKTL | NSP3 | 733 | 741 | No |
| 2071 | RTIKVFTTV | NSP3 | 748 | 756 | No |
| 2072 | NINLHTQVV | NSP3 | 758 | 766 | No |
| 2073 | QVVDMSMTY | NSP3 | 764 | 772 | No |
| 2074 | MSMTYGQQF | NSP3 | 768 | 776 | No |
| 2075 | LRVEAFEYY | NSP3 | 809 | 817 | No |
| 2076 | HTTDPSFLGR | NSP3 | 818 | 827 | No |
| 2077 | FLGRYMSAL | NSP3 | 824 | 832 | No |
| 2078 | YMSALNHTK | NSP3 | 828 | 836 | No |
| 2079 | MSALNHTKK | NSP3 | 829 | 837 | No |
| 2080 | MSALNHTKKW | NSP3 | 829 | 838 | No |
| 2081 | SALNHTKKW | NSP3 | 830 | 838 | No |
| 2082 | WKYPQVNGL | NSP3 | 838 | 846 | No |
| 2083 | YPQVNGLTSI | NSP3 | 840 | 849 | No |
| 2084 | ARAGEAANF | NSP3 | 884 | 892 | No |
| 2085 | GEAANFCAL | NSP3 | 887 | 895 | No |
| 2086 | LGDVRETMSY | NSP3 | 907 | 916 | No |
| 2087 | VRETMSYLF | NSP3 | 910 | 918 | No |
| 2088 | VMYMGTLSY | NSP3 | 950 | 958 | No |
| 2089 | YTGNYQCGHY | NSP3 | 1009 | 1018 | No |
| 2090 | DVFYKENSY | NSP3 | 1047 | 1055 | No |
| 2091 | YTTTIKPVTY | NSP3 | 1055 | 1064 | No |
| 2092 | EIDPKLDNY | NSP3 | 1073 | 1081 | No |
| 2093 | NYYKKDNSY | NSP3 | 1080 | 1088 | No |
| 2094 | YYKKDNSYF | NSP3 | 1081 | 1089 | No |
| 2095 | ASFDNFKFV | NSP3 | 1105 | 1113 | No |
| 2096 | KFADDLNQL | NSP3 | 1118 | 1126 | No |
| 2097 | ASRELKVTF | NSP3 | 1133 | 1141 | No |
| 2098 | DVVAIDYKHY | NSP3 | 1148 | 1157 | No |
| 2099 | VVAIDYKHY | NSP3 | 1149 | 1157 | No |
| 2100 | DYKHYTPSF | NSP3 | 1153 | 1161 | No |
| 2101 | LHKPIVWHV | NSP3 | 1168 | 1176 | No |
| 2102 | NKATYKPNTW | NSP3 | 1181 | 1190 | No |
| 2103 | SEDAQGMDNL | NSP3 | 1212 | 1221 | No |
| 2104 | EEVVENPTI | NSP3 | 1231 | 1239 | No |
| 2105 | TEVVGDIIL | NSP3 | 1251 | 1259 | No |
| 2106 | AYVDNSSLTI | NSP3 | 1280 | 1289 | No |
| 2107 | LTIKKPNEL | NSP3 | 1287 | 1295 | No |
| 2108 | KPNELSRVL | NSP3 | 1291 | 1299 | No |
| 2109 | NELSRVLGL | NSP3 | 1293 | 1301 | No |
| 2110 | SRVLGLKTL | NSP3 | 1296 | 1304 | No |
| 2111 | SVPWDTIANY | NSP3 | 1314 | 1323 | No |
| 2112 | DTIANYAKPF | NSP3 | 1318 | 1327 | No |
| 2113 | YAKPFLNKV | NSP3 | 1323 | 1331 | No |
| 2114 | RIKASMPTT | NSP3 | 1371 | 1379 | No |
| 2115 | KASMPTTIA | NSP3 | 1373 | 1381 | No |
| 2116 | MPTTIAKNTV | NSP3 | 1376 | 1385 | No |
| 2117 | TTIAKNTVK | NSP3 | 1378 | 1386 | No |
| 2118 | NTVKSVGKF | NSP3 | 1383 | 1391 | No |
| 2119 | KFCLEASFNY | NSP3 | 1390 | 1399 | No |
| 2120 | CLEASFNYL | NSP3 | 1392 | 1400 | No |
| 2121 | KLINIIIWF | NSP3 | 1407 | 1415 | No |
| 2122 | SLIYSTAAL | NSP3 | 1424 | 1432 | No |
| 2123 | STAALGVLM | NSP3 | 1428 | 1436 | No |
| 2124 | MSNLGMPSY | NSP3 | 1436 | 1444 | No |
| 2125 | EGYLNSTNV | NSP3 | 1450 | 1458 | No |
| 2126 | NSTNVTIATY | NSP3 | 1454 | 1463 | No |
| 2127 | STNVTIATY | NSP3 | 1455 | 1463 | No |
| 2128 | ETIQITISSF | NSP3 | 1487 | 1496 | No |
| 2129 | IQITISSFK | NSP3 | 1489 | 1497 | No |
| 2130 | LTAFGLVAEW | NSP3 | 1500 | 1509 | No |
| 2131 | LVAEWFLAY | NSP3 | 1505 | 1513 | No |
| 2132 | AEWFLAYIL | NSP3 | 1507 | 1515 | No |
| 2133 | AEWFLAYILF | NSP3 | 1507 | 1516 | No |
| 2134 | AYILFTRFF | NSP3 | 1512 | 1520 | No |
| 2135 | AVHFISNSW | NSP3 | 1537 | 1545 | No |
| 2136 | HFISNSWLMW | NSP3 | 1539 | 1548 | No |
| 2137 | WLMWLIINL | NSP3 | 1545 | 1553 | No |
| 2138 | LVQMAPISAM | NSP3 | 1553 | 1562 | No |
| 2139 | SAMVRMYIF | NSP3 | 1560 | 1568 | No |
| 2140 | RMYIFFASFY | NSP3 | 1564 | 1573 | No |
| 2141 | SFYYVWKSY | NSP3 | 1571 | 1579 | No |
| 2142 | FYYVWKSYV | NSP3 | 1572 | 1580 | No |
| 2143 | YVYANGGKGF | NSP3 | 1617 | 1626 | No |
| 2144 | DTFCAGSTF | NSP3 | 1638 | 1646 | No |
| 2145 | EVARDLSLQF | NSP3 | 1650 | 1659 | No |
| 2146 | VARDLSLQF | NSP3 | 1651 | 1659 | No |
| 2147 | TVKNGSIHLY | NSP3 | 1677 | 1686 | No |
| 2148 | VKNGSIHLY | NSP3 | 1678 | 1686 | No |
| 2149 | YFDKAGQKTY | NSP3 | 1686 | 1695 | No |
| 2150 | TYERHSLSHF | NSP3 | 1694 | 1703 | No |
| 2151 | YERHSLSHF | NSP3 | 1695 | 1703 | No |
| 2152 | ERHSLSHFV | NSP3 | 1696 | 1704 | No |
| 2153 | SSAKSASVY | NSP3 | 1734 | 1742 | No |
| 2154 | SAKSASVYY | NSP3 | 1735 | 1743 | No |
| 2155 | DSAEVAVKM | NSP3 | 1764 | 1772 | No |
| 2156 | EVAVKMFDAY | NSP3 | 1767 | 1776 | No |
| 2157 | KMFDAYVNTF | NSP3 | 1771 | 1780 | No |
| 2158 | MFDAYVNTF | NSP3 | 1772 | 1780 | No |
| 2159 | AYVNTFSSTF | NSP3 | 1775 | 1784 | No |
| 2160 | YVNTFSSTF | NSP3 | 1776 | 1784 | No |
| 2161 | VPMEKLKTL | NSP3 | 1786 | 1794 | No |
| 2162 | AEAELAKNV | NSP3 | 1798 | 1806 | No |
| 2163 | AELAKNVSL | NSP3 | 1800 | 1808 | No |
| 2164 | SLDNVLSTF | NSP3 | 1807 | 1815 | No |
| 2165 | TFISAARQGF | NSP3 | 1814 | 1823 | No |
| 2166 | DSCNNYMLTY | NSP3 | 1850 | 1859 | No |
| 2167 | VENMTPRDL | NSP3 | 1862 | 1870 | No |
| 2168 | TPRDLGACI | NSP3 | 1866 | 1874 | No |
| 2169 | VAKSHNIAL | NSP3 | 1885 | 1893 | No |
| 2170 | AKSHNIALIW | NSP3 | 1886 | 1895 | No |
| 2171 | QVVNVVTTK | NSP3 | 1931 | 1939 | No |
| 2172 | VVTTKIALK | NSP3 | 1935 | 1943 | No |
| 2173 | KQLIKVTLVF | NSP4 | 8 | 17 | No |
| 2174 | FYLITPVHV | NSP4 | 24 | 32 | No |
| 2175 | YLITPVHVM | NSP4 | 25 | 33 | No |
| 2176 | SEIIGYKAI | NSP4 | 41 | 49 | No |
| 2177 | IAAVITREV | NSP4 | 91 | 99 | No |
| 2178 | FVVPGLPGT | NSP4 | 101 | 109 | No |
| 2179 | VPGLPGTIL | NSP4 | 103 | 111 | No |
| 2180 | RTTNGDFLHF | NSP4 | 112 | 121 | No |
| 2181 | VLAAECTIF | NSP4 | 151 | 159 | No |
| 2182 | DASGKPVPY | NSP4 | 161 | 169 | No |
| 2183 | TNVLEGSVAY | NSP4 | 173 | 182 | No |
| 2184 | GSVAYESLR | NSP4 | 178 | 186 | No |
| 2185 | RPDTRYVLM | NSP4 | 186 | 194 | No |
| 2186 | SIIQFPNTY | NSP4 | 197 | 205 | No |
| 2187 | WVLNNDYYR | NSP4 | 241 | 249 | No |
| 2188 | SLPGVFCGV | NSP4 | 250 | 258 | No |
| 2189 | DAVNLLTNM | NSP4 | 259 | 267 | No |
| 2190 | IVAGGIVAI | NSP4 | 284 | 292 | No |
| 2191 | LAYYFMRFR | NSP4 | 297 | 305 | No |
| 2192 | YFMRFRRAF | NSP4 | 300 | 308 | No |
| 2193 | FGEYSHVVAF | NSP4 | 308 | 317 | No |
| 2194 | SFLPGVYSV | NSP4 | 336 | 344 | No |
| 2195 | IYLYLTFYL | NSP4 | 345 | 353 | No |
| 2196 | YLTNDVSFLA | NSP4 | 352 | 361 | No |
| 2197 | FLAHIQWMV | NSP4 | 359 | 367 | No |
| 2198 | MFTPLVPFW | NSP4 | 368 | 376 | No |
| 2199 | VPFWITIAY | NSP4 | 373 | 381 | No |
| 2200 | WFFSNYLKR | NSP4 | 392 | 400 | No |
| 2201 | DVLLPLTQY | NSP4 | 433 | 441 | No |
| 2202 | LPLTQYNRY | NSP4 | 436 | 444 | No |
| 2203 | GAMDTTSYR | NSP4 | 456 | 464 | No |
| 2204 | SNSGSDVLY | NSP4 | 479 | 487 | No |
| 2205 | LYQPPQTSI | NSP4 | 486 | 494 | No |
| 2206 | AVLQSGFRK | NSP5 | -3 | 5 | No |
| 2207 | TANPKTPKY | NSP5 | 93 | 101 | No |
| 2208 | NPKTPKYKF | NSP5 | 95 | 103 | No |
| 2209 | IQPGQTFSV | NSP5 | 106 | 114 | No |
| 2210 | QPGQTFSVL | NSP5 | 107 | 115 | No |
| 2211 | SPSGVYQCAM | NSP5 | 121 | 130 | No |
| 2212 | ILTSLLVLV | NSP6 | 18 | 26 | No |
| 2213 | FLYENAFLP | NSP6 | 36 | 44 | No |
| 2214 | LPFAMGIIAM | NSP6 | 43 | 52 | No |
| 2215 | MFVKHKHAF | NSP6 | 58 | 66 | No |
| 2216 | FVKHKHAFL | NSP6 | 59 | 67 | No |
| 2217 | LFLLPSLATV | NSP6 | 69 | 78 | No |
| 2218 | FLLPSLATVA | NSP6 | 70 | 79 | No |
| 2219 | VYMPASWVM | NSP6 | 84 | 92 | No |
| 2220 | RIMTWLDMV | NSP6 | 93 | 101 | No |
| 2221 | WLDMVDTSL | NSP6 | 97 | 105 | No |
| 2222 | VMYASAVVLL | NSP6 | 114 | 123 | No |
| 2223 | MYASAVVLL | NSP6 | 115 | 123 | No |
| 2224 | YASAVVLLI | NSP6 | 116 | 124 | No |
| 2225 | TLMNVLTLV | NSP6 | 141 | 149 | No |
| 2226 | FLARGIVFM | NSP6 | 184 | 192 | No |
| 2227 | IFFITGNTL | NSP6 | 199 | 207 | No |
| 2228 | GVYDYLVST | NSP6 | 240 | 248 | No |
| 2229 | YDYLVSTQEF | NSP6 | 242 | 251 | No |
| 2230 | FRYMNSQGL | NSP6 | 251 | 259 | No |
| 2231 | GLLPPKNSI | NSP6 | 258 | 266 | No |
| 2232 | KLNIKLLGV | NSP6 | 270 | 278 | No |
| 2233 | EAFEKMVSL | NSP7 | 47 | 55 | No |
| 2234 | SLLSVLLSM | NSP7 | 54 | 62 | No |
| 2235 | TFTYASALW | NSP8 | 146 | 154 | No |
| 2236 | AWPLIVTAL | NSP8 | 181 | 189 | No |
| 2237 | GPKVKYLYF | NSP9 | 82 | 90 | No |
| 2238 | YFIKGLNNL | NSP9 | 89 | 97 | No |
| 2239 | YINVFAFPF | ORF10 | 3 | 11 | No |
| 2240 | NVFAFPFTI | ORF10 | 5 | 13 | No |
| 2241 | FTIGTVTLK | ORF3a | 8 | 16 | No |
| 2242 | ATIPIQASL | ORF3a | 33 | 41 | No |
| 2243 | ALLAVFQSA | ORF3a | 51 | 59 | No |
| 2244 | QSASKIITL | ORF3a | 57 | 65 | No |
| 2245 | SASKIITLK | ORF3a | 58 | 66 | No |
| 2246 | ITLKKRWQL | ORF3a | 63 | 71 | No |
| 2247 | TLKKRWQLA | ORF3a | 64 | 72 | No |
| 2248 | NLLLLFVTV | ORF3a | 82 | 90 | No |
| 2249 | FVTVYSHLL | ORF3a | 87 | 95 | No |
| 2250 | TVYSHLLLV | ORF3a | 89 | 97 | No |
| 2251 | VAAGLEAPF | ORF3a | 97 | 105 | No |
| 2252 | AAGLEAPFLY | ORF3a | 98 | 107 | No |
| 2253 | AGLEAPFLY | ORF3a | 99 | 107 | No |
| 2254 | GLEAPFLYL | ORF3a | 100 | 108 | No |
| 2255 | LEAPFLYLY | ORF3a | 101 | 109 | No |
| 2256 | APFLYLY AL | ORF3a | 103 | 111 | No |
| 2257 | FLYLYALVY | ORF3a | 105 | 113 | No |
| 2258 | LYLYALVYF | ORF3a | 106 | 114 | No |
| 2259 | FVRIIMRLW | ORF3a | 120 | 128 | No |
| 2260 | VRIIMRLWL | ORF3a | 121 | 129 | No |
| 2261 | CRSKNPLLY | ORF3a | 133 | 141 | No |
| 2262 | NPLLYDANY | ORF3a | 137 | 145 | No |
| 2263 | IPYNSVTSSI | ORF3a | 158 | 167 | No |
| 2264 | TTSPISEHDY | ORF3a | 175 | 184 | No |
| 2265 | HSYFTSDYY | ORF3a | 204 | 212 | No |
| 2266 | YFTSDYYQL | ORF3a | 206 | 214 | No |
| 2267 | YFTSDYYQLY | ORF3a | 206 | 215 | No |
| 2268 | DYYQLYSTQL | ORF3a | 210 | 219 | No |
| 2269 | HVTFFIYNK | ORF3a | 227 | 235 | No |
| 2270 | EEHVQIHTI | ORF3a | 241 | 249 | No |
| 2271 | IYDEPTTTT | ORF3a | 263 | 271 | No |
| 2272 | HLVDFQVTIA | ORF6 | 3 | 12 | No |
| 2273 | VTIAEILLI | ORF6 | 9 | 17 | No |
| 2274 | LIIMRTFKV | ORF6 | 16 | 24 | No |
| 2275 | TFKVSIWNL | ORF6 | 21 | 29 | No |
| 2276 | LDYIINLII | ORF6 | 29 | 37 | No |
| 2277 | QECVRGTTVL | ORF7a | 21 | 30 | No |
| 2278 | YEGNSPFHPL | ORF7a | 40 | 49 | No |
| 2279 | EGNSPFHPL | ORF7a | 41 | 49 | No |
| 2280 | HPLADNKFAL | ORF7a | 47 | 56 | No |
| 2281 | SPIFLIVAA | ORF7a | 98 | 106 | No |
| 2282 | QSCTQHQPY | ORF8 | 23 | 31 | No |
| 2283 | VDDPCPIHFY | ORF8 | 33 | 42 | No |
| 2284 | RVGARKSAPL | ORF8 | 48 | 57 | No |
| 2285 | EPKLGSLVV | ORF8 | 92 | 100 | No |
| 2286 | EYHDVRVVL | ORF8 | 110 | 118 | No |

•CD4R* (D) = 69 (Table 6). CD4R (D) Megapool

| SEQ ID NO: | Description | Antigen | Start | End | Dominant |
|---|---|---|---|---|---|
| 2287 | FYVYSRVKNLNSSRV | E | 56 | 70 | Yes |
| 2288 | QFAYANRNRFLYIIK | M | 36 | 50 | Yes |
| 2289 | VLAAVYRINWITGGI | M | 66 | 80 | Yes |
| 2290 | YRINWITGGIAIAMA | M | 71 | 85 | Yes |
| 2291 | CLVGLMWLSYFIASF | M | 86 | 100 | Yes |
| 2292 | MWLSYFIASFRLFAR | M | 91 | 105 | Yes |
| 2293 | TNILLNVPLHGTILT | M | 116 | 130 | Yes |
| 2294 | SELVIGAVILRGHLR | M | 136 | 150 | Yes |
| 2295 | GAVILRGHLRIAGHHLGR | M | 141 | 158 | Yes |
| 2296 | LRGHLRIAGHHLGRC | M | 145 | 159 | Yes |
| 2297 | RGHLRIAGHHLGRCD | M | 146 | 160 | Yes |
| 2298 | IAGHHLGRCDIKDLP | M | 151 | 165 | Yes |
| 2299 | LGRCDIKDLPKEITV | M | 156 | 170 | Yes |
| 2300 | IKDLPKEITVATSRT | M | 161 | 175 | Yes |
| 2301 | KEITVATSRTLSYYK | M | 166 | 180 | Yes |
| 2302 | TSRTLSYYKLGASQRVA | M | 172 | 188 | Yes |
| 2303 | SRTLSYYKLGASQRV | M | 173 | 187 | Yes |
| 2304 | LSYYKLGASQRVAGD | M | 176 | 190 | Yes |
| 2305 | SYYKLGASQRVAGDS | M | 177 | 191 | Yes |
| 2306 | LGASQRVAGDSGFAA | M | 181 | 195 | Yes |
| 2307 | SGFAAYSRYRIGNYK | M | 191 | 205 | Yes |
| 2308 | ASWFTALTQHGKEDL | N | 50 | 64 | Yes |
| 2309 | SWFTALTQHGKEDLK | N | 51 | 65 | Yes |
| 2310 | DDQIGYYRRATRRIR | N | 81 | 95 | Yes |
| 2311 | IGYYRRATRRIRGGD | N | 84 | 98 | Yes |
| 2312 | YYRRATRRIRGGDGK | N | 86 | 100 | Yes |
| 2313 | MKDLSPRWYFYYLGT | N | 101 | 115 | Yes |
| 2314 | RWYFYYLGTGPEAGL | N | 107 | 121 | Yes |
| 2315 | NKDGIIWVATEGALN | N | 126 | 140 | Yes |
| 2316 | KDGIIWVATEGALNT | N | 127 | 141 | Yes |
| 2317 | AIVLQLPQGTTLPKG | N | 156 | 170 | Yes |
| 2318 | AGNGGDAALALLLLD | N | 211 | 225 | Yes |
| 2319 | DAALALLLLDRLNQL | N | 216 | 230 | Yes |
| 2320 | LLLLDRLNQLESKMS | N | 221 | 235 | Yes |
| 2321 | AAEASKKPRQKRTAT | N | 251 | 265 | Yes |
| 2322 | KKPRQKRTATKAYNV | N | 256 | 270 | Yes |
| 2323 | KRTATKAYNVTQAFG | N | 261 | 275 | Yes |
| 2324 | KAYNVTQAFGRRGPE | N | 266 | 280 | Yes |
| 2325 | LIRQGTDYKHWPQIA | N | 291 | 305 | Yes |
| 2326 | YKHWPQIAQFAPSAS | N | 298 | 312 | Yes |
| 2327 | WPQIAQFAPSASAFF | N | 301 | 315 | Yes |
| 2328 | ASAFFGMSRIGMEVT | N | 311 | 325 | Yes |
| 2329 | GMEVTPSGTWLTYTGAIKLD | N | 321 | 340 | Yes |
| 2330 | PSGTWLTYTGAIKLD | N | 326 | 340 | Yes |
| 2331 | GTWLTYTGAIKLDDK | N | 328 | 342 | Yes |
| 2332 | FKDQVILLNKHIDAY | N | 346 | 360 | Yes |
| 2333 | ILLNKHIDAYKTFPP | N | 351 | 365 | Yes |
| 2334 | LMIERFVSLAIDAYP | NSP12 | 854 | 868 | Yes |
| 2335 | LDDFVEIIKSQDLSV | NSP15 | 299 | 313 | Yes |
| 2336 | KVTFFPDLNGDVVAI | NSP3 | 1138 | 1152 | Yes |
| 2337 | KHFYWFFSNYLKRRV | NSP4 | 388 | 402 | Yes |
| 2338 | NRYFRLTLGVYDYLV | NSP6 | 232 | 246 | Yes |
| 2339 | INVFAFPFTIYSLLL | ORF10 | 4 | 18 | Yes |
| 2340 | FMRIFTIGTVTLKQG | ORF3a | 4 | 18 | Yes |
| 2341 | KKRWQLALSKGVHFV | ORF3a | 66 | 80 | Yes |
| 2342 | LYLYALVYFLQSINF | ORF3a | 106 | 120 | Yes |
| 2343 | QSINFVRIIMRLWLC | ORF3a | 116 | 130 | Yes |
| 2344 | IWNLDYIINLIIKNL | ORF6 | 26 | 40 | Yes |
| 2345 | QEEVQELYSPIFLIV | ORF7a | 90 | 104 | Yes |
| 2346 | TQHQPYVVDDPCPIH | ORF8 | 26 | 40 | Yes |
| 2347 | YVVDDPCPIHFYSKW | ORF8 | 31 | 45 | Yes |
| 2348 | PCPIHFYSKWYIRVG | ORF8 | 36 | 50 | Yes |
| 2349 | FYSKWYIRVGARKSA | ORF8 | 41 | 55 | Yes |
| 2350 | SKWYIRVGARKSAPL | ORF8 | 43 | 57 | Yes |
| 2351 | YIRVGARKSAPLIEL | ORF8 | 46 | 60 | Yes |
| 2352 | IGNYTVSCLPFTINC | ORF8 | 76 | 90 | Yes |
| 2353 | FTINCQEPKLGSLVV | ORF8 | 86 | 100 | Yes |
| 2354 | GSLVVRCSFYEDFLE | ORF8 | 96 | 110 | Yes |
| 2355 | RCSFYEDFLEYHDVR | ORF8 | 101 | 115 | Yes |

•CD4R* (ND)= 215 (Table 7). CD4R (ND) Megapool

| SEQ ID NO: | Description | Antigen | Start | End | Dominant |
|---|---|---|---|---|---|
| 2356 | SEETGTLIVNSVLLF | E | 6 | 20 | No |
| 2357 | FLLVTLAILTALRLC | E | 26 | 40 | No |
| 2358 | LAILTALRLCAYCCN | E | 31 | 45 | No |
| 2359 | EELKKLLEQWNLVIG | M | 11 | 25 | No |
| 2360 | LLEQWNLVIGFLFLT | M | 16 | 30 | No |
| 2361 | NLVIGFLFLTWICLL | M | 21 | 35 | No |
| 2362 | FLFLTWICLLQFAYA | M | 26 | 40 | No |
| 2363 | WICLLQFAYANRNRF | M | 31 | 45 | No |
| 2364 | NRNRFLYIIKLIFLW | M | 41 | 55 | No |
| 2365 | LYIIKLIFLWLLWPV | M | 46 | 60 | No |
| 2366 | LIFLWLLWPVTLACF | M | 51 | 65 | No |
| 2367 | LLWPVTLACFVLAAV | M | 56 | 70 | No |
| 2368 | ITGGIAIAMACLVGL | M | 76 | 90 | No |
| 2369 | AIAMACLVGLMWLSY | M | 81 | 95 | No |
| 2370 | FIASFRLFARTRSMW | M | 96 | 110 | No |
| 2371 | RLFARTRSMWSFNPE | M | 101 | 115 | No |
| 2372 | TRSMWSFNPETNILL | M | 106 | 120 | No |
| 2373 | SFNPETNILLNVPLH | M | 111 | 125 | No |
| 2374 | NVPLHGTILTRPLLE | M | 121 | 135 | No |
| 2375 | GTILTRPLLESELVI | M | 126 | 140 | No |
| 2376 | RPLLESELVIGAVIL | M | 131 | 145 | No |
| 2377 | GAVILRGHLRIAGHH | M | 141 | 155 | No |
| 2378 | LRIAGHHLGRCDIKD | M | 149 | 163 | No |
| 2379 | ATSRTLSYYKLGASQ | M | 171 | 185 | No |
| 2380 | RVAGDSGFAAYSRYR | M | 186 | 200 | No |
| 2381 | YSRYRIGNYKLNTDH | M | 196 | 210 | No |
| 2382 | IGNYKLNTDHSSSSD | M | 201 | 215 | No |
| 2383 | LNTDHSSSSDNIALL | M | 206 | 220 | No |
| 2384 | PQNQRNAPRITFGGP | N | 6 | 20 | No |
| 2385 | NAPRITFGGPSDSTG | N | 11 | 25 | No |
| 2386 | TFGGPSDSTGSNQNG | N | 16 | 30 | No |
| 2387 | LTQHGKEDLKFPRGQ | N | 56 | 70 | No |
| 2388 | FPRGQGVPINTNSSP | N | 66 | 80 | No |
| 2389 | GVPINTNSSPDDQIG | N | 71 | 85 | No |
| 2390 | TRRIRGGDGKMKDLS | N | 91 | 105 | No |
| 2391 | GGDGKMKDLSPRWYF | N | 96 | 110 | No |
| 2392 | MKDLSPRWYFYYLGTGPEAG | N | 101 | 120 | No |
| 2393 | PRWYFYYLGTGPEAG | N | 106 | 120 | No |
| 2394 | LPYGANKDGIIWVAT | N | 121 | 135 | No |
| 2395 | IWVATEGALNTPKDH | N | 131 | 145 | No |
| 2396 | EGALNTPKDHIGTRN | N | 136 | 150 | No |
| 2397 | GKGQQQQGQTVTKKS | N | 236 | 250 | No |
| 2398 | KPRQKRTATKAYNVT | N | 257 | 271 | No |
| 2399 | TQAFGRRGPEQTQGN | N | 271 | 285 | No |
| 2400 | RRGPEQTQGNFGDQE | N | 276 | 290 | No |
| 2401 | FGDQELIRQGTDYKH | N | 286 | 300 | No |
| 2402 | TDYKHWPQIAQFAPS | N | 296 | 310 | No |
| 2403 | QFAPSASAFFGMSRI | N | 306 | 320 | No |
| 2404 | AFFGMSRIGMEVTPS | N | 313 | 327 | No |
| 2405 | GMEVTPSGTWLTYTG | N | 321 | 335 | No |
| 2406 | TWLTYTGAIKLDDKDPNF | N | 329 | 346 | No |
| 2407 | LTYTGAIKLDDKDPN | N | 331 | 345 | No |
| 2408 | AIKLDDKDPNFKDQV | N | 336 | 350 | No |
| 2409 | PNFKDQVILLNKHIDAYK | N | 344 | 361 | No |
| 2410 | HIDAYKTFPPTEPKK | N | 356 | 370 | No |
| 2411 | QKKQQTVTLLPAADL | N | 386 | 400 | No |
| 2412 | KQQTVTLLPAADLDDF | N | 388 | 403 | No |
| 2413 | TVTLLPAADLDDFSK | N | 391 | 405 | No |
| 2414 | AADLDDFSKQLQQSM | N | 397 | 411 | No |
| 2415 | VLSFCAFAVDAAKAY | NSP10 | 13 | 27 | No |
| 2416 | PDILRVYANLGERVR | NSP12 | 169 | 183 | No |
| 2417 | SLLMPILTLTRALTA | NSP12 | 239 | 253 | No |
| 2418 | HCANFNVLFSTVFPP | NSP12 | 309 | 323 | No |
| 2419 | NVLFSTVFPPTSFGP | NSP12 | 314 | 328 | No |
| 2420 | QDALFAYTKRNVIPT | NSP12 | 524 | 538 | No |
| 2421 | KLLKSIAATRGATVV | NSP12 | 574 | 588 | No |
| 2422 | IAATRGATVVIGTSK | NSP12 | 579 | 593 | No |
| 2423 | YPKCDRAMPNMLRIM | NSP12 | 619 | 633 | No |
| 2424 | RAMPNMLRIMASLVL | NSP12 | 624 | 638 | No |
| 2425 | SHRFYRLANECAQVL | NSP12 | 649 | 663 | No |
| 2426 | SEMVMCGGSLYVKPG | NSP12 | 664 | 678 | No |
| 2427 | FNICQAVTANVNALL | NSP12 | 694 | 708 | No |
| 2428 | AVTANVNALLSTDGN | NSP12 | 699 | 713 | No |
| 2429 | EFYAYLRKHFSMMIL | NSP12 | 744 | 758 | No |
| 2430 | LRKHFSMMILSDDAV | NSP12 | 749 | 763 | No |
| 2431 | GLVASIKNFKSVLYY | NSP12 | 774 | 788 | No |
| 2432 | KTDGTLMIERFVSLA | NSP12 | 849 | 863 | No |
| 2433 | FVSLAIDAYPLTKHP | NSP12 | 859 | 873 | No |
| 2434 | IDAYPLTKHPNQEYA | NSP12 | 864 | 878 | No |
| 2435 | DVFHLYLQYIRKLHD | NSP12 | 879 | 893 | No |
| 2436 | TSHKLVLSVNPYVCN | NSP13 | 37 | 51 | No |
| 2437 | ELHLSWEVGKPRPPL | NSP13 | 162 | 176 | No |
| 2438 | PRPPLNRNYVFTGYR | NSP13 | 172 | 186 | No |
| 2439 | FTGYRVTKNSKVQIG | NSP13 | 182 | 196 | No |
| 2440 | VTKNSKVQIGEYTFE | NSP13 | 187 | 201 | No |
| 2441 | VNARLRAKHYVYIGD | NSP13 | 387 | 401 | No |
| 2442 | ISPYNSQNAVASKIL | NSP13 | 512 | 526 | No |
| 2443 | NVNRFNVAITRAKVG | NSP13 | 557 | 571 | No |
| 2444 | NMFITREEAIRHVRA | NSP14 | 71 | 85 | No |
| 2445 | REEAIRHVRAWIGFD | NSP14 | 76 | 90 | No |
| 2446 | PLMYKGLPWNVVRIK | NSP14 | 151 | 165 | No |
| 2447 | EIIKSQDLSVVSKVV | NSP15 | 304 | 318 | No |
| 2448 | TQLCQYLNTLTLAVP | NSP16 | 48 | 62 | No |
| 2449 | AVMSLKEGQINDMIL | NSP16 | 258 | 272 | No |
| 2450 | KEGQINDMILSLLSK | NSP16 | 263 | 277 | No |
| 2451 | RENNRVVISSDVLVN | NSP16 | 283 | 297 | No |
| 2452 | PLNSIIKTIQPRVEK | NSP2 | 96 | 110 | No |
| 2453 | EEIAIILASFSASTS | NSP2 | 291 | 305 | No |
| 2454 | SPLYAFASEAARVVR | NSP2 | 351 | 365 | No |
| 2455 | AITILDGISQYSLRL | NSP2 | 386 | 400 | No |
| 2456 | QTFFKLVNKFLALCA | NSP2 | 496 | 510 | No |
| 2457 | GETFVTHSKGLYRKC | NSP2 | 526 | 540 | No |
| 2458 | ADAVIKTLQPVSELL | NSP3 | 58 | 72 | No |
| 2459 | ESDDYIATNGPLKVG | NSP3 | 268 | 282 | No |
| 2460 | IATNGPLKVGGSCVL | NSP3 | 273 | 287 | No |
| 2461 | SGHNLAKHCLHVVGP | NSP3 | 288 | 302 | No |
| 2462 | NLYDKLVSSFLEMKS | NSP3 | 363 | 377 | No |
| 2463 | ENLLLYIDINGNLHP | NSP3 | 433 | 447 | No |
| 2464 | KSAFYILPSIISNEK | NSP3 | 532 | 546 | No |
| 2465 | RFYFYTSKTTVASLI | NSP3 | 603 | 617 | No |
| 2466 | EAARYMRSLKVPATV | NSP3 | 643 | 657 | No |
| 2467 | LPNDDTLRVEAFEYY | NSP3 | 803 | 817 | No |
| 2468 | TLRVEAFEYYHTTDP | NSP3 | 808 | 822 | No |
| 2469 | HTTDPSFLGRYMSAL | NSP3 | 818 | 832 | No |
| 2470 | SFLGRYMSALNHTKK | NSP3 | 823 | 837 | No |
| 2471 | YMSALNHTKKWKYPQ | NSP3 | 828 | 842 | No |
| 2472 | NHTKKWKYPQVNGLT | NSP3 | 833 | 847 | No |
| 2473 | ESPFVMMSAPPAQYE | NSP3 | 983 | 997 | No |
| 2474 | YCIDGALLTKSSEYK | NSP3 | 1028 | 1042 | No |
| 2475 | DNFKFVCDNIKFADD | NSP3 | 1108 | 1122 | No |
| 2476 | LNQLTGYKKPASREL | NSP3 | 1123 | 1137 | No |
| 2477 | GYKKPASRELKVTFF | NSP3 | 1128 | 1142 | No |
| 2478 | ASRELKVTFFPDLNG | NSP3 | 1133 | 1147 | No |
| 2479 | PDLNGDVVAIDYKHY | NSP3 | 1143 | 1157 | No |
| 2480 | TPSFKKGAKLLHKPI | NSP3 | 1158 | 1172 | No |
| 2481 | VWHVNNATNKATYKP | NSP3 | 1173 | 1187 | No |
| 2482 | MAAYVDNSSLTIKKP | NSP3 | 1278 | 1292 | No |
| 2483 | NELSRVLGLKTLATH | NSP3 | 1293 | 1307 | No |
| 2484 | TFTRSTNSRIKASMP | NSP3 | 1363 | 1377 | No |
| 2485 | TNSRIKASMPTTIAK | NSP3 | 1368 | 1382 | No |
| 2486 | NTVKSVGKFCLEASF | NSP3 | 1383 | 1397 | No |
| 2487 | LEASFNYLKSPNFSK | NSP3 | 1393 | 1407 | No |
| 2488 | PNFSKLINIIIWFLL | NSP3 | 1403 | 1417 | No |
| 2489 | GSLIYSTAALGVLMS | NSP3 | 1423 | 1437 | No |
| 2490 | ISSFKWDLTAFGLVA | NSP3 | 1493 | 1507 | No |
| 2491 | WDLTAFGLVAEWFLA | NSP3 | 1498 | 1512 | No |
| 2492 | FGLVAEWFLAYILFT | NSP3 | 1503 | 1517 | No |
| 2493 | FDAYVNTFSSTFNVP | NSP3 | 1773 | 1787 | No |
| 2494 | SHNIALIWNVKDFMS | NSP3 | 1888 | 1902 | No |
| 2495 | KGGKIVNNWLKQLIK | NSP4 | −2 | 12 | No |
| 2496 | LFVAAIFYLITPVHV | NSP4 | 18 | 32 | No |
| 2497 | AVITREVGFVVPGLP | NSP4 | 93 | 107 | No |
| 2498 | VPGLPGTILRTTNGD | NSP4 | 103 | 117 | No |
| 2499 | FLHFLPRVFSAVGNI | NSP4 | 118 | 132 | No |
| 2500 | DTRYVLMDGSIIQFP | NSP4 | 188 | 202 | No |
| 2501 | SIVAGGIVAIVVTCL | NSP4 | 283 | 297 | No |
| 2502 | FGEYSHVVAFNTLLF | NSP4 | 308 | 322 | No |
| 2503 | NTLLFLMSFTVLCLT | NSP4 | 318 | 332 | No |
| 2504 | PVYSFLPGVYSVIYL | NSP4 | 333 | 347 | No |
| 2505 | YLTFYLTNDVSFLAH | NSP4 | 348 | 362 | No |
| 2506 | SFLAHIQWMVMFTPL | NSP4 | 358 | 372 | No |
| 2507 | IQWMVMFTPLVPFWI | NSP4 | 363 | 377 | No |
| 2508 | MFTPLVPFWITIAYI | NSP4 | 368 | 382 | No |
| 2509 | TIAYIICISTKHFYW | NSP4 | 378 | 392 | No |
| 2510 | CTFLLNKEMYLKLRS | NSP4 | 418 | 432 | No |
| 2511 | LTQYNRYLALYNKYK | NSP4 | 438 | 452 | No |
| 2512 | RYLALYNKYKYFSGA | NSP4 | 443 | 457 | No |
| 2513 | YREAACCHLAKALND | NSP4 | 463 | 477 | No |
| 2514 | CCHLAKALNDFSNSG | NSP4 | 468 | 482 | No |
| 2515 | FSNSGSDVLYQPPQT | NSP4 | 478 | 492 | No |
| 2516 | SDVLYQPPQTSITSA | NSP4 | 483 | 497 | No |
| 2517 | NHNFLVQAGNVQLRV | NSP5 | 63 | 77 | No |
| 2518 | QNCVLKLKVDTANPK | NSP5 | 83 | 97 | No |
| 2519 | LLVLVQSTQWSLFFF | NSP6 | 22 | 36 | No |
| 2520 | SLFFFLYENAFLPFA | NSP6 | 32 | 46 | No |
| 2521 | LCLFLLPSLATVAYF | NSP6 | 67 | 81 | No |
| 2522 | TLVYKVYYGNALDQA | NSP6 | 147 | 161 | No |
| 2523 | DAFKLNIKLLGVGGK | NSP6 | 267 | 281 | No |
| 2524 | RVESSSKLWAQCVQL | NSP7 | 21 | 35 | No |
| 2525 | SKLWAQCVQLHNDIL | NSP7 | 26 | 40 | No |
| 2526 | VLKKLKKSLNVAKSE | NSP8 | 34 | 48 | No |
| 2527 | LIVTALRANSAVKLQ | NSP8 | 184 | 198 | No |
| 2528 | SDFVRATATIPIQAS | ORF3a | 26 | 40 | No |
| 2529 | ALLAVFQSASKIITL | ORF3a | 51 | 65 | No |
| 2530 | KIITLKKRWQLALSK | ORF3a | 61 | 75 | No |
| 2531 | CNLLLLFVTVYSHLL | ORF3a | 81 | 95 | No |
| 2532 | LVAAGLEAPFLYLYA | ORF3a | 96 | 110 | No |
| 2533 | LEAPFLYLYALVYFL | ORF3a | 101 | 115 | No |
| 2534 | LVYFLQSINFVRIIM | ORF3a | 111 | 125 | No |
| 2535 | VRIIMRLWLCWKCRS | ORF3a | 121 | 135 | No |
| 2536 | RLWLCWKCRSKNPLL | ORF3a | 126 | 140 | No |
| 2537 | KNPLLYDANYFLCWH | ORF3a | 136 | 150 | No |
| 2538 | YDANYFLCWHTNCYD | ORF3a | 141 | 155 | No |
| 2539 | FLCWHTNCYDYCIPY | ORF3a | 146 | 160 | No |
| 2540 | TNCYDYCIPYNSVTS | ORF3a | 151 | 165 | No |
| 2541 | YFTSDYYQLYSTQLS | ORF3a | 206 | 220 | No |
| 2542 | TDTGVEHVTFFIYNK | ORF3a | 221 | 235 | No |
| 2543 | EHVTFFIYNKIVDEP | ORF3a | 226 | 240 | No |
| 2544 | FIYNKIVDEPEEHVQ | ORF3a | 231 | 245 | No |
| 2545 | GSSGVVNPVMEPIYD | ORF3a | 251 | 265 | No |
| 2546 | MFHLVDFQVTIAEIL | ORF6 | 1 | 15 | No |
| 2547 | IAEILLIIMRTFKVS | ORF6 | 11 | 25 | No |
| 2548 | AEILLIIMRTFKVSI | ORF6 | 12 | 26 | No |
| 2549 | LIIMRTFKVSIWNLD | ORF6 | 16 | 30 | No |
| 2550 | TFKVSIWNLDYIINL | ORF6 | 21 | 35 | No |
| 2551 | YIINLIIKNLSKSLT | ORF6 | 31 | 45 | No |
| 2552 | MKIILFLALITLATC | ORF7a | 1 | 15 | No |
| 2553 | IILFLALITLATCEL | ORF7a | 3 | 17 | No |
| 2554 | DGVKHVYQLRARSVSPKL | ORF7a | 69 | 86 | No |
| 2555 | VKHVYQLRARSVSPK | ORF7a | 71 | 85 | No |
| 2556 | LYSPIFLIVAAIVFI | ORF7a | 96 | 110 | No |
| 2557 | SPIFLIVAAIVFITL | ORF7a | 98 | 112 | No |
| 2558 | DFYLCFLAFLLFLVL | ORF7b | 8 | 22 | No |
| 2559 | MKFLVFLGIITTVAA | ORF8 | 1 | 15 | No |
| 2560 | FLGIITTVAAFHQEC | ORF8 | 6 | 20 | No |
| 2561 | TTVAAFHQECSLQSC | ORF8 | 11 | 25 | No |
| 2562 | FHQECSLQSCTQHQP | ORF8 | 16 | 30 | No |
| 2563 | SLQSCTQHQPYVVDD | ORF8 | 21 | 35 | No |
| 2564 | ARKSAPLIELCVDEA | ORF8 | 51 | 65 | No |
| 2565 | PLIELCVDEAGSKSP | ORF8 | 56 | 70 | No |
| 2566 | CVDEAGSKSPIQYID | ORF8 | 61 | 75 | No |
| 2567 | IQYIDIGNYTVSCLP | ORF8 | 71 | 85 | No |
| 2568 | QEPKLGSLVVRCSFY | ORF8 | 91 | 105 | No |
| 2569 | EDFLEYHDVRVVLDF | ORF8 | 106 | 120 | No |
| 2570 | DFLEYHDVRVVLDFI | ORF8 | 107 | 121 | No |

Where D = dominant and ND = nondominant

The novel pools described herein augment previously described pools consisting of 1) overlapping 15-mers spanning the entire S antigen, 2) predicted HLA class II binding 15-mers from the remainder of the proteme, and 3) epitopes derived from both S and non-S predicted to bind common HLA class I.

These new pools are based on the analysis that originally capitalizes and synthetizes information fragmented in different reports in the published literature. This allows for a most comprehensive inventory of experimentally defined epitopes, and for the generation of peptide epitope pools associated with superior sensitivity and specificity to detect and analyze responses from infected and vaccinated individuals, and also identifies epitope sets useful for vaccine applications. Different variations are also disclosed to illustrate the flexibility of the approach; for example, pools can be designed based on classification of epitopes as dominant or non-dominant, further facilitating their use for characterization of immune responses to SARS-CoV-2. The peptide pools described herein can be utilized to detect and characterize immune responses to SARS-CoV-2, and facilitate the design of novel vaccines and therapeutics.

### Example 2: Definition and Recognition of SARS-CoV-2-Derived T Cell Epitopes in Humans

Over the last year, a large amount of information has been produced by the scientific community related to SARS-CoV-2 infection and the associated COVID-19 disease. Studies in the peer-reviewed and pre-print literature have addressed a variety of different virology, epidemiological and clinical aspects. In particular, a large number of studies have analyzed the immune response to the virus and the role these responses play in protection and disease, and also their importance in the context of vaccine development and evaluations. Several excellent reviews, some also in the present special issue, cover these topics (1-6).

Here, the inventors focus on the current state of knowledge related to definition and recognition of SARS-CoV-2-derived T cell epitopes in humans. While the data related to this topic was initially sparse, 25 different studies have now been published as of Mar. 15, 2021 (7-34), and collectively report data from 1197 human subjects (870 COVID-19 and 327 unexposed controls), leading to the identification of over 1400 different CD4 (n=382) and CD8 (n=1052) T cell epitopes. These studies are listed in Table 1, which also captures whether the studies defined class I/CD8 epitopes and/or class II/CD4 epitopes.

The relevant papers were selected based on the objective curation process implemented over almost 20 years ago by the Immune Epitope Database (IEDB; www.iedb.org), based on the combined use of general broad PubMed queries, combined with automated text classifiers and manual curation, as described in more detail elsewhere (35, 36). In addition, the results of the IEDB curation were manually inspected by the coauthors to guard against papers missed by the IEDB curation workflow, but no additional papers were identified.

Taken together, this disclosure focuses on the overall theme of cataloging and describing SARS-CoV-2 epitopes recognized by human T cells. The data collected is derived from the 25 studies referred above. Accordingly, the data is organized into a number of following examples, initially describing epitope definitions, screening methodologies and assay readouts. Subsequent examples describe the number of epitopes identified in the various studies, the antigens recognized and the distribution of epitopes within them, eventually leading to the definition of immunodominant regions and immunodominant epitopes. Additional sections are devoted to discussion of epitope identification in different populations and cohorts, and the related topics of HLA coverage and immunodominant HLA alleles. An overall discussion of breadth of the T cell repertoire informs discussion of pre-existing reactivity and cross-reactivity with common cold corona and other viruses, cross-reactivity with MERS, SARS-CoV-1, and potential implications for immune escape by SARS-CoV-2 variants. This disclosure is therefore relevant to the definition in molecular terms of the targets of adaptive human T cell responses to SARS-CoV-2.

### Example 3: Epitope Definitions

A detailed review of the available epitope data requires clear definition of concepts and terminology, to allow combination of different studies utilizing different methodologies. This in turn allows integration of the information in a coherent fashion. According to classical textbook definitions, “A T-cell epitope is a short peptide derived from a protein antigen. It binds to an MHC molecule and is recognized by a particular T cell” (43). And, similarly, “The parts of complex antigens that are specifically recognized by lymphocytes are called determinants or epitopes” (44).

T cell epitopes are usually peptides composed of the 20 naturally occurring amino acids, although recognition of haptens, sugars and post-translationally modified peptides has also been described (45, 46). The topic of post-translationally modified epitopes has been reviewed elsewhere (45). While many post-translationally modified epitopes have been described in the cancer setting and autoimmunity, few have been described in the case of viral antigens. However, one topic of particular interest, also in the context of SARS-CoV-2, will be to evaluate if glycosylated sites are differentially recognized, also in the context of N>D modifications associated with removal of the polysaccharide moiety in the course of cellular processing. But thus far, in the case of SARS-CoV-2, no reports have appeared of post-translationally modified or glycosylated peptides being recognized by T cell responses.

T cells recognize a bimolecular complex of an epitope bound to a specific class I or class II MHC molecule (HLA in humans), which is called its restriction element. HLA class I restricted epitopes are generally 9-10 residues in size, with several also being 8 or 11 residues, depending on HLA-restriction, while class II restricted epitopes are typically 13-17 residues, although shorter and longer peptides have also been described. By the late 1980s it was appreciated that a given peptide can bind multiple HLA allelic variants, especially if those variants are structurally or genetically related (47, 48). The HLA variants or types associated with overlapping peptide binding repertoires are classified into so called HLA supertypes (49, 50). Epitopes that bind multiple HLAs are referred to as promiscuous (51, 52). In general, any given HLA/peptide complex can be recognized by a multitude of different T cell receptors, which often share a discernible pattern of sequence similarity (53, 54).

Viral genomes and proteomes are composed of multiple protein antigens. Each of these antigens is recognized in a human population to varying degrees (55, 56). The concept of immunodominance usually refers to how strongly a given antigen is recognized, while immunoprevalence refers to how often the antigen is recognized (57-59), although in practice the two terms are frequently used somewhat interchangeably.

Immunodominance of a given antigen within a genome or proteome is influenced by variables such as levels of transcription and expression, stability, and patterns of expression in different cell types or anatomical sites. In the context of SARS-CoV-2, Poran et al. point out the potential of leveraging proteomic data to infer relative viral protein abundance (23, 24). Several other studies have eluted SARS-CoV-2-derived peptides bound to HLA (39-41), but have not shown that the epitopes are actually recognized by T cell responses. Future studies will examine the correspondence between eluted ligands and T cell recognition.

The fact that HLA binding is a necessary but not sufficient requisite for T cell recognition has been well established (56, 60-62), as it does not guarantee that the peptide will be generated by antigen processing, and does not ensure and the availability of a repertoire of T cells capable of recognizing the corresponding epitope/HLA complex (63, 64). In the case of eluted ligands (65, 66), factors to be considered are whether the assay used to detect eluted ligands has sensitivity comparable to T cell activation (a few epitope copies have been shown to be sufficient to activate T cells (67, 68), and again the availability of TCR repertoire, which is also modulated by previous infection history, as discussed in more detail below.

Immunodominance and immunoprevalence within a given antigen indicates, of all possible peptide epitopes contained in the antigen, how frequently and vigorously a particular epitope is recognized (55, 56). Immunodominance/prevalence hierarchies within an antigen are influenced by variables such as HLA binding capacity, antigen processing, and the repertoire of TCR recognizing a given HLA/epitope combination. Finally, the term breadth of responses is defined on the basis of how many antigens or epitopes are recognized, either at the level of a given individual or in a population as a whole (55, 56).

### Example 4: Screening Methodologies

The process of epitope identification requires testing collections of candidate peptides in an assay of choice. The peptide collections utilized can span the entire genome or proteome, or focus on selected antigens of interest. Furthermore, the peptide collections may correspond to either sets of overlapping peptides (a popular choice is 15-mers overlapping by 10 residues) spanning a sequence, or peptides predicted to bind to one or more different HLA types, as indicated in Table 1. In general, and in the case of SARS-CoV-2 in particular, overlapping peptides are more often used in the case of defining class II restricted epitopes (4 of 9 studies; 44%), at least in part due to the lower predictive efficacy of HLA class II predictions (69), than in the case of class I epitopes (6/25 studies; 24%), where predicted binders are more often used to probe responses (21 of 25 studies; 85%). While the length of HLA class II restricted epitopes varies, the use of 15-mers overlapping by 10 residues ensures that any possible 10-mer is represented in the peptide set, with the addition of flanking residues at either or both ends. Given the fact that the critical core of class II epitopes is 9 residues in size, this ensures that most if not all epitopes are identified, without having to rely on bioinformatic predictions.

Another issue of relevance is whether responses are measured directly ex vivo or if an in vitro culture restimulation step is introduced. A restimulation step is often used to expand low frequency T cell specificity which would otherwise be difficult to detect. A number of different methodologies are used to detect or expand T cells, ranging from stimulation with whole antigens or antigen fragments, to the use of peptide pools or isolated individual peptides. However, in vitro restimulation is known to substantially alter the phenotypes and/or relative frequency of responding T cells. Expansion of naïve T cells can also occur. In the case of SARS-CoV-2, studies have shown that when PBMCs are expanded for 10-14 days before the assessment of SARS-CoV-2 responses, CD4⁺ T cells expand to a much greater extent than CD8⁺ T cells (10, 19).

To overcome these caveats, it is preferable to assay T cells ex vivo whenever possible. In the case of SARS-CoV-2 T cell epitopes, 14 studies have used direct ex vivo assays (Table 1), and 12 utilized in vitro culture (one study utilized both in vitro and ex vivo approaches). Alternatively, once the epitopes are identified, they can be used to conduct secondary epitope validation experiments with direct ex vivo modalities, as shown by 2 studies (7, 30). Of note, Keller et al. showed that expansion of SARS-CoV-2 T cells can be accomplished in controlled conditions, and raised the possibility that epitope expanded T cells may be used for adoptive therapy (13). The principle and conditions for adoptive therapy have been described and reviewed elsewhere (70).

Thus, in general, each assay methodology has its own advantages and disadvantages. Whole blood and ELISPOT assays are simplest, and require less sophisticated equipment, but yield less granular information. In vitro culture assays allow expansion of relatively rare T cell specificities, while ex vivo assays allow to detect responses without manipulations that can be associated with phenotypic and functional alterations.

### Example 5: Assay Readouts

Regardless of whether T cell responses are detected ex vivo or after in vitro expansion, a variety of different assay methodologies are available to investigate specific T cell responses. In selecting an approach, several considerations apply, including ease of implementation, throughput, and comprehensiveness and functionality. Certain assays, such as enzyme-linked immunospot (ELISpot), supernatant determination, and whole blood assays are relatively easier to employ and more amenable to high throughput testing. However, they are associated with less granular information. For example, the CD4 vs CD8 phenotype (and the expression of other cell markers) of the responding cells is not readily established by these methods, compared to others such as Intracellular Cytokine Staining (ICS) or Activation Induced Marker (AIM) assays. The methodologies utilized by the various studies include AIM, degranulation, proliferation, ELISA, ELISpot, ICS, cytotoxicity, and multimer-based assays (for 3, 2, 2, 1, 5, 10, 1 and 13 studies, respectively).

Over a dozen studies (8-10, 12, 21, 23-32) performed high-resolution analysis of SARS-CoV-2-specific CD8⁺ T cells using HLA multimers. However, none of the studies reported similar multimer analyses for CD4⁺ T cells, despite the fact that, in general, HLA class II restricted SARS-CoV-2-specific T cell responses are more pronounced compared HLA class I restricted T cell responses (20, 71). This reflects the relatively higher availability of HLA class I multimeric reagents, as compared to their HLA class II counterparts. Some studies analyzed epitope specific responses not only in blood but also in tissues, such as tonsil and lung tissue from uninfected donors (10). Analyzing tissue-derived T cells can contribute particular insight into disease, such as for example defining characteristics of Tissue Resident Memory T cells, which may differ from those circulating in the peripheral blood (72).

An issue encountered with ELISpot and ICS and related assays is that while they, by definition, identify T cells capable of a functional response, they only (also by definition) detect T cells producing a cytokine of choice; therefore, they are “blind” to T cells producing different cytokines or that do not produce cytokines in large amounts within the window of time of the assay (e.g., T follicular helper [Tfh]CD4 T cells generally produce very low amounts of cytokines). Both AIM (73-75) and HLA tetramer/multimer assays are “agnostic” in this respect, as they detect all cells activated by the epitope (AIM), or all cells expressing a TCR capable of binding a given epitope/HiLA complex (tetramer/multimer). Accordingly, it is frequently observed that AIM and tetramer assays have higher sensitivity because they detect larger numbers of T cells, as compared to ELISpot assays. Sahin et al. note that comparison of data from MHC multimers with bulk IFNγ⁺ CD8⁺ T cell responses indicated that a functional T cell assay may underestimate the total cellular immune response (27). Conversely, T cells captured by tetramers might not be functional or exhausted, and therefore might overestimate the cellular response relevant for immunity and control of infection. However, for SARS-CoV-2, it has indeed been observed that CD8 T cells identified by HLA-multimers in COVID-19 subjects are functional and not exhausted (26). In conclusion, a variety of epitope screening and assay strategies have been utilized, each with its own features and potential advantages/disadvantages.

### Example 6: Epitopes Identified

Table 1 lists the total number of characterized canonical CD4 and CD8 epitopes identified in each study, which ranged from 1 to 734 (median of 12). It should be noted that it is not possible to estimate the total number of unique identified epitopes by simply adding these numbers, because the same epitope might be identified independently in multiple studies (as addressed below in the immunodominance section), and/or, especially in the case of CD4 epitope studies utilizing overlapping peptides, essentially the same epitope might be identified by two largely overlapping peptides.

To address this point, to assess CD4 epitope redundancy the data were further analyzed taking advantage of the clustering tool provided by the IEDB (76), which automatically removes duplications and largely overlapping entries, as well as additional manual curation. The clustering tool is an algorithm that generates clusters from a set of input epitopes based on representative or consensus sequences. This tool allows the user to cluster peptide sequences on the basis of a specified level of identity by selecting among three different method options. For these purposes, the inventors utilized the default “cluster-break” settings which generates clusters where all component epitopes share at minimum a specified level of homology (70%), and no epitope is present in more than one cluster. Because of the closed ends of the class I MHC binding groove, and hence the incapacity of class I binding peptides to assume alternate frames, overlapping CD8 epitopes are considered unique epitopes by default.

Accordingly, the studies listed in Table 1 encompass 1434 unique epitopes, including 1052 different class I and 382 different class II non-redundant epitopes (versus 416 leaving in redundancies).

### Example 7: Antigenic Targets and Epitope Distribution

Ten of the 25 epitope identification studies (8, 9, 12, 19, 20, 25, 28-30, 33, 34) screened peptides derived from the entire SARS-CoV-2 proteome (seventh column of Table 1). The main antigenic targets of CD4 and CD8 SARS-CoV-2 T cell responses have been defined by several studies utilizing overlapping peptides, mostly not resolving the actual epitopes (34, 71) and also reviewed elsewhere (1, 78). These studies determined that structural proteins (S, M and N) are dominant targets of T cell responses, with ORF3, ORF8, and nsp3, 4, 6, 7, 12 and 13 (ORF1ab) also being frequently targeted. Other studies focused on specific subsets of SARS-CoV-2 antigens, as also detailed in the seventh column of Table 1.

The various studies differ widely in the depth of screening, number of antigens tested, HLA alleles targeted, and number of peptides screened. For example, Peng et al. (22) screened the whole proteome, with the exception of ORF1ab, using 423 peptides assayed in 42 infected and 16 non-exposed subjects and reported broad CD4 and CD8 responses. Conversely, Schulien et al. (30) only tested 5 peptides predicted to bind each of ten different HLAs. Tarke et al. (34), using PBMC from 99 donors, probed for CD4 responses using 1,925 peptides spanning the entire SARS-CoV-2 proteome, and for CD8 responses tested an additional 5,600 peptides predicted to bind one or more of 28 prominent HLA class I alleles. Snyder et al. (33) screened 545 peptides distributed over the SARS-CoV-2 proteome for 26 class I alleles, testing about 20 peptides/allele. Nelde et al. (20) screened a large number of donors (220 in total) with peptides spanning the breadth of antigens (i.e., whole proteome) predicted to bind six HLA class I alleles or various HLA-DR class II. Le Bert (16) focused on peptides derived from N, nsp7 and nsp13, while Ferretti (8) screened predicted peptides from the entire proteome for 6 HLA alleles in 5 to 9 donors per each HLA.

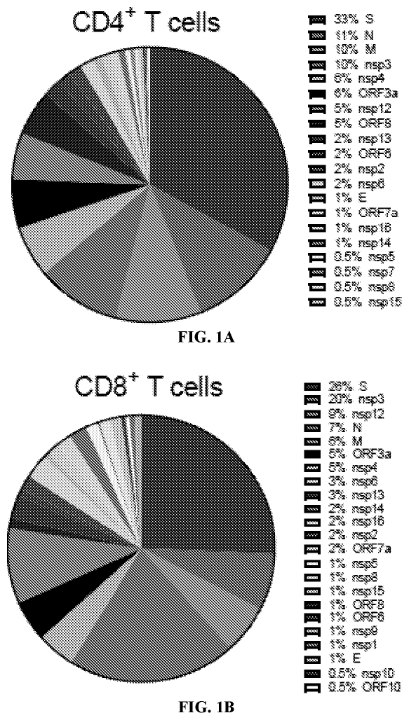

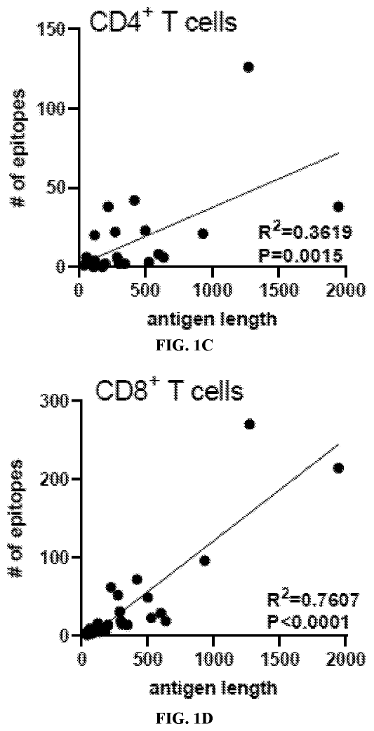

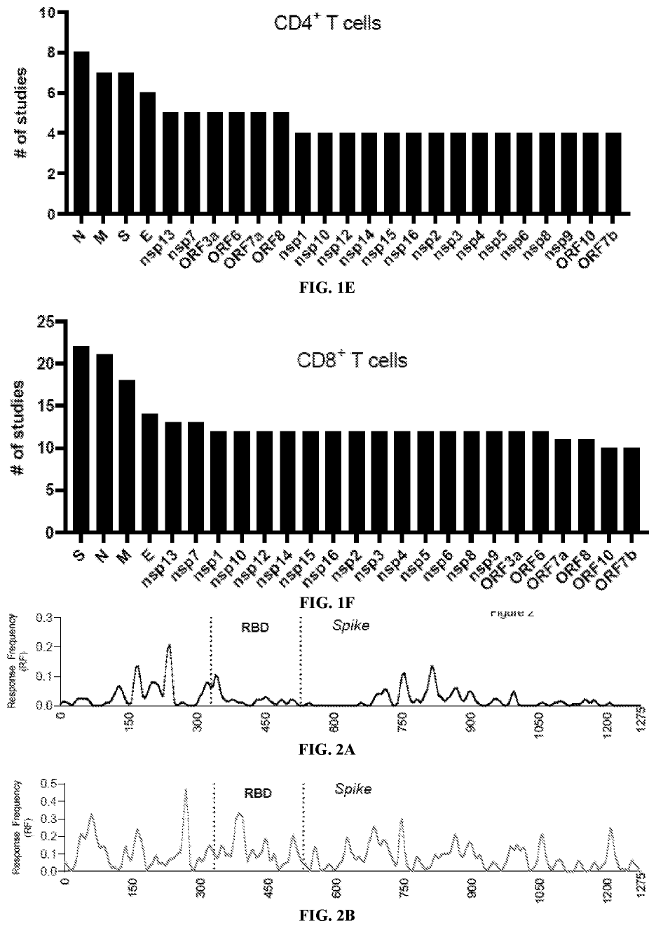

The epitope distribution along the SARS-CoV-2 proteome is analyzed in more detail in FIGS. 1A-1B, where the number of epitopes identified in each antigen is shown for CD4 and CD8 epitopes, respectively. FIGS. 1C-1D shows the correlation between the number of epitopes and the total number of residues (size) of each antigen. A significant correlation exists between antigen size and the number of epitopes identified for both CD4 (p=0.0015 and r\hat{ }=0.36) and CD8 epitopes (p<0.0001 and r\hat{ }2=0.76). Certain antigens (N, M, S and E) were studied in more detail (more studies) (FIGS. 1E-1F), so that this is a significant factor, in addition to antigen length, in influencing the number of epitopes identified. Additionally, it was recognized early that the immunodominance pattern of the CD4 and CD8 T cell response to SARS-CoV-2 largely tracks with the expression level of each of the 25 viral proteins (71). S, M, and N sgRNAs are highly expressed by SARS-CoV-2 infected cells, and those three proteins are the most immunodominant targets of human CD4 and CD8 T cell responses to SARS-CoV-2 (71).

In conclusion, T cell responses are multi-antigenic, with the structural antigens being broadly recognized, but other proteins, such as nsp3, nsp4, nsp12 and ORF3a, are also vigorously recognized. This difference is not unexpected, given the fact that structural proteins are present in high concentrations in the virus, and accessible to the exogenous processing pathway and HLA class II molecules. Conversely, non-structural proteins are produced in infected cells and have, together with the structural proteins, access to the endogenous processing pathway and HLA class I molecules.

### Example 8: Immunome Browser Analysis Identifies General Patterns of Immunodominance

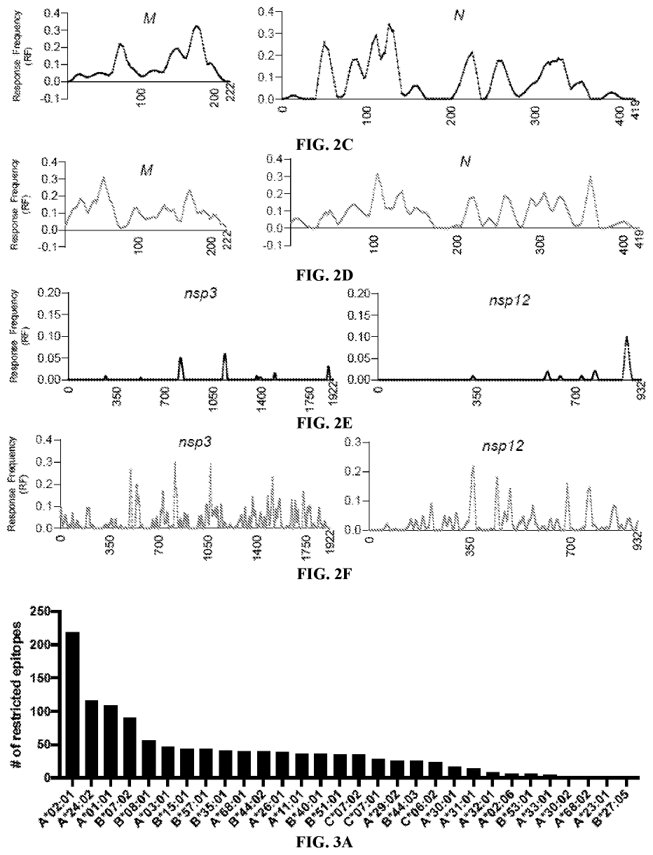

In the next series of analyses, the inventors addressed whether discrete immunodominant regions would be apparent when the data derived from the different studies was globally considered. To perform this meta-analysis, the inventors utilized the Immunome Browser tool (79, 80), developed and hosted by the IEDB (www.iedb.org). This tool allows visualization of patterns of immunodominance across the entire SARS-CoV-2 proteome by plotting for each residue the 95% confidence interval (CI) of the Response Frequency (RF), defined as the number of individuals and assays reporting positive responses to a peptide encompassing the particular residue. The lower bound RF values, using an average across a sliding 10 residue window, are plotted for human CD4 and CD8 epitopes in FIGS. 2A to 2F for S, M, N, nsp3 and nsp12, as these antigens are the ones from which epitopes were described in sufficient numbers to allow delineation of discrete immunodominant regions.

In the case of spike protein, several immunodominant regions were observed for CD4 (residues 154-254, 296-370 and 682-925; FIG. 2A), compared to a more homogenous distribution for CD8 (FIG. 2B). For the other structural proteins, namely membrane and nucleocapsid, similar immunodominant regions for CD4 (FIG. 2C) and CD8 (FIG. 2D) were noted, with the 7-101 and 131-213 residue range being more prominent for the membrane protein, and the 31-173 and 201-371 range for nucleocapsid. More marked differences in CD4 and CD8 immunodominant regions, as well as overall response frequency, are observed in the cases of nsp3 (FIG. 2E) and nsp12 (FIG. 2F). Here, for both proteins, defined immunodominant regions for CD4 (789-843, 1118-1158 and 1873-1903 for nsp3 and 863-903 for nsp12) were evident, versus more homogenous patterns of CD8 recognition, similar to what was noted for spike (FIG. 2B). In conclusion, CD4⁺ T cells in general recognize more defined immunodominant regions than the corresponding CD8⁺ counterpart.

### Example 9: Epitope Identification in Different Populations and Cohorts

As a whole, the different studies considered here have reported epitope identification results from a total of 1197 donors (median=34, range 2 to 220; see the eighth and ninth columns of Table 1). Of those, 870 donors were SARS-CoV-2 infected, and 327 unexposed. It should be noted that these reflect the maximum number of donors utilized in each epitope identification and characterization study, as some assays and some epitopes have been tested in a different number of donors. For example, in some cases 20 donors were tested in ELISpot, but only 10 were evaluated using MHC multimers. Similarly, in several instances, because of the need to match peptide candidates to specific predicted HLA alleles (e.g., HLA-A*02:01 candidate epitopes may only have been tested in HLA-A*02:01 positive donors), the actual number of donors in which each peptide was tested may be significantly lower in comparison to other peptides.

Several studies have analyzed differences between the infected and unexposed cohorts, and also in the context of potential cross-reactivity of SARS-CoV-2 epitopes with homologous sequences from common cold coronaviruses or other viruses, as discussed in more detail below. Also, as noted elsewhere (5), considerable heterogeneity exists in SARS-CoV-2 infection and immune responses, as a function of different variables such as age, gender, disease severity, ethnicity and time since symptom onset. To date, the epitope identification studies as a whole do not yet answer the question whether differences in the types of epitopes recognized exist as a function of these variables. However, the epitopes defined in these studies will undoubtedly be key, alongside data generated with peptide pools, to probe variables such as age, gender, disease severity, ethnicity and time since onset of symptoms.

One aspect to consider, and touched on further below, is to ensure that different ethnicities are adequately represented. Thus far, most studies have been performed in donor cohorts that are, or are expected to be, mostly composed of Caucasians, and relatively under-representative of other races and ethnic groups.

### Example 10: HLA Coverage and Epitope Identification Results

It is well appreciated that HLA molecules are associated with an outstanding degree of diversity. Class I molecules are encoded by 3 main loci (A, B and C), and class II molecules are encoded by four main loci (DRB1, DRB3/4/5, DP and DQ). Each locus is highly polymorphic, and because of heterozygosity each individual may express close to 14 different HLA molecules, and minimum of 7 (if homozygous at all loci). Not only are the various HLA loci highly polymorphic, but the frequencies of respective alleles vary, sometimes dramatically, across different ethnicities (81, 82). Establishing the extent that epitope identification efforts provide adequate coverage of the worldwide population is both a key and non-trivial issue (49, 83, 84).

To meaningfully discuss population coverage considering HLA allelic variants in the context of epitope identification efforts, it is necessary to define what is meant by population coverage. The total phenotypic coverage provided by a set of HLA alleles represents the fraction of individuals that express at least one of a given set of alleles, while genotypic coverage corresponds to the fraction of genes at a specific locus the set of allelic variants covers. By way of example, an analysis targeting the HLA-A*01:01, B*07:02 and DRB1*01:01 molecules will give a phenotypic coverage (probability that an individual in the average worldwide population will express at least one of these alleles) of approximately 35%. However, these three allelic variants represent only about 5-10% of the gene variants each at the three different respective loci. This is important because in an individual that is “covered,” in the sense of expressing one HLA, the bulk of the T cell response will likely be directed to the other, up to thirteen, class I and class II alleles, leading to gross misrepresentation of the total response magnitude and target specificity.

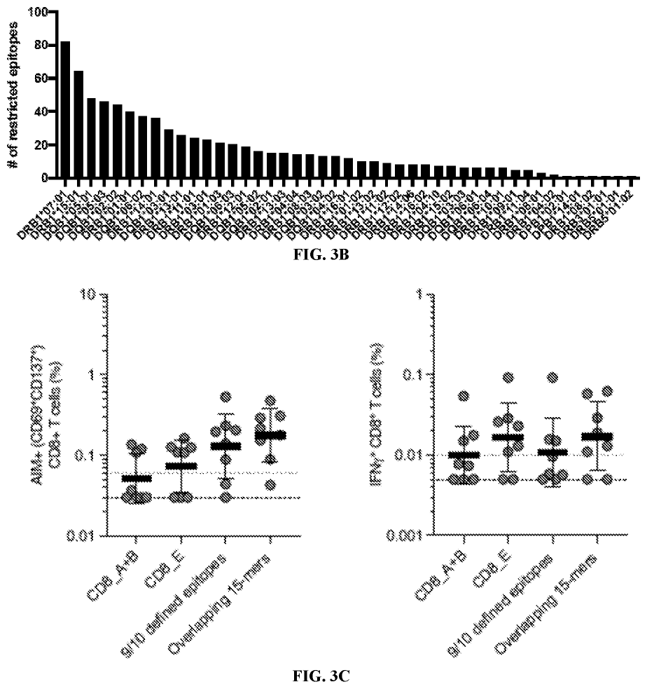

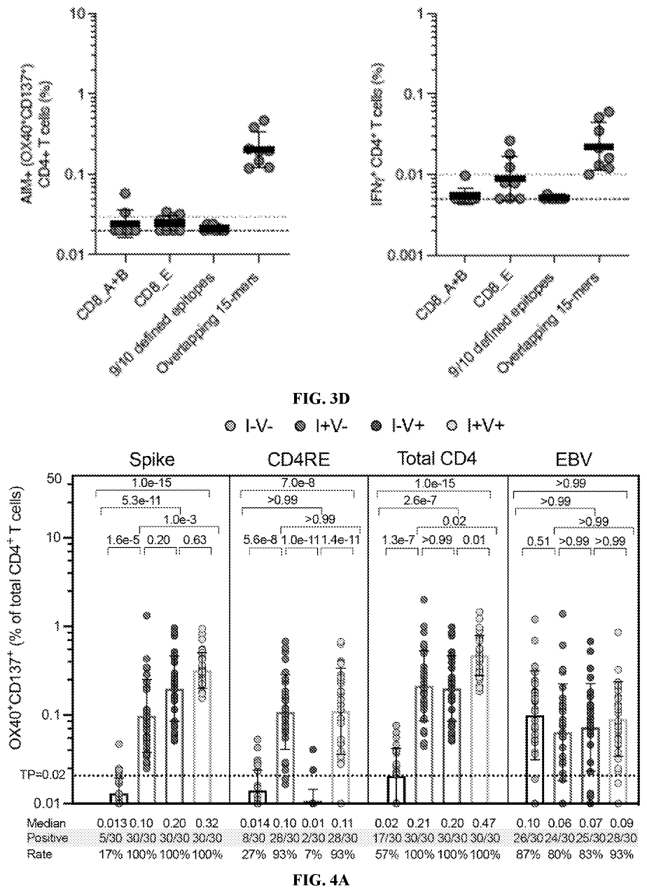

In previous studies, the inventors devoted significant efforts to analyze the number of different HLA alleles associated with good genotypic and phenotypic coverage, and found that about 25 different HLA class II and about 25 different HLA class I alleles are required to cover 90% or more individuals in an idealized population (43, 61, 62). In the case of SARS-CoV-2 epitope identification studies, HLA restricted epitopes have been identified for 30 HLA class I and 45 HLA class II alleles (FIGS. 3A-3D), including, in both cases, the vast majority of the most common specificities in the general worldwide population (49, 81, 85). FIGS. 3A to 3D show a non-limiting example of the Defined HLA class I and class II restrictions. HLA restricted epitopes have been identified for 30 class I (FIG. 3A) and 45 class II (FIG. 3B) molecules. The number of epitopes associated with each allele is plotted. FIG. 3C shows CD8 responses and FIG. 3D shows CD4 responses induced by Spike CD8 and CD4 megapools, respectively.

The median number of epitopes per allele is 35 (range 1 to 219) for class I, and 12 for class II (range 1 to 82). In the case of class I, as might be expected, the most restrictions have been identified in the contexts of A*02:01, A*24:02, A*01:01 and B*07:02, as these are the most common class I alleles worldwide. Similarly, the most class II restrictions are for DRB1*07:01 and DRB1*15:01, the most common DRB1 specificities worldwide. In both cases, the number of restrictions generally corresponds to overall allele frequency in the respective cohorts. This data exemplifies how the number of epitopes associated with a particular allelic specificity may not necessarily reflect immunodominance, but rather bias due to the availability of corresponding donor samples. Thus, the limited number of epitopes identified for several alleles is because they are rarer, and therefore reflective of investigational bias. Additional studies are required to enable fully unbiased investigation of SARS-CoV-2 on a global scale. The number of allelic restrictions identified by the different studies is summarized in the tenth and eleventh columns of Table 1.

Overall, the 25 different studies mapped or inferred 1191 class I restrictions, including 1019 unique epitope/allele combinations (Table 1), with individual studies defining between 1 and 523 (median 8). For class II, 783 restrictions were mapped or inferred, with 760 representing unique epitope/allele combinations (Table 1). Only 9 studies investigated CD4 responses, with just 3 identifying class II restrictions (see Table 1). Thus, experimentally defined HLA restrictions are fewer in the case of class II as compared to class I, consistent with the fact that class I restrictions are more easily inferred or determined, and that multimers/tetramers (which implicitly assign restriction) are more broadly available for HLA class I as compared to HLA class II.

### Example 11: Immunodominance at the Level of Specific Epitopes and Alleles

Different studies report numerous peptides as being immunodominant, although each study also used different subjective definitions of immunodominance. While some peptides are repeatedly and independently identified, differences in the screening procedures utilized, HLA alleles considered, antigens targeted, sampling of small numbers of individuals, and how “immunodominance” is defined by the various authors, all contribute to differences in outcomes. For example, Peng et al. (22) reports several immunodominant peptides which they defined as being recognized by 6 or more of the up to 16 subjects screened. Tarke et al. (34) also highlight some epitopes as more dominant, with 49 class II epitopes being recognized in 3 or more donors from an average of 10 donors tested, and 41 class I epitopes recognized in 50% or more of the HLA matched donors tested. The same study also finds that the response is broad and multi-specific, with approximately 8-9 different antigens required to cover about 80% of the total CD4 and CD8 response (34). Nielsen et al. also concludes that the response is broad, since the top three immunogenic epitopes are derived from separate SARS CoV-2 proteins (21). Keller et al. reports immunodominant epitopes defined as epitopes being recognized in multiple donors from M, N and S (13).

Some specific epitopes are highlighted as immunodominant in multiple studies. For example, in the context of the HLA-A*02:01 class I molecule, which is the most studied for CD8 SARS-CoV-2 responses, the S 269-277 epitope (sequence YLQPRTFLL (SEQ ID NO:1266)) is detected in 81% of HLA-A2+ individuals in the Nielsen study (21). The same A2 dominant epitope is also reported by Shomuradova et al., who tested 13 A2 peptides in total, and also identified a less strongly recognized epitope (32). In the Habel et al. study, of the 14 peptides screened, S 269-277 generated the strongest IFN-f response, with S 976-984 and ORF1ab 3183-3191 less prominently recognized (10). Ferretti et al. identified 3 epitopes recognized in 3 or more subjects (67% of the subjects tested), including S 269-277 (8). The study by Sahin et al. reports S 269-277 as most dominant epitope, and also identifies epitopes strongly recognized in the context of HLA-A*24:02 and HLA-B*35:01 (27). Rha et al. detected S 269-277 responses in 37 of 112 (33%) patients, while S 1220-1228 was detected in only 2 of 40 (5%) patients (26), though other studies have observed higher response rates for this latter epitope. Overall, the S 269-277 epitope was found to be positive in 11 independent studies. In one embodiment, the present invention excludes SEQ ID NO:1266.

Another example of an immunodominant epitope is provided by the HLA-A*01:01 restricted nsp3 819-828 epitope (sequence TTDPSFLGRY (SEQ ID NO:661)). This epitope was reported by Nelde et al. as positive in 83% of the donors tested (20). This study also identified a large number of additional dominant CD4 and CD8 restricted epitopes. The same A1 restricted epitope was also reported by Saini et al., who tested over 3,000 peptides for 10 alleles (28, 29), and found 214 peptides that were recognized in 16 out of the 18 samples analyzed. Two additional HLA-A*01:01 epitopes that overlap with TTDPSFLGRY (nsp3 818-828 (SEQ ID NO:661), sequence HTTDPSFLGRY (SEQ ID NO:660), and nsp3 819-829, sequence TTDPSFLGRYM (SEQ ID NO:662) were also identified as particularly dominant. The study by Gangaev et al. screened 50 epitopes for 10 alleles using tetramers (500 total) in 18 donors and identified nine epitopes in total, including the immunodominant nsp3 epitope restricted by HLA-A*01:01 (9). In one embodiment, the present invention excludes SEQ ID NO:660, 661, and 662.

### Example 12: Global Analysis of Immunodominant Epitopes

The overall data was further inspected to determine whether particular HLA alleles and epitopes are dominantly recognized. In the case of HLA class II, because of the technical issues discussed above, dominant alleles are less readily assigned as restriction elements. In the case of HLA class I, certain alleles, such as HLA-A*01:01, B*07:02, B*08:01 and B*44:01 were associated with dominant responses (34). Other alleles, such as HLA A*02:01, were associated with numerous epitopes, but with responses of lower magnitude on average, and alleles such as A*30:01 and A*32:01 were associated with weak and infrequent responses. This HLA-allele-specific variation in response frequency/magnitude has been observed previously in the contexts of HIV and Dengue virus, where responses mediated by particular HLA allelic variants were associated with protection or susceptibility to disease (85, 86). Whether HLA types play a role in influencing disease severity in the context of SARS-CoV-2 will have to be established as larger data sets become available.

For the present purposes, the inventors have defined the most dominant CD4 and CD8 epitopes as those recognized in 3 or more donors/studies, consistent with the definitions utilized by Mateus et al. and Tarke et al. (19, 34). The inventors utilized this threshold based on previous experience in this matter. Selecting epitopes that have been recognized in multiple different experiments in separate donors allow to narrow the number of epitopes and focus on more dominant/prevalent responses, while still preserving the goal of representing epitopes presented by a wide variety of HLA alleles. That is because less common HLA are found, by definition, in a fewer individuals, and the studies considered involved a median of 34 donors. Therefore, raising the “bar” further would restrict “immunodominant epitopes” to just those restricted by alleles that are very common in the Caucasians.

The immunodominant epitopes identified accordingly are highlighted in Supplemental Table 1. In total, 399 epitopes (110 CD4 epitopes, and 289 CD8 epitopes), have been highlighted. It is important to note, and consistent with what was observed in other systems, that in no case was a given epitope that was tested in more than two donors recognized in 100% of the cases. This is of relevance, as it argues against using single epitope tetramers to measure responses, because of the likelihood of false negative results. Conversely, the results argue for the use of peptide pools or multiplexing strategies (12, 20, 31, 32) to ensure broad coverage of responses.

Another important consideration, as noted above, is the influence of investigational bias. It is apparent that epitopes from the spike protein, and those restricted by the most common HLA alleles, are overrepresented, likely a reflection that the spike antigen and those particular HLA alleles are more frequently studied (FIGS. 2E-2F).

### Example 13: Breadth of the T Cell Repertoire

As summarized above in FIGS. 1A-1F, a total of 1434 unique, non-redundant, CD4 and CD8 epitopes have been defined, with the top 10 antigens accounting for 86% of the total. In these 10 most dominant antigens, a median of 87 epitopes (range of 33 to 396) is recognized. The data presented above demonstrates that T cell responses are multi-antigenic, with structural antigens being broadly recognized, but other proteins such as nsp3, nsp12, ORF3a and ORF8 also being vigorously recognized. Furthermore, data from Tarke et al. show that each individual is conservatively estimated to recognize on average 19 different CD4 and 17 different CD8 epitopes (34). Although individuals target multiple epitopes, the efficacy of the responses and number of epitopes targeted may vary substantially, dependent on HLA, the severity of disease and other factors.

This breadth of response is apparently at variance with other reports describing only a limited number of epitopes (7, 12, 16, 17, 21, 26, 27, 31). In some cases, in vitro expansion with artificial antigens was utilized, and/or a limited number of subjects, cells, and/or epitope candidates were screened. Furthermore, several of the reported narrow repertoire epitopes are different in the different studies, consistent with a stochastic selection effect. Overall, the data curated in the IEDB as of Mar. 15, 2021, reveals that over 1400 different SARS-CoV-2-derived peptide sequences have been reported as recognized by human T cell responses, to include 382 CD4 and 1052 CD8 epitopes.

### Example 14: Pre-Existing Reactivity and Cross-Reactivity with Common Cold Corona and Other Viruses, Cross-Reactivity with MERS and SARS-CoV-1

Several studies have detected responses to SARS-CoV-2 sequences in unexposed controls (4, 5). In some cases, it is possible that these responses might correspond to infections associated with lack of antibodies or a transient antibody response (20, 31). However, in other cases these responses appear to be linked to pre-existing memory responses, which at least in some instances, have been shown to map to cross-reactive recognition of the SARS-CoV-2 sequences by T cells induced by endemic “common cold” coronaviruses (17) or potentially other viral species (16, 87). This phenomenon has received considerable attention because of its potential to influence disease severity, vaccination outcomes, and potential implications for herd immunity (4, 5, 87-89).

Epitopes recognized in non-exposed individuals have been defined in 12 studies. It has been shown that, at least in some cases, the SARS-CoV-2 epitopes have significant homology to common cold coronavirus sequences, and cross-reactivity was demonstrated at the molecular level in several instances (19). Other studies, as discussed in more detail below, have examined whether SARS-CoV-2 specific T cells might cross-react on other more closely related viruses, such as SARS-CoV-1 and Middle East Respiratory Syndrome virus (MERS) (see also below). This issue is of relevance in the context of the potential for development of vaccines eliciting T cell responses broadly recognizing coronaviruses of pandemic potential.

The topic of pre-existing immune responses and cross-reactivity with common cold coronaviruses was addressed by several studies, with a range of findings. Schulien et al. detected cross-reactive T cells in longitudinal samples pre-and-post infection, and reported that these cells were expanded post in vitro restimulation (30). Sekine et al. also detected widespread reactivity in non-exposed individuals using peptide pools (31). Shomuradova et al. detected pre-existing T cell reactivity in unexposed donors using HLA-A2 tetramers, but at much lower levels compared to what was seen in exposed individuals (32). Nelde et al. tested reactivity of non-exposed donors to epitopes identified in exposed individuals, and detected reactivity, albeit at lower levels, for several epitopes (20). Keller et al. detected T cells with minimal cross reactivity with two homologous nucleocapsid peptides from NL63 and OC43 (13). Ferretti detected reactivity to OC43 and HKU1 sequences for 2 of 29 dominant epitopes, and no reactivity for NL63 and 229E (8). Rha et al. reported that the SARS-CoV-2 S 269-277 and S 1220-1228 epitopes had low homology to OC43, HKU1, 229E, and NL63, and that MHC class I multimer+ cells were not detected in unexposed subjects (26). Prakash identified 24 epitopes, and of those, 11 recalled memory CD8+ T cells from unexposed healthy individuals (25).

By way of explanation, but not a limitation of the present invention, a potential explanation for the differences observed in the degree of cross-reactivity of epitope repertoires detected in infected and unexposed subjects is provided by the studies of Mateus et al. and Tarke et al. These studies demonstrated that, overall, 50% of the epitopes defined in unexposed donors were also recognized in SARS-CoV-2 infected subjects (19, 34), but also that the viral infection created a new repertoire of epitopes recognized only in infected subjects. Conversely, more than 80% of the epitopes defined in SARS-CoV-2 infected subjects were not recognized in unexposed donors. This suggests that a pre-existing repertoire of cross-reactive T cells is present in unexposed donors, but that the SARS-CoV-2 infection generates a largely novel repertoire of T cells in addition to the pre-existing one. Consistent with this view, the antigens dominantly recognized in exposed donors tend to only partially overlap with those dominant in non-exposed donors (16).

The issue of how preexisting memory reactivity might influence immunity has been debated, and a firm conclusion has not been reached as of yet (4, 88, 90). While it is not expected that preexisting T cell reactivity might protect against infection, it is possible that preexisting SARS-CoV-2 cross-reactive T cells might modulate disease severity, as reported by a recent study (91), or even modulate vaccine responsiveness, allowing for a faster or more vigorous response.

The study of protective versus detrimental T cell responses is important to determine the optimal T cell engagement strategies for vaccines. In addition to understanding the relationship between pre-existing immunity to human coronaviruses and host defense against SARS-CoV-2, it is relevant to also consider the contribution of COVID-19 vaccine-boosted cross-reactive immune responses to vaccine-induced protective immunity.

As mentioned above, several studies have addressed whether SARS-CoV-2 T cells might cross-react with more closely related viruses such as SARS-CoV-1 and MERS. This issue is relevant in the context of development of vaccines eliciting T cell responses broadly recognizing coronaviruses of pandemic potential.

As might be expected on the basis of the higher degree of sequence homology, cross-reactivity between SARS-CoV-2 responses and SARS-CoV-1 and MERS was more frequently detected, as compared to common cold coronaviruses. More specifically, Le Bert et al. analyzed a cohort of 23 patients who recovered from SARS-1, and found long lasting memory T cells 17 years after the SARS-1 outbreak of 2003 (16). Habel et al. reported that T cells recognizing selected A2/SARS-CoV-2 CD8+ T cell epitopes can cross-react with SARS-CoV-1 and MERS, while they did not share homology with the common cold coronaviruses (10). Rha et al. reported that the S 269-277 epitope was specific to SARS-CoV-2, whereas the S 1220-1228 epitope was conserved in SARS-CoV-1 (26). In the study of Gangaev, of the 9 CD8 T cell epitopes they identified, 5 were unique for SARS-CoV-2 and 4 were shared between SARS-CoV-2 and SARS-CoV-1 (9). Prakash et al. also studied conserved pan-species epitope sequences considering all coronaviruses, including those responsible for zoonotic infections (25).

### Example 15: Potential for Immune Escape by SARS-CoV-2 Variants

Another topic of relevance is the effect of naturally occurring mutations on epitope recognition. SARS-CoV-2 does mutate, and one question is whether it will mutate to escape T cell responses. The large breadth of T cell epitopes recognized, and the fact that, dependent on HLA polymorphism, each individual tends to recognize its own unique sets of epitopes, has profound implications in the context of immune escape. A recent study showed that mutations selected for predicted negative impact on epitope binding to HLA were indeed associated with reduced T cell activity (92). Other analyses of mutations associated with several variants of concern suggest that the vast majority of defined epitopes are conserved in SARS-CoV-2 variants (93, 94).

The topic of potential immune escape by variants has been elevated by the observation that several recent SARS-CoV-2 variants of concern have accumulated unusually large numbers of mutations and exhibit significant evidence of escape from neutralizing antibodies (95-97). This evolution appears to be due to extended replication in immunocompromised individuals, at least in some cases (98). Given that immunity against COVID-19 consists of both antibody and T cell responses, there has been concern as to whether the variants escape T cell immunity.

The study of sequence variation and epitope recognition is of particular importance in the context of several well described Variants of Concern (VOCs). Two independent studies (93, 94) show that most of the epitopes defined by Tarke et al. (34) or Kared et al. (12) are conserved within VOCs. Consistent with these observations, it has been shown that the sequence variations associated with the B.1.1.7, B.1.351, P.1, and CAL.20C variants had impact on T cell responses induced by natural infection or vaccination with the ancestral Wuhan sequence limited to decreases in overall activity of less than 30% at the population level (93, 94). Because of the large number of different epitopes reported, as noted above, and of the large breadth of epitopes recognized in any given individual (again, estimated to be an average of 19 class II and 17 class I epitopes per person, genome-wide, and 9 if spike only is considered), as suggested by one study (34), it appears unlikely that the new variants will have the capacity to escape T cell recognition, at both the population and individual levels.

In light of the data indicating that T cell escape is not occurring (93), it is also worthwhile to discuss the immunological and virological features that make T cell escape by SARS-CoV-2 unlikely. First, as noted, the broader the T cell response, in terms of epitopes, the less likely viral escape becomes because any individual epitope escape mutation by the virus would represent a small fraction of the overall immunity, and thus represent a small selective pressure. Given that SARS-CoV-2 is a large RNA virus (i.e., encoding a large amount of sequence space), the breadth of the CD4 and CD8 T cell responses is not surprising per se.

Second, there are few examples in the literature of T cell epitope escape in humans for a virus that causes acute infections. In contrast, viruses that cause chronic viral infections, such as HIV and HCV, are well known to escape T cell epitope recognition. This is due to a fundamental difference in selective pressure. Within a single person, there is strong selective pressure for a chronic viral infection to escape T cell responses over time. In contrast, in a population of people, the diversity of HLA alleles presents a fundamental challenge for viral escape. That phenomenon is a basic premise in the understanding of the evolutionary value of human HLA diversity. Escape of one or more T cell epitopes in one individual is unlikely to give the virus a selection advantage in the next host; indeed, the escape mutations are more likely to be a disadvantage because the original viral protein sequence was selected for functionality. However, as observed in the influenza system (99) where restoration of viral fitness was obtained by multiple compensatory co-mutations in the nucleoprotein, generation of SARS-CoV-2 cytotoxic T-lymphocyte escape mutants by a similar mechanism is possible. Potential selection of viral T cell escape variants will be dependent on how well the spread of SARS-CoV-2 is controlled for, and even though the selection for T cell escape variants may be highly restricted due to factors discussed above, it cannot be ruled out at this time.

Third, a cornerstone feature of SARS-CoV-2 is the rapidity of replication and transmission within the human upper respiratory tract. Approximately half of SARS-CoV-2 transmissions occur in the pre-symptomatic phase of infection, before a T cell response has been mounted (in a previously unexposed or unvaccinated individual). The kinetics of SARS-CoV-2 replication and transmission are inconsistent with T cell pressure being a major component of intra-host selection in most individuals and evolutionarily relevant pressure, even though viral escape mutation may arise quickly, in acute infection, during the viremic phase. Combined, these virological, immunological, and epidemiological factors make it unlikely that SARS-CoV-2 will escape human T cell responses at the population level. All of that being said, it is still possible that escape from T cell epitope recognition may occur in individual immunocompromised patients, some of whom have high levels of viral replication for >120 days, and that the virus can undergo extensive mutation in the individual during that time.

### Example 16: TCR Repertoires

Several studies also addressed TCR repertoires and attempted to establish a link between epitope recognition and particular TCR sequences. More specifically, a seminal study by Gittelman et al. (101) obtained TCR sequence information from the entire municipality of Vo′ (Italy) during the initial surge of SARS-CoV-2 infections, and detected notable correlations with disease severity and other characteristics. Snyder et al. (33) expanded the approach and inferred several epitopes linked to recognition by specific TCRs, and also built a classifier to diagnose infection based solely on TCR sequencing from blood samples. Along the same lines, Shomuradova et al. (32) also observed specific TCR motifs, in some cases shared across multiple donors, and Ferretti et al. (8) sorted epitope specific T cells and used single cell sequencing to define paired TCR a and TCR R chains expressed by these T cells. Gangaev et al. also provided TCR sequences recognizing a defined SARS-CoV-2 epitope (9).

In conclusion, given the large number of different epitopes recognized in the context of a myriad of different HLA types, it will be necessary to compile an extensive catalog of TCR sequences to completely capture the TCR repertoire associated with SARS-CoV-2 responses in humans. In parallel, focusing on the most dominant HLA and epitope combinations is also of interest. Early reports promise that this approach might lead to very interesting diagnostic applications, and yield additional insights on pathogenesis, also in light of the recent Emergency Use Authorization of a TCR-based diagnostic developed by Adaptive Biotech (see: www.fda.gov/media/146478/download).

Discussion. The inventors reviewed 25 different studies describing the identification of SARS-CoV-2 epitopes recognized by human T cells. The studies defined over 1400 different unique epitopes (382 for CD4 and 1052 for CD8), which are herein annotated in terms of available metadata. The epitope data described here derives from studies with 1197 human subjects (870 COVID-19 and 327 unexposed controls). Twenty studies defined class I/CD8 epitopes, and 9 defined class II/CD4 epitopes. A variety of screening designs and assay methodologies were utilized. Nearly half of the class II studies use overlapping peptides (4/9 studies), and predicted binders were often used for investigating class I epitopes (21/25 studies). A total of 16 studies used ex vivo assays at some stage, and 12 utilized in vitro restimulations, with a few employing both approaches.

Ten epitope identification studies screened peptides derived from the entire proteome. However, fifteen other studies concentrated on specific subsets of antigens, based on the fact that the main antigenic targets of CD4 and CD8 SARS-CoV-2 T cell responses have been defined by studies utilizing pools of overlapping peptides. Those studies showed that structural proteins (S, M and N) are dominant targets of T cell responses, but ORF3, ORF8, nsp3, nsp4 and nsp12 are also frequently targeted. Within the main antigens, the inventors have used the IEDB's Immunome Browser tool to identify immunodominant regions. These regions are typically pronounced in the case of CD4 recognition, but less so in the case of CD8 responses, which tend to be more evenly distributed across the dominant antigens.

Epitope identification was performed in different populations and cohorts, to include both SARS-CoV-2 infected and unexposed donors. These cohorts represent considerable heterogeneity as a function of age, gender, disease severity (with severe disease less represented) and time since symptoms onset. However, different ethnicities were not broadly represented and this will be an important knowledge gap to be addressed in future investigations. Related to this issue, HLA restricted epitopes were identified for 30 class I and 45 class II molecules. The median number of epitopes per allele is 15, but ranging from 1 to 219, with a large bias toward the HLA alleles that are more frequently encountered in the general population.

As mentioned above, over 1400 different epitopes have been identified to date in the peer-reviewed and pre-print literature. A set of 399 more prevalent epitopes are defined by being recognized by 3 or more donors/different studies (110 CD4, 289 CD8). Considering that several antigens and many HLA types are under studied, this highlights a remarkably broad epitope repertoire. From a study by Tarke et al. (34), each individual is conservatively estimated to recognize 15-20 different CD4 and 15-20 different CD8 epitopes. Furthermore, the epitopes recognized are largely different from one individual to the next because of HLA polymorphism. This remarkable breadth of epitope repertoire suggests that immune escape by SARS-CoV-2 variants from T cell recognition at the population level is not a likely scenario.

### Example 17. Potential for Immune Escape by SARS-CoV-2 Variants

This example relates in general to the field of peptides that are T cell epitopes for coronavirus, including epitopes of SARS-CoV-2 variants such as the Omicron variant, and more particularly, to compositions and methods for the prevention, treatment, diagnosis, kits, and uses of such T cell epitopes, including megapools, for use in detecting and characterizing SARS-CoV-2 specific responses in infection and following vaccination. Table 8 includes SARS-CoV-2 variants from the Omicron variant.

TABLE 8
SARS-CoV-2 variants from the
Omicron variant B.1.1.529.

| SEQ | | |
|---|---|---|
| ID | B.1.1.529 | B1.1.529 |
| NO: | Start | sequence |
| 2571 | 1 | MFVFLVLLPLVSSQC |
| 2572 | 6 | VLLPLVSSQCVNLTT |
| 2573 | 11 | VSSQCVNLTTRTQLP |
| 2574 | 16 | VNLTTRTQLPPAYTN |
| 2575 | 21 | RTQLPPAYTNSFTRG |
| 2576 | 26 | PAYTNSFTRGVYYPD |
| 2577 | 31 | SFTRGVYYPDKVFRS |
| 2578 | 36 | VYYPDKVFRSSVLHS |
| 2579 | 41 | KVFRSSVLHSTQDLF |
| 2580 | 46 | SVLHSTQDLFLPFFS |
| 2581 | 51 | TQDLFLPFFSNVTWF |
| 2582 | 56 | LPFFSNVTWFHVISG |
| 2583 | 61 | NVTWFHVISGTNGTK |
| 2584 | 66 | HVISGTNGTKRFDNP |
| 2585 | 69 | SGTNGTKRFDNPVLP |
| 2586 | 74 | TKRFDNPVLPFNDGV |
| 2587 | 79 | NPVLPFNDGVYFASI |
| 2588 | 84 | FNDGVYFASIEKSNI |
| 2589 | 89 | YFASIEKSNIIRGWI |
| 2590 | 94 | EKSNIIRGWIFGTTL |
| 2591 | 99 | IRGWIFGTTLDSKTQ |
| 2592 | 104 | FGTTLDSKTQSLLIV |
| 2593 | 109 | DSKTQSLLIVNNATN |
| 2594 | 114 | SLLIVNNATNVVIKV |
| 2595 | 119 | NNATNVVIKVCEFQF |
| 2596 | 124 | VVIKVCEFQFCNDPF |
| 2597 | 129 | CEFQFCNDPFLDHKN |
| 2598 | 134 | CNDPFLDHKNNKSWM |
| 2599 | 139 | LDHKNNKSWMESEFR |
| 2600 | 142 | KNNKSWMESEFRVYS |
| 2601 | 146 | SWMESEFRVYSSANN |
| 2602 | 151 | EFRVYSSANNCTFEY |
| 2603 | 156 | SSANNCTFEYVSQPF |
| 2604 | 161 | CTFEYVSQPFLMDLE |
| 2605 | 166 | VSQPFLMDLEGKQGN |
| 2606 | 171 | LMDLEGKQGNFKNLR |
| 2607 | 176 | GKQGNFKNLREFVFK |
| 2608 | 181 | FKNLREFVFKNIDGY |
| 2609 | 186 | EFVFKNIDGYFKIYS |
| 2610 | 191 | NIDGYFKIYSKHTPI |
| 2611 | 196 | FKIYSKHTPIIVREP |
| 2612 | 201 | KHTPIIVREPEDLPQ |
| 2613 | 206 | IVREPEDLPQGFSAL |
| 2614 | 211 | EDLPQGFSALEPLVD |
| 2615 | 216 | GFSALEPLVDLPIGI |

### Example 18. T Cell-Based Immunodiagnostic System to Effectively Distinguish SARS-CoV-2 Infection and COVID-19 Vaccination Status

The present inventors developed an immunodiagnostic T cell assay using a pool of overlapping peptides spanning the entire spike protein in combination with experimentally defined non-spike pools to classify subjects based on their vaccination and infection history. This tool showed high predictive power to discriminate responses based on distinctive COVID-19 immune profiles, including hybrid immunity from breakthrough infections. Using a validation cohort, the inventors demonstrated the clinical applicability of this tool for assessing immune responses in diverse individuals, including those who received different vaccine platforms and at different lengths of time post-vaccination and infection.

Cohorts associated with known infection and vaccination history. 239 participants were enrolled in the study and classified into five groups based on known vaccination and infection history: (50 non-infected, non-vaccinated (I−V−); 50 infected and non-vaccinated (I+V−); 66 infected and then vaccinated (I+V+); 50 non-infected and vaccinated (I-V+); and 23 vaccinated and then infected (V+I+). For the I+V−, I+V+ and V+I+ groups, SARS-CoV-2 infection was determined by PCR-based testing during the acute phase of infection or verified by serological detection of antibodies against the SARS-CoV-2 Spike protein RBD region at the time of blood donation.

The study primarily consisted of subjects recruited in San Diego, California (see material and methods for more details). Among individuals with history of COVID-19 disease, the majority were symptomatic mild disease cases, owing to the nature of the study recruitment design. Specifically, 44 donors (88%) for I+V−, 45 donors (90%) for I+V+, and 23 donors (100%) for V+I+ had mild symptoms, 3 donors (6%) of I+V− and I+V+ groups had moderate symptoms, and 3 (6%) and 2 donors (4%) from the I+V− and I+V+ groups, respectively, had severe symptoms. The median days of blood collection post symptom onset (PSO) were 119 (20-308), 354 (57-508) and 32 (18-93) for I+V−, I+V+ and V+I+ groups respectively. For the I−V+, I+V+ and V+I+ groups, the vaccinated subjects received two doses of mRNA vaccines BNT162b2 (Pfizer/BioNTech) or mRNA-1273 (Moderna), as verified by vaccination records and positive plasma SARS-CoV-2 spike protein RBD IgG titers. Similar distribution of Pfizer or Moderna administered vaccines (45%-55%) were present in vaccinated subjects from either the I−V+ or I+V+ group, while in the V+I+ group, 15 (65%) subjects had received the BNT162b2 vaccine, and 8 (35%) the mRNA-1273 vaccine.

The median days of blood collection post second dose of vaccination (PVD) were 16 (13-190), 32 (7-188) and 163 (55-271) for I−V+, I+V+ and V+I+ groups, respectively. All the I−V− subjects were collected before the attributed pandemic period (2013-2019) and confirmed seronegative with undetectable SARS-CoV-2 Spike protein RBD IgG titers. In all cohorts, the median ages were relatively young (25 (17-64), 42 (19-67), 40 (21-74), 38 (21-73), 30 (22-68) for I−V−, I+V−, I−V+, I+V+ and V+I+ groups respectively), with the female gender well represented and different ethnicities represented. In this study, participants were further divided in an exploratory cohort (120 donors), an independent validation cohort (96 donors) and a third cohort of breakthrough infections (V+I+; 23 donors).

Differential SARS-CoV-2 CD4+ T cell responses in unexposed, convalescent, and vaccinated subjects. To detect SARS-CoV-2 T-cell reactivity, the inventors previously routinely utilized a pool of overlapping peptides spanning the entire spike (S) sequence (253 peptides) and a pool of predicted HLA Class II binders from the Remainder (R) of the genome (CD4R; (221 peptides) (Grifoni et al., 2020b). Here to further optimize detection of non-Spike reactivity, the inventors designed epitope pools based on Experimentally (E) defined epitopes, from the non-spike sequences of the SARS-CoV-2 proteome. The CD4RE and CD8RE megapools (MP) consisted of 284 and 621 peptides respectively. A pool of epitopes derived from an unrelated ubiquitous pathogen (EBV) (Carrasco Pro et al., 2015) was used as a specificity control.

TABLE 9
Detailed peptide sequences information
of CD4RE megapool.

| SEQ | | | | | |
|---|---|---|---|---|---|
| ID | | Anti- | | | Domi- |
| NO: | Description | gen | Start | End | nant |
| 2616 | FYVYSRVKNLNSSRV | E | 56 | 70 | Yes |
| 2617 | QFAYANRNRFLYIIK | M | 36 | 50 | Yes |
| 2618 | VLAAVYRINWITGGI | M | 66 | 80 | Yes |
| 2619 | YRINWITGGIAIAMA | M | 71 | 85 | Yes |
| 2620 | CLVGLMWLSYFIASF | M | 86 | 100 | Yes |
| 2621 | MWLSYFIASFRLFAR | M | 91 | 105 | Yes |
| 2622 | TNILLNVPLHGTILT | M | 116 | 130 | Yes |
| 2623 | SELVIGAVILRGHLR | M | 136 | 150 | Yes |
| 2624 | GAVILRGHLRIAGHHLGR | M | 141 | 158 | Yes |
| 2625 | LRGHLRIAGHHLGRC | M | 145 | 159 | Yes |
| 2626 | RGHLRIAGHHLGRCD | M | 146 | 160 | Yes |
| 2627 | IAGHHLGRCDIKDLP | M | 151 | 165 | Yes |
| 2628 | LGRCDIKDLPKEITV | M | 156 | 170 | Yes |
| 2629 | IKDLPKEITVATSRT | M | 161 | 175 | Yes |
| 2630 | KEITVATSRTLSYYK | M | 166 | 180 | Yes |
| 2631 | TSRTLSYYKLGASQRVA | M | 172 | 188 | Yes |
| 2632 | SRTLSYYKLGASQRV | M | 173 | 187 | Yes |
| 2633 | LSYYKLGASQRVAGD | M | 176 | 190 | Yes |
| 2634 | SYYKLGASQRVAGDS | M | 177 | 191 | Yes |
| 2635 | LGASQRVAGDSGFAA | M | 181 | 195 | Yes |
| 2636 | SGFAAYSRYRIGNYK | M | 191 | 205 | Yes |
| 2637 | ASWFTALTQHGKEDL | N | 50 | 64 | Yes |
| 2638 | SWFTALTQHGKEDLK | N | 51 | 65 | Yes |
| 2639 | DDQIGYYRRATRRIR | N | 81 | 95 | Yes |
| 2640 | IGYYRRATRRIRGGD | N | 84 | 98 | Yes |
| 2641 | YYRRATRRIRGGDGK | N | 86 | 100 | Yes |
| 2642 | MKDLSPRWYFYYLGT | N | 101 | 115 | Yes |
| 2643 | RWYFYYLGTGPEAGL | N | 107 | 121 | Yes |
| 2644 | NKDGIIWVATEGALN | N | 126 | 140 | Yes |
| 2645 | KDGIIWVATEGALNT | N | 127 | 141 | Yes |
| 2646 | AIVLQLPQGTTLPKG | N | 156 | 170 | Yes |
| 2647 | AGNGGDAALALLLLD | N | 211 | 225 | Yes |
| 2648 | DAALALLLLDRLNQL | N | 216 | 230 | Yes |
| 2649 | LLLLDRLNQLESKMS | N | 221 | 235 | Yes |
| 2650 | AAEASKKPRQKRTAT | N | 251 | 265 | Yes |
| 2651 | KKPRQKRTATKAYNV | N | 256 | 270 | Yes |
| 2652 | KRTATKAYNVTQAFG | N | 261 | 275 | Yes |
| 2653 | KAYNVTQAFGRRGPE | N | 266 | 280 | Yes |
| 2654 | LIRQGTDYKHWPQIA | N | 291 | 305 | Yes |
| 2655 | YKHWPQIAQFAPSAS | N | 298 | 312 | Yes |
| 2656 | WPQIAQFAPSASAFF | N | 301 | 315 | Yes |
| 2657 | ASAFFGMSRIGMEVT | N | 311 | 325 | Yes |
| 2658 | GMEVTPSGTWLTYTGAIKLD | N | 321 | 340 | Yes |
| 2659 | PSGTWLTYTGAIKLD | N | 326 | 340 | Yes |
| 2660 | GTWLTYTGAIKLDDK | N | 328 | 342 | Yes |
| 2661 | FKDQVILLNKHIDAY | N | 346 | 360 | Yes |
| 2662 | ILLNKHIDAYKTFPP | N | 351 | 365 | Yes |
| 2663 | LMIERFVSLAIDAYP | NSP12 | 854 | 868 | Yes |
| 2664 | LDDFVEIIKSQDLSV | NSP15 | 299 | 313 | Yes |
| 2665 | KVTFFPDLNGDVVAI | NSP3 | 1138 | 1152 | Yes |
| 2666 | KHFYWFFSNYLKRRV | NSP4 | 388 | 402 | Yes |
| 2667 | NRYFRLTLGVYDYLV | NSP6 | 232 | 246 | Yes |
| 2668 | INVFAFPFTIYSLLL | ORF10 | 4 | 18 | Yes |
| 2669 | FMRIFTIGTVTLKQG | ORF3a | 4 | 18 | Yes |
| 2670 | KKRWQLALSKGVHFV | ORF3a | 66 | 80 | Yes |
| 2671 | LYLYALVYFLQSINF | ORF3a | 106 | 120 | Yes |
| 2672 | QSINFVRIIMRLWLC | ORF3a | 116 | 130 | Yes |
| 2673 | IWNLDYIINLIIKNL | ORF6 | 26 | 40 | Yes |
| 2674 | QEEVQELYSPIFLIV | ORF7a | 90 | 104 | Yes |
| 2675 | TQHQPYVVDDPCPIH | ORF8 | 26 | 40 | Yes |
| 2676 | YVVDDPCPIHFYSKW | ORF8 | 31 | 45 | Yes |
| 2677 | PCPIHFYSKWYIRVG | ORF8 | 36 | 50 | Yes |
| 2678 | FYSKWYIRVGARKSA | ORF8 | 41 | 55 | Yes |
| 2679 | SKWYIRVGARKSAPL | ORF8 | 43 | 57 | Yes |
| 2680 | YIRVGARKSAPLIEL | ORF8 | 46 | 60 | Yes |
| 2681 | IGNYTVSCLPFTINC | ORF8 | 76 | 90 | Yes |
| 2682 | FTINCQEPKLGSLVV | ORF8 | 86 | 100 | Yes |
| 2683 | GSLVVRCSFYEDFLE | ORF8 | 96 | 110 | Yes |
| 2684 | RCSFYEDFLEYHDVR | ORF8 | 101 | 115 | Yes |
| 2686 | SEETGTLIVNSVLLF | E | 6 | 20 | No |
| 2687 | FLLVTLAILTALRLC | E | 26 | 40 | No |
| 2688 | LAILTALRLCAYCCN | E | 31 | 45 | No |
| 2689 | EELKKLLEQWNLVIG | M | 11 | 25 | No |
| 2690 | LLEQWNLVIGFLFLT | M | 16 | 30 | No |
| 2691 | NLVIGFLFLTWICLL | M | 21 | 35 | No |
| 2692 | FLFLTWICLLQFAYA | M | 26 | 40 | No |
| 2693 | WICLLQFAYANRNRF | M | 31 | 45 | No |
| 2694 | NRNRFLYIIKLIFLW | M | 41 | 55 | No |
| 2695 | LYIIKLIFLWLLWPV | M | 46 | 60 | No |
| 2696 | LIFLWLLWPVTLACF | M | 51 | 65 | No |
| 2697 | LLWPVTLACFVLAAV | M | 56 | 70 | No |
| 2698 | ITGGIAIAMACLVGL | M | 76 | 90 | No |
| 2699 | AIAMACLVGLMWLSY | M | 81 | 95 | No |
| 2700 | FIASFRLFARTRSMW | M | 96 | 110 | No |
| 2701 | RLFARTRSMWSFNPE | M | 101 | 115 | No |
| 2702 | TRSMWSFNPETNILL | M | 106 | 120 | No |
| 2703 | SFNPETNILLNVPLH | M | 111 | 125 | No |
| 2704 | NVPLHGTILTRPLLE | M | 121 | 135 | No |
| 2705 | GTILTRPLLESELVI | M | 126 | 140 | No |
| 2706 | RPLLESELVIGAVIL | M | 131 | 145 | No |
| 2707 | GAVILRGHLRIAGHH | M | 141 | 155 | No |
| 2708 | LRIAGHHLGRCDIKD | M | 149 | 163 | No |
| 2709 | ATSRTLSYYKLGASQ | M | 171 | 185 | No |
| 2710 | RVAGDSGFAAYSRYR | M | 186 | 200 | No |
| 2711 | YSRYRIGNYKLNTDH | M | 196 | 210 | No |
| 2712 | IGNYKLNTDHSSSSD | M | 201 | 215 | No |
| 2713 | LNTDHSSSSDNIALL | M | 206 | 220 | No |
| 2714 | PQNQRNAPRITFGGP | N | 6 | 20 | No |
| 2715 | NAPRITFGGPSDSTG | N | 11 | 25 | No |
| 2716 | TFGGPSDSTGSNQNG | N | 16 | 30 | No |
| 2717 | LTQHGKEDLKFPRGQ | N | 56 | 70 | No |
| 2718 | FPRGQGVPINTNSSP | N | 66 | 80 | No |
| 2719 | GVPINTNSSPDDQIG | N | 71 | 85 | No |
| 2720 | TRRIRGGDGKMKDLS | N | 91 | 105 | No |
| 2721 | GGDGKMKDLSPRWYF | N | 96 | 110 | No |
| 2722 | MKDLSPRWYFYYLGTGPEAG | N | 101 | 120 | No |
| 2723 | PRWYFYYLGTGPEAG | N | 106 | 120 | No |
| 2724 | LPYGANKDGIIWVAT | N | 121 | 135 | No |
| 2725 | IWVATEGALNTPKDH | N | 131 | 145 | No |
| 2726 | EGALNTPKDHIGTRN | N | 136 | 150 | No |
| 2727 | GKGQQQQGQTVTKKS | N | 236 | 250 | No |
| 2728 | KPRQKRTATKAYNVT | N | 257 | 271 | No |
| 2729 | TQAFGRRGPEQTQGN | N | 271 | 285 | No |
| 2730 | RRGPEQTQGNFGDQE | N | 276 | 290 | No |
| 2731 | FGDQELIRQGTDYKH | N | 286 | 300 | No |
| 2732 | TDYKHWPQIAQFAPS | N | 296 | 310 | No |
| 2733 | QFAPSASAFFGMSRI | N | 306 | 320 | No |
| 2734 | AFFGMSRIGMEVTPS | N | 313 | 327 | No |
| 2735 | GMEVTPSGTWLTYTG | N | 321 | 335 | No |
| 2736 | TWLTYTGAIKLDDKDPNF | N | 329 | 346 | No |
| 2737 | LTYTGAIKLDDKDPN | N | 331 | 345 | No |
| 2738 | AIKLDDKDPNFKDQV | N | 336 | 350 | No |
| 2739 | PNFKDQVILLNKHIDAYK | N | 344 | 361 | No |
| 2740 | HIDAYKTFPPTEPKK | N | 356 | 370 | No |
| 2741 | QKKQQTVTLLPAADL | N | 386 | 400 | No |
| 2742 | KQQTVTLLPAADLDDF | N | 388 | 403 | No |
| 2743 | TVILLPAADLDDFSK | N | 391 | 405 | No |
| 2744 | AADLDDFSKQLQQSM | N | 397 | 411 | No |
| 2745 | VLSFCAFAVDAAKAY | NSP10 | 13 | 27 | No |
| 2746 | PDILRVYANLGERVR | NSP12 | 169 | 183 | No |
| 2747 | SLLMPILTLTRALTA | NSP12 | 239 | 253 | No |
| 2748 | HCANFNVLFSTVFPP | NSP12 | 309 | 323 | No |
| 2749 | NVLFSTVFPPTSFGP | NSP12 | 314 | 328 | No |
| 2750 | QDALFAYTKRNVIPT | NSP12 | 524 | 538 | No |
| 2751 | KLLKSIAATRGATVV | NSP12 | 574 | 588 | No |
| 2752 | IAATRGATVVIGTSK | NSP12 | 579 | 593 | No |
| 2753 | YPKCDRAMPNMLRIM | NSP12 | 619 | 633 | No |
| 2754 | RAMPNMLRIMASLVL | NSP12 | 624 | 638 | No |
| 2755 | SHRFYRLANECAQVL | NSP12 | 649 | 663 | No |
| 2756 | SEMVMCGGSLYVKPG | NSP12 | 664 | 678 | No |
| 2757 | FNICQAVTANVNALL | NSP12 | 694 | 708 | No |
| 2758 | AVTANVNALLSTDGN | NSP12 | 699 | 713 | No |
| 2759 | EFYAYLRKHFSMMIL | NSP12 | 744 | 758 | No |
| 2760 | LRKHFSMMILSDDAV | NSP12 | 749 | 763 | No |
| 2761 | GLVASIKNFKSVLYY | NSP12 | 774 | 788 | No |
| 2762 | KTDGTLMIERFVSLA | NSP12 | 849 | 863 | No |
| 2763 | FVSLAIDAYPLTKHP | NSP12 | 859 | 873 | No |
| 2764 | IDAYPLTKHPNQEYA | NSP12 | 864 | 878 | No |
| 2765 | DVFHLYLQYIRKLHD | NSP12 | 879 | 893 | No |
| 2766 | TSHKLVLSVNPYVCN | NSP13 | 37 | 51 | No |
| 2767 | ELHLSWEVGKPRPPL | NSP13 | 162 | 176 | No |
| 2768 | PRPPLNRNYVFTGYR | NSP13 | 172 | 186 | No |
| 2769 | FTGYRVTKNSKVQIG | NSP13 | 182 | 196 | No |
| 2770 | VTKNSKVQIGEYTFE | NSP13 | 187 | 201 | No |
| 2771 | VNARLRAKHYVYIGD | NSP13 | 387 | 401 | No |
| 2772 | ISPYNSQNAVASKIL | NSP13 | 512 | 526 | No |
| 2773 | NVNRFNVAITRAKVG | NSP13 | 557 | 571 | No |
| 2774 | NMFITREEAIRHVRA | NSP14 | 71 | 85 | No |
| 2775 | REEAIRHVRAWIGFD | NSP14 | 76 | 90 | No |
| 2776 | PLMYKGLPWNVVRIK | NSP14 | 151 | 165 | No |
| 2777 | EIIKSQDLSVVSKVV | NSP15 | 304 | 318 | No |
| 2778 | TQLCQYLNTLTLAVP | NSP16 | 48 | 62 | No |
| 2779 | AVMSLKEGQINDMIL | NSP16 | 258 | 272 | No |
| 2780 | KEGQINDMILSLLSK | NSP16 | 263 | 277 | No |
| 2781 | RENNRVVISSDVLVN | NSP16 | 283 | 297 | No |
| 2782 | PLNSIIKTIQPRVEK | NSP2 | 96 | 110 | No |
| 2783 | EEIAIILASFSASTS | NSP2 | 291 | 305 | No |
| 2784 | SPLYAFASEAARVVR | NSP2 | 351 | 365 | No |
| 2785 | AITILDGISQYSLRL | NSP2 | 386 | 400 | No |
| 2786 | QTFFKLVNKFLALCA | NSP2 | 496 | 510 | No |
| 2787 | GETFVTHSKGLYRKC | NSP2 | 526 | 540 | No |
| 2788 | ADAVIKTLQPVSELL | NSP3 | 58 | 72 | No |
| 2789 | ESDDYIATNGPLKVG | NSP3 | 268 | 282 | No |
| 2790 | IATNGPLKVGGSCVL | NSP3 | 273 | 287 | No |
| 2791 | SGHNLAKHCLHVVGP | NSP3 | 288 | 302 | No |
| 2792 | NLYDKLVSSFLEMKS | NSP3 | 363 | 377 | No |
| 2793 | ENLLLYIDINGNLHP | NSP3 | 433 | 447 | No |
| 2794 | KSAFYILPSIISNEK | NSP3 | 532 | 546 | No |
| 2795 | RFYFYTSKTTVASLI | NSP3 | 603 | 617 | No |
| 2796 | EAARYMRSLKVPATV | NSP3 | 643 | 657 | No |
| 2797 | LPNDDTLRVEAFEYY | NSP3 | 803 | 817 | No |
| 2798 | TLRVEAFEYYHTTDP | NSP3 | 808 | 822 | No |
| 2799 | HTTDPSFLGRYMSAL | NSP3 | 818 | 832 | No |
| 2800 | SFLGRYMSALNHTKK | NSP3 | 823 | 837 | No |
| 2801 | YMSALNHTKKWKYPQ | NSP3 | 828 | 842 | No |
| 2802 | NHTKKWKYPQVNGLT | NSP3 | 833 | 847 | No |
| 2803 | ESPFVMMSAPPAQYE | NSP3 | 983 | 997 | No |
| 2804 | YCIDGALLTKSSEYK | NSP3 | 1028 | 1042 | No |
| 2805 | DNFKFVCDNIKFADD | NSP3 | 1108 | 1122 | No |
| 2806 | LNQLTGYKKPASREL | NSP3 | 1123 | 1137 | No |
| 2807 | GYKKPASRELKVTFF | NSP3 | 1128 | 1142 | No |
| 2808 | ASRELKVTFFPDLNG | NSP3 | 1133 | 1147 | No |
| 2809 | PDLNGDVVAIDYKHY | NSP3 | 1143 | 1157 | No |
| 2810 | TPSFKKGAKLLHKPI | NSP3 | 1158 | 1172 | No |
| 2811 | VWHVNNATNKATYKP | NSP3 | 1173 | 1187 | No |
| 2812 | MAAYVDNSSLTIKKP | NSP3 | 1278 | 1292 | No |
| 2813 | NELSRVLGLKTLATH | NSP3 | 1293 | 1307 | No |
| 2814 | TFTRSTNSRIKASMP | NSP3 | 1363 | 1377 | No |
| 2815 | TNSRIKASMPTTIAK | NSP3 | 1368 | 1382 | No |
| 2816 | NTVKSVGKFCLEASF | NSP3 | 1383 | 1397 | No |
| 2817 | LEASFNYLKSPNFSK | NSP3 | 1393 | 1407 | No |
| 2818 | PNFSKLINIIIWFLL | NSP3 | 1403 | 1417 | No |
| 2819 | GSLIYSTAALGVLMS | NSP3 | 1423 | 1437 | No |
| 2820 | ISSFKWDLTAFGLVA | NSP3 | 1493 | 1507 | No |
| 2821 | WDLTAFGLVAEWFLA | NSP3 | 1498 | 1512 | No |
| 2822 | FGLVAEWFLAYILFT | NSP3 | 1503 | 1517 | No |
| 2823 | FDAYVNTFSSTFNVP | NSP3 | 1773 | 1787 | No |
| 2824 | SHNIALIWNVKDFMS | NSP3 | 1888 | 1902 | No |
| 2825 | KGGKIVNNWLKQLIK | NSP4 | -2 | 12 | No |
| 2826 | LFVAAIFYLITPVHV | NSP4 | 18 | 32 | No |
| 2827 | AVITREVGFVVPGLP | NSP4 | 93 | 107 | No |
| 2828 | VPGLPGTILRTTNGD | NSP4 | 103 | 117 | No |
| 2829 | FLHFLPRVFSAVGNI | NSP4 | 118 | 132 | No |
| 2830 | DTRYVLMDGSIIQFP | NSP4 | 188 | 202 | No |
| 2831 | SIVAGGIVAIVVTCL | NSP4 | 283 | 297 | No |
| 2832 | FGEYSHVVAFNTLLF | NSP4 | 308 | 322 | No |
| 2833 | NTLLFLMSFTVLCLT | NSP4 | 318 | 332 | No |
| 2834 | PVYSFLPGVYSVIYL | NSP4 | 333 | 347 | No |
| 2835 | YLTFYLTNDVSFLAH | NSP4 | 348 | 362 | No |
| 2836 | SFLAHIQWMVMFTPL | NSP4 | 358 | 372 | No |
| 2837 | IQWMVMFTPLVPFWI | NSP4 | 363 | 377 | No |
| 2838 | MFTPLVPFWITIAYI | NSP4 | 368 | 382 | No |
| 2839 | TIAYIICISTKHFYW | NSP4 | 378 | 392 | No |
| 2840 | CTFLLNKEMYLKLRS | NSP4 | 418 | 432 | No |
| 2841 | LTQYNRYLALYNKYK | NSP4 | 438 | 452 | No |
| 2842 | RYLALYNKYKYFSGA | NSP4 | 443 | 457 | No |
| 2843 | YREAACCHLAKALND | NSP4 | 463 | 477 | No |
| 2844 | CCHLAKALNDFSNSG | NSP4 | 468 | 482 | No |
| 2845 | FSNSGSDVLYQPPQT | NSP4 | 478 | 492 | No |
| 2846 | SDVLYQPPQTSITSA | NSP4 | 483 | 497 | No |
| 2847 | NHNFLVQAGNVQLRV | NSP5 | 63 | 77 | No |
| 2848 | QNCVLKLKVDTANPK | NSP5 | 83 | 97 | No |
| 2849 | LLVLVQSTQWSLFFF | NSP6 | 22 | 36 | No |
| 2850 | SLFFFLYENAFLPFA | NSP6 | 32 | 46 | No |
| 2851 | LCLFLLPSLATVAYF | NSP6 | 67 | 81 | No |
| 2852 | TLVYKVYYGNALDQA | NSP6 | 147 | 161 | No |
| 2853 | DAFKLNIKLLGVGGK | NSP6 | 267 | 281 | No |
| 2854 | RVESSSKLWAQCVQL | NSP7 | 21 | 35 | No |
| 2855 | SKLWAQCVQLHNDIL | NSP7 | 26 | 40 | No |
| 2856 | VLKKLKKSLNVAKSE | NSP8 | 34 | 48 | No |
| 2857 | LIVTALRANSAVKLQ | NSP8 | 184 | 198 | No |
| 2858 | SDFVRATATIPIQAS | ORF3a | 26 | 40 | No |
| 2859 | ALLAVFQSASKIITL | ORF3a | 51 | 65 | No |
| 2860 | KIITLKKRWQLALSK | ORF3a | 61 | 75 | No |
| 2861 | CNLLLLFVTVYSHLL | ORF3a | 81 | 95 | No |
| 2862 | LVAAGLEAPFLYLYA | ORF3a | 96 | 110 | No |
| 2863 | LEAPFLYLYALVYFL | ORF3a | 101 | 115 | No |
| 2864 | LVYFLQSINFVRIIM | ORF3a | 111 | 125 | No |
| 2865 | VRIIMRLWLCWKCRS | ORF3a | 121 | 135 | No |
| 2866 | RLWLCWKCRSKNPLL | ORF3a | 126 | 140 | No |
| 2867 | KNPLLYDANYFLCWH | ORF3a | 136 | 150 | No |
| 2868 | YDANYFLCWHTNCYD | ORF3a | 141 | 155 | No |
| 2869 | FLCWHTNCYDYCIPY | ORF3a | 146 | 160 | No |
| 2870 | TNCYDYCIPYNSVTS | ORF3a | 151 | 165 | No |
| 2871 | YFTSDYYQLYSTQLS | ORF3a | 206 | 220 | No |
| 2872 | TDTGVEHVTFFIYNK | ORF3a | 221 | 235 | No |
| 2873 | EHVTFFIYNKIVDEP | ORF3a | 226 | 240 | No |
| 2874 | FIYNKIVDEPEEHVQ | ORF3a | 231 | 245 | No |
| 2875 | GSSGVVNPVMEPIYD | ORF3a | 251 | 265 | No |
| 2876 | MFHLVDFQVTIAEIL | ORF6 | 1 | 15 | No |
| 2877 | IAEILLIIMRTFKVS | ORF6 | 11 | 25 | No |
| 2878 | AEILLIIMRTFKVSI | ORF6 | 12 | 26 | No |
| 2879 | LIIMRTFKVSIWNLD | ORF6 | 16 | 30 | No |
| 2880 | TFKVSIWNLDYIINL | ORF6 | 21 | 35 | No |
| 2881 | YIINLIIKNLSKSLT | ORF6 | 31 | 45 | No |
| 2882 | MKIILFLALITLATC | ORF7a | 1 | 15 | No |
| 2883 | IILFLALITLATCEL | ORF7a | 3 | 17 | No |
| 2884 | DGVKHVYQLRARSVSPKL | ORF7a | 69 | 86 | No |
| 2885 | VKHVYQLRARSVSPK | ORF7a | 71 | 85 | No |
| 2886 | LYSPIFLIVAAIVFI | ORF7a | 96 | 110 | No |
| 2887 | SPIFLIVAAIVFITL | ORF7a | 98 | 112 | No |
| 2888 | DFYLCFLAFLLFLVL | ORF7b | 8 | 22 | No |
| 2889 | MKFLVFLGIITTVAA | ORF8 | 1 | 15 | No |
| 2890 | FLGIITTVAAFHQEC | ORF8 | 6 | 20 | No |
| 2891 | TTVAAFHQECSLQSC | ORF8 | 11 | 25 | No |
| 2892 | FHQECSLQSCTQHQP | ORF8 | 16 | 30 | No |
| 2893 | SLQSCTQHQPYVVDD | ORF8 | 21 | 35 | No |
| 2894 | ARKSAPLIELCVDEA | ORF8 | 51 | 65 | No |
| 2895 | PLIELCVDEAGSKSP | ORF8 | 56 | 70 | No |
| 2896 | CVDEAGSKSPIQYID | ORF8 | 61 | 75 | No |
| 2897 | IQYIDIGNYTVSCLP | ORF8 | 71 | 85 | No |
| 2898 | QEPKLGSLVVRCSFY | ORF8 | 91 | 105 | No |
| 2899 | EDFLEYHDVRVVLDF | ORF8 | 106 | 120 | No |
| 2900 | DFLEYHDVRVVLDFI | ORF8 | 107 | 121 | No |

TABLE 10
Detailed peptide sequences information
of CD8RE megapool.

| SEQ | | | | |
|---|---|---|---|---|
| ID | | | | |
| NO: | Description | Antigen | Dominant | |
| 2901 | SLVKPSFYV | E | Yes | Allele(s) |
| 2902 | YVYSRVKNL | E | Yes | C*06:02 |
| 2903 | GTITVEELK | M | Yes | A*68:01 |
| 2904 | KLLEQWNLV | M | Yes | A*02:01 |
| 2905 | FLFLTWICL | M | Yes | A*02:01 |
| 2906 | NRFLYIIKL | M | Yes | B*08:01, C*07:02 |
| 2907 | TLACFVLAAV | M | Yes | A*02:01 |
| 2908 | FVLAAVYRI | M | Yes | A*02:01, A*68:02 |
| 2909 | RLFARTRSMW | M | Yes | |
| 2910 | SMWSFNPET | M | Yes | A*02:01 |
| 2911 | SELVIGAVIL | M | Yes | B*40:01 |
| 2912 | AVILRGHLR | M | Yes | A*68:01 |
| 2913 | ATSRTLSYY | M | Yes | A*01:01, A*11:01, B*57:01 |
| 2914 | ATSRTLSYYK | M | Yes | A*11:01, A*30:01 |
| 2915 | RYRIGNYKL | M | Yes | A*24:02, A*30:01 |
| 2916 | QRNAPRITF | N | Yes | B*27:05, C*07:01, C*07:02 |
| 2917 | NTASWFTAL | N | Yes | A*02:01 |
| 2918 | KFPRGQGVPI | N | Yes | |
| 2919 | FPRGQGVPI | N | Yes | B*07:02, B*08:01 |
| 2920 | YYRRATRRIR | N | Yes | |
| 2921 | RIRGGDGKMK | N | Yes | |
| 2922 | KMKDLSPRW | N | Yes | B*57:01 |
| 2923 | LSPRWYFYYL | N | Yes | |
| 2924 | SPRWYFYYL | N | Yes | B*07:02, B*08:01 |
| 2925 | GTGPEAGLPY | N | Yes | |
| 2926 | ATEGALNTPK | N | Yes | A*11:01 |
| 2927 | NPANNAAIVL | N | Yes | B*07:02 |
| 2928 | VLQLPQGTTL | N | Yes | A*02:01 |
| 2929 | LALLLLDRL | N | Yes | A*02:01 |
| 2930 | LLLLDRLNQL | N | Yes | A*02:01 |
| 2931 | LLLDRLNQL | N | Yes | A*02:01 |
| 2932 | QQQQGQTVTK | N | Yes | |
| 2933 | KPRQKRTAT | N | Yes | B*07:02, B*08:01 |
| 2934 | KAYNVTQAF | N | Yes | B*35:01, B*57:01 |
| 2935 | AQFAPSASA | N | Yes | A*02:01 |
| 2936 | AQFAPSASAF | N | Yes | A*24:02, B*15:01 |
| 2937 | ASAFFGMSR | N | Yes | A*11:01, A*68:01 |
| 2938 | GMSRIGMEV | N | Yes | A*02:01 |
| 2939 | MEVTPSGTW | N | Yes | B*44:03 |
| 2940 | MEVTPSGTWL | N | Yes | B*40:01 |
| 2941 | ILLNKHIDA | N | Yes | A*02:01 |
| 2942 | KTFPPTEPK | N | Yes | A*03:01, A*11:01, A*68:01 |
| 2943 | KTFPPTEPKK | N | Yes | A*03:01, A*11:01 |
| 2944 | LPAADLDDF | N | Yes | B*35:01 |
| 2945 | FSKQLQQSM | N | Yes | |
| 2946 | VLSEARQHL | NSP1 | Yes | A*02:01 |
| 2947 | TVLSFCAFAV | NSP10 | Yes | |
| 2948 | DLKGKYVQI | NSP10 | Yes | B*08:01 |
| 2949 | YTMADLVYAL | NSP12 | Yes | |
| 2950 | TMADLVYAL | NSP12 | Yes | A*02:01 |
| 2951 | LLMPILTLT | NSP12 | Yes | |
| 2952 | KLFDRYFKY | NSP12 | Yes | A*03:01 |
| 2953 | FVDGVPFVV | NSP12 | Yes | A*02:01 |
| 2954 | ISDYDYYRY | NSP12 | Yes | |
| 2955 | RQLLFVVEV | NSP12 | Yes | A*02:01 |
| 2956 | VVDKYFDCY | NSP12 | Yes | |
| 2957 | RLYYDSMSY | NSP12 | Yes | B*15:01 |
| 2958 | MVMCGGSLYV | NSP12 | Yes | |
| 2959 | DTDFVNEFY | NSP12 | Yes | A*01:01 |
| 2960 | FYAYLRKHF | NSP12 | Yes | A*24:02 |
| 2961 | RILGAGCFV | NSP12 | Yes | |
| 2962 | LMIERFVSL | NSP12 | Yes | A*02:01 |
| 2963 | LYLQYIRKL | NSP12 | Yes | C*07:02 |
| 2964 | VLQAVGACV | NSP13 | Yes | |
| 2965 | LVLSVNPYV | NSP13 | Yes | |
| 2966 | KLFAAETLK | NSP13 | Yes | A*03:01 |
| 2967 | KLSYGIATV | NSP13 | Yes | A*02:01 |
| 2968 | VVYRGTTTY | NSP13 | Yes | |
| 2969 | VVYRGTTTYK | NSP13 | Yes | A*03:01, A*11:01 |
| 2970 | VYRGTTTYKL | NSP13 | Yes | A*24:02 |
| 2971 | KLNVGDYFV | NSP13 | Yes | A*02:01 |
| 2972 | SSNVANYQK | NSP13 | Yes | |
| 2973 | VYIGDPAQL | NSP13 | Yes | A*24:02, C*07:01 |
| 2974 | IVDTVSALV | NSP13 | Yes | |
| 2975 | IPRRNVATL | NSP13 | Yes | B*07:02, B*08:01 |
| 2976 | VLWAHGFEL | NSP14 | Yes | |
| 2977 | TYACWHHSI | NSP14 | Yes | A*24:02 |
| 2978 | AIMTRCLAV | NSP14 | Yes | |
| 2979 | IEYPIIGDEL | NSP14 | Yes | B*40:01 |
| 2980 | ALLADKFPV | NSP14 | Yes | A*02:01 |
| 2981 | SYATHSDKF | NSP14 | Yes | A*24:02 |
| 2982 | YLDAYNMMI | NSP14 | Yes | A*02:01 |
| 2983 | MMISAGFSL | NSP14 | Yes | A*02:01 |
| 2984 | KQFDTYNLW | NSP14 | Yes | B*15:01 |
| 2985 | NLWNTFTRL | NSP14 | Yes | A*02:01 |
| 2986 | KVDGVDVEL | NSP15 | Yes | A*02:01 |
| 2987 | LLLDDFVEII | NSP15 | Yes | |
| 2988 | MLWCKDGHV | NSP15 | Yes | |
| 2989 | GVAMPNLYK | NSP16 | Yes | |
| 2990 | KMQRMLLEK | NSP16 | Yes | |
| 2991 | YLNTLTLAV | NSP16 | Yes | |
| 2992 | TLIGDCATV | NSP16 | Yes | |
| 2993 | KTIQPRVEK | NSP2 | Yes | A*03:01, A*11:01 |
| 2994 | KLVNKFLAL | NSP2 | Yes | A*02:01 |
| 2995 | VTNNTFTLK | NSP2 | Yes | A*03:01, A*11:01 |
| 2996 | FGDDTVIEV | NSP3 | Yes | A*02:01 |
| 2997 | PTDNYITTY | NSP3 | Yes | A*01:01 |
| 2998 | QEILGTVSW | NSP3 | Yes | B*44:03 |
| 2999 | TTDPSFLGRY | NSP3 | Yes | A*01:01 |
| 3000 | YLATALLTL | NSP3 | Yes | A*02:01 |
| 3001 | SAPPAQYEL | NSP3 | Yes | C*07:01 |
| 3002 | NYMPYFFTL | NSP3 | Yes | A*24:02 |
| 3003 | ASMPTTIAK | NSP3 | Yes | A*11:01, A*30:01 |
| 3004 | ILFTRFFYV | NSP3 | Yes | A*02:01 |
| 3005 | YIFFASFYY | NSP3 | Yes | A*29:02 |
| 3006 | QLMCQPILLL | NSP3 | Yes | |
| 3007 | STFNVPMEK | NSP3 | Yes | A*11:01 |
| 3008 | FLPRVFSAV | NSP4 | Yes | A*02:01 |
| 3009 | FSAVGNICY | NSP4 | Yes | A*01:01 |
| 3010 | SLRPDTRYVL | NSP4 | Yes | C*07:02 |
| 3011 | FLLNKEMYL | NSP4 | Yes | A*02:01 |
| 3012 | TSEDMLNPNY | NSP5 | Yes | A*01:01 |
| 3013 | TPKYKFVRI | NSP5 | Yes | B*08:01 |
| 3014 | VRIQPGQTF | NSP5 | Yes | C*07:01 |
| 3015 | FLNGSCGSV | NSP5 | Yes | |
| 3016 | GTDLEGNFY | NSP5 | Yes | A*01:01 |
| 3017 | VLAWLYAAV | NSP5 | Yes | |
| 3018 | FLNRFTTTL | NSP5 | Yes | |
| 3019 | SAFAMMFVK | NSP6 | Yes | A*11:01 |
| 3020 | FLLPSLATV | NSP6 | Yes | A*02:01 |
| 3021 | MPASWVMRI | NSP6 | Yes | B*07:02 |
| 3022 | KLKDCVMYA | NSP6 | Yes | A*02:01 |
| 3023 | SMWALIISV | NSP6 | Yes | A*02:01 |
| 3024 | KLWAQCVQL | NSP7 | Yes | A*02:01 |
| 3025 | SEFSSLPSY | NSP8 | Yes | B*44:03 |
| 3026 | NTCDGTTFTY | NSP8 | Yes | A*01:01 |
| 3027 | FTYASALWEI | NSP8 | Yes | |
| 3028 | SALWEIQQVV | NSP8 | Yes | A*02:01 |
| 3029 | ALWEIQQVV | NSP8 | Yes | A*02:01 |
| 3030 | ALRANSAVK | NSP8 | Yes | |
| 3031 | CTDDNALAY | NSP9 | Yes | A*01:01 |
| 3032 | CTDDNALAYY | NSP9 | Yes | A*01:01 |
| 3033 | YTELEPPCRF | NSP9 | Yes | A*01:01 |
| 3034 | VTDTPKGPK | NSP9 | Yes | A*11:01 |
| 3035 | MDLFMRIFTI | ORF3a | Yes | |
| 3036 | FVRATATIPI | ORF3a | Yes | |
| 3037 | IPIQASLPF | ORF3a | Yes | B*35:01, B*51:01 |
| 3038 | IVGVALLAVF | ORF3a | Yes | |
| 3039 | ALSKGVHFV | ORF3a | Yes | A*02:01 |
| 3040 | YLYALVYFL | ORF3a | Yes | A*02:01 |
| 3041 | VYFLQSINF | ORF3a | Yes | A*24:02 |
| 3042 | LLYDANYFL | ORF3a | Yes | A*02:01 |
| 3043 | FTSDYYQLY | ORF3a | Yes | A*01:01, A*24:02 |
| 3044 | YYQLYSTQL | ORF3a | Yes | A*24:02, C*07:02 |
| 3045 | HLVDFQVTI | ORF6 | Yes | A*02:01 |
| 3046 | QLRARSVSPK | ORF7a | Yes | A*03:01 |
| 3047 | RARSVSPKL | ORF7a | Yes | B*07:02 |
| 3048 | KLFIRQEEV | ORF7a | Yes | |
| 3049 | VFITLCFTLK | ORF7a | Yes | |
| 3050 | IMLIIFWFSL | ORF7b | Yes | |
| 3051 | IQYIDIGNY | ORF8 | Yes | |
| 3052 | KLGSLVVRC | ORF8 | Yes | A*02:01 |
| 3053 | LEYHDVRVVL | ORF8 | Yes | B*40:01 |
| 3055 | SEETGTLIV | E | No | |
| 3056 | FLLVTLAIL | E | No | A*02:01 |
| 3057 | LVKPSFYVY | E | No | C*07:02 |
| 3058 | FYVYSRVKNL | E | No | A*24:02 |
| 3059 | NGTITVEELK | M | No | A*68:01 |
| 3060 | WICLLQFAY | M | No | |
| 3061 | FAYANRNRF | M | No | B*15:01, B*35:01 |
| 3062 | AYANRNRFL | M | No | A*24:02 |
| 3063 | YANRNRFLY | M | No | A*01:01, B*35:01 |
| 3064 | ANRNRFLYI | M | No | B*08:01 |
| 3065 | RNRFLYIIK | M | No | A*30:01 |
| 3066 | RNRFLYIIKL | M | No | C*07:01 |
| 3067 | RFLYIIKLIF | M | No | A*24:02 |
| 3068 | FLWLLWPVTL | M | No | A*02:01 |
| 3069 | WLLWPVTLA | M | No | A*02:01 |
| 3070 | LLWPVTLAC | M | No | A*02:01 |
| 3071 | LAAVYRINW | M | No | B*57:01 |
| 3072 | LAAVYRINWI | M | No | |
| 3073 | MACLVGLMW | M | No | B*57:01 |
| 3074 | GLMWLSYFI | M | No | A*02:01 |
| 3075 | LSYFIASFR | M | No | A*31:01, A*68:01 |
| 3076 | IASFRLFAR | M | No | A*33:01 |
| 3077 | FRLFARTRSM | M | No | B*08:01 |
| 3078 | RLFARTRSM | M | No | A*30:01 |
| 3079 | SFNPETNIL | M | No | B*08:01 |
| 3080 | SELVIGAVI | M | No | B*44:02 |
| 3081 | ELVIGAVILR | M | No | A*68:01 |
| 3082 | LVIGAVILR | M | No | A*68:01 |
| 3083 | HLRIAGHHL | M | No | B*08:01 |
| 3084 | RIAGHHLGR | M | No | A*03:01 |
| 3085 | LPKEITVAT | M | No | B*07:02 |
| 3086 | TVATSRTLSY | M | No | A*01:01 |
| 3087 | VATSRTLSY | M | No | A*01:01, B*35:01 |
| 3088 | VATSRTLSYY | M | No | A*01:01 |
| 3089 | SQRVAGDSGF | M | No | B*15:01 |
| 3090 | AGDSGFAAY | M | No | A*01:01 |
| 3091 | DSGFAAYSR | M | No | A*68:01 |
| 3092 | YSRYRIGNYK | M | No | A*30:01 |
| 3093 | SSSDNIALL | M | No | A*68:02 |
| 3094 | RPQGLPNNTA | N | No | B*07:02 |
| 3095 | QGLPNNTASW | N | No | B*57:01 |
| 3096 | LPNNTASWF | N | No | B*07:02 |
| 3097 | SPDDQIGYY | N | No | B*35:01 |
| 3098 | MKDLSPRWY | N | No | C*07:01 |
| 3099 | YLGTGPEAGL | N | No | A*02:01 |
| 3100 | AGLPYGANK | N | No | A*30:01 |
| 3101 | LPYGANKDGI | N | No | B*51:01 |
| 3102 | YGANKDGIIW | N | No | B*57:01 |
| 3103 | IIWVATEGA | N | No | A*02:01 |
| 3104 | GTRNPANNA | N | No | A*30:01 |
| 3105 | AEGSRGGSQA | N | No | |
| 3106 | SQASSRSSSR | N | No | |
| 3107 | SSRGTSPAR | N | No | |
| 3108 | KSAAEASKK | N | No | A*11:01 |
| 3109 | RTATKAYNV | N | No | A*02:01 |
| 3110 | QELIRQGTDY | N | No | B*44:02 |
| 3111 | ELIRQGTDY | N | No | A*26:01 |
| 3112 | QFAPSASAFF | N | No | A*24:02 |
| 3113 | FAPSASAFF | N | No | B*35:01 |
| 3114 | APSASAFFGM | N | No | B*07:02 |
| 3115 | SASAFFGMSR | N | No | A*68:01 |
| 3116 | VTPSGTWLTY | N | No | A*30:02 |
| 3117 | TPSGTWLTY | N | No | B*35:01 |
| 3118 | KLDDKDPNF | N | No | A*02:01 |
| 3119 | LLNKHIDAY | N | No | B*15:01 |
| 3120 | YKTFPPTEPK | N | No | A*68:01 |
| 3121 | KKQQTVTLL | N | No | C*07:01 |
| 3122 | HLKDGTCGL | NSP1 | No | B*08:01 |
| 3123 | APHGHVMVEL | NSP1 | No | B*07:02 |
| 3124 | VMVELVAEL | NSP1 | No | A*02:01 |
| 3125 | VPHVGEIPV | NSP1 | No | B*07:02 |
| 3126 | HVGEIPVAY | NSP1 | No | B*15:01 |
| 3127 | IPVAYRKVLL | NSP1 | No | B*07:02 |
| 3128 | VPANSTVLSF | NSP10 | No | B*07:02 |
| 3129 | YLASGGQPI | NSP10 | No | A*02:01 |
| 3130 | VVYRAFDIY | NSP12 | No | B*15:01 |
| 3131 | KVAGFAKFL | NSP12 | No | A*32:01 |
| 3132 | KVAGFAKFLK | NSP12 | No | A*11:01 |
| 3133 | NLIDSYFVV | NSP12 | No | A*02:01 |
| 3134 | YFVVKRHTF | NSP12 | No | A*24:02, B*08:01 |
| 3135 | VPHISRQRL | NSP12 | No | B*07:02 |
| 3136 | TLKEILVTY | NSP12 | No | A*29:02 |
| 3137 | FVENPDILRV | NSP12 | No | A*02:06 |
| 3138 | VENPDILRV | NSP12 | No | B*44:03 |
| 3139 | VENPDILRVY | NSP12 | No | B*44:02 |
| 3140 | VRQALLKTV | NSP12 | No | C*06:02 |
| 3141 | DAMRNAGIV | NSP12 | No | B*51:01 |
| 3142 | SLLMPILTL | NSP12 | No | A*02:01 |
| 3143 | HVDTDLTKPY | NSP12 | No | A*01:01 |
| 3144 | KPYIKWDLL | NSP12 | No | B*07:02 |
| 3145 | RYFKYWDQTY | NSP12 | No | A*24:02 |
| 3146 | ILHCANFNV | NSP12 | No | |
| 3147 | STVFPPTSF | NSP12 | No | B*57:01 |
| 3148 | FPPTSFGPL | NSP12 | No | |
| 3149 | TSFGPLVRK | NSP12 | No | A*03:01 |
| 3150 | SFGPLVRKI | NSP12 | No | A*24:02 |
| 3151 | KIFVDGVPFV | NSP12 | No | A*02:01 |
| 3152 | FVVSTGYHFR | NSP12 | No | A*68:01 |
| 3153 | VVSTGYHFR | NSP12 | No | A*11:01 |
| 3154 | NLHSSRLSF | NSP12 | No | B*08:01 |
| 3155 | QTVKPGNFNK | NSP12 | No | A*11:01 |
| 3156 | FAVSKGFFK | NSP12 | No | A*11:01 |
| 3157 | AAISDYDYY | NSP12 | No | A*01:01 |
| 3158 | AAISDYDYYR | NSP12 | No | A*68:01 |
| 3159 | AISDYDYYR | NSP12 | No | A*11:01 |
| 3160 | YRYNLPTMC | NSP12 | No | C*06:02 |
| 3161 | FVVEVVDKY | NSP12 | No | B*15:01 |
| 3162 | KSAGFPFNK | NSP12 | No | A*03:01 |
| 3163 | KSAGFPFNKW | NSP12 | No | B*57:01 |
| 3164 | ARLYYDSMSY | NSP12 | No | C*07:02 |
| 3165 | FAYTKRNVI | NSP12 | No | B*51:01 |
| 3166 | NVIPTITQM | NSP12 | No | C*07:01 |
| 3167 | IPTITQMNL | NSP12 | No | B*07:02 |
| 3168 | TITQMNLKY | NSP12 | No | A*01:01 |
| 3169 | YAISAKNRAR | NSP12 | No | A*68:01 |
| 3170 | SAKNRARTV | NSP12 | No | C*06:02 |
| 3171 | SICSTMTNR | NSP12 | No | A*33:01 |
| 3172 | IAATRGATV | NSP12 | No | B*51:01 |
| 3173 | ATVVIGTSK | NSP12 | No | A*11:01 |
| 3174 | VENPHLMGWD | NSP12 | No | B*44:02 |
| 3175 | NMLRIMASL | NSP12 | No | |
| 3176 | LRIMASLVL | NSP12 | No | C*07:02 |
| 3177 | IMASLVLAR | NSP12 | No | A*33:01 |
| 3178 | MASLVLARK | NSP12 | No | A*68:01 |
| 3179 | RLANECAQV | NSP12 | No | A*02:01 |
| 3180 | TSSGDATTAY | NSP12 | No | A*01:01 |
| 3181 | VRNLQHRLY | NSP12 | No | C*07:01 |
| 3182 | FVNEFYAYL | NSP12 | No | A*02:01 |
| 3183 | FVNEFYAYLR | NSP12 | No | A*33:01 |
| 3184 | LRKHFSMMI | NSP12 | No | C*06:02 |
| 3185 | LYYQNNVFM | NSP12 | No | A*24:02 |
| 3186 | LVKQGDDYVY | NSP12 | No | B*15:01 |
| 3187 | KQGDDYVYL | NSP12 | No | A*02:01 |
| 3188 | YLPYPDPSRI | NSP12 | No | B*51:01 |
| 3189 | LPYPDPSRIL | NSP12 | No | B*07:02, B*51:01 |
| 3190 | QEYADVFHLY | NSP12 | No | A*29:02, B*44:03 |
| 3191 | YADVFHLYL | NSP12 | No | C*07:02 |
| 3192 | LTNDNTSRYW | NSP12 | No | B*57:01 |
| 3193 | WEPEFYEAM | NSP12 | No | B*40:01 |
| 3194 | AMYTPHTVL | NSP12 | No | A*32:01 |
| 3195 | TPHTVLQAV | NSP12 | No | B*51:01 |
| 3196 | QLYLGGMSYY | NSP13 | No | B*15:01 |
| 3197 | YRGTTTYKL | NSP13 | No | C*06:02 |
| 3198 | TLVPQEHYV | NSP13 | No | |
| 3199 | VPQEHYVRI | NSP13 | No | B*08:01 |
| 3200 | YQKVGMQKY | NSP13 | No | |
| 3201 | FAIGLALYY | NSP13 | No | C*07:02 |
| 3202 | YYPSARIVY | NSP13 | No | A*24:02 |
| 3203 | IPARARVEC | NSP13 | No | B*07:02 |
| 3204 | IPARARVECF | NSP13 | No | B*07:02 |
| 3205 | YVFCTVNAL | NSP13 | No | |
| 3206 | VVNARLRAK | NSP13 | No | A*11:01 |
| 3207 | RPQIGVVREF | NSP13 | No | B*15:01 |
| 3208 | AVASKILGL | NSP13 | No | A*02:01 |
| 3209 | ILGLPTQTV | NSP13 | No | A*02:01 |
| 3210 | EEAIRHVRAW | NSP14 | No | B*44:03 |
| 3211 | LQLGFSTGV | NSP14 | No | |
| 3212 | MYKGLPWNV | NSP14 | No | C*06:02 |
| 3213 | KNLSDRVVFV | NSP14 | No | A*02:01 |
| 3214 | PFMIDVQQW | NSP14 | No | A*24:02 |
| 3215 | LLADKFPVL | NSP14 | No | B*08:01 |
| 3216 | YKIEELFYSY | NSP14 | No | A*01:01 |
| 3217 | KIEELFYSY | NSP14 | No | B*15:01 |
| 3218 | FTDGVCLFW | NSP14 | No | A*01:01 |
| 3219 | SLYVNKHAF | NSP14 | No | B*08:01 |
| 3220 | AGFSLWVYK | NSP14 | No | A*11:01 |
| 3221 | YNLWNTFTRL | NSP14 | No | A*02:01 |
| 3222 | NTFTRLQSL | NSP14 | No | C*07:01 |
| 3223 | SLENVAFNV | NSP15 | No | A*02:01 |
| 3224 | TTLPVNVAF | NSP15 | No | |
| 3225 | TICAPLTVF | NSP15 | No | B*15:01 |
| 3226 | GRVDGQVDL | NSP15 | No | C*07:01 |
| 3227 | KVDGVVQQL | NSP15 | No | A*02:01 |
| 3228 | VVQQLPETY | NSP15 | No | B*15:01 |
| 3229 | KPRSQMEIDF | NSP15 | No | B*07:02 |
| 3230 | FIERYKLEGY | NSP15 | No | A*01:01 |
| 3231 | LLLDDFVEI | NSP15 | No | A*02:01 |
| 3232 | SVVSKVVKV | NSP15 | No | A*02:01 |
| 3233 | VAMPNLYKM | NSP16 | No | B*57:01 |
| 3234 | LPKGIMMNV | NSP16 | No | B*07:02 |
| 3235 | CATVHTANKW | NSP16 | No | B*57:01 |
| 3236 | KLMGHFAWW | NSP16 | No | A*32:01 |
| 3237 | YVMHANYIF | NSP16 | No | A*32:01 |
| 3238 | FWRNTNPIQL | NSP16 | No | C*07:01 |
| 3239 | WRNTNPIQL | NSP16 | No | C*07:01 |
| 3240 | NPIQLSSYSL | NSP16 | No | B*07:02 |
| 3241 | SYSLFDMSKF | NSP16 | No | A*24:02 |
| 3242 | FPLKLRGTA | NSP16 | No | B*07:02 |
| 3243 | FPLKLRGTAV | NSP16 | No | B*08:01 |
| 3244 | LRGTAVMSL | NSP16 | No | C*07:01 |
| 3245 | TFNGECPNF | NSP2 | No | A*24:02 |
| 3246 | GFMGRIRSV | NSP2 | No | C*06:02 |
| 3247 | EEIAIILASF | NSP2 | No | B*44:03 |
| 3248 | ILSPLYAFA | NSP2 | No | A*02:01 |
| 3249 | VRSIFSRTL | NSP2 | No | C*06:02 |
| 3250 | ITILDGISQY | NSP2 | No | B*15:01 |
| 3251 | RLIDAMMFT | NSP2 | No | A*02:01 |
| 3252 | TVYEKLKPV | NSP2 | No | A*02:01 |
| 3253 | EIKESVQTF | NSP2 | No | B*15:01 |
| 3254 | GETLPTEVL | NSP2 | No | B*40:01 |
| 3255 | DTVIEVQGYK | NSP3 | No | A*68:01 |
| 3256 | QGYKSVNITF | NSP3 | No | A*24:02 |
| 3257 | FELDERIDKV | NSP3 | No | A*02:01 |
| 3258 | ELDERIDKV | NSP3 | No | A*02:01 |
| 3259 | VLNEKCSAY | NSP3 | No | B*15:01 |
| 3260 | VELGTEVNEF | NSP3 | No | B*44:02 |
| 3261 | SELLTPLGI | NSP3 | No | B*40:01 |
| 3262 | YLFDESGEF | NSP3 | No | B*15:01 |
| 3263 | FEPSTQYEY | NSP3 | No | B*44:02 |
| 3264 | DDYQGKPLEF | NSP3 | No | A*24:02 |
| 3265 | LEFGATSAAL | NSP3 | No | B*40:01 |
| 3266 | VEVQPQLEM | NSP3 | No | B*40:01 |
| 3267 | LEMELTPVV | NSP3 | No | B*40:01 |
| 3268 | MELTPVVQTI | NSP3 | No | B*40:01 |
| 3269 | TPVVQTIEV | NSP3 | No | B*07:02 |
| 3270 | TIEVNSFSGY | NSP3 | No | A*01:01 |
| 3271 | IEVNSFSGY | NSP3 | No | B*44:02 |
| 3272 | NSFSGYLKL | NSP3 | No | C*06:02 |
| 3273 | YLKLTDNVY | NSP3 | No | B*15:01 |
| 3274 | EAKKVKPTV | NSP3 | No | B*51:01 |
| 3275 | VVVNAANVY | NSP3 | No | B*35:01 |
| 3276 | YIATNGPLK | NSP3 | No | A*11:01 |
| 3277 | YENFNQHEV | NSP3 | No | B*40:01 |
| 3278 | LLSAGIFGA | NSP3 | No | |
| 3279 | GADPIHSLR | NSP3 | No | A*68:01 |
| 3280 | RTNVYLAVF | NSP3 | No | B*57:01 |
| 3281 | AVFDKNLYDK | NSP3 | No | A*03:01, A*11:01 |
| 3282 | LYDKLVSSF | NSP3 | No | A*24:02 |
| 3283 | KLVSSFLEM | NSP3 | No | B*15:01 |
| 3284 | KIAEIPKEEV | NSP3 | No | A*02:01 |
| 3285 | EVKPFITESK | NSP3 | No | A*68:01 |
| 3286 | ESKPSVEQR | NSP3 | No | A*68:01 |
| 3287 | FLTENLLLYI | NSP3 | No | A*02:01 |
| 3288 | LVSDIDITF | NSP3 | No | B*35:01 |
| 3289 | APYIVGDVV | NSP3 | No | B*51:01 |
| 3290 | LTAVVIPTK | NSP3 | No | A*68:01 |
| 3291 | ALRKVPTDNY | NSP3 | No | B*15:01 |
| 3292 | KQEILGTVSW | NSP3 | No | B*44:02, B*44:03 |
| 3293 | MLAHAEETR | NSP3 | No | A*68:01 |
| 3294 | AHAEETRKL | NSP3 | No | C*06:02 |
| 3295 | KLMPVCVET | NSP3 | No | A*02:01 |
| 3296 | AIVSTIQRK | NSP3 | No | A*03:01 |
| 3297 | VVDYGARFY | NSP3 | No | A*01:01 |
| 3298 | SLINTLNDL | NSP3 | No | A*02:01 |
| 3299 | VSSPDAVTAY | NSP3 | No | A*01:01, B*57:01 |
| 3300 | TISLAGSYK | NSP3 | No | A*03:01, A*11:01, A*68:01 |
| 3301 | ISLAGSYKDW | NSP3 | No | B*57:01 |
| 3302 | YYTSNPTTF | NSP3 | No | A*24:02 |
| 3303 | TSNPTTFHL | NSP3 | No | B*57:01 |
| 3304 | HLDGEVITF | NSP3 | No | C*07:02 |
| 3305 | GEVITFDNL | NSP3 | No | B*40:01 |
| 3306 | ITFDNLKTL | NSP3 | No | B*57:01 |
| 3307 | RTIKVFTTV | NSP3 | No | A*02:01 |
| 3308 | NINLHTQVV | NSP3 | No | B*08:01 |
| 3309 | QVVDMSMTY | NSP3 | No | A*01:01, A*11:01 |
| 3310 | MSMTYGQQF | NSP3 | No | B*57:01 |
| 3311 | LRVEAFEYY | NSP3 | No | C*07:01 |
| 3312 | HTTDPSFLGR | NSP3 | No | A*68:01 |
| 3313 | FLGRYMSAL | NSP3 | No | |
| 3314 | YMSALNHTK | NSP3 | No | A*03:01 |
| 3315 | MSALNHTKK | NSP3 | No | A*30:01 |
| 3316 | MSALNHTKKW | NSP3 | No | B*57:01 |
| 3317 | SALNHTKKW | NSP3 | No | B*57:01 |
| 3318 | WKYPQVNGL | NSP3 | No | C*07:01 |
| 3319 | YPQVNGLTSI | NSP3 | No | B*51:01 |
| 3320 | ARAGEAANF | NSP3 | No | C*07:01 |
| 3321 | GEAANFCAL | NSP3 | No | B*40:01 |
| 3322 | LGDVRETMSY | NSP3 | No | A*01:01 |
| 3323 | VRETMSYLF | NSP3 | No | C*07:01 |
| 3324 | VMYMGTLSY | NSP3 | No | A*03:01 |
| 3325 | YTGNYQCGHY | NSP3 | No | A*01:01 |
| 3326 | DVFYKENSY | NSP3 | No | A*26:01 |
| 3327 | YTTTIKPVTY | NSP3 | No | A*01:01, A*26:01 |
| 3328 | EIDPKLDNY | NSP3 | No | A*01:01, A*26:01 |
| 3329 | NYYKKDNSY | NSP3 | No | C*07:02 |
| 3330 | YYKKDNSYF | NSP3 | No | A*24:02 |
| 3331 | ASFDNFKFV | NSP3 | No | A*02:06, C*06:02 |
| 3332 | KFADDLNQL | NSP3 | No | C*07:02 |
| 3333 | ASRELKVTF | NSP3 | No | A*30:01, B*57:01 |
| 3334 | DVVAIDYKHY | NSP3 | No | A*26:01 |
| 3335 | VVAIDYKHY | NSP3 | No | B*15:01 |
| 3336 | DYKHYTPSF | NSP3 | No | A*24:02 |
| 3337 | LHKPIVWHV | NSP3 | No | C*06:02 |
| 3338 | NKATYKPNTW | NSP3 | No | B*57:01 |
| 3339 | SEDAQGMDNL | NSP3 | No | B*40:01 |
| 3340 | EEVVENPTI | NSP3 | No | B*44:03 |
| 3341 | TEVVGDIIL | NSP3 | No | B*40:01 |
| 3342 | AYVDNSSLTI | NSP3 | No | A*24:02 |
| 3343 | LTIKKPNEL | NSP3 | No | B*08:01 |
| 3344 | KPNELSRVL | NSP3 | No | B*07:02, B*08:01 |
| 3345 | NELSRVLGL | NSP3 | No | B*40:01, B*44:02 |
| 3346 | SRVLGLKTL | NSP3 | No | C*07:01 |
| 3347 | SVPWDTIANY | NSP3 | No | A*26:01 |
| 3348 | DTIANYAKPF | NSP3 | No | A*26:01 |
| 3349 | YAKPFLNKV | NSP3 | No | C*06:02 |
| 3350 | RIKASMPTT | NSP3 | No | A*30:01 |
| 3351 | KASMPTTIA | NSP3 | No | A*30:01 |
| 3352 | MPTTIAKNTV | NSP3 | No | B*51:01 |
| 3353 | TTIAKNTVK | NSP3 | No | A*30:01 |
| 3354 | NTVKSVGKF | NSP3 | No | A*26:01 |
| 3355 | KFCLEASFNY | NSP3 | No | A*29:02 |
| 3356 | CLEASFNYL | NSP3 | No | A*02:01 |
| 3357 | KLINIIIWF | NSP3 | No | A*32:01 |
| 3358 | SLIYSTAAL | NSP3 | No | A*02:01 |
| 3359 | STAALGVLM | NSP3 | No | A*26:01 |
| 3360 | MSNLGMPSY | NSP3 | No | B*15:01, B*57:01 |
| 3361 | EGYLNSTNV | NSP3 | No | B*51:01 |
| 3362 | NSTNVTIATY | NSP3 | No | A*26:01 |
| 3363 | STNVTIATY | NSP3 | No | A*01:01, A*32:01 |
| 3364 | ETIQITISSF | NSP3 | No | A*26:01 |
| 3365 | IQITISSFK | NSP3 | No | A*03:01 |
| 3366 | LTAFGLVAEW | NSP3 | No | B*57:01 |
| 3367 | LVAEWFLAY | NSP3 | No | A*26:01, A*29:02 |
| 3368 | AEWFLAYIL | NSP3 | No | B*40:01, B*44:02 |
| 3369 | AEWFLAYILF | NSP3 | No | B*44:02 |
| 3370 | AYILFTRFF | NSP3 | No | A*24:02 |
| 3371 | AVHFISNSW | NSP3 | No | B*57:01 |
| 3372 | HFISNSWLMW | NSP3 | No | A*24:02 |
| 3373 | WLMWLIINL | NSP3 | No | A*02:01 |
| 3374 | LVQMAPISAM | NSP3 | No | B*15:01 |
| 3375 | SAMVRMYIF | NSP3 | No | B*08:01 |
| 3376 | RMYIFFASFY | NSP3 | No | A*03:01 |
| 3377 | SFYYVWKSY | NSP3 | No | A*29:02 |
| 3378 | FYYVWKSYV | NSP3 | No | C*06:02, C*07:02 |
| 3379 | YVYANGGKGF | NSP3 | No | A*26:01, B*15:01 |
| 3380 | DTFCAGSTF | NSP3 | No | A*26:01 |
| 3381 | EVARDLSLQF | NSP3 | No | A*26:01 |
| 3382 | VARDLSLQF | NSP3 | No | B*57:01 |
| 3383 | TVKNGSIHLY | NSP3 | No | A*26:01 |
| 3384 | VKNGSIHLY | NSP3 | No | C*06:02 |
| 3385 | YFDKAGQKTY | NSP3 | No | C*07:02 |
| 3386 | TYERHSLSHF | NSP3 | No | A*24:02 |
| 3387 | YERHSLSHF | NSP3 | No | B*44:02 |
| 3388 | ERHSLSHFV | NSP3 | No | C*06:02 |
| 3389 | SSAKSASVY | NSP3 | No | B*15:01 |
| 3390 | SAKSASVYY | NSP3 | No | B*57:01 |
| 3391 | DSAEVAVKM | NSP3 | No | A*26:01 |
| 3392 | EVAVKMFDAY | NSP3 | No | A*26:01 |
| 3393 | KMFDAYVNTF | NSP3 | No | A*24:02 |
| 3394 | MFDAYVNTF | NSP3 | No | B*08:01 |
| 3395 | AYVNTFSSTF | NSP3 | No | A*24:02 |
| 3396 | YVNTFSSTF | NSP3 | No | A*26:01 |
| 3397 | VPMEKLKTL | NSP3 | No | B*51:01 |
| 3398 | AEAELAKNV | NSP3 | No | B*44:02, B*44:03 |
| 3399 | AELAKNVSL | NSP3 | No | B*44:02 |
| 3400 | SLDNVLSTF | NSP3 | No | A*32:01 |
| 3401 | TFISAARQGF | NSP3 | No | A*24:02 |
| 3402 | DSCNNYMLTY | NSP3 | No | A*01:01 |
| 3403 | VENMTPRDL | NSP3 | No | B*44:03 |
| 3404 | TPRDLGACI | NSP3 | No | B*07:02 |
| 3405 | VAKSHNIAL | NSP3 | No | B*07:02 |
| 3406 | AKSHNIALIW | NSP3 | No | B*57:01 |
| 3407 | QVVNVVTTK | NSP3 | No | A*03:01 |
| 3408 | VVTTKIALK | NSP3 | No | A*03:01 |
| 3409 | KQLIKVTLVF | NSP4 | No | B*15:01 |
| 3410 | FYLITPVHV | NSP4 | No | C*07:02 |
| 3411 | YLITPVHVM | NSP4 | No | A*02:01, B*15:01, C*07:01 |
| 3412 | SEIIGYKAI | NSP4 | No | B*40:01 |
| 3413 | IAAVITREV | NSP4 | No | B*51:01 |
| 3414 | FVVPGLPGT | NSP4 | No | A*02:06 |
| 3415 | VPGLPGTIL | NSP4 | No | B*07:02 |
| 3416 | RTTNGDFLHF | NSP4 | No | B*57:01 |
| 3417 | VLAAECTIF | NSP4 | No | B*15:01 |
| 3418 | DASGKPVPY | NSP4 | No | B*35:01 |
| 3419 | TNVLEGSVAY | NSP4 | No | B*35:01 |
| 3420 | GSVAYESLR | NSP4 | No | A*31:01 |
| 3421 | RPDTRYVLM | NSP4 | No | B*07:02, B*35:01 |
| 3422 | SIIQFPNTY | NSP4 | No | B*35:01 |
| 3423 | WVLNNDYYR | NSP4 | No | A*31:01 |
| 3424 | SLPGVFCGV | NSP4 | No | A*02:01 |
| 3425 | DAVNLLTNM | NSP4 | No | B*51:01 |
| 3426 | IVAGGIVAI | NSP4 | No | A*02:01 |
| 3427 | LAYYFMRFR | NSP4 | No | A*31:01 |
| 3428 | YFMRFRRAF | NSP4 | No | A*24:02 |
| 3429 | FGEYSHVVAF | NSP4 | No | B*40:01 |
| 3430 | SFLPGVYSV | NSP4 | No | A*24:02 |
| 3431 | IYLYLTFYL | NSP4 | No | A*24:02 |
| 3432 | YLTNDVSFLA | NSP4 | No | A*02:01 |
| 3433 | FLAHIQWMV | NSP4 | No | A*02:01, A*02:06 |
| 3434 | MFTPLVPFW | NSP4 | No | A*24:02 |
| 3435 | VPFWITIAY | NSP4 | No | B*35:01 |
| 3436 | WFFSNYLKR | NSP4 | No | A*31:01 |
| 3437 | DVLLPLTQY | NSP4 | No | B*35:01 |
| 3438 | LPLTQYNRY | NSP4 | No | B*35:01 |
| 3439 | GAMDTTSYR | NSP4 | No | A*31:01 |
| 3440 | SNSGSDVLY | NSP4 | No | A*01:01 |
| 3441 | LYQPPQTSI | NSP4 | No | A*24:02, C*07:01 |
| 3442 | AVLQSGFRK | NSP5 | No | |
| 3443 | TANPKTPKY | NSP5 | No | C*07:01 |
| 3444 | NPKTPKYKF | NSP5 | No | B*07:02 |
| 3445 | IQPGQTFSV | NSP5 | No | |
| 3446 | QPGQTFSVL | NSP5 | No | B*07:02 |
| 3447 | SPSGVYQCAM | NSP5 | No | B*07:02 |
| 3448 | ILTSLLVLV | NSP6 | No | A*02:01 |
| 3449 | FLYENAFLP | NSP6 | No | A*02:01 |
| 3450 | LPFAMGIIAM | NSP6 | No | B*07:02 |
| 3451 | MFVKHKHAF | NSP6 | No | C*07:02 |
| 3452 | FVKHKHAFL | NSP6 | No | B*08:01 |
| 3453 | LFLLPSLATV | NSP6 | No | A*02:01 |
| 3454 | FLLPSLATVA | NSP6 | No | A*02:01 |
| 3455 | VYMPASWVM | NSP6 | No | A*24:02 |
| 3456 | RIMTWLDMV | NSP6 | No | A*02:01 |
| 3457 | WLDMVDTSL | NSP6 | No | A*02:01 |
| 3458 | VMYASAVVLL | NSP6 | No | A*24:02 |
| 3459 | MYASAVVLL | NSP6 | No | C*07:02 |
| 3460 | YASAVVLLI | NSP6 | No | C*06:02 |
| 3461 | TLMNVLTLV | NSP6 | No | A*02:01 |
| 3462 | FLARGIVFM | NSP6 | No | A*02:01 |
| 3463 | IFFITGNTL | NSP6 | No | A*24:02 |
| 3464 | GVYDYLVST | NSP6 | No | A*02:01 |
| 3465 | YDYLVSTQEF | NSP6 | No | C*07:02 |
| 3466 | FRYMNSQGL | NSP6 | No | |
| 3467 | GLLPPKNSI | NSP6 | No | A*02:01 |
| 3468 | KLNIKLLGV | NSP6 | No | A*02:01 |
| 3469 | EAFEKMVSL | NSP7 | No | B*08:01 |
| 3470 | SLLSVLLSM | NSP7 | No | A*02:01 |
| 3471 | TFTYASALW | NSP8 | No | A*24:02 |
| 3472 | AWPLIVTAL | NSP8 | No | A*24:02 |
| 3473 | GPKVKYLYF | NSP9 | No | B*08:01 |
| 3474 | YFIKGLNNL | NSP9 | No | C*07:02 |
| 3475 | YINVFAFPF | ORF10 | No | A*02:01 |
| 3476 | NVFAFPFTI | ORF10 | No | A*02:01 |
| 3477 | FTIGTVTLK | ORF3a | No | A*68:01 |
| 3478 | ATIPIQASL | ORF3a | No | B*57:01 |
| 3479 | ALLAVFQSA | ORF3a | No | A*02:01 |
| 3480 | QSASKIITL | ORF3a | No | B*08:01 |
| 3481 | SASKIITLK | ORF3a | No | A*03:01, A*11:01 |
| 3482 | ITLKKRWQL | ORF3a | No | B*08:01, B*57:01 |
| 3483 | TLKKRWQLA | ORF3a | No | B*08:01 |
| 3484 | NLLLLFVTV | ORF3a | No | A*02:01 |
| 3485 | FVTVYSHLL | ORF3a | No | A*02:01 |
| 3486 | TVYSHLLLV | ORF3a | No | A*02:01 |
| 3487 | VAAGLEAPF | ORF3a | No | B*35:01 |
| 3488 | AAGLEAPFLY | ORF3a | No | A*01:01 |
| 3489 | AGLEAPFLY | ORF3a | No | A*29:02 |
| 3490 | GLEAPFLYL | ORF3a | No | A*02:01 |
| 3491 | LEAPFLYLY | ORF3a | No | A*29:02 |
| 3492 | APFLYLYAL | ORF3a | No | B*07:02, B*08:01 |
| 3493 | FLYLYALVY | ORF3a | No | C*07:02 |
| 3494 | LYLYALVYF | ORF3a | No | A*24:02 |
| 3495 | FVRIIMRLW | ORF3a | No | B*57:01 |
| 3496 | VRIIMRLWL | ORF3a | No | C*07:02 |
| 3497 | CRSKNPLLY | ORF3a | No | C*06:02 |
| 3498 | NPLLYDANY | ORF3a | No | B*53:01 |
| 3499 | IPYNSVTSSI | ORF3a | No | |
| 3500 | TTSPISEHDY | ORF3a | No | |
| 3501 | HSYFTSDYY | ORF3a | No | A*29:02 |
| 3502 | YFTSDYYQL | ORF3a | No | C*07:02 |
| 3503 | YFTSDYYQLY | ORF3a | No | A*01:01, A*29:02 |
| 3504 | DYYQLYSTQL | ORF3a | No | A*24:02 |
| 3505 | HVTFFIYNK | ORF3a | No | A*68:01 |
| 3506 | EEHVQIHTI | ORF3a | No | |
| 3507 | IYDEPTTTT | ORF3a | No | C*07:02 |
| 3508 | HLVDFQVTIA | ORF6 | No | A*02:01 |
| 3509 | VTIAEILLI | ORF6 | No | |
| 3510 | LIIMRTFKV | ORF6 | No | B*08:01 |
| 3511 | TFKVSIWNL | ORF6 | No | B*08:01 |
| 3512 | LDYIINLII | ORF6 | No | |
| 3513 | QECVRGTTVL | ORF7a | No | |
| 3514 | YEGNSPFHPL | ORF7a | No | B*40:01 |
| 3515 | EGNSPFHPL | ORF7a | No | B*08:01 |
| 3516 | HPLADNKFAL | ORF7a | No | B*08:01 |
| 3517 | SPIFLIVAA | ORF7a | No | B*07:02 |
| 3518 | QSCTQHQPY | ORF8 | No | A*01:01 |
| 3519 | VDDPCPIHFY | ORF8 | No | A*01:01 |
| 3520 | RVGARKSAPL | ORF8 | No | |
| 3521 | EPKLGSLVV | ORF8 | No | B*07:02 |
| 3522 | EYHDVRVVL | ORF8 | No | A*24:02 |

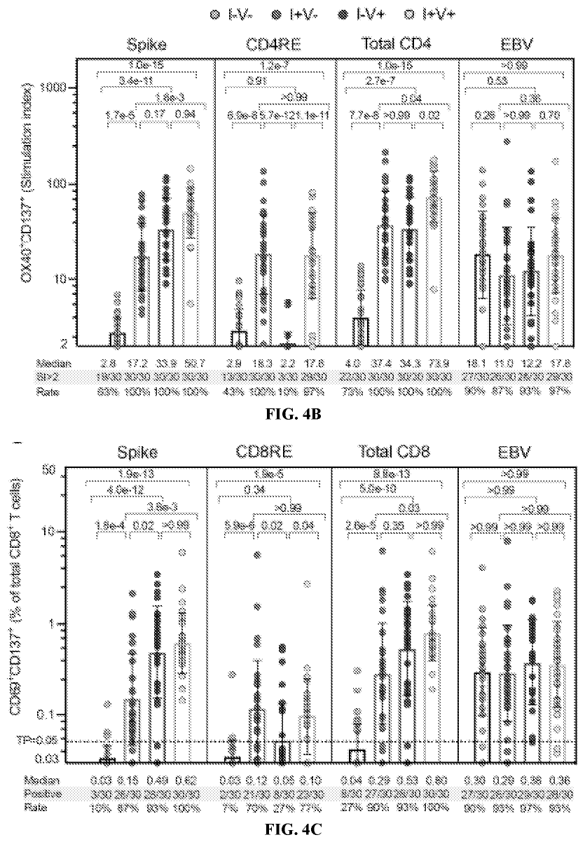

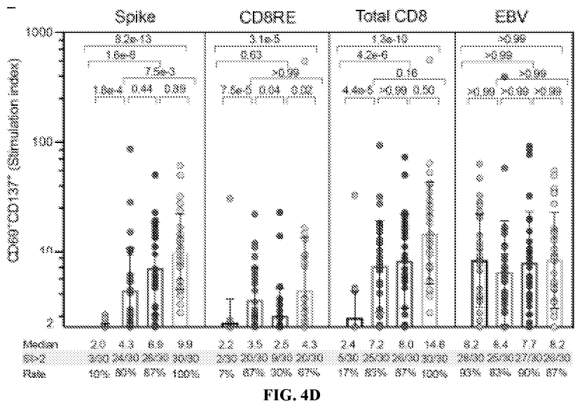

T cell reactivity was assessed by the Activation Induced Marker (AIM) assays (da Silva Antunes et al., 2021) and data represented as either absolute magnitude or stimulation index (SI). As shown in FIG. 4A SARS-CoV-2-specific CD4+ T cell responses were detected in all convalescent and/or vaccinated individuals and approximately 50% of non-infected, non-vaccinated individuals. Similar results were observed when responses were plotted as SI (FIG. 4B). Unexposed subjects were associated with significantly lower reactivity as compared to all the other groups (p-values ranging 1.3e-7 to 1.0e-15) and convalescent and vaccinated (I+V+) subjects exhibited higher responses than convalescent (I+V−) subjects (p=0.02 and p=0.04 for absolute magnitude and SI, respectively) or vaccinated (I-V+) subjects (p=0.01 and p=0.02 for absolute magnitude and SI, respectively) (FIGS. 4A, 4B). Importantly, CD4RE responses were able to differentiate convalescent subjects (I+V− or I+V+) from unexposed and vaccinated (I-V+) subjects with p-values ranging 5.6e-8 to 5.7e-12 and vaccinated (I-V+) from infected and vaccinated (I+V+) subjects (p=1.4e-11 and p=1.1e-11 for absolute magnitude and SI, respectively) (FIGS. 4A, 4B). As expected, no statistically significant difference in EBV reactivity was observed when the four groups were compared (FIGS. 4A, 4B).

Differential SARS-CoV-2 CD8+ T cell and IFNγ FluoroSpot responses in unexposed, convalescent, and vaccinated subjects. SARS-CoV-2 specific CD8+ T cell responses were also broadly detected among all the cohorts studied. CD8+ T cell responses were detected in 90-100% of the convalescent and/or vaccinated individuals and approximately in ¼ of non-infected, non-vaccinated individuals (FIG. 4C). Similar responses were observed when plotted as SI (FIG. 4D). As observed for CD4+ T cell responses, CD8+ T cell responses of unexposed subjects (I−V−) were discriminated from all the other groups (p-values ranging 2.6e-5 to 8.8e-13) and I+V+ infected/vaccinated subjects exhibited higher responses than I+V− convalescent (p=0.03 and p=0.16 for absolute magnitude and SI respectively). Identical results were observed parsing spike-specific responses with CD8RE able to differentiate convalescent (I+V−) from unexposed and vaccinated (I-V+) subjects (p-values ranging 0.02 to 5.9e-6) and vaccinated from infected/vaccinated (I+V+) subjects (p=0.04 and p=0.02 for absolute magnitude and SI, respectively) (FIG. 4C, 4D). When the four groups were compared, no statistically significant difference in EBV reactivity was observed (FIG. 4C, 4D).

In parallel, an IFN-γ FluoroSpot assay was also employed to evaluate the CD4+ and CD8+ T cell responses using a threshold of 20 IFN γ spot forming cells (SFC) per million PBMC. Responses were detected in many infected or vaccinated individuals, and similar results were observed for Spike, CD4RE or CD8RE when considering both the absolute magnitude or stimulation index, albeit with predictably lower sensitivity and specificity than AIM.

Improved performance of the CD4RE pool based on experimentally defined epitopes. Results from both AIM and IFN γ FluoroSpot assay demonstrated that the newly developed CD4RE pool had both improved sensitivity and specificity, compared to the previously used CD4R pool of predicted epitopes. In more detail, higher positive CD4+ T cell responses in I+V− (28/30 (93%) vs 26/30 (87%), p=2.0e-4) and I+V+(28/30 (93%) vs 23/30 (77%), p=5.0e-6), and lower non-specific response in I−V− (8/30 (27%) vs 14/30 (47%), p=0.037) and I-V+(2/30 (7%) vs 4/30 (13%), p=0.031) were detected using CD4RE when compared to CD4R in the AIM assay. Similar results were shown by IFN γ FluoroSpot, assay albeit with lower sensitivity compared to AIM. These results demonstrate that the use of experimentally defined, as opposed to predicted epitopes provides higher signal in SARS-CoV-2 exposed subjects, while lowering responses from non-exposed subjects. The fact that experimentally defined epitopes yield better results is consistent with mass spectrometry studies showing the divergence of predicted from HLA-eluted SARS-CoV-2 immunopeptidome (Knierman et al., 2020; Pan et al., 2021; Weingarten-Gabbay et al., 2021).

Classification of subjects with different exposure history based on Spike and CD4RE reactivity. The inventors reasoned that unexposed (I−V−) subjects would be unreactive to experimentally defined SARS-CoV-2 peptide pools, while uninfected vaccinated (I−V+) subjects should react only to the S pool. The inventors further reasoned that infected (I+V−) subjects should recognize both S and CD4RE, but infected and vaccinated (I+V+) subjects would have a higher relative S reactivity than infected only (I+V−), as is often the case with hybrid immunity (Crotty, 2021), due to exposure to S twice, once during infection and the other during vaccination.

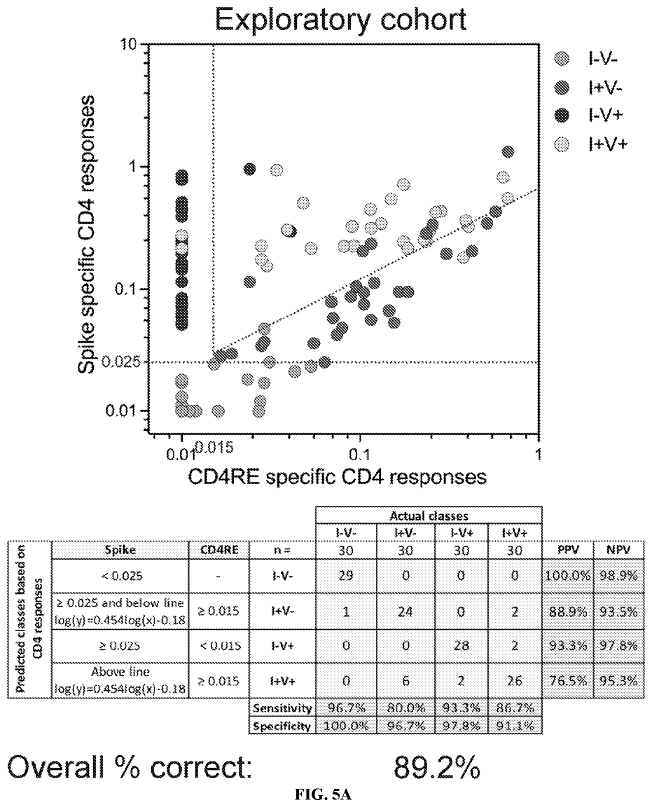

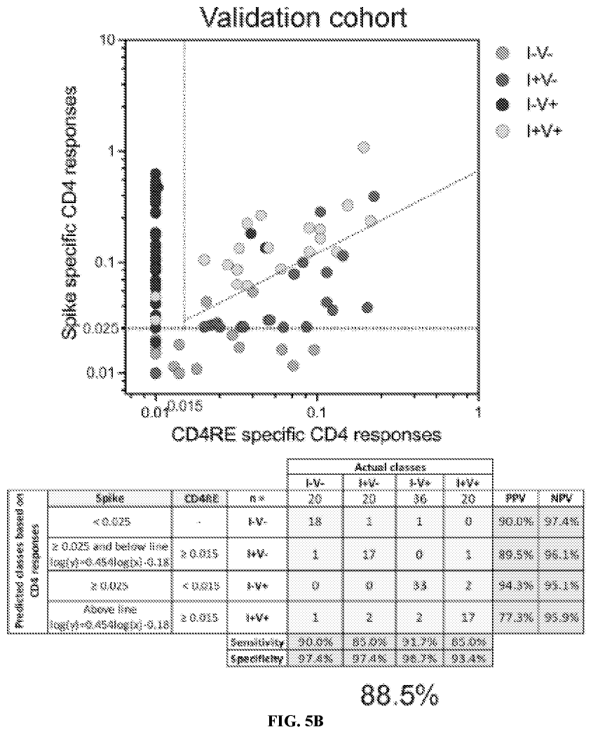

As shown in FIG. 5A, spike- and CD4RE-specific CD4+ T cell responses derived from the AIM assay were arranged in a two-dimensional plot. Each dot represents a single subject from a total of 120 donors (30 for each of the 4 groups). Optimal cutoffs were established to discriminate the four groups and the positive predictive value (PPV), negative predictive value (NPV), sensitivity and specificity were calculated for each individual group.

Subjects with spike responses lower than 0.025% were classified predictively as unexposed (I−V−) (FIG. 5A). 29 out of 29 subjects with responses matching this criterion were correctly classified (100% of PPV), while nearly all the actual I−V− subjects (29 out of 30) were found to be associated with responses below the threshold, corresponding to a sensitivity of 96.7% (FIG. 5A, first column in each box). Subjects with spike responses greater than 0.025% and CD4RE responses lower than 0.015% were classified predictively as I−V+. Twenty-eight out of 30 subjects with responses matching this threshold were correctly classified (93.3% of PPV), and 28 out of the 30 I-V+ subjects detected within this threshold (93.3% of sensitivity) (FIG. 5A, third column in each box).

Lastly, subjects with spike and CD4RE responses above 0.025% and 0.015% respectively, and above or below a diagonal line (log(y)=0.454 log(x)−0.18) were classified as I+V+ or I+V− respectively. 24 out of 27 subjects with responses matching the lower compartment (I+V−) were correctly classified (88.9% of PPV) while 24 out of the 30 I+V− subjects were found to be associated with this threshold (80% of sensitivity) (FIG. 5A, second column in each box). Conversely, the majority of subjects (26 out of 34) with responses matching the upper compartment (I+V+) were correctly classified (76.5% of PPV), while 26 out of the 30 I+V+ subjects studied were found to be associated with this threshold, corresponding to a sensitivity of 86.7% (FIG. 5A, fourth column in each box). Further statistical examinations to assess the robustness of the classification scheme as a potential diagnostic test were performed, specifically assessments of specificity and negative predictive value (PPV). High specificity and NPV were observed for each individual group with a range of 91.1-100% and 93.5-98.9% respectively (FIG. 5A). In summary, good PPV, NPV, sensitivity and specificity values were observed across all the groups with an overall classification accuracy of 89.2%.

Validation of the classifier in an independent cohort. To confirm the accuracy of this classification scheme, the inventors assessed CD4+ T cell responses in an independent validation cohort of 96 donors (20 for I−V−, I+V−, I+V+, and 36 for I−V). As shown in FIG. 5B, using the same cutoffs as described above for spike and CD4RE responses, similar PPV, NPV, sensitivity and specificity to the experimental cohort was observed across all the groups in the validation cohort with an overall classification accuracy of 88.5%. To further validate the robustness of this classification scheme, the same data (FIG. 5A to 5B) was plotted as a function of the stimulation index. Strikingly, these results paralleled the observations using the absolute magnitude, with a similar overall classification accuracy (86.7% and 85.4% for the exploratory and validation cohorts, respectively).

Applying the same classification scheme using either absolute magnitude or stimulation index for IFN γ responses yielded an overall classification accuracy of 72.5% and 60.0% respectively. A lower accuracy was observed when CD8+ T cell responses from AIM assay were analyzed, as compared to CD4+ T cell responses (data not shown). Overall, these results demonstrate the feasibility of an integrated classification scheme in assessing CD4+ T cell responses as a clinical immunodiagnostic tool. Importantly, it also displays the potential to discriminate previously undetected infection, including in vaccinated individuals.

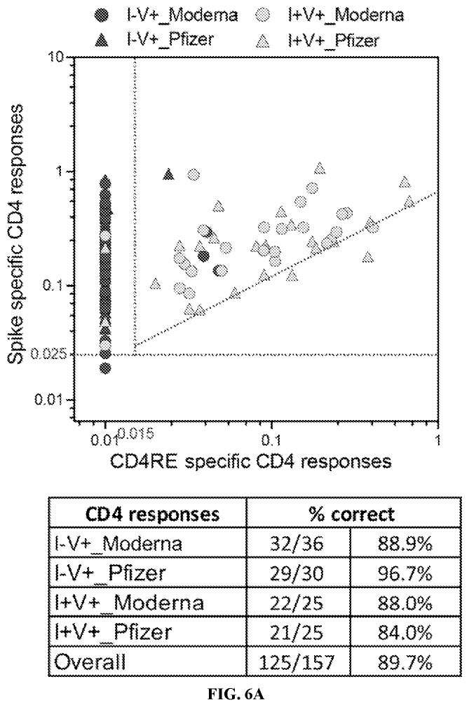

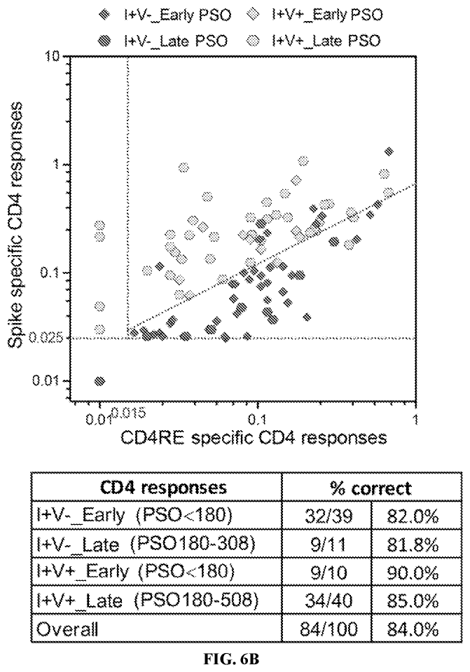

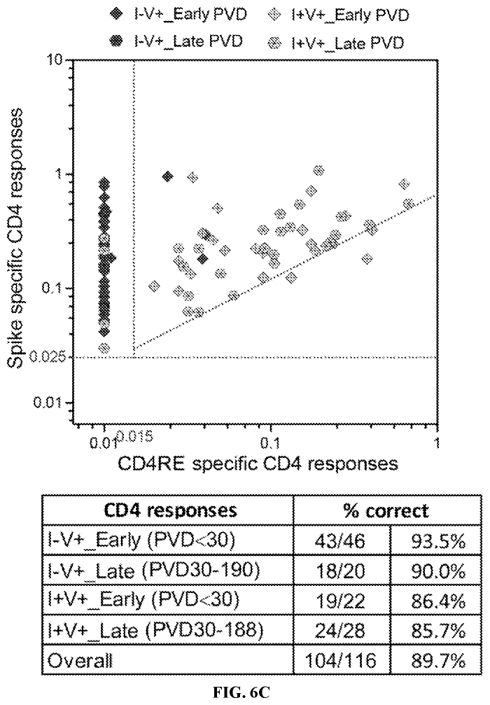

The classification scheme is applicable to different vaccine platforms, and different lengths of time post-infection/post-vaccination. To gain further insights into the applicability of the classification scheme, the inventors sought to further test and validate this tool across vaccine platforms, and longer timepoints post-symptom onset (PSO) or post-vaccination. First, the inventors looked at the response classification as a function of whether vaccinated subjects received BNT 162b2 or mRNA-1273 vaccines. As shown in FIG. 6A the overall classification accuracy when using the different mRNA vaccines was of 89.7%. Specifically, both vaccines showed similar magnitude for both total CD4+ and CD8+ T cell responses in the I−V+ or I+V+ groups (Figure S3A and B). The accuracy of the classification scheme for the different types of vaccines in the combined I-V+ or I+V+ groups was almost identical (88.5% and 90.9% for the mRNA-1273 and BNT162b2 vaccines, respectively) (Table 15).

Next, the inventors looked at the response classification as a function of the length of time PSO. The overall classification accuracy was of 84.0% (FIG. 6B). No differences were observed in the magnitude of both total CD4+ and CD8+ T cell responses between early (≤180 days) and late (>180 days) timepoints from PSO in either the I+V− or the I+V+ groups. CD4+ T cell reactivity associated with different time from PSO was also plotted as a continuous variable. The accuracy of the classification scheme when considering the different PSO timepoints was 82.0% and 81.8% in the I+V− group and 90.0% and 85.0% in the I+V+ group for the early and late timepoints, respectively (FIG. 6B).

The inventors also looked at the responses as a function of the length of time from the 2^{nd} dose of vaccination. The overall classification accuracy was of 89.7% (FIG. 6C). No differences were observed in the magnitude of both total CD4+ or CD8+ T cell responses between early (30 days) or late (>30 days) timepoints from the last dose of vaccination in either the I−V+ or the I+V+ groups. CD4+ T cell reactivity associated with different post vaccination dates was also plotted as a continuous variable. The accuracy of the classification scheme when considering the different vaccine timepoints was 93.5% and 90.0% in the I−V+ group and 86.4% and 85.7% in the I+V+ group for the early and late timepoints respectively (FIG. 6C).

Lastly, as an alternative to the T cell classification scheme, the inventors classified subjects based on spike RBD and nucleocapsid (N) antibody responses. An overall classification accuracy of 69% was observed when previously described standard clinical cutoffs were employed (Dan et al., 2021; Grifoni et al., 2020b; Tarke et al., 2021a). The attempt to classify infected individuals at late PSO timepoints resulted in even lower accuracies, consistent with reports that N positivity is relatively short lived (Dan et al., 2021; Ibarrondo et al., 2020; Ortega et al., 2021). The inventors next examined the possibility that this low classification accuracy might be reflective of suboptimal thresholds. By setting more stringent cutoffs based on the optimal classification of the exploratory cohort, the inventors achieved an overall classification accuracy of 84.2%. However, when the same classification scheme was applied to the validation cohort, the overall accuracy decreased to 52.1%, indicating that the previous value was likely a result of data overfitting. Overall, the use of antibody responses failed to yield a useful classification scheme, unlike the classification scheme using CD4+ T cell responses, which proved to be a robust tool that can accurately classify subjects regardless of the days post-infection/post-vaccination or vaccine administered.

CD4+ T cell reactivity of subjects associated with breakthrough infections. Breakthrough infections are defined as cases of previously COVID-19 vaccinated individuals associated with positive SARS-CoV-2 PCR tests (Bergwerk et al., 2021; Kustin et al., 2021; Mizrahi et al., 2021). Studies of antibody or T cell responses associated with breakthrough infection are scarce (Collier et al., 2021; Rovida et al., 2021). Breakthrough infection might be associated with increased immune responses as a result of the re-exposure (hybrid immunity) (Collier et al., 2021). In other cases, subjects experiencing breakthrough infections might be associated with general weaker immune responsiveness or decrease of vaccine effectiveness (Klompas, 2021; Mizrahi et al., 2021).

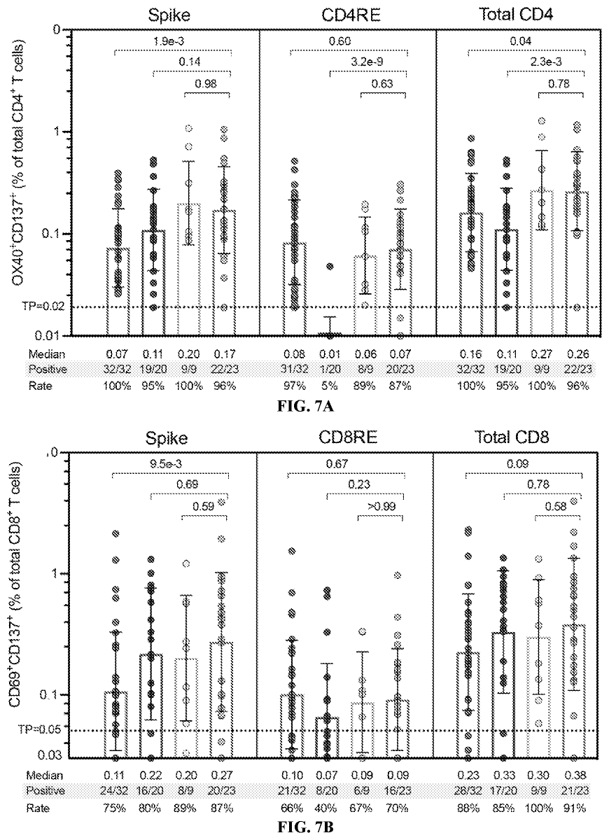

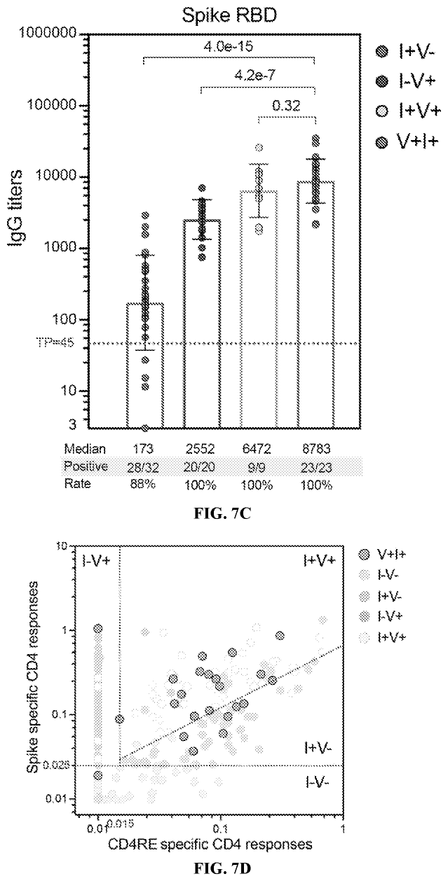

Here, the inventors assessed spike and CD4RE T cell responses in a group (n=23) of breakthrough infected individuals (V+I+). Responses were compared to the vaccinated (I-V+), infected (I+V−) or infected and then vaccinated (I+V+) groups matching the V+I+ intervals of vaccination and infection (55-271 and 18-93 days, respectively). As shown in FIG. 7A, CD4+ T cell responses from V+I+ subjects were associated with significant higher levels compared to I+V− (p=0.04) and I-V+(p=2.3e-3) subjects and similar magnitude as the I+V+ subjects. CD8+ T cell responses had comparable levels across all the groups (FIG. 7B). Similar to CD4+ T cell responses, spike RBD IgG titers from V+I+ subjects were equivalent to I+V+ subjects and significantly higher than I+V− (p=4.2e-7) and I-V+(p=4.0e-15) subjects (FIG. 7C). Thus, at the population level breakthrough infections are associated with CD4+ T cell and spike IgG responses that resemble hybrid immunity.

The classification scheme captures heterogeneity in breakthrough infections. At the level of the T cell response classification scheme, individuals who had COVID-19 were effectively segregated from non-infected groups (unexposed and vaccinated). (FIG. 7D). The inventors further expected that the V+I+ breakthrough infections would be classified in the same manner of I+V+ hybrid immunity samples. Approximately two thirds (15/23 subjects) were identified by the same thresholds associated with responses from the I+V+ group (“High responders”), while the remaining third were classified similarly to I+V− subjects (“Low responders”). No obvious difference in terms of age, PSO, PVD, disease severity or length of infection from vaccination was detected between these donors and the high responders sub-group of 15 donors.

In summary, while T cell responses following breakthrough infections (V+I+) are effectively segregated from the responses of uninfected donors (vaccinated or not) and follow the same pattern of responses of individuals vaccinated following natural infection (I+V+) in the majority of the cases, the classification scheme revealed heterogeneity in the CD4+ T cell responses of breakthrough donors.

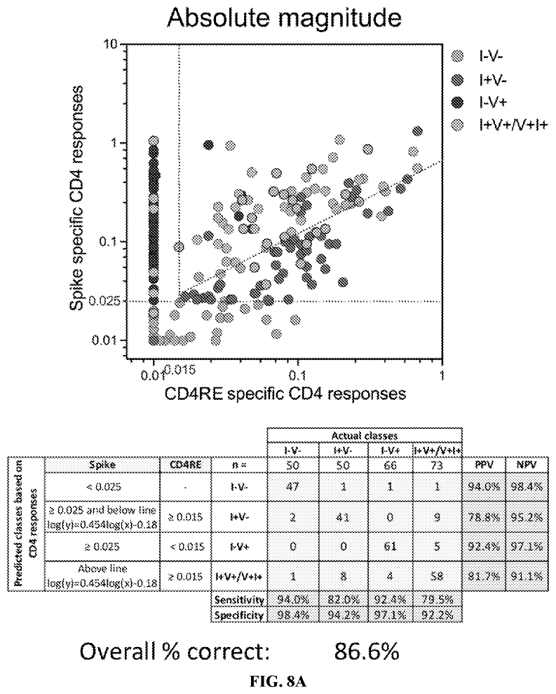

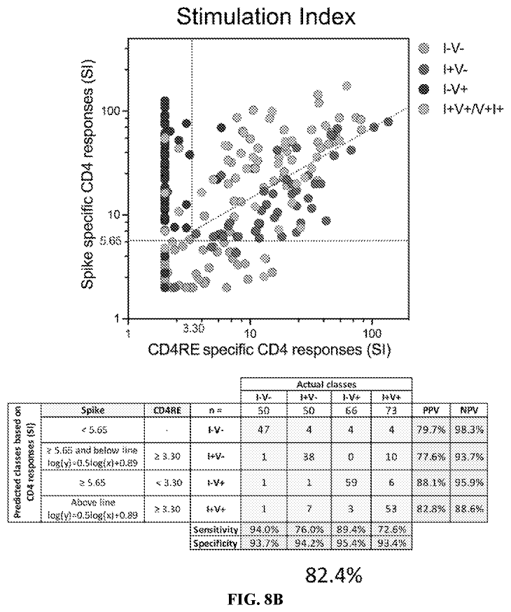

Validation of the classification scheme with whole study cohort. Finally, the inventors summarized the overall accuracy of the classification scheme across the five cohorts used in this study including breakthrough infections. For this purpose, the inventors clustered individuals that had been infected and vaccinated, irrespectively of the event that occurred first, into a single group, i.e. I+V/V+I+ (FIG. 8). When the 239 subjects with distinct COVID-19 status of infection and/or vaccination were combined, the classification scheme achieved a high overall accuracy, either as function of absolute magnitude (86.6%) or SI (82.4%). Also, high specificity and NPV were retained for each individual group with a range of 92.2-98.4% and 88.6-98.4% respectively. These results further illustrate the highly predictive power of this classification scheme and its broad clinical applicability.

There is a need to understand the roles of SARS-CoV-2 T cell responses as potential correlates of disease outcome, and/or correlates of vaccine protection from infection or severe disease. Herein, the inventors show the results of T cell quantitation based on the determination of relative activity directed against spike and the rest of the genome, by the use of optimized pools of experimentally defined epitopes (CD4RE and CD8RE). The inventors successfully classification of subjects with different COVID-19 vaccination or natural infection history in the 85-90% range of accuracy. The inventors further show that the strategy is applicable to characterizing immune responses in a group of infected vaccinees (i.e., breakthrough infections).

Although previous reports studied responses to SARS-CoV-2 in either unexposed, COVID-19 infected or vaccinated individuals (da Silva Antunes et al., 2021; Dan et al., 2021; Goel et al., 2021; Grifoni et al., 2020b; Le Bert et al., 2020; Mateus et al., 2021), this is the first demonstration, to the best of the inventors' knowledge, that a simple assay strategy can classify T-cell responses measured simultaneously in five different groups of known COVID-19 status of infection, and/or vaccination. The improved sensitivity and specificity resulted from the concept of considering the relative magnitude of responses against the spike and “rest of the genome” components, which overcomes issues related to the fact that magnitude of responses may wane over time, and also by the inclusion of experimentally defined epitopes, which the inventors show are associated with improved signal and selectivity as compared to previously utilized predicted epitopes.

The combined use of overlapping spike and CD4RE pools can be used to detect differential and relative reactivity to different SARS-CoV-2 antigens and therefore classify individuals based on SARS-CoV-2 infection and COVID-19 vaccine status, and based on this determination, can be used to inform further diagnostic or therapeutic options for said invidivual(s). More importantly, this approach allows to identify bone fide exposition to SARS-CoV-2 even in individuals that have been vaccinated and thus effectively distinguishing COVID-19 vaccine and infection history. This is of importance, as current COVID-19 diagnostic practices rely heavily on subjectively reported history, clinical records and lab modalities with imperfect performance, leading to limited reliability. For example, in longitudinal vaccination studies it will be important to monitor whether subjects enrolled in the studies might have been associated with asymptomatic infection (Kustin et al., 2021; Mizrahi et al., 2021; Pouwels et al., 2021), or even associated with abortive seronegative infections (Swadling et al., 2021). Also, diagnosis of COVID-19 past infections based on T cell reactivity could be an element considered in the context of booster vaccinations. Monitoring the differential T cell reactivity associated with vaccination versus infection might provide important information in terms of correlating T cell immunity with protection from infection and disease, in a setting where an increasingly high fraction of the general population might have been associated with both vaccination and infection. Continued monitoring of vaccine versus infection-induced T cell responses might be of interest in light of the ongoing controversy over whether vaccination protects against long COVID (Massey et al., Preprint-a; Massey et al., Preprint-b; Taquet et al., Preprint) or immunocompromised vulnerable subjects. Distinguishing T cell responses induced by vaccination versus infection might be also of interest in the context of individual COVID-19 certifications (e.g., “health passes”) and to further characterize individuals that might have been exposed but have not tested positive or had false-negative results for COVID-19 using a molecular or antigen diagnostic test. Finally, distinguishing T cell responses induced by vaccination versus infection is useful in the context of informing further therapy decisions for individuals requiring further vaccination, boosters, or other prophylactic or therapeutic anti-SARS-CoV-2 therapies or treatments as determined by their T cell response levels.

This study builds on the well-known fact that infected individuals mount a T cell response against multiple SARS-CoV-2 antigens and that individuals vaccinated with mRNA vaccines are mounting only a T cell response to Spike. A detailed classification of T cells response in different categories of vaccinated/infected individuals have not been described and compared as in the current study. Indeed, the use of the developed pools, spanning all the antigens from SARS-CoV-2, allowed for detection of SARS-CoV-2 responses with increased sensitivity and specificity compared to other studies performing T cell assays using only spike or other SARS-CoV-2 antigens (Krishna, Preprint; Kruse et al., 2021; Martinez-Gallo, 2021; Murugesan et al., 2020; Tan et al., 2021; Zelba et al., 2021).

The inventors also show that similar results were observed when relative versus absolute determinations were employed to measure T cell responses (i.e., using stimulation index or absolute magnitude), which allows for a more generalized use of the classification tool in different flow-cytometer platforms. The robustness of the T cell-based classification scheme was validated in an independent cohort exhibiting identical performances and was applicable to different types of mRNA vaccines, even when considering extended periods of time elapsed from infection and/or vaccination. T cell responses might differ according to the vaccine platform. Also, despite the wide range of time intervals following 2nd vaccine dose between groups, and even when considering extended periods of time elapsed from infection and/or vaccination, the classification scheme performance remained unchanged.

The strength of the approach is further demonstrated by the fact that T cell responses act as a better classifier than antibody responses, consistent with the notion that antibody responses to N protein are short-lived (Dan et al., 2021; Ibarrondo et al., 2020; Ortega et al., 2021). Also, while applicable to data generated by FluoroSpot cytokine assays, despite the lower intrinsic sensitivity of this assay, the inventors anticipate that this assay strategy will be broadly applicable to other readouts, such as ICS (Cohen et al., 2021; Mateus et al., 2021), and whole blood in an interferon-gamma release assay (IGRA) (Murugesan et al., 2020; Petrone et al., 2021; Tan et al., 2021).

T cell responses from breakthrough infections were also evaluated, and high levels of CD4+ and CD8+ T cell reactivity were observed. Elevated T cell responsiveness was paralleled by high levels of spike RBD IgG. Interestingly, these responses were of similar magnitude as responses from a group of individuals infected and then vaccinated (I+V+ in this study), whose features are commonly associated with hybrid immunity (Crotty, 2021). Notably, breakthrough infections were also associated with higher CD4+ T cell and spike RBD IgG responses compared to infected only or vaccinated only subjects. These results show that T and B cell reactivity associated with breakthrough infections is increased as a result of re-exposure. However, the classification tool system, also revealed significant heterogeneity in responses in some subjects, possibly linking some breakthrough infections to lower adaptive responses.

Human Subjects and PBMC isolation. The Institutional Review Boards of the University of California, San Diego (UCSD; 200236X) and the La Jolla Institute for Immunology (LJI; VD-214) approved the protocols used for blood collection for all the subjects who donated at all sites. The vast majority of the blood donations were collected through the UC San Diego Health Clinic and at the La Jolla Institute for Immunology (LJI). Additional samples were obtained from contract research organizations (CRO) under the same LJI IRB approval. All samples with the exception of the I−V− study group were collected during COVID-19 pandemic from 2020-2021. Pre-pandemic blood donations of the I−V− group were performed from 2013-2019. Each participant provided informed consent and was assigned a study identification number with clinical information recorded. Subjects who had a medical history and/or symptoms consistent with COVID-19, but lacked positive PCR-based testing for SARS-CoV-2 and subsequently had negative laboratory-based serologic testing for SARS-CoV-2, were then excluded; i.e., all COVID-19 cases in this study were confirmed cases by SARS-CoV-2 PCR or SARS-CoV-2 serodiagnostics, or both. Adults of all races, ethnicities, ages, and genders were eligible to participate, but the association of gender on the results of the study was not explicitly measured. Study exclusion criteria included lack of willingness to participate, lack of ability to provide informed consent, or a medical contraindication to blood donation (e.g., severe anemia). In all cases, PBMCs were isolated from whole blood by density gradient centrifugation according to manufacturer instructions (Ficoll-Hypaque, Amersham Biosciences, Uppsala, Sweden). Cells were cryopreserved in liquid nitrogen suspended in FBS containing 10% (vol/vol) DMSO (Sigma-Aldrich). Plasma was obtained by centrifugation (400 g for 15 minutes at 4° C.) of whole blood and collection of the upper layer, prior to PBMC isolation and cryopreserved at −80° C.

Design and production of new SARS-CoV-2 epitope pools. To study T cell responses against SARS-CoV-2, the inventors used a megapool (MP) of 15-mer peptides overlapping by 10 spanning the entire spike protein sequence (253 peptides) as previously described (Grifoni et al., 2020b). For the rest of the SARS-CoV-2 proteome, and in order to design epitope pools with increased HLA coverage and broadly recognized by demographically and geographically diverse populations, experimental defined epitopes from non-spike (R) region of SARS-CoV-2 were selected based on the recent meta-analysis (Grifoni et al., 2021). Briefly, peptides were synthesized and pooled to include both dominant (recognized in 3 or more donors/studies) and subdominant epitopes. To improve specificity, overly short or long ligands which could cause “false positive” signals (Paul et al., 2018), were excluded and only peptides of sizes ranging 15-20 and 9-10 amino acids, respectively in CD4RE and CD8RE pools were included, resulting in the generation of CD4RE and CD8RE MPs with 284 and 621 peptides, respectively. Epitopes were further classified in dominant and subdominant based on the frequency of individual responses as previously described (Grifoni et al., 2021). In addition, detailed information of the MPs composition with peptide sequences, length, ORFs of origin, and HLA coverages. Alternatively, a MP for the remainder genome consisting of dominant HLA class II predicted CD4+ T-cell epitopes (221 peptides), as previously described (Grifoni et al., 2020b) was also used as control. In addition, an EBV pool of previously reported experimental class I and class II epitopes (Carrasco Pro et al., 2015) with 301 peptides was used as positive control. All peptides were synthesized by TC peptide lab (San Diego, CA), pooled and resuspended at a final concentration of 1 mg/mL in DMSO.

SARS-CoV-2 RBD Spike and Nucleocapsid ELISAs. The SARS-CoV-2 ELISAs have been described in detail previously (Dan et al., 2021). Briefly, 96-well half-area plates (ThermoFisher 3690) were coated with 1 μg/mL of antigen and incubated at 4° C. overnight. Antigens included recombinant SARS-CoV-2 RBD protein obtained from the Saphire laboratory at LJI or recombinant nucleocapsid protein (GenScript Z03488). The next day, plates were blocked with 3% milk in phosphate-buffered saline (PBS) containing 0.05% Tween-20 for 1.5 hours at room temperature. Plasma was heat inactivated at 56° C. for 30 to 60 min. Plasma was diluted in 1% milk containing 0.05% Tween-20 in PBS starting at a 1:3 dilution followed by serial dilutions by three and incubated for 1.5 hours at room temperature. Plates were washed five times with 0.05% PBS-Tween-20. Secondary antibodies were diluted in 1% milk containing 0.05% Tween-20 in PBS. Anti-human IgG peroxidase antibody produced in goat (Sigma A6029) was used at a 1:5,000 dilution. Subsequently, plates were read on Spectramax Plate Reader at 450 nm, and data analysis was performed using SoftMax Pro. End-point titers were plotted for each sample, using background-subtracted data. Negative and positive controls were used to standardize each assay and normalize across experiments. Limit of detection (LOD) was defined as 1:3 of IgG. Spike RBD IgG or nucleocapsid IgG thresholds of positivity (TP) for SARS-CoV-2 infected or COVID-19 vaccinated individuals were established based on uninfected and unvaccinated subjects (I−V−).

Activation induced cell marker (AIM) assay. The AIM assay was performed as previously described (Mateus et al., 2020). Cryopreserved PBMCs were thawed by diluting the cells in 10 mL complete RPMI 1640 with 5% human AB serum (Gemini Bioproducts) in the presence of benzonase [20 ml/10 ml]. Cells were cultured for 20 to 24 hours in the presence of SARS-CoV-2 specific and EBV pools (1 ug/ml) in 96-wells U bottom plates with 1×10⁶ PBMC per well. An equimolar amount of DMSO was added as a negative control and phytohemagglutinin (PHA, Roche (San Diego, CA) 1 mg/ml) was used as the positive control. The cells were stained with CD3 AF532, CD4 BV605, CD8 BUV496, and Live/Dead Aqua. Activation was measured by the following markers: CD137 APC, OX40 PE-Cy7, and CD69 PE. All samples were acquired on a ZE5 cell analyzer (Biorad laboratories, Hercules, CA) and analyzed with FlowJo software (Tree Star, Ashland, OR).

CD4+ and CD8+ T cells responses were calculated as percent of total CD4+ (OX40+CD137+) or CD8+ (CD69+CD137+) T cells. The background was removed from the data by subtracting the wells stimulated with DMSO. The Stimulation Index (SI) was calculated by dividing the counts of AIM+ cells after SARS-CoV-2 pools stimulation with the ones in the negative control. A positive response was defined as SI≥2 and AIM⁺ response above the threshold of positivity after background subtraction. The limit of detection (0.01% and 0.03 for CD4+ and CD8+ T cells, respectively) was calculated based on 2 times 95% CI of geomean of negative control (DMSO), and the threshold of positivity (0.02% for CD4+ and 0.05% for CD8+ T cells) was calculated based on 2 times standard deviation of background signals according to previous published studies (Dan et al., 2021; Mateus et al., 2020).

IFN γ FluoroSpot assay. The FluoroSpot assay was performed as previously described (Tarke et al., 2021a). PBMCs derived from 80 subjects from 4 clinical cohorts (20 each for I−V−, I+V−, I-V+, and I+V+ cohorts) were stimulated in triplicate at a single density of 2×10⁵ cells/well. The cells were stimulated with the different MPs analyzed (1 ug/mL), PHA (10 mg/mL), and DMSO (0.1%) in 96-well plates previously coated with anti-cytokine antibodies for IFN γ, (mAbs 1-D1K; Mabtech, Stockholm, Sweden) at a concentration of 10 ug/mL. After 20-24 hours of incubation at 37° C., 5% CO2, cells were discarded and FluoroSpot plates were washed and further incubated for 2 hours with cytokine antibodies (mAbs 7-B6-1-BAM; Mabtech, Stockholm, Sweden). Subsequently, plates were washed again with PBS/0.05% Tween20 and incubated for 1 hour with fluorophore-conjugated antibodies (Anti-BAM-490). Computer-assisted image analysis was performed by counting fluorescent spots using an AID iSPOT FluoroSpot reader (AIS-diagnostika, Germany). Each megapool was considered positive compared to the background based on the following three criteria: 20 or more IFN γ spot forming cells (SFC) per 10⁶ PBMC after background subtraction (Threshold defined as 2 times standard deviation of background signals), a stimulation index (SI) greater than 2, and statistically different from the background (p<0.05) in either a Poisson or t test as previously described (Oseroff et al., 2005).

Statistical Analysis. Experimental data were analyzed by GraphPad Prism Version 9 (La Jolla, CA) and Microsoft Excel Version 16.16.27 (Microsoft, Redmond, WA). The statistical details of the experiments are provided in the respective figure legends. Data were analyzed by Wilcoxon test (two-tailed) to compare between two paired groups, and Kruskal-Wallis test adjusted with Dunn's test for multiple comparisons to compare between multiple groups. Data were plotted as geometric mean with geometric SD. p values<0.05 (after adjustment if indicated) were considered statistically significant. For the classification scheme, statistical determinations and metrics were executed as previously described (Trevethan, 2017). Briefly, for each individual group the following calculations were performed: 1) positive predictive value (PPV)=(True Positives)/(True Positives+False Positives); 2) negative predictive value (NPV)=(True Negatives)/(True Negatives+False Negatives); 3) sensitivity=(True Positives)/(True Positives+False Negatives); and 4) specificity=(True Negatives)/(True Negatives+False Positives).

Study Approval. This study was approved by the Human Subjects Protection Program of the UC San Diego Health under IRB approved protocols (UCSD; 200236X), or under IRB approval (LJI; VD-214) at the La Jolla Institute for Immunology. All donors were able to provide informed consent, or had a legal guardian or representative able to do so. Each participant provided informed consent and was assigned a study identification number with clinical information recorded.

The present inventors recognized that defining a comprehensive set of epitope specificities is important for several reasons. First, it allows the determination of whether within different SARS-CoV-2 antigens certain regions are immunodominant. This will be important for vaccine design, so as to ensure that vaccine constructs include not only regions targeted by neutralizing antibodies, such as the receptor binding domain (RBD) in the spike (S) region, but also include regions capable of delivering sufficient T cell help and are suitable targets of CD4+ T cell activity. Second, a comprehensive set of epitopes helps define the breadth of responses, in terms of the average number of different CD4+ and CD8+ T cell SARS-CoV-2 epitopes generally recognized by each individual. This is key because some reports have described a T cell repertoire focused on few viral epitopes (Ferretti et al., 2020), which would be concerning for potential viral escape from immune recognition via accumulated mutations that can occur during replication or through viral reassortment. Third, a comprehensive survey of epitopes restricted by a set of different HLAs representative of the diversity present in the general population is important to ensure that results obtained are generally applicable across different ethnicities and racial groups, and also to lay the foundations to examine the potential associations of certain HLAs with COVID-19 severity. Finally, the definition of the epitopes recognized in SARS-CoV-2 infection is relevant in the context of the debate on the potential influence of SARS-CoV-2 cross-reactivity with endemic “Common Cold” Coronaviruses (CCC) (Braun et al., 2020; Le Bert et al., 2020). Several studies have defined the repertoire of SARS-CoV-2 epitopes recognized in unexposed individuals (Braun et al., 2020; Mateus et al., 2020; Nelde et al., 2020), but the correspondence between that repertoire and the epitope repertoire elicited by SARS-CoV-2 infection has not been previously evaluated.

The present inventors provide a comprehensive map of epitopes recognized by CD4+ and CD8+ T cell responses across the entire SARS-CoV-2 viral proteome. Importantly, these epitopes have been characterized in the context of a broad set of HLA alleles using a direct ex vivo, cytokine-independent, approach.

The present inventors used a combined experimental and bioinformatics approach to address T cell reactivity to SARS-CoV-2 VOCs. T cell responses from persons recovered from COVID-19 were directly assessed, and T cell responses from recent Moderna mRNA-1273 or Pfizer/BioNTech BNT162b2 vaccinees, for their capacity to recognize peptides derived from the ancestral reference sequence and the B.1.1.7, B1.351, P.1 and CAL.20C variants. As a complementary approach, bioinformatic analyses were used to predict the impact of mutations in the VOCs with sets of previously reported CD4⁺ and CD8⁺ T cell epitopes derived from the ancestral reference sequence.

The present disclosure describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in Tables 1 to 10 (SEQ ID NOS: 1 to 3522) or polynucleotides encoding the same, covering different HLA Class II and/or MHC Class I alleles, capable of generating a treatment acting broadly on a population level are disclosed herein. As uses throughout the specification when referring to the use peptide epitopes, the composition can comprise or express 1, 2, 3, 4, 5, 6, 7, 8, 9, 10, 11, 12, 13, 14, 15, 16, 17, 18, 19, 20, 20, 25, 30, 40, 50, 60, 70, 75, 89, 90, 100, 110, 120, 125, 130, 140, 150, 160, 179, 175, 180, 190, 200, 225, 250, 275, 300, 325, or 350, 400, 450, 500, 600, 700, 750, 800, 900, 1000, 1250, 15,500, 1,750, 2000, 2,250, 2,500, 2,750, 3,000, 3,250, 3,500, or 3522 peptides. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

The present disclosure further provides the following methods:

A method for monitoring an immune response relevant to a coronavirus infection comprising one or more steps of:
i) providing one or more MHC/peptide multimers or a composition comprising at least one MHC/peptide multimer according to the disclosure,
ii) providing a sample comprising a population of T cells, and
iii) measuring the presence, frequency, number, activity and/or state of T cells specific for said one or more MHC/peptide multimers,
   thereby monitoring said immune response relevant to a coronavirus infection.
   A method for diagnosing a coronavirus infection comprising one or more steps of:
i) providing one or more MHC/peptide multimers or a composition comprising at least one MHC/peptide multimer according to the disclosure,
ii) providing a sample comprising a population of T cells, and
iii) measuring the presence, frequency, number, activity and/or state of T cells specific for said one or more MHC/peptide multimers,
   thereby diagnosing said coronavirus infection.
   A method for isolation of one or more antigen-specific T cells, said method comprising one or more steps of
i) providing a sample comprising a population of T cells,
ii) providing one or more MHC/peptide multimers or a composition comprising at least one MHC/peptide multimer according to the disclosure,
iii) contacting said MHC/peptide multimers or composition with said sample comprising a population of T cells, and
iv) isolating T cells specific for said MHC/peptide multimers or composition.
   A method for detecting an antigen-specific T cell response comprising one or more steps of:
i) providing a sample comprising a population of T cells,
ii) providing one or more MHC/peptide multimers or a composition comprising at least one MHC/peptide multimer according to the disclosure,
iii) contacting said MHC/peptide multimers or composition with said sample, and
iv) measuring the presence, frequency, number, activity and/or state of T cells specific for said MHC/peptide multimers or composition, thereby detecting said antigen-specific T cell response.

The present disclosure provides improved epitope or peptide combinations for modulating an immune response, for treating a subject for an infection or aberrant immune response, and for use in diagnostic methods and kits comprising such peptide combinations. It is another object of the disclosure to provide epitope or peptide combinations exhibiting very good HLA Class I and Class II coverage in a worldwide population and being immunologically potent in a worldwide population. It is another object of the disclosure to provide epitope or peptide combinations having good cross reactivity to other viral strains, including co-circulating strains (for example, mutants) of coronaviruses, including SARS-CoV-2, common cold coronaviruses, as well as SARS-CoV, MERS, etc. It is another object of the disclosure to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a SARS-CoV-2 protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the disclosure is to provide epitope combinations which are so immunologically potent that even at very low doses of epitopes, the percentage of responding donors can be retained at a very high level in a donor cohort representative of a worldwide population. Another object of the disclosure is to provide epitope combinations which have minor risk of inducing IgE-mediated adverse events. An additional object of the disclosure is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the disclosure is to select epitopes for use in the compositions and methods described herein, based on one or both of their immunodominance or immunoprevalence. A still further object of the disclosure is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a coronavirus, including methods and compositions of detecting an immune response or immune cells relevant to a coronavirus infection. These methods and compositions include vaccines, diagnostics, therapies, reagents and kits, for modulating, eliciting, or detecting T cells responsive to one or more coronavirus peptides or proteins. The proteins and peptides described herein comprise, consist of, or consist essentially of: one or more amino acid sequences selected from those sequences set forth in Tables 1 to 10 (SEQ ID NOS: 1 to 3522), or a subsequence, portion, homologue, variant or derivative thereof; a fusion protein comprising one or more amino acid sequences selected from those sequences set forth in Tables 1 to 10 (SEQ ID NOS: 1 to 3522); a pool of 2 or more peptides selected from the amino acid sequences set forth in Tables 1 to 10 (SEQ ID NOS: 1 to 3522), or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof. In certain preferred embodiments, the coronavirus is one or more of SARS-CoV-2 or a variant thereof, or SARS, MERS, or a common cold coronavirus strain (e.g., 229E, NL63, HKU1, OC43). Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the disclosure.

The present invention also includes a method of distinguishing an immune response from a subject that has been vaccinated but not exposed to COVID, or the subject was exposed to COVID but not vaccinated, the method comprising, consisting of, or consisting essentially of: contacting a biological sample from a subject with a composition of any one of claims a fusion protein comprising one or more amino acid sequences selected from those sequences set forth in Tables 1 to 10 (SEQ ID NOS: 1 to 3522); or a pool of 2 or more peptides comprising, consisting of, or consisting essentially of amino acid sequences selected from those sequences set forth in Tables 1 to 10 (SEQ ID NOS: 1 to 3522); or a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof; and determining if the composition elicits an immune response from the contacted cells, wherein the presence of an immune response indicates that the subject has been exposed to or infected with SARS-CoV-2 by determining that the immune response is predominantly to a Spike protein, or is to one or more viral antigens other than the Spike protein, wherein a predominant response to Spike protein is indicative that a subject has been vaccinated, or if the response is to one or more viral antigens other than the Spike protein then the subject has been exposed to SARS-CoV-2 but not vaccinated, wherein the peptide or peptides include amino acid sequences set forth in Tables 1 to 10 (SEQ ID NOS: 1 to 3522). The method comprises determining whether the subject has been infected by or exposed to SARS-CoV-2 more than once by determining if the subject elicits a secondary T cell immune response profile that is different from a primary T cell immune response profile. The method further comprises diagnosing a SARS-CoV-2 infection or exposure in a subject, the method comprising contacting a biological sample from a subject with a composition described hereinabove; and determining if the composition elicits a T cell immune response, wherein the T cell immune response identifies that the subject has been infected with or exposed to SARS-CoV-2. The method can be conducted three or more days following the date of suspected infection by or exposure to a coronavirus.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### References—Example 1 TO 16

1. DiPiazza A T, Graham B S, Ruckwardt T J. T cell immunity to SARS-CoV-2 following natural infection and vaccination. Biochem Biophys Res Commun. 2020.
2. Jordan S C. Innate and Adaptive Immune Responses to SARS-CoV-2 in Humans: Relevance to Acquired Immunity and Vaccine Responses. Clin Exp Immunol. 2021.
3. Karlsson A C, Humbert M, Buggert M. The known unknowns of T cell immunity to COVID-19. Sci Immunol. 2020; 5(53).
4. Sette A, Crotty S. Pre-existing immunity to SARS-CoV-2: the knowns and unknowns. Nat Rev Immunol. 2020; 20(8):457-8.
5. Sette A, Crotty S. Adaptive immunity to SARS-CoV-2 and COVID-19. Cell. 2021.
6. Swadling L, Maini M K. T cells in COVID-19 united in diversity. Nature Immunology. 2020; 21(11):1307-8.
7. Chen Z, Ruan P, Wang L, Nie X, Ma X, Tan Y. T and B cell Epitope analysis of SARS-CoV-2 S protein based on immunoinformatics and experimental research. J Cell Mol Med. 2021; 25(2):1274-89.
8. Ferretti A P, Kula T, Wang Y, Nguyen D M V, Weinheimer A, Dunlap G S, et al. Unbiased Screens Show CD8(+) T Cells of COVID-19 Patients Recognize Shared Epitopes in SARS-CoV-2 that Largely Reside outside the Spike Protein. Immunity. 2020; 53(5):1095-107 e3.
9. Gangaev A, Ketelaars S L C, Isaeva O I, Patiwael S, Dopler A, Hoefakker K, et al. Identification and characterization of an immunodominant SARS-CoV-2-specific CD8 T cell response. Journal for ImmunoTherapy of Cancer. 2020; 8(Suppl 3):A513-A4.
10. Habel J R, Nguyen T H O, van de Sandt C E, Juno J A, Chaurasia P, Wragg K, et al. Suboptimal SARS-CoV-2-specific CD8(+) T cell response associated with the prominent HLA-A*02:01 phenotype. Proc Natl Acad Sci USA. 2020; 117(39):24384-91.
11. Joag V, Wijeyesinghe S, Stolley J M, Quarnstrom C F, Dileepan T, Soerens A G, et al. Cutting Edge: Mouse SARS-CoV-2 Epitope Reveals Infection and Vaccine-Elicited CD8 T Cell Responses. J Immunol. 2021.
12. Kared H, Redd A D, Bloch E M, Bonny T S, Sumatoh H R, Kairi F, et al. SARS-CoV-2-specific CD8+ T cell responses in convalescent COVID-19 individuals. J Clin Invest. 2021.
13. Keller M D, Harris K M, Jensen-Wachspress M A, Kankate V, Lang H, Lazarski C A, et al. SARS-CoV-2 specific T-cells Are Rapidly Expanded for Therapeutic Use and Target Conserved Regions of Membrane Protein. Blood. 2020.
14. Le Bert N, Clapham H E, Tan A T, Chia W N, Tham C Y L, Lim J M, et al. Highly functional virus-specific cellular immune response in asymptomatic SARS-CoV-2 infection. J Exp Med. 2021; 218(5).
15. Le Bert N, Clapham H E, Tan A T, Chia W N, Tham C Y L, Lim J M, et al. Highly functional virus-specific cellular immune response in asymptomatic SARS-CoV-2 infection. bioRxiv. 2020.
16. Le Bert N, Tan A T, Kunasegaran K, Tham C Y L, Hafezi M, Chia A, et al. SARS-CoV-2-specific T cell immunity in cases of COVID-19 and SARS, and uninfected controls. Nature. 2020; 584(7821):457-62.
17. Lee E, Sandgren K, Duette G, Stylianou V V, Khanna R, Eden J S, et al. Identification of SARS-CoV-2 Nucleocapsid and Spike T-cell Epitopes for Assessing T-cell Immunity. J Virol. 2020.
18. Mahajan S, Kode V, Bhojak K, Magdalene C M, Lee K, Manoharan M, et al. Immunodominant T-cell epitopes from the SARS-CoV-2 spike antigen reveal robust pre-existing T-cell immunity in unexposed individuals. bioRxiv. 2020:2020.11.03.367375.
19. Mateus J, Grifoni A, Tarke A, Sidney J, Ramirez S I, Dan J M, et al. Selective and cross-reactive SARS-CoV-2 T cell epitopes in unexposed humans. Science. 2020; 370(6512):89-94.
20. Nelde A, Bilich T, Heitmann J S, Maringer Y, Salih H R, Roerden M, et al. SARS-CoV-2-derived peptides define heterologous and COVID-19-induced T cell recognition. Nat Immunol. 2021; 22(1):74-85.
21. Nielsen S S, Vibholm L K, Monrad I, Olesen R, Frattari G S, Pahus M H, et al. SARS-CoV-2 Elicits Robust Adaptive Immune Responses Regardless of Disease Severity. bioRxiv. 2020:2020.10.08.331645.
22. Peng Y, Mentzer A J, Liu G, Yao X, Yin Z, Dong D, et al. Broad and strong memory CD4(+) and CD8(+) T cells induced by SARS-CoV-2 in UK convalescent individuals following COVID-19. Nat Immunol. 2020; 21(11):1336-45.
23. Poran A, Harjanto D, Malloy M, Rooney M S, Srinivasan L, Gaynor R B. Sequence-based prediction of vaccine targets for inducing T cell responses to SARS-CoV-2 utilizing the bioinformatics predictor RECON. bioRxiv. 2020:2020.04.06.027805.
24. Poran A, Harjanto D, Malloy M, Arieta C M, Rothenberg D A, Lenkala D, et al. Sequence-based prediction of SARS-CoV-2 vaccine targets using a mass spectrometry-based bioinformatics predictor identifies immunogenic T cell epitopes. Genome medicine. 2020; 12(1):70.
25. Prakash S, Srivastava R, Coulon P-G, Dhanushkodi N R, Chentoufi A A, Tifrea D F, et al. Genome-Wide Asymptomatic B-Cell, CD4<sup>+</sup> and CD8<sup>+</sup> T-Cell Epitopes, that are Highly Conserved Between Human and Animal Coronaviruses, Identified from SARS-CoV-2 as Immune Targets for Pre-Emptive Pan-Coronavirus Vaccines. bioRxiv. 2020:2020.09.27.316018.
26. Rha M S, Jeong H W, Ko J H, Choi S J, Seo I H, Lee J S, et al. PD-1-Expressing SARS-CoV-2-Specific CD8(+) T Cells Are Not Exhausted, but Functional in Patients with COVID-19. Immunity. 2021; 54(1):44-52 e3.
27. Sahin U, Muik A, Vogler I, Derhovanessian E, Kranz L M, Vormehr M, et al. BNT162b2 induces SARS-CoV-2-neutralising antibodies and T cells in humans. medRxiv. 2020.
28. Saini S K, Hersby D S, Tamhane T, Povlsen H R, Hernandez S P A, Nielsen M, et al. SARS-CoV-2 genome-wide mapping of CD8 T cell recognition reveals strong immunodominance and substantial CD8 T cell activation in COVID-19 patients. bioRxiv. 2020:2020.10.19.344911.
29. Saini S K, Hersby D S, Tamhane T, Povlsen H R, Amaya Hernandez S P, Nielsen M, et al. SARS-CoV-2 genome-wide T cell epitope mapping reveals immunodominance and substantial CD8(+) T cell activation in COVID-19 patients. Sci Immunol. 2021; 6(58).
30. Schulien I, Kemming J, Oberhardt V, Wild K, Seidel L M, Killmer S, et al. Characterization of pre-existing and induced SARS-CoV-2-specific CD8(+) T cells. Nat Med. 2021; 27(1):78-85.
31. Sekine T, Perez-Potti A, Rivera-Ballesteros O, Stralin K, Gorin J B, Olsson A, et al. Robust T Cell Immunity in Convalescent Individuals with Asymptomatic or Mild COVID-19. Cell. 2020; 183(1):158-68 e14.
32. Shomuradova A S, Vagida M S, Sheetikov S A, Zornikova K V, Kiryukhin D, Titov A, et al. SARS-CoV-2 Epitopes Are Recognized by a Public and Diverse Repertoire of Human T Cell Receptors. Immunity. 2020; 53(6):1245-57 e5.
33. Snyder™, Gittelman R M, Klinger M, May D H, Osborne E J, Taniguchi R, et al. Magnitude and Dynamics of the T-Cell Response to SARS-CoV-2 Infection at Both Individual and Population Levels. medRxiv. 2020:2020.07.31.20165647.
34. Tarke A, Sidney J, Kidd C K, Dan J M, Ramirez S I, Yu E D, et al. Comprehensive analysis of T cell immunodominance and immunoprevalence of SARS-CoV-2 epitopes in COVID-19 cases. Cell Rep Med. 2021:100204.
35. Fleri W, Vaughan K, Salimi N, Vita R, Peters B, Sette A. The Immune Epitope Database: How Data Are Entered and Retrieved. Journal of immunology research. 2017; 2017:5974574.
36. Salimi N, Fleri W, Peters B, Sette A. The immune epitope database: a historical retrospective of the first decade. Immunology. 2012; 137(2):117-23.
37. Hassert M, Geerling E, Stone E T, Steffen T L, Feldman M S, Dickson A L, et al. mRNA induced expression of human angiotensin-converting enzyme 2 in mice for the study of the adaptive immune response to severe acute respiratory syndrome coronavirus 2. PLoS Pathog. 2020; 16(12):e1009163.
38. Takagi A, Matsui M. Identification of HLA-A*02:01-Restricted Candidate Epitopes Derived from the Nonstructural Polyprotein 1a of SARS-CoV-2 That May Be Natural Targets of CD8<sup>+</sup> T Cell Recognition <em>In Vivo</em>. Journal of Virology. 2021; 95(5):e01837-20.
39. Knierman M D, Lannan M B, Spindler L J, McMillian C L, Konrad R J, Siegel R W. The Human Leukocyte Antigen Class II Immunopeptidome of the SARS-CoV-2 Spike Glycoprotein. Cell Rep. 2020; 33(9):108454.
40. Parker R, Partridge T, Wormald C, Kawahara R, Stalls V, Aggelakopoulou M, et al. Mapping the SARS-CoV-2 spike glycoprotein-derived peptidome presented by HLA class II on dendritic cells. bioRxiv. 2020.
41. Weingarten-Gabbay S, Klaeger S, Sarkizova S, Pearlman L R, Chen D Y, Bauer M R, et al. SARS-CoV-2 infected cells present HLA-I peptides from canonical and out-of-frame ORFs. bioRxiv. 2020.
42. Prachar M, Justesen S, Steen-Jensen D B, Thorgrimsen S, Jurgons E, Winther O, et al. Identification and validation of 174 COVID-19 vaccine candidate epitopes reveals low performance of common epitope prediction tools. Sci Rep. 2020; 10(1):20465.
43. Murphey K, Paul Travers, Mark Walport, and Charles Janeway. Janeway's Immunobiology. 8th ed. New York: Garland Science; 2012 2012.
44. Abbas Ak, Andrew H Lichtman, and Shiv Pillai. Cellular and Molecular Immunology. 6 th ed. Piladelphia: Saunders Elsevier; 2007.
45. Petersen J, Purcell A W, Rossjohn J. Post-translationally modified T cell epitopes: immune recognition and immunotherapy. J Mol Med (Berl). 2009; 87(11):1045-51.
46. Sun L, Middleton D R, Wantuch P L, Ozdilek A, Avci F Y. Carbohydrates as T-cell antigens with implications in health and disease. Glycobiology. 2016; 26(10):1029-40.
47. McMichael A J, Gotch F M, Santos-Aguado J, Strominger J L. Effect of mutations and variations of HLA-A2 on recognition of a virus peptide epitope by cytotoxic T lymphocytes. Proc Natl Acad Sci USA. 1988; 85(23):9194-8.
48. O'Sullivan D, Arrhenius T, Sidney J, Del Guercio M F, Albertson M, Wall M, et al. On the interaction of promiscuous antigenic peptides with different D R alleles. Identification of common structural motifs. J Immunol. 1991; 147(8):2663-9.
49. Greenbaum J, Sidney J, Chung J, Brander C, Peters B, Sette A. Functional classification of class II human leukocyte antigen (HLA) molecules reveals seven different supertypes and a surprising degree of repertoire sharing across supertypes. Immunogenetics. 2011; 63(6):325-35.
50. Sidney J, Peters B, Frahm N, Brander C, Sette A. HLA class I supertypes: a revised and updated classification. BMC Immunol. 2008; 9:1.
51. Kilgus J, Jardetzky T, Gorga J C, Trzeciak A, Gillessen D, Sinigaglia F. Analysis of the permissive association of a malaria T cell epitope with D R molecules. J Immunol. 1991; 146(1):307-15.
52. Panina-Bordignon P, Tan A, Termijtelen A, Demotz S, Corradin G, Lanzavecchia A. Universally immunogenic T cell epitopes: promiscuous binding to human MHC class II and promiscuous recognition by T cells. Eur J Immunol. 1989; 19(12):2237-42.
53. Dash P, Fiore-Gartland A J, Hertz T, Wang G C, Sharma S, Souquette A, et al. Quantifiable predictive features define epitope-specific T cell receptor repertoires. Nature. 2017; 547(7661):89-93.
54. Glanville J, Huang H, Nau A, Hatton 0, Wagar L E, Rubelt F, et al. Identifying specificity groups in the T cell receptor repertoire. Nature. 2017; 547(7661):94-8.
55. Sidney J, Peters B, Sette A. Epitope prediction and identification-adaptive T cell responses in humans. Semin Immunol. 2020; 50:101418.
56. Yewdell J W, Bennink J R. Immunodominance in major histocompatibility complex class I-restricted T lymphocyte responses. Annu Rev Immunol. 1999; 17:51-88.
57. Oseroff C, Peters B, Pasquetto V, Moutaftsi M, Sidney J, Panchanathan V, et al. Dissociation between epitope hierarchy and immunoprevalence in CD8 responses to vaccinia virus western reserve. J Immunol. 2008; 180(11):7193-202.
58. Tan A T, Sodsai P, Chia A, Moreau E, Chng M H, Tham C Y, et al. Immunoprevalence and immunodominance of HLA-Cw*0801-restricted T cell response targeting the hepatitis B virus envelope transmembrane region. J Virol. 2014; 88(2):1332-41.
59. Wang X F, Kerzerho J, Adotevi O, Nuyttens H, Badoual C, Munier G, et al. Comprehensive analysis of HLA-D R- and HLA-DP4-restricted CD4+ T cell response specific for the tumor-shared antigen survivin in healthy donors and cancer patients. J Immunol. 2008; 181(1):431-9.
60. Assarsson E, Sidney J, Oseroff C, Pasquetto V, Bui H H, Frahm N, et al. A quantitative analysis of the variables affecting the repertoire of T cell specificities recognized after vaccinia virus infection. J Immunol. 2007; 178(12):7890-901.
61. Kotturi M F, Peters B, Buendia-Laysa F, Jr., Sidney J, Oseroff C, Botten J, et al. The CD8+ T-cell response to lymphocytic choriomeningitis virus involves the L antigen: uncovering new tricks for an old virus. J Virol. 2007; 81(10):4928-40.
62. Yewdell J W. Confronting complexity: real-world immunodominance in antiviral CD8+ T cell responses. Immunity. 2006; 25(4):533-43.
63. Hataye J, Moon J J, Khoruts A, Reilly C, Jenkins M K. Naive and memory CD4+ T cell survival controlled by clonal abundance. Science. 2006; 312(5770):114-6.
64. Kotturi M F, Scott I, Wolfe T, Peters B, Sidney J, Cheroutre H, et al. Naive precursor frequencies and MHC binding rather than the degree of epitope diversity shape CD8+ T cell immunodominance. J Immunol. 2008; 181(3):2124-33.
65. Croft N P, Smith S A, Pickering J, Sidney J, Peters B, Faridi P, et al. Most viral peptides displayed by class I MHC on infected cells are immunogenic. Proc Natl Acad Sci USA. 2019; 116(8):3112-7.
66. Paul S, Croft N P, Purcell A W, Tscharke D C, Sette A, Nielsen M, et al. Benchmarking predictions of MHC class I restricted T cell epitopes in a comprehensively studied model system. PLoS Comput Biol. 2020; 16(5):e1007757.
67. Demotz S, Grey H M, Sette A. The minimal number of class II MHC-antigen complexes needed for T cell activation. Science. 1990; 249(4972):1028-30.
68. Sykulev Y, Joo M, Vturina I, Tsomides T J, Eisen H N. Evidence that a single peptide-MHC complex on a target cell can elicit a cytolytic T cell response. Immunity. 1996; 4(6):565-71.
69. Peters B, Nielsen M, Sette A. T Cell Epitope Predictions. Annu Rev Immunol. 2020; 38:123-45.
70. Riddell S R, Greenberg P D. Principles for adoptive T cell therapy of human viral diseases. Annu Rev Immunol. 1995; 13:545-86.
71. Grifoni A, Weiskopf D, Ramirez S I, Mateus J, Dan J M, Moderbacher C R, et al. Targets of T Cell Responses to SARS-CoV-2 Coronavirus in Humans with COVID-19 Disease and Unexposed Individuals. Cell. 2020; 181(7):1489-501 e15.
72. Masopust D, Soerens A G. Tissue-Resident T Cells and Other Resident Leukocytes. Annu Rev Immunol. 2019; 37:521-46.
73. Dan J M, Lindestam Arlehamn C S, Weiskopf D, da Silva Antunes R, Havenar-Daughton C, Reiss S M, et al. A Cytokine-Independent Approach To Identify Antigen-Specific Human Germinal Center T Follicular Helper Cells and Rare Antigen-Specific CD4+ T Cells in Blood. J Immunol. 2016; 197(3):983-93.
74. Locci M, Havenar-Daughton C, Landais E, Wu J, Kroenke M A, Arlehamn C L, et al. Human circulating PD-1+CXCR3-CXCR5+ memory Tfh cells are highly functional and correlate with broadly neutralizing HIV antibody responses. Immunity. 2013; 39(4):758-69.
75. Reiss S, Baxter A E, Cirelli K M, Dan J M, Morou A, Daigneault A, et al. Comparative analysis of activation induced marker (AIM) assays for sensitive identification of antigen-specific CD4 T cells. PLoS One. 2017; 12(10):e0186998.
76. Dhanda S K, Vaughan K, Schulten V, Grifoni A, Weiskopf D, Sidney J, et al. Development of a novel clustering tool for linear peptide sequences. Immunology. 2018; 155(3):331-45.
77. Paul S, Karosiene E, Dhanda S K, Jurtz V, Edwards L, Nielsen M, et al. Determination of a Predictive Cleavage Motif for Eluted Major Histocompatibility Complex Class II Ligands. Frontiers in immunology. 2018; 9:1795.
78. Altmann D M, Boyton R J. SARS-CoV-2 T cell immunity: Specificity, function, durability, and role in protection. Sci Immunol. 2020; 5(49).
79. Vita R, Mahajan S, Overton J A, Dhanda S K, Martini S, Cantrell J R, et al. The Immune Epitope Database (IEDB): 2018 update. Nucleic Acids Res. 2019; 47(D1):D339-D43.
80. Dhanda S K, Vita R, Ha B, Grifoni A, Peters B, Sette A. ImmunomeBrowser: a tool to aggregate and visualize complex and heterogeneous epitopes in reference proteins. Bioinformatics. 2018; 34(22):3931-3.
81. Gonzalez-Galarza F F, McCabe A, Santos E, Jones J, Takeshita L, Ortega-Rivera N D, et al. Allele frequency net database (AFND) 2020 update: gold-standard data classification, open access genotype data and new query tools. Nucleic Acids Res. 2020; 48(D1):D783-D8.
82. Robinson J, Barker D J, Georgiou X, Cooper M A, Flicek P, Marsh S G E. IPD-IMGT/HLA Database. Nucleic Acids Res. 2020; 48(D1):D948-D55.
83. McKinney D M, Southwood S, Hinz D, Oseroff C, Arlehamn C S, Schulten V, et al. A strategy to determine HLA class II restriction broadly covering the D R, D P, and D Q allelic variants most commonly expressed in the general population. Immunogenetics. 2013; 65(5):357-70.
84. Sette A, Sidney J. Nine major HLA class I supertypes account for the vast preponderance of HLA-A and -B polymorphism. Immunogenetics. 1999; 50(3-4):201-12.
85. Weiskopf D, Angelo M A, de Azeredo E L, Sidney J, Greenbaum J A, Fernando A N, et al. Comprehensive analysis of dengue virus-specific responses supports an HLA-linked protective role for CD8+ T cells. Proc Natl Acad Sci USA. 2013; 110(22):E2046-53.
86. Goulder P J, Walker B D. HIV and HLA class I: an evolving relationship. Immunity. 2012; 37(3):426-40.
87. Bacher P, Rosati E, Esser D, Martini G R, Saggau C, Schiminsky E, et al. Low-Avidity CD4(+) T Cell Responses to SARS-CoV-2 in Unexposed Individuals and Humans with Severe COVID-19. Immunity. 2020; 53(6):1258-71 e5.
88. Lipsitch M, Grad Y H, Sette A, Crotty S. Cross-reactive memory T cells and herd immunity to SARS-CoV-2. Nat Rev Immunol. 2020; 20(11):709-13.
89. Sagar M, Reifler K, Rossi M, Miller N S, Sinha P, White L F, et al. Recent endemic coronavirus infection is associated with less-severe COVID-19. The Journal of Clinical Investigation. 2021; 131(1).
90. Sette A, Crotty S. Author Correction: Pre-existing immunity to SARS-CoV-2: the knowns and unknowns. Nat Rev Immunol. 2020; 20(10):644.
91. Sagar M, Reifler K, Rossi M, Miller N S, Sinha P, White L F, et al. Recent endemic coronavirus infection is associated with less-severe COVID-19. J Clin Invest. 2021; 131(1).
92. Agerer B, Koblischke M, Gudipati V, Montano-Gutierrez L F, Smyth M, Popa A, et al. SARS-CoV-2 mutations in MHC-I-restricted epitopes evade CD8(+) T cell responses. Sci Immunol. 2021; 6(57).
93. Tarke A, Sidney J, Methot N, Zhang Y, Dan J M, Goodwin B, et al. Negligible impact of SARS-CoV-2 variants on CD4 (+) and CD8 (+) T cell reactivity in COVID-19 exposed donors and vaccinees. bioRxiv. 2021.
94. Redd A D, Nardin A, Kared H, Bloch E M, Pekosz A, Laeyendecker 0, et al. CD8+ T cell responses in COVID-19 convalescent individuals target conserved epitopes from multiple prominent SARS-CoV-2 circulating variants. medRxiv. 2021.
95. Tegally H, Wilkinson E, Giovanetti M, Iranzadeh A, Fonseca V, Giandhari J, et al. Emergence of a SARS-CoV-2 variant of concern with mutations in spike glycoprotein. Nature. 2021.
96. Wang P, Nair M S, Liu L, Iketani S, Luo Y, Guo Y, et al. Antibody Resistance of SARS-CoV-2 Variants B.1.351 and B.1.1.7. Nature. 2021.
97. Thomson E C, Rosen L E, Shepherd J G, Spreafico R, da Silva Filipe A, Wojcechowskyj J A, et al. Circulating SARS-CoV-2 spike N439K variants maintain fitness while evading antibody-mediated immunity. Cell. 2021; 184(5):1171-87 e20.
98. Avanzato V A, Matson M J, Seifert S N, Pryce R, Williamson B N, Anzick S L, et al. Case Study: Prolonged Infectious SARS-CoV-2 Shedding from an Asymptomatic Immunocompromised Individual with Cancer. Cell. 2020; 183(7):1901-12 e9.
99. Rimmelzwaan G F, Berkhoff E G M, Nieuwkoop N J, Smith D J, Fouchier R A M, Osterhaus A. Full restoration of viral fitness by multiple compensatory co-mutations in the nucleoprotein of influenza A virus cytotoxic T-lymphocyte escape mutants. J Gen Virol. 2005; 86(Pt 6):1801-5.
100. Bilich T, Nelde A, Heitmann J S, Maringer Y, Roerden M, Bauer J, et al. T cell and antibody kinetics delineate SARS-CoV-2 peptides mediating long-term immune responses in COVID-19 convalescent individuals. Sci Transl Med. 2021.
101. Gittelman R M, Lavezzo E, Snyder™, Zahid H J, Elyanow R, Dalai S, et al. Diagnosis and Tracking of SARS-CoV-2 Infection By T-Cell Receptor Sequencing. medRxiv. 2021.
102. Shaan Lakshmanappa Y, Elizaldi S R, Roh J W, Schmidt B A, Carroll T D, Weaver K D, et al. SARS-CoV-2 induces robust germinal center CD4 T follicular helper cell responses in rhesus macaques. Nat Commun. 2021; 12(1):541.

### References—Examples 17-18

Bergwerk, M., Gonen, T., Lustig, Y., Amit, S., Lipsitch, M., Cohen, C., Mandelboim, M., Levin, E. G., Rubin, C., Indenbaum, V., et al. (2021). Covid-19 Breakthrough Infections in Vaccinated Health Care Workers. N Engl J Med 385, 1474-1484.
Bertoletti, A., Le Bert, N., Qui, M., and Tan, A. T. (2021). SARS-CoV-2-specific T cells in infection and vaccination. Cell Mol Immunol 18, 2307-2312.
Carrasco Pro, S., Sidney, J., Paul, S., Lindestam Arlehamn, C., Weiskopf, D., Peters, B., and Sette, A. (2015). Automatic Generation of Validated Specific Epitope Sets. J Immunol Res 2015, 763461.
Cohen, K. W., Linderman, S. L., Moodie, Z., Czartoski, J., Lai, L., Mantus, G., Norwood, C., Nyhoff, L. E., Edara, V. V., Floyd, K., et al. (2021). Longitudinal analysis shows durable and broad immune memory after SARS-CoV-2 infection with persisting antibody responses and memory B and T cells. Cell Rep Med 2, 100354.
Collier, A. Y., Brown, C. M., McMahan, K., Yu, J., Liu, J., Jacob-Dolan, C., Chandrashekar, A., Tierney, D., Ansel, J. L., Rowe, M., et al. (2021). Immune Responses in Fully Vaccinated Individuals Following Breakthrough Infection with the SARS-CoV-2 Delta Variant in Provincetown, Massachusetts. medRxiv.
Crotty, S. (2021). Hybrid immunity. Science 372, 1392-1393.
da Silva Antunes, R., Pallikkuth, S., Williams, E., Dawen Yu, E., Mateus, J., Quiambao, L., Wang, E., Rawlings, S. A., Stadlbauer, D., Jiang, K., et al. (2021). Differential T-Cell Reactivity to Endemic Coronaviruses and SARS-CoV-2 in Community and Health Care Workers. J Infect Dis 224, 70-80.
Dan, J. M., Mateus, J., Kato, Y., Hastie, K. M., Yu, E. D., Faliti, C. E., Grifoni, A., Ramirez, S. I., Haupt, S., Frazier, A., et al. (2021). Immunological memory to SARS-CoV-2 assessed for up to 8 months after infection. Science 371.
Feng, S., Phillips, D. J., White, T., Sayal, H., Aley, P. K., Bibi, S., Dold, C., Fuskova, M., Gilbert, S. C., Hirsch, I., et al. (2021). Correlates of protection against symptomatic and asymptomatic SARS-CoV-2 infection. Nat Med.
Goel, R. R., Painter, M. M., Apostolidis, S. A., Mathew, D., Meng, W., Rosenfeld, A. M., Lundgreen, K. A., Reynaldi, A., Khoury, D. S., Pattekar, A., et al. (2021). mRNA vaccines induce durable immune memory to SARS-CoV-2 and variants of concern. Science, eabm0829.
Grifoni, A., Sidney, J., Vita, R., Peters, B., Crotty, S., Weiskopf, D., and Sette, A. (2021). SARS-CoV-2 human T cell epitopes: Adaptive immune response against COVID-19. Cell Host Microbe 29, 1076-1092.
Grifoni, A., Sidney, J., Zhang, Y., Scheuermann, R. H., Peters, B., and Sette, A. (2020a). A Sequence Homology and Bioinformatic Approach Can Predict Candidate Targets for Immune Responses to SARS-CoV-2. Cell Host Microbe 27, 671-680 e672.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020b). Targets of T Cell Responses to SARS-CoV-2 Coronavirus in Humans with COVID-19 Disease and Unexposed Individuals. Cell 181, 1489-1501 e1415.
Ibarrondo, F. J., Fulcher, J. A., Goodman-Meza, D., Elliott, J., Hofmann, C., Hausner, M. A., Ferbas, K. G., Tobin, N. H., Aldrovandi, G. M., and Yang, 0.0. (2020). Rapid Decay of Anti-SARS-CoV-2 Antibodies in Persons with Mild Covid-19. N Engl J Med 383, 1085-1087.
Khoury, D. S., Cromer, D., Reynaldi, A., Schlub, T. E., Wheatley, A. K., Juno, J. A., Subbarao, K., Kent, S. J., Triccas, J. A., and Davenport, M. P. (2021). Neutralizing antibody levels are highly predictive of immune protection from symptomatic SARS-CoV-2 infection. Nat Med 27, 1205-1211.
Klompas, M. (2021). Understanding Breakthrough Infections Following mRNA SARS-CoV-2 Vaccination. JAMA.
Knierman, M. D., Lannan, M. B., Spindler, L. J., McMillian, C. L., Konrad, R. J., and Siegel, R. W. (2020). The Human Leukocyte Antigen Class II Immunopeptidome of the SARS-CoV-2 Spike Glycoprotein. Cell Rep 33, 108454.
Koup, R. A., Donis, R. O., Gilbert, P. B., Li, A. W., Shah, N. A., and Houchens, C. R. (2021). A government-led effort to identify correlates of protection for COVID-19 vaccines. Nat Med 27, 1493-1494.
Krammer, F. (2021a). A correlate of protection for SARS-CoV-2 vaccines is urgently needed. Nat Med 27, 1147-1148.
Krammer, F. (2021b). Correlates of protection from SARS-CoV-2 infection. Lancet 397, 1421-1423.
Krishna, B. A. (Preprint). Retrospective diagnosis of SARS-CoV-2 infection in patients with Long COVID by measuring specific T cell mediated IL-2 release.
Kruse, M., Dark, C., Aspden, M., Cochrane, D., Competiello, R., Peltz, M., Torres, L., Wrighton-Smith, P., and Dudek, M. (2021). Performance of the T-SPOT®. COVID test for detecting SARS-CoV-2-responsive T cells. Int J Infect Dis.
Kustin, T., Harel, N., Finkel, U., Perchik, S., Harari, S., Tahor, M., Caspi, I., Levy, R., Leshchinsky, M., Ken Dror, S., et al. (2021). Evidence for increased breakthrough rates of SARS-CoV-2 variants of concern in BNT162b2-mRNA-vaccinated individuals. Nat Med 27, 1379-1384.
Le Bert, N., Tan, A. T., Kunasegaran, K., Tham, C. Y. L., Hafezi, M., Chia, A., Chng, M. H. Y., Lin, M., Tan, N., Linster, M., et al. (2020). SARS-CoV-2-specific T cell immunity in cases of COVID-19 and SARS, and uninfected controls. Nature 584, 457-462.
Lind Enoksson, S., Bergman, P., Klingstrom, J., Bostrom, F., Da Silva Rodrigues, R., Winerdal, M. E., and Marits, P. (2021). A flow cytometry-based proliferation assay for clinical evaluation of T-cell memory against SARS-CoV-2. J Immunol Methods 499, 113159.
Lozano-Ojalvo, D., Camara, C., Lopez-Granados, E., Nozal, P., Del Pino-Molina, L., Bravo-Gallego, L. Y., Paz-Artal, E., Pion, M., Correa-Rocha, R., Ortiz, A., et al. (2021). Differential effects of the second SARS-CoV-2 mRNA vaccine dose on T cell immunity in naive and COVID-19 recovered individuals. Cell Rep, 109570.
Lucas, C., Vogels, C. B. F., Yildirim, I., Rothman, J. E., Lu, P., Monteiro, V., Gelhausen, J. R., Campbell, M., Silva, J., Tabachikova, A., et al. (2021). Impact of circulating SARS-CoV-2 variants on mRNA vaccine-induced immunity. Nature.
Martinez-Gallo, M. (2021). Commercialized kits to assess T-cell responses against SARS-CoV-2 S peptides. A pilot study in health care workers. Medicina Clinica.
Massey, D., Berrent, D., Akrami, A., Assaf, G., Davis, H., Harris, K., McCorkell, L., Ring, A. M., Schulz, W. L., Wei, H., et al. (Preprint-a). Change in Symptoms and Immune Response in People with Post-Acute Sequelae of SARS-Cov-2 Infection (PASC) After SARS-Cov-2 Vaccination.
Massey, D., Berrent, D., and Krumholz, H. (Preprint-b). Breakthrough Symptomatic COVID-19 Infections Leading to Long Covid: Report from Long Covid Facebook Group Poll.
Mateus, J., Dan, J. M., Zhang, Z., Rydyznski Moderbacher, C., Lammers, M., Goodwin, B., Sette, A., Crotty, S., and Weiskopf, D. (2021). Low-dose mRNA-1273 COVID-19 vaccine generates durable memory enhanced by cross-reactive T cells. Science 374, eabj9853.
Mateus, J., Grifoni, A., Tarke, A., Sidney, J., Ramirez, S. I., Dan, J. M., Burger, Z. C., Rawlings, S. A., Smith, D. M., Phillips, E., et al. (2020). Selective and cross-reactive SARS-CoV-2 T cell epitopes in unexposed humans. Science 370, 89-94.
Mizrahi, B., Lotan, R., Kalkstein, N., Peretz, A., Perez, G., Ben-Tov, A., Chodick, G., Gazit, S., and Patalon, T. (2021). Correlation of SARS-CoV-2-breakthrough infections to time-from-vaccine. Nat Commun 12, 6379.
Murugesan, K. (Preprint). Long term accuracy of SARS-CoV-2 Interferon-gamma release assays and its apllication in household investigation.
Murugesan, K., Jagannathan, P., Pham, T. D., Pandey, S., Bonilla, H. F., Jacobson, K., Parsonnet, J., Andrews, J. R., Weiskopf, D., Sette, A., et al. (2020). Interferon-gamma release assay for accurate detection of SARS-CoV-2 T cell response. Clin Infect Dis.
Niessl, J., Sekine, T., and Buggert, M. (2021). T cell immunity to SARS-CoV-2. Semin Immunol, 101505.
Ogbe, A., Kronsteiner, B., Skelly, D. T., Pace, M., Brown, A., Adland, E., Adair, K., Akhter, H. D., Ali, M., Ali, S. E., et al. (2021). T cell assays differentiate clinical and subclinical SARS-CoV-2 infections from cross-reactive antiviral responses. Nat Commun 12, 2055.
Ortega, N., Ribes, M., Vidal, M., Rubio, R., Aguilar, R., Williams, S., Barrios, D., Alonso, S., Hernandez-Luis, P., Mitchell, R. A., et al. (2021). Seven-month kinetics of SARS-CoV-2 antibodies and role of pre-existing antibodies to human coronaviruses. Nat Commun 12, 4740.
Oseroff, C., Kos, F., Bui, H. H., Peters, B., Pasquetto, V., Glenn, J., Palmore, T., Sidney, J., Tscharke, D. C., Bennink, J. R., et al. (2005). HLA class I-restricted responses to vaccinia recognize a broad array of proteins mainly involved in virulence and viral gene regulation. Proc Natl Acad Sci U S A 102, 13980-13985.
Painter, M. M., Mathew, D., Goel, R. R., Apostolidis, S. A., Pattekar, A., Kuthuru, O., Baxter, A. E., Herati, R. S., Oldridge, D. A., Gouma, S., et al. (2021). Rapid induction of antigen-specific CD4(+) T cells is associated with coordinated humoral and cellular immunity to SARS-CoV-2 mRNA vaccination. Immunity 54, 2133-2142 e2133.
Pan, K., Chiu, Y., Huang, E., Chen, M., Wang, J., Lai, I., Singh, S., Shaw, R. M., MacCoss, M. J., and Yee, C. (2021). Mass spectrometric identification of immunogenic SARS-CoV-2 epitopes and cognate TCRs. Proc Natl Acad Sci USA 118.
Paul, S., Karosiene, E., Dhanda, S. K., Jurtz, V., Edwards, L., Nielsen, M., Sette, A., and Peters, B. (2018). Determination of a Predictive Cleavage Motif for Eluted Major Histocompatibility Complex Class II Ligands. Front Immunol 9, 1795.
Peeling, R. W., and Olliaro, P. (2021). Rolling out COVID-19 antigen rapid diagnostic tests: the time is now. Lancet Infect Dis 21, 1052-1053.
Petrone, L., Petruccioli, E., Vanini, V., Cuzzi, G., Najafi Fard, S., Alonzi, T., Castilletti, C., Palmieri, F., Gualano, G., Vittozzi, P., et al. (2021). A whole blood test to measure SARS-CoV-2-specific response in COVID-19 patients. Clin Microbiol Infect 27, 286 e287-286 e213.
Pouwels, K. B., Pritchard, E., Matthews, P. C., Stoesser, N., Eyre, D. W., Vihta, K. D., House, T., Hay, J., Bell, J. I., Newton, J. N., et al. (2021). Effect of Delta variant on viral burden and vaccine effectiveness against new SARS-CoV-2 infections in the UK. Nat Med.
Rovida, F., Cassaniti, I., Paolucci, S., Percivalle, E., Sarasini, A., Piralla, A., Giardina, F., Sammartino, J. C., Ferrari, A., Bergami, F., et al. (2021). SARS-CoV-2 vaccine breakthrough infections with the alpha variant are asymptomatic or mildly symptomatic among health care workers. Nat Commun 12, 6032.
Rydyznski Moderbacher, C., Ramirez, S. I., Dan, J. M., Grifoni, A., Hastie, K. M., Weiskopf, D., Belanger, S., Abbott, R. K., Kim, C., Choi, J., et al. (2020). Antigen-Specific Adaptive Immunity to SARS-CoV-2 in Acute COVID-19 and Associations with Age and Disease Severity. Cell 183, 996-1012 e1019.
Sekine, T., Perez-Potti, A., Rivera-Ballesteros, O., Stralin, K., Gorin, J. B., Olsson, A., Llewellyn-Lacey, S., Kamal, H., Bogdanovic, G., Muschiol, S., et al. (2020). Robust T Cell Immunity in Convalescent Individuals with Asymptomatic or Mild COVID-19. Cell 183, 158-168 el 14.
Sette, A., and Crotty, S. (2020). Pre-existing immunity to SARS-CoV-2: the knowns and unknowns. Nat Rev Immunol 20, 457-458.
Sette, A., and Crotty, S. (2021). Adaptive immunity to SARS-CoV-2 and COVID-19. Cell 184, 861-880.
Swadling, L., Diniz, M. O., Schmidt, N. M., Amin, O. E., Chandran, A., Shaw, E., Pade, C., Gibbons, J. M., Le Bert, N., Tan, A. T., et al. (2021). Pre-existing polymerase-specific T cells expand in abortive seronegative SARS-CoV-2. Nature.
Tan, A. T., Lim, J. M., Le Bert, N., Kunasegaran, K., Chia, A., Qui, M. D., Tan, N., Chia, W. N., de Alwis, R., Ying, D., et al. (2021). Rapid measurement of SARS-CoV-2 spike T cells in whole blood from vaccinated and naturally infected individuals. J Clin Invest 131.
Taquet, M., Dercon, Q., and Harrison, P. J. (Preprint). Six-month sequelae of post-vaccination SARS-CoV-2 infection: a retrospective cohort study of 10,024 breakthrough infections.
Tarke, A., Sidney, J., Kidd, C. K., Dan, J. M., Ramirez, S. I., Yu, E. D., Mateus, J., da Silva Antunes, R., Moore, E., Rubiro, P., et al. (2021a). Comprehensive analysis of T cell immunodominance and immunoprevalence of SARS-CoV-2 epitopes in COVID-19 cases. Cell Rep Med 2, 100204.
Tarke, A., Sidney, J., Methot, N., Yu, E. D., Zhang, Y., Dan, J. M., Goodwin, B., Rubiro, P., Sutherland, A., Wang, E., et al. (2021b). Impact of SARS-CoV-2 variants on the total CD4(+) and CD8(+) T cell reactivity in infected or vaccinated individuals. Cell Rep Med 2, 100355.
Tormo, N. (Pre-proof). Commercial interferpon-gamma release assay to assesss the immune response to first and second doses of mRNA vaccine.
Trevethan, R. (2017). Sensitivity, Specificity, and Predictive Values: Foundations, Pliabilities, and Pitfalls in Research and Practice. Front Public Health 5, 307.
Vandenberg, O., Martiny, D., Rochas, O., van Belkum, A., and Kozlakidis, Z. (2021). Considerations for diagnostic COVID-19 tests. Nat Rev Microbiol 19, 171-183.
Weingarten-Gabbay, S., Klaeger, S., Sarkizova, S., Pearlman, L. R., Chen, D. Y., Gallagher, K. M. E., Bauer, M. R., Taylor, H. B., Dunn, W. A., Tarr, C., et al. (2021). Profiling SARS-CoV-2 HLA-I peptidome reveals T cell epitopes from out-of-frame ORFs. Cell 184, 3962-3980 e3917.
Zelba, H., Worbs, D., Harter, J., Pieper, N., Kyzirakos-Feger, C., Kayser, S., Seibold, M., Bartsch, O., Kodding, J., and Biskup, S. (2021). A Highly Specific Assay for the Detection of SARS-CoV-2-Reactive CD4(+) and CD8(+) T Cells in COVID-19 Patients. J Immunol 206, 580-587.

1.-16. (canceled)

17. A composition comprising monomers or multimers of:
one or more peptides or proteins comprising:
one or more amino acid sequences selected from those sequences set forth in Tables 1 to 10 (SEQ ID NOS: 1 to 3522),
concatemers, subsequences, portions, homologues, variants or derivatives thereof;
a fusion protein comprising one or more amino acid sequences selected from those sequences set forth in Tables 1 to 10 (SEQ ID NOS: 1 to 3522); or
a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof;
one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 to 10 (SEQ ID NOS: 1 to 3522), in a groove of the MHC monomer or multimer;
one or more peptides or proteins comprising, consisting of, or consisting essentially of an amino acid sequence selected from those sequences set forth in Tables 1 to 10 (SEQ ID NOS: 1 to 3522), or a subsequence, portion, homologue, variant or derivative thereof;
a fusion protein comprising one or more amino acid sequences selected from those sequences set forth in Tables 1 to 10 (SEQ ID NOS: 1 to 3522);
a pool of 2 or more peptides selected from those sequences set forth in Tables 1 to 10 (SEQ ID NOS: 1 to 3522); or
a polynucleotide that encodes one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof.

18.-34. (canceled)

35. The composition of claim 1, wherein the composition comprises one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 to 10 (SEQ ID NOS: 1 to 3522), in a groove of the (MHC) monomer or multimer, or wherein the compositions include those amino acid sequences selected from Tables 1, 2, 3, 4, 5, 6, 7, 8, 9, or 10 (SEQ ID NOS: 1 to 3522).

36. (canceled)

37. A method for detecting the presence of: (i) a coronavirus or (ii) an immune response relevant to coronavirus infections, vaccines or therapies, including T cells responsive to one or more coronavirus peptides, comprising:
providing one or more proteins or peptides for detection of an amount or a relative amount of, and/or the activity of, and/or the state of antigen-specific T-cells;
contacting a biological sample suspected of having coronavirus-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in Tables 1 to 10 (SEQ ID NOS: 1 to 3522), or comprise a pool of 2 or more amino acid sequences set forth in Tables 1 to 10 (SEQ ID NOS: 1 to 3522).

38. The method of claim 37, further comprising at least one of:
detecting the amount or a relative amount of, and/or activity of antigen-specific T-cells comprises one or more steps of identification or detection of the antigen-specific T-cells and measuring the amount of the antigen-specific T-cells; or
detecting the presence or amount of the one or more peptides in a biological sample, or a response thereto, which is diagnostic of a coronavirus infection;
detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay;
detecting an immune response relevant to the coronavirus comprises the following steps:
providing an MHC monomer or an MHC multimer;
contacting a population T-cells to the MHC monomer or MHC multimer; and
measuring the number, activity or state of T-cells specific for the MHC monomer or MHC multimer; or
further comprises administering a treatment comprising the composition to the subject from which the biological sample was drawn that increases the amount or relative amount of, and/or activity of the antigen-specific T-cells;
the one or more peptides or proteins comprises 2 or more amino acid sequences selected from Tables 1 to 10 (SEQ ID NOS: 1 to 3522);
the one or more peptides or proteins comprises 2 or more amino acid sequences selected from Tables 1 to 10 (SEQ ID NOS: 1 to 3522);
the detecting the amount or a relative amount of, and/or activity of antigen-specific T-cells comprises indirect detection and/or direct detection;
the MHC monomer or MHC multimer comprises a protein or peptide of the coronavirus;
the protein or peptide comprises a CD8+ or CD4+ T cell epitope;
the T cell epitope is not conserved in another coronavirus;
the T cell epitope is conserved in another coronavirus;
the protein or peptide has a length from about 9-15, 15-20, 20-25, 25-30, 30-40, 40-50, 50-75 or 75-100 amino acids;
the proteins or peptides comprise 2 or more amino acid sequences selected from those sequences set forth in Tables 1, 2, 3, 4, 5, 6, 7, 8, 9, or 10 (SEQ ID NOS: 1 to 3522), or a subsequence, portion, homologue, variant or derivative thereof; or
detecting the presence of: (i) SARS-CoV-2 or (ii) an immune response relevant to SARS-CoV-2 infections, vaccines or therapies, including T cells responsive to one or more SARS-CoV-2 peptides, comprising:
providing one or more proteins or peptides for detection of an amount or a relative amount of, and/or the activity of, and/or the state of antigen-specific T-cells;
contacting a biological sample suspected of having SARS-CoV-2-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in those sequences set forth in Tables 1 to 10 (SEQ ID NOS: 1 to 3522), or comprise a pool of 2 or more amino acid sequences set forth in those sequences set forth in Tables 1 to 10 (SEQ ID NOS: 1 to 3522).

39.-64. (canceled)

65. The method of claim 37, wherein detecting a coronavirus infection or exposure in a subject, the method comprising, consisting of, or consisting essentially of:
contacting a biological sample from a subject with a peptide monomer or multimer composition of any one of SEQ ID NOS: 1 to 3522; and
determining if the composition elicits an immune response from the contacted cells, wherein the presence of an immune response indicates that the subject has been exposed to or infected with coronavirus; or
wherein the sample comprises T cells and the response comprises inducing, increasing, promoting or stimulating anti-coronavirus activity of T cells.
the T cells are CD8+ or CD4+ T cells;
determining whether the subject has been infected by or exposed to the coronavirus more than once by determining if the subject elicits a secondary T cell immune response profile that is different from a primary T cell immune response profile;
diagnosing a coronavirus infection or exposure in a subject, the method comprising contacting a biological sample from a subject with the composition, and determining if the composition elicits a T cell immune response, wherein the T cell immune response identifies that the subject has been infected with or exposed to a coronavirus;
wherein the method is conducted three or more days following the date of suspected infection by or exposure to a coronavirus; or
determining if the composition elicits an immune response from the contacted cells, wherein the presence of an immune response indicates that the subject has been exposed to or infected with SARS-CoV-2.

66.-78. (canceled)

79. A kit for the detection of coronavirus or an immune response to coronavirus in a subject comprising, consisting of or consisting essentially of:
one or more T cells that specifically detect the presence of:
one or more amino acid sequences selected from those sequences set forth in Tables 1 to 10 (SEQ ID NOS: 1 to 3522), or a subsequence, portion, homologue, variant or derivative thereof; or
a fusion protein comprising one or more amino acid sequences selected from those sequences set forth in Tables 1 to 10 (SEQ ID NOS: 1 to 3522); or
a pool of 2 or more peptides selected from the amino acid sequences set forth in Tables 1 to 10 (SEQ ID NOS: 1 to 3522).

80. The kit of claim 79, wherein at least one of:
the one or more amino acid sequences are selected from a coronavirus T cell epitope set forth in Tables 1 to 10 (SEQ ID NOS: 1 to 3522);
the composition comprises: one or more amino acid sequences selected from those sequences set forth in Tables 1 to 10 (SEQ ID NOS: 1 to 3522), or a subsequence, portion, homologue, variant or derivative thereof;
a fusion protein comprising one or more amino acid sequences selected from those sequences set forth in Tables 1 to 10 (SEQ ID NOS: 1 to 3522); or
a pool of 2 or more peptides selected from the amino acid sequences set forth in those sequences set forth in Tables 1 to 10 (SEQ ID NOS: 1 to 3522).
the amino acid sequence comprises a coronavirus CD8+ or CD4+ T cell epitope;
the T cell epitope is not conserved in another coronavirus;
the T cell epitope is conserved in another coronavirus;
the fusion protein has a length from about 9-15, 15-20, 20-25, 25-30, 30-40, 40-50, 50-75 or 75-100 amino acids;
the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus;
the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay;
the kit includes reagents for determining a Human Leukocyte Antigen (HLA) profile of a subject, and selecting peptides that are presented by the HLA profile of the subject for detecting an immune response to coronavirus; or
wherein detection of SARS-CoV-2 or an immune response to SARS-CoV-2 in a subject comprising, consisting of or consisting essentially of:
one or more T cells that specifically detect the presence of:
one or more amino acid sequences selected from those sequences set forth in Tables 1 to 10 (SEQ ID NOS: 1 to 3522), or a subsequence, portion, homologue, variant or derivative thereof;
a fusion protein comprising one or more amino acid sequences selected from those sequences set forth in Tables 1 to 10 (SEQ ID NOS: 1 to 3522); or
a pool of 2 or more peptides selected from the amino acid sequences set forth in those sequences set forth in Tables 1 to 10 (SEQ ID NOS: 1 to 3522).

81.-98. (canceled)

99. A method of stimulating, inducing, promoting, increasing, or enhancing an immune response against a coronavirus in a subject, comprising:
administering a composition of one or more monomers or multimers selected from any one of SEQ ID NOS: 1 to 3522, in an amount sufficient to stimulate, induce, promote, increase, or enhance an immune response against the coronavirus in the subject.

100. The method of claim 99, wherein at least one of:
the immune response provides the subject with protection against a coronavirus infection or pathology, or one or more physiological conditions, disorders, illnesses, diseases or symptoms caused by or associated with coronavirus infection or pathology; or the immune response is specific to: one or more SARS-CoV-2 peptides selected from the amino acid sequences set forth in Tables 1 to 10 (SEQ ID NOS: 1 to 3522), or a subsequence, portion, homologue, variant or derivative thereof;
wherein the one or more monomers or multimers selected from any one of SEQ ID NOS: 1 to 3522 provides the subject with protection against a SARS-CoV-2 infection or pathology, or one or more physiological conditions, disorders, illnesses, diseases or symptoms caused by or associated with SARS-CoV-2 infection or pathology; or
wherein stimulating, inducing, promoting, increasing, or enhancing an immune response against SARS-CoV-2 in a subject, comprises administering to a subject an amount of a protein or peptide or a polynucleotide that expresses the protein or peptide comprising, consisting of or consisting essentially of an amino acid sequence of the SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in Tables 1 to 10 (SEQ ID NOS: 1 to 3522), or both or a subsequence, portion, homologue, variant or derivative thereof, in an amount sufficient to prevent, stimulate, induce, promote, increase, immunize against, or enhance an immune response against SARS-CoV-2 in the subject.

101.-109. (canceled)

110. The method of claim 99, wherein at least one of:
the anti-SARS-CoV-2 T cell response is a CD8+, a CD4+ T cell response, or both;
the T cell epitope is conserved across two or more clinical isolates of SARS-CoV-2, two or more circulating forms of SARS-CoV-2, or two or more coronaviruses;
the SARS-CoV-2 infection is an acute infection;
the subject is a mammal or a human;
reducing SARS-CoV-2 viral titer, increases or stimulates SARS-CoV-2 viral clearance,
reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid;
reducing one or more adverse physiological conditions, disorders, illness, diseases, symptoms or complications caused by or associated with SARS-CoV-2 infection or pathology;
improving one or more adverse physiological conditions, disorders, illness, diseases, symptoms or complications caused by or associated with SARS-CoV-2 infection or pathology; =, and wherein the symptom is fever or chills, cough, shortness of breath or difficulty breathing, fatigue, muscle or body aches, headache, new loss of taste or smell, sore throat, congestion or runny nose, nausea or vomiting, or diarrhea;
reducing or inhibiting susceptibility to SARS-CoV-2 infection or pathology;
the protein or peptide, or a subsequence, portion, homologue, variant or derivative thereof, is administered prior to, substantially contemporaneously with or following exposure to or infection of the subject with SARS-CoV-2;
a plurality of SARS-CoV-2 T cell epitopes are administered prior to, substantially contemporaneously with or following exposure to or infection of the subject with SARS-CoV-2;
the protein or peptide, or a subsequence, portion, homologue, variant or derivative thereof is administered within 2-72 hours, 2-48 hours, 4-24 hours, 4-18 hours, or 6-12 hours after a symptom
the protein or peptide, or a subsequence, portion, homologue, variant or derivative thereof is administered prior to exposure to or infection of the subject with SARS-CoV-2;
the method further comprises administering a modulator of immune response prior to, substantially contemporaneously with or following the administration to the subject of an amount of a protein or peptide;
wherein the modulator of immune response is a modulator of the innate immune response;
the modulator is IL-6, IFN-γ, TGF-β, or IL-10, or an agonist or antagonist thereof; or
the one or amino acid sequences include amino acid sequences selected from Tables 1 to 10 (SEQ ID NOS: 1 to 3522).

111.-126. (canceled)

127. A method of treating, preventing, or immunizing a subject against SARS-CoV-2 infection, comprising administering to a subject one or more monomers or multimers selected from any one of SEQ ID NOS: 1 to 3522 in an amount sufficient to treat, prevent, or immunize the subject for SARS-CoV-2 infection.

128. The method of claim 127, wherein at least one of:
the SARS-CoV-2 infection is an acute infection;
the composition reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid;
the composition reduces one or more adverse physiological conditions, disorders, illness, diseases, symptoms or complications caused by or associated with SARS-CoV-2 infection or pathology;
the composition improves one or more adverse physiological conditions, disorders, illness, diseases, symptoms or complications caused by or associated with SARS-CoV-2 infection or pathology, wherein the symptom is fever or chills, cough, shortness of breath or difficulty breathing, fatigue, muscle or body aches, headache, new loss of taste or smell, sore throat, congestion or runny nose, nausea, vomiting, or diarrhea;
the composition method reduces or inhibits susceptibility to SARS-CoV-2 infection or pathology;
the composition is administered prior to, substantially contemporaneously with or following exposure to or infection of the subject with SARS-CoV-2;
the composition is administered prior to, substantially contemporaneously with or following exposure to or infection of the subject with SARS-CoV-2;
the composition is administered within 2-72 hours, 2-48 hours, 4-24 hours, 4-18 hours, or 6-12 hours after a symptom of SARS-CoV-2 infection or exposure develops; or
the composition is administered prior to exposure to or infection of the subject with SARS-CoV-2.

129.-137. (canceled)

138. The composition of claim 1, further defined as a peptide or peptides that are immunoprevalent or immunodominant in a virus obtained by a method consisting of, or consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus;
determining which pools triggered cytokine release by the T cells; and
deconvoluting from the pool of peptides that elicited cytokine release by the T cells, which peptide or peptides are immunoprevalent or immunodominant in the pool; or
the virus is a SARS-CoV-2 coronavirus and the immunodominant peptides are selected from 1, 2 or more peptides selected from the amino acid sequences set forth in Tables 1 to 10 (SEQ ID NOS: 1 to 3522);
wherein the immunodominant peptides are selected from 1, 2 or more peptides selected from the amino acid sequences set forth in those sequences set forth in Tables 1 to 10 (SEQ ID NOS: 1 to 3522); or
the peptide or peptides include amino acid sequences set forth in Tables 1 to 10 (SEQ ID NOS: 1 to 3522).

139.-143. (canceled)

144. A method of selecting an immunoprevalent or immunodominant peptide or protein of a virus comprising, consisting of, or consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus;
determining which pools triggered cytokine release by the T cells; and
deconvoluting from the pool of peptides that elicited cytokine release by the T cells, which peptide or peptides are immunoprevalent or immunodominant in the pool.

145. The method of claim 144, wherein at least one of:
the virus is a coronavirus;
the coronavirus is SARS-CoV-2;
the immunodominant peptides are selected from 1, 2 or more peptides selected from the amino acid sequences set forth in Tables 1 to 10 (SEQ ID NOS: 1 to 3522);
wherein the immunodominant peptides are selected from 1, 2 or more peptides selected from the amino acid sequences set forth in those sequences set forth in Tables 1 to 10 (SEQ ID NOS: 1 to 3522); or
the peptide or peptides include amino acid sequences set forth in Tables 1 to 10 (SEQ ID NOS: 1 to 3522).

146.-149. (canceled)

150. A polynucleotide that expresses one or more peptides or proteins, comprising, consisting of, or consisting essentially of an amino acid sequence selected from those sequences set forth in Tables 1 to 10 (SEQ ID NOS: 1 to 3522), or a subsequence, portion, homologue, variant or derivative thereof;
a fusion protein comprising one or more amino acid sequences selected from those sequences set forth in Tables 1 to 10 (SEQ ID NOS: 1 to 3522); or
a pool of 2 or more peptides comprising, consisting of, or consisting essentially of amino acid sequences selected from those sequences set forth in Tables 1 to 10 (SEQ ID NOS: 1 to 3522).

151. The polynucleotide of claim 150, further comprising a vector or a viral vector.

152. (canceled)

153. The polynucleotide of claim 151, wherein the vector or viral vector is in a host cell.

154.-157. (canceled)

158. A peptide-major histocompatibility complex (MHC)/peptide multimer comprising at least two MHC/peptide monomers, wherein at least one MHC/peptide monomer comprises a peptide derived from SARS-CoV-2.

159. The peptide-major histocompatibility complex (MHC)/peptide multimer comprising one or more monomers or multimers selected from any one of SEQ ID NOS: 1 to 3522, wherein at least one MHC/peptide monomer comprises a peptide that comprises, consists of, or consists essentially of an amino acid sequence selected from the sequences set forth in Tables 1 to 10 (SEQ ID NOS: 1 to 3522);
wherein at least one MHC/peptide monomer comprises a peptide derived from SARS-CoV-2 Spike (S) protein such as a SARS-CoV-2 Spike (S) protein derived peptide set forth in Tables 1 to 10 (SEQ ID NOS: 1 to 3522);
wherein at least one MHC/peptide monomer comprises a peptide derived from SARS-CoV-2 Membrane (M) protein such as a SARS-CoV-2 Membrane (M) protein derived peptide set forth in Tables 1 to 10 (SEQ ID NOS: 1 to 3522);
wherein at least one MHC/peptide monomer comprises a peptide derived from SARS-CoV-2 Nucleocapsid (N) protein such as a SARS-CoV-2 Nucleocapsid (N) protein derived peptide set forth in Tables 1 to 10 (SEQ ID NOS: 1 to 3522);
wherein at least one MHC/peptide monomer comprises a peptide derived from SARS-CoV-2 Envelope (E) protein such as a SARS-CoV-2 Envelope (E) protein derived peptide set forth in Tables 1 to 10 (SEQ ID NOS: 1 to 3522);
wherein at least one NHC/peptide monomer comprises a peptide derived from SARS-CoV-2 ORF3a protein such as a SARS-CoV-2 ORF3a protein derived peptide set forth in Tables 1 to 10 (SEQ ID NOS: 1 to 3522);
wherein at least one MHC/peptide monomer comprises a peptide derived from SARS-CoV-2 ORF6 protein such as a SARS-CoV-2 ORF6 protein derived peptide set forth in Tables 1 to 10 (SEQ ID NOS: 1 to 3522);
wherein at least one MHC/peptide monomer comprises a peptide derived from SARS-CoV-2 ORF7a protein such as a SARS-CoV-2 ORF7a protein derived peptide set forth in Tables 1 to 10 (SEQ ID NOS: 1 to 3522);
wherein at least one MHC/peptide monomer comprises a peptide derived from SARS-CoV-2 ORF7b protein such as a SARS-CoV-2 ORF7b protein derived peptide set forth in Tables 1 to 10 (SEQ ID NOS: 1 to 3522);
wherein at least one MHC/peptide monomer comprises a peptide derived from SARS-CoV-2 ORF8 protein such as a SARS-CoV-2 ORF8 protein derived peptide set forth in Tables 1 to 10 (SEQ ID NOS: 1 to 3522);
wherein at least one MHC/peptide monomer comprises a peptide derived from SARS-CoV-2 ORF10 protein such as a SARS-CoV-2 ORF10 protein derived peptide set forth in Tables 1 to 10 (SEQ ID NOS: 1 to 3522);
wherein at least one MHC/peptide monomer comprises a peptide derived from SARS-CoV-2 nsp1 protein such as a SARS-CoV-2 nsp1 protein derived peptide set forth in Tables 1 to 10 (SEQ ID NOS: 1 to 3522);
wherein at least one MHC/peptide monomer comprises a peptide derived from SARS-CoV-2 nsp2 protein such as a SARS-CoV-2 nsp2 protein derived peptide set forth in Tables 1 to 10 (SEQ ID NOS: 1 to 3522);
wherein at least one MHC/peptide monomer comprises a peptide derived from SARS-CoV-2 nsp3 protein such as a SARS-CoV-2 nsp3 protein derived peptide set forth in Tables 1 to 10 (SEQ ID NOS: 1 to 3522);
wherein at least one MHC/peptide monomer comprises a peptide derived from SARS-CoV-2 nsp4 protein such as a SARS-CoV-2 nsp4 protein derived peptide set forth in Tables 1 to 10 (SEQ ID NOS: 1 to 3522);
wherein at least one MHC/peptide monomer comprises a peptide derived from SARS-CoV-2 nsp5 protein such as a SARS-CoV-2 nsp5 protein derived peptide set forth in Tables 1 to 10 (SEQ ID NOS: 1 to 3522);
wherein at least one MHC/peptide monomer comprises a peptide derived from SARS-CoV-2 nsp6 protein such as a SARS-CoV-2 nsp6 protein derived peptide set forth in Tables 1 to 10 (SEQ ID NOS: 1 to 3522);
wherein at least one MHC/peptide monomer comprises a peptide derived from SARS-CoV-2 nsp7 protein such as a SARS-CoV-2 nsp7 protein derived peptide set forth in Tables 1 to 10 (SEQ ID NOS: 1 to 3522);
wherein at least one MHC/peptide monomer comprises a peptide derived from SARS-CoV-2 nsp8 protein such as a SARS-CoV-2 nsp8 protein derived peptide set forth in Tables 1 to 10 (SEQ ID NOS: 1 to 3522);
wherein at least one MHC/peptide monomer comprises a peptide derived from SARS-CoV-2 nsp9 protein such as a SARS-CoV-2 nsp9 protein derived peptide set forth in Tables 1 to 10 (SEQ ID NOS: 1 to 3522);
wherein at least one MHC/peptide monomer comprises a peptide derived from SARS-CoV-2 nsp10 protein such as a SARS-CoV-2 nsp10 protein derived peptide set forth in Tables 1 to 10 (SEQ ID NOS: 1 to 3522);
wherein at least one MHC/peptide monomer comprises a peptide derived from SARS-CoV-2 nsp12 protein such as a SARS-CoV-2 nsp12 protein derived peptide set forth in Tables 1 to 10 (SEQ ID NOS: 1 to 3522);
wherein at least one MHC/peptide monomer comprises a peptide derived from SARS-CoV-2 nsp13 protein such as a SARS-CoV-2 nsp13 protein derived peptide set forth in Tables 1 to 10 (SEQ ID NOS: 1 to 3522);
wherein at least one MHC/peptide monomer comprises a peptide derived from SARS-CoV-2 nsp14 protein such as a SARS-CoV-2 nsp14 protein derived peptide set forth in Tables 1 to 10 (SEQ ID NOS: 1 to 3522);
wherein at least one MHC/peptide monomer comprises a peptide derived from SARS-CoV-2 nsp15 protein such as a SARS-CoV-2 nsp15 protein derived peptide set forth in Tables 1 to 10 (SEQ ID NOS: 1 to 3522);
wherein at least one MHC/peptide monomer comprises a peptide derived from SARS-CoV-2 nsp16 protein such as a SARS-CoV-2 nsp16 protein derived peptide set forth in Tables 1 to 10 (SEQ ID NOS: 1 to 3522);
wherein at least one MHC/peptide monomer comprises a peptide comprising, consisting of, or consisting essentially of an amino acid sequence selected from the SARS-CoV-2 B.1.1.529 derived sequences set forth in Table 8 (SEQ ID NOS: 2571 to 2615);
wherein at least one MHC/peptide monomer comprises a peptide comprising, consisting of, or consisting essentially of an amino acid sequence selected from the SARS-CoV-2 derived sequences set forth in Table 1 (SEQ ID NOS: 1 to 1468);
wherein at least one MHC/peptide monomer comprises a peptide comprising, consisting of, or consisting essentially of an amino acid sequence selected from the SARS-CoV-2 derived sequences set forth in Table 2 (SEQ ID NOS: 1469 to 1521) (CD8S(D) megapool);
wherein at least one MHC/peptide monomer comprises a peptide comprising, consisting of, or consisting essentially of an amino acid sequence selected from the SARS-CoV-2 derived sequences set forth in Table 3 (SEQ ID NOS: 1522 to 1665) (CD8S (ND) megapool);
wherein at least one MHC/peptide monomer comprises a peptide comprising, consisting of, or consisting essentially of an amino acid sequence selected from the SARS-CoV-2 derived sequences set forth in Table 4 (SEQ ID NOS: 1666 to 1818) (CD8R(D) megapool);
wherein at least one MHC/peptide monomer comprises a peptide comprising, consisting of, or consisting essentially of an amino acid sequence selected from the SARS-CoV-2 derived sequences set forth in Table 5 (SEQ ID NOS: 1819 to 2286) (CD8R(ND) megapool);
wherein at least one MHC/peptide monomer comprises a peptide comprising, consisting of, or consisting essentially of an amino acid sequence selected from the SARS-CoV-2 derived sequences set forth in Table 6 (SEQ ID NOS: 2287 to 2355) (CD4R(D) megapool);
wherein at least one MHC/peptide monomer comprises a peptide comprising, consisting of, or consisting essentially of an amino acid sequence selected from the SARS-CoV-2 derived sequences set forth in Table 7 (SEQ ID NOS: 2356 to 2570) (CD4R(ND) megapool);
wherein at least one MHC/peptide monomer comprises a peptide comprising, consisting of, or consisting essentially of an amino acid sequence selected from the SARS-CoV-2 derived sequences set forth in Table 9 (SEQ ID NOS: 2616 to 2900) (CD4RE megapool);
wherein at least one MHC/peptide monomer comprises a peptide comprising, consisting of, or consisting essentially of an amino acid sequence selected from the SARS-CoV-2 derived sequences set forth in Table 10 (SEQ ID NOS: 2901 to 3522) (CD8RE megapool);
wherein the at least two MHC/peptide monomers are identical;
wherein the MHC/peptide multimer comprise at least 3, such as at least 4, 5, 6, 7, 8, 9, 10, 11, 12, 13, 14, 15, 16, 17, 18, 19 or 20 MHC/peptide monomers;
wherein the MHC/peptide multimer comprise at least 3, such as at least 4, 5, 6, 7, 8, 9, 10, 11, 12, 13, 14, 15, 16, 17, 18, 19 or 20 identical MHC/peptide monomers;
wherein the MHC/peptide multimer comprise at least 3, such as at least 4, 5, 6, 7, 8, 9, 10, 11, 12, 13, 14, 15, 16, 17, 18, 19 or 20 different MHC/peptide monomers;
wherein at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the sequences set forth in Tables 1 to 10 (SEQ ID NOS: 1 to 3522);
wherein at least 3, 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 B.1.1.529 derived sequences set forth in Table 8 (SEQ ID NOS: 2571 to 2615);
wherein at least 3, 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 Spike (S) protein, Membrane (M) protein, Nucleocapsid (N) protein, Envelope (E) protein, ORF3a, ORF7a, ORF8, nsp1, nsp2, nsp3, nsp6, nsp9, nsp10, nsp12, nsp13, nsp14 and/or nsp15 derived sequences set forth in Tables 1 to 10 (SEQ ID NOS: 1 to 3522);
wherein each MHC/peptide monomer of the MHC/peptide multimer is associated with one or more multimerization domains such as a multimerization domain selected from the group consisting of proteins, peptides, albumins, immunoglobulins, coiled-coil helixes, polynucleotides, IgG, streptavidin, avidin, streptactin, micelles, cells, polymers, dextran, polysaccharides, beads and other types of solid support, and small organic molecules carrying reactive groups or carrying chemical motifs that can bind MHC/peptide monomers;
wherein the multimer comprises no more than 30 MHC/peptide monomers in total, such as no more than 25 MHC/peptide monomers, such as no more than 20 MHC/peptide monomers, such as no more than 15 MHC/peptide monomers, or no more than 10 MHC/peptide monomers in total;
wherein the MHC/peptide multimer comprises from 2 to 50 MHC/peptide monomers, such as from 2 to 4 MHC/peptide monomers, such as from 4 to 6 MHC/peptide monomers, such as from 6 to 8 MHC/peptide monomers, such as from 8 to 10 MHC/peptide monomers, such as from 10 to 12 MHC/peptide monomers, such as from 12 to 14 MHC/peptide monomers, such as from 14 to 16 MHC/peptide monomers, such as from 16 to 18 MHC/peptide monomers, such as from 18 to 20 MHC/peptide monomers, such as from 20 to 25 MHC/peptide monomers, such as from 25 to 30 MHC/peptide monomers, such as from 30 to 40 MHC/peptide monomers, such as from 40 to 50 MHC/peptide monomers, such as from 10 to 20 MHC/peptide monomers or any combination of these intervals;
wherein the MHC/peptide multimer comprises at least 2, 3, 4, 5, 6, 7, 8, 9, 10, 11, 12, 13, 14, 15, 16, 17, 18, 19, 20, 21, 22, 23, 24 or 25 MHC/peptide monomers or has 2, 3, 4, 5, 6, 7, 8, 9, 10, 11, 12, 13, 14, 15, 16, 17, 18, 19, 20, 21, 22, 23, 24 or 25 MHC/peptide monomers in total;
wherein the MHC/peptide multimer comprises MHC Class I/peptide monomers or wherein all MHC monomers of the MHC/peptide multimer are MHC Class I/peptide monomers;
wherein the MHC/peptide multimer comprises MHC Class II/peptide monomers or wherein all MHC/peptide monomers of the MHC/peptide multimer are MHC Class II/peptide monomers;
wherein the MHC/peptide multimer comprises MHC Class I/peptide and MHC Class II/peptide monomers or wherein all MHC/peptide monomers of the MHC/peptide multimer are either MHC Class I/peptide monomers or MHC Class II/peptide monomers;
wherein some of the MHC/peptide monomers or all of the MHC/peptide monomers have identical peptides;
wherein some of the MHC/peptide monomers or all of the MHC/peptide monomers have different peptides;
wherein at least 2, such as at least 3, 4, 5, 6, 7, 8, 9, 10, 11, 12, 13, 1, 15, 16, 17, 18, 19 or 20 of the MHC/peptide monomers comprise different peptides;
wherein the MHC/peptide multimer further comprises one or more labels such as at least two labels;
wherein the labels are different or at least some of the labels are different;
wherein the labels comprise at least one fluorescent label;
2wherein the labels comprise at least one oligonucleotide label such as a nucleic acid molecule comprises or consists of DNA, RNA, and/or artificial nucleotides such as PLA or LNA;
wherein the labels comprise at least one fluorescent label and at least one oligonucleotide label;
wherein the label is an oligonucleotide comprising one or more of: barcode region. 5′ first primer region (forward), 3′ second primer region (reverse), random nucleotide region, connector molecule, stability-increasing components, short nucleotide linkers in between any of the above-mentioned components, adaptors for sequencing, and annealing region;
wherein the labels comprise at least one such as one or more labels selected from the group consisting of APC, APC-Cy7, ABC-H7, APC-R700, Alexa Flours™ 488, Alexa Flours™555, Alexa Flours™647, Alexa Flours™700, AmCyan, BB151, BB700, BUV395, BUV496, BUV563, BUV615, BUV661, BUV737, BUV805, BV421, BV480, BV510, BV605, BV711, BV750, BV786, FITC, PE, PE-CF594, PE-Cy5, PE-CY5.5, PE-cy7, Pasific Blue, PERCP, pPerCp-Cy5.5, PE, R718, RY586, V450, V500, cFluor®B515, cFluor®B532, cFluor®B548, cFluor®B675, cFluor®B690, cFluor®BY575, cFluor®BY610, cFluor®BY667, cFluor®BY710, cFluor®BY750, cFluor®BY781, cFluor®B250, cFluor®R659, cFluor®R668, cFluor®R685, cFluor®R720, cFluor®R780, cFluor®R840, cFluor®v420, cFluor®v547, cFluor®v450, cFluor®v610 and cFluor®YG610;
wherein the one or more labels is a chemiluminescent label such as a label selected from the group consisting of luminol, isoluminol, theromatic acridinium ester, imidazole, acridinium salt and oxalate ester;
wherein the one or more labels is a bioluminescent label such as a label selected from the group consisting of luciferin, luciferase and aequorin;
wherein the one or more labels is an enzyme label, such as an enzyme label selected from the group peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase;
wherein the one or more labels is a chromophore label;
wherein the one or more labels is a metal label; or
wherein the one or more labels is a radioactive label such as a label selected from the group consisting of a radionuclide, an isotope, a label comprising α rays, a label comprising β rays or a label comprising γ rays.

160-224. (canceled)

225. The peptide-major histocompatibility complex (NMC)/peptide multimer of claim 158, further defined as comprising at least one of:
at least 2, 3, 4, 5, 6, 7, 8, 9, 10, 20, 30, 40, 50, 100 or 1000 MHC/peptide multimers;
at least two different MHC/peptide multimers, such as at least 2, 3, 4, 5, 6, 7, 8, 9, 10, 15, 20, 25, 50, 100, 500 or 1000 different MHC/peptide multimers; or
at least 2, 3, 4, 5, 6, 7, 8, 9, 10, 20, 30, 40, 50, 100 or 1000 MHC/peptide multimers of the composition are different each comprising one or more peptides selected from one or more of the following groups:
i) one or more peptides derived from SARS-CoV-2 B.1.1.7, such as one or more SARS-CoV-2 B.1.1.7 derived peptides set forth in Tables 1 to 10 (SEQ ID NOS: 1 to 3522), such as one or more peptides set forth in Table 1, such as one or more peptides set forth in Table 2, such as one or more peptides set forth in Table 3, such as one or more peptides set forth in Table 4, such as one or more peptides set forth in Table 5, such as one or more peptides set forth in Table 6, such as one or more peptides set forth in Table 7, such as one or more peptides set forth in Table 8, such as one or more peptides set forth in Table 9 and/or such as one or more peptides set forth in Table 10, or any combination thereof,
ii) one or more peptides derived from SARS-CoV-2 B1.351. such as one or more SARS-CoV-2 B1.351 derived peptides set forth in Tables 1 to 10 (SEQ ID NOS: 1 to 3522),
iii) one or more peptides derived from SARS-CoV-2 P.1, such as one or more SARS-CoV-2 P.1 derived peptides set forth in Tables 1 to 10 (SEQ ID NOS: 1 to 3522),
iv) one or more peptides derived from SARS-CoV-2 CAL.20C, such as one or more SARS-CoV-2 CAL.20C derived peptides set forth in Tables 1 to 10 (SEQ ID NOS: 1 to 3522),
v) one or more peptides set forth in Tables 1 to 10 (SEQ ID NOS: 1 to 3522)
vi) one or more peptides derived from the SARS-CoV-2 Spike (S) protein such as one or more SARS-CoV-2 Spike (S) protein derived peptides set forth in Tables 1 to 10 (SEQ ID NOS: 1 to 3522),
vii) one or more peptides derived from the SARS-CoV-2 Membrane (M) protein such as one or more SARS-CoV-2 Membrane (M) protein derived peptides set forth in Tables 1 to 10 (SEQ ID NOS: 1 to 3522),
viii) one or more peptides derived from the SARS-CoV-2 Nucleocapsid (N) protein such as one or more SARS-CoV-2 Nucleocapsid (N) protein derived peptides set forth in Tables 1 to 10 (SEQ ID NOS: 1 to 3522),
ix) one or more peptides derived from the SARS-CoV-2 Envelope (E) protein such as one or more SARS-CoV-2 Envelope (E) protein derived peptides set forth in Tables 1 to 10 (SEQ ID NOS: 1 to 3522),
x) one or more peptides derived from the SARS-CoV-2 ORF3a protein such as one or more SARS-CoV-2 ORF3a protein derived peptides set forth in Tables 1 to 10 (SEQ ID NOS: 1 to 3522),
xi) one or more peptides derived from the SARS-CoV-2 ORF6 protein such as one or more SARS-CoV-2 ORF6 protein derived peptides set forth in Tables 1 to 10 (SEQ ID NOS: 1 to 3522),
xii) one or more peptides derived from the SARS-CoV-2 ORF7a protein such as one or more SARS-CoV-2 ORF7a protein derived peptides set forth in Tables 1 to 10 (SEQ ID NOS: 1 to 3522),
xiii) one or more peptides derived from the SARS-CoV-2 ORF7b protein such as one or more SARS-CoV-2 ORF7b protein derived peptides set forth in Tables 1 to 10 (SEQ ID NOS: 1 to 3522),
xiv) one or more peptides derived from the SARS-CoV-2 ORF8 protein such as one or more SARS-CoV-2 ORF8 protein derived peptides set forth in Tables 1 to 10 (SEQ ID NOS: 1 to 3522),
xv) one or more peptides derived from the SARS-CoV-2 ORF10 protein such as one or more SARS-CoV-2 ORF10 protein derived peptides set forth in Tables 1 to 10 (SEQ ID NOS: 1 to 3522),
xvi) one or more peptides derived from the SARS-CoV-2 nsp1 protein such as one or more SARS-CoV-2 nsp1 protein derived peptides Tables 1 to 10 (SEQ ID NOS: 1 to 3522),
xvii) one or more peptides derived from the SARS-CoV-2 nsp2 protein such as one or more SARS-CoV-2 nsp2 protein derived peptides set forth in Tables 1 to 10 (SEQ ID NOS: 1 to 3522),
xviii) one or more peptides derived from the SARS-CoV-2 nsp3 protein such as one or more SARS-CoV-2 nsp3 protein derived peptides set forth in Tables 1 to 10 (SEQ ID NOS: 1 to 3522),
xix) one or more peptides derived from the SARS-CoV-2 nsp4 protein such as one or more SARS-CoV-2 nsp4 protein derived peptides set forth in Tables 1 to 10 (SEQ ID NOS: 1 to 3522),
xx) one or more peptides derived from the SARS-CoV-2 nsp5 protein such as one or more SARS-CoV-2 nsp5 protein derived peptides set forth in Tables 1 to 10 (SEQ ID NOS: 1 to 3522),
xxi) one or more peptides derived from the SARS-CoV-2 nsp6 protein such as one or more SARS-CoV-2 nsp6 protein derived peptides set forth in Tables 1 to 10 (SEQ ID NOS: 1 to 3522),
xxii) one or more peptides derived from the SARS-CoV-2 nsp7 protein such as one or more SARS-CoV-2 nsp7 protein derived peptides set forth in Tables 1 to 10 (SEQ ID NOS: 1 to 3522),
xxiii) one or more peptides derived from the SARS-CoV-2 nsp8 protein such as one or more SARS-CoV-2 nsp8 protein derived peptides set forth in Tables 1 to 10 (SEQ ID NOS: 1 to 3522),
xxiv) one or more peptides derived from the SARS-CoV-2 nsp9 protein such as one or more SARS-CoV-2 nsp9 protein derived peptides set forth in Tables 1 to 10 (SEQ ID NOS: 1 to 3522),
xxv) one or more peptides derived from the SARS-CoV-2 nsp10 protein such as one or more SARS-CoV-2 nsp10 protein derived peptide set forth in Tables 1 to 10 (SEQ ID NOS: 1 to 3522),
xxvi) one or more peptides derived from the SARS-CoV-2 nsp12 protein such as one or more SARS-CoV-2 nsp12 protein derived peptides set forth in Tables 1 to 10 (SEQ ID NOS: 1 to 3522),
xxvii) one or more peptides derived from the SARS-CoV-2 nsp13 protein such as one or more SARS-CoV-2 nsp13 protein derived peptides set forth in Tables 1 to 10 (SEQ ID NOS: 1 to 3522),
xxviii) one or more peptides derived from the SARS-CoV-2 nsp14 protein such as one or more SARS-CoV-2 nsp14 protein derived peptides set forth in Tables 1 to 10 (SEQ ID NOS: 1 to 3522),
xxix) one or more peptides derived from the SARS-CoV-2 nsp15 protein such as one or more SARS-CoV-2 nsp15 protein derived peptides set forth in Tables 1 to 10 (SEQ ID NOS: 1 to 3522), and
xxx) one or more peptides derived from the SARS-CoV-2 nsp16 protein such as one or more SARS-CoV-2 nsp16 protein derived peptides set forth in Tables 1 to 10 (SEQ ID NOS: 1 to 3522).

226.-227. (canceled)

228. The method of claim 225, further comprising monitoring an immune response relevant to a coronavirus infection comprising one or more steps of:
i) providing one or more MHC/peptide monomers or multimers,
ii) providing a sample comprising a population of T cells, and
iii) measuring the presence, frequency, number, activity and/or state of T cells specific for said one or more MHC/peptide multimers,
thereby monitoring said immune response relevant to a coronavirus infection; or
i) providing one or more MHC/peptide monomers or multimers,
ii) providing a sample comprising a population of T cells, and
iii) measuring the presence, frequency, number, activity and/or state of T cells specific for said one or more MHC/peptide multimers,
thereby diagnosing said coronavirus infection.

229. (canceled)

230. A method for isolation of one or more antigen-specific T cells, said method comprising one or more steps of:
i) providing a sample comprising a population of T cells,
ii) providing one or more MHC/peptide multimers,
iii) contacting said MHC/peptide multimers or composition with said sample comprising a population of T cells, and
iv) isolating T cells specific for said MHC/peptide multimers or composition.

231. The method of claim 230, further comprising detecting an antigen-specific T cell response comprising one or more steps of:
i) providing a sample comprising a population of T cells,
ii) providing one or more MHC/peptide multimers,
iii) contacting said MHC/peptide multimers or composition with said sample, and
iv) measuring the presence, frequency, number, activity and/or state of T cells specific for said MHC/peptide multimers or composition, thereby detecting said antigen-specific T cell response.

232. The method of claim 230, further comprising distinguishing an immune response from a subject that has been: a) vaccinated against but not exposed to SARS-COV-2, b) exposed to SARS-COV-2 but not vaccinated against SARS-COV-2, c) vaccinated against and exposed to SARS-COV-2, or d) neither vaccinated against nor exposed to SARS-COV-2, the method comprising, consisting of, or consisting essentially of:
contacting a biological sample from a subject with one or more monomers or multimers selected from any one of SEQ ID NOS: 1 to 3522; and
determining if the composition elicits an immune response from the contacted cells, wherein the level of elicited immune response indicates whether the subject falls into category a), b), c), or d); or
determining whether the subject falls into category a), b), c), or d) further comprises determining whether the immune response is predominantly to a SARS-CoV-2 Spike protein, or is to one or more SARS-CoV-2antigens other than the Spike protein, wherein at least one of:
i) a predominant response to SARS-CoV-2 Spike protein and minimal response to one or more SARS-CoV-2 antigens other than Spike is indicative that a subject falls into category a),
ii) a response to coronavirus Spike protein and one or more SARS-CoV-2 antigens other than Spike is indicative that the subject falls into category b),
iii) a strong response to SARS-CoV-2 Spike protein and one or more SARS-CoV-2 antigens other than Spike is indicative that the subject falls into category c), and
iv) a weak or no response to SARS-CoV-2 Spike or one or more SARS-CoV-2 antigens other than Spike is indicative that the subject falls into category d);
wherein the SARS-CoV-2 Spike protein or SARS-CoV-2 antigen is a protein or peptide comprising an amino acid sequence set forth in Tables 1 to 10 (SEQ ID NOS: 1 to 3522);
wherein the sample comprises T cells;
wherein the response comprises inducing, increasing, promoting or stimulating anti-SARS-CoV-2 activity of T cells;
wherein the T cells are CD8+ or CD4+ T cells;
wherein the method comprises determining whether the subject has been infected by or exposed to SARS-CoV-2 more than once by determining if the subject elicits a secondary T cell immune response profile that is different from a primary T cell immune response profile;
further comprising diagnosing a SARS-CoV-2 infection or exposure in a subject, the method comprising contacting a biological sample from a subject with a composition; and determining if the composition elicits a T cell immune response, wherein the T cell immune response identifies that the subject has been infected with or exposed to SARS-CoV-2; or
wherein the method is conducted three or more days following the date of suspected infection by or exposure to a coronavirus.

233.-240. (canceled)
